(19) United States
(12) Patent Application Publication
(10) Pub. No.: US 2026/0102346 A1
(43) Pub. Date: Apr. 16, 2026

(54) LIPID COMPOSITION

(71) Applicant: STEMIRNA THERAPEUTICS CO., LTD., Shanghai (CN)

(72) Inventors: Lei HUANG, Shanghai (CN); Mingyun SHEN, Shanghai (CN); Hangwen LI, Shanghai (CN); Haifa SHEN, Shanghai (CN)

(21) Appl. No.: 19/102,765

(22) PCT Filed: Aug. 8, 2023
(86) PCT No.: PCT/CN2023/111745

§ 371 Date: Feb. 10, 2025

(30) Foreign Application Priority Data
Aug. 9, 2022 (CN) 202210948906.0

(51) Int. Cl. A61K 9/1272; A61K 9/51; A61K 47/59; A61K 47/64

(52) U.S. Cl. CPC A61K 9/1272; A61K 9/5123; A61K 47/595; A61K 47/6455

(57) ABSTRACT

Provided is a drug delivery system, which in particular relates to a lipid composition. The shown lipid composition including a therapeutic agent and/or a prophylactic agent such as an RNA can be used for delivering the therapeutic agent and/or the prophylactic agent to a mammalian cell or organ, so as to, for example, regulate polypeptide, protein, or gene expression.

### REFERENCE TO AN ELECTRONIC SEQUENCE LISTING

The content of the electronic sequence listing (WO2024032611-3124US2534-sequnce list.xml; Size: 57,013 bytes; and Date of Creation: Feb. 5, 2025) is herein incorporated by reference in its entirety.

### CROSS-REFERENCE TO RELATED APPLICATIONS

The present application claims priority to Chinese Patent Application No. 202210948906.0 filed on Aug. 9, 2022, which is incorporated herein by reference in their entirety.

### TECHNICAL FIELD

The present disclosure relates to a drug delivery system, and in particular, to a lipid composition, a related product, and use in intramuscular injection.

### BACKGROUND

A lipid-containing nanoparticle composition, a liposome and a lipoplex, as a transport vehicle, can effectively transport a bioactive substance such as a small molecule drug, a protein and a nucleic acid into a cell and/or an intracellular compartment. These lipid compositions generally include cationic lipids, structured lipids, helper lipids and/or surfactants.

Existing lipid-based drug delivery systems, such as liposome and lipid nanoparticle (LNP) drug delivery systems, have been widely used in these years. However, in practical use, it is found that these lipid-based drug delivery systems have many problems, for example, the liposome production method is complex and needs to use an organic solvent, and the drug encapsulation efficiency is low; for example, the unmodified LNP system has poor stability in vivo and poor targeting effect. Furthermore, in the existing lipid delivery systems, after a nucleic acid is delivered to an organism, the expression amount of the nucleic acid in vivo is relatively low, and in the process of lyophilization and storage, there is a problem of stability, and the expression amount of the nucleic acid after lyophilization and reconstitution is significantly reduced, thereby affecting use. To improve the stability, a stabilizer such as N-(methoxy-poly(ethylene glycol)-oxycarbonyl)-distearoylphosphatidylethanolamine is also generally required to be added, so that the preparation process is complicated and the costs are increased. There is therefore a need to investigate more efficient and stable lipid delivery systems.

At present, many novel efficient lipid delivery systems have been developed. For example, CN110974954A provides a lipid nanoparticle for enhancing the immune effect of nucleic acid vaccines, which has the advantages of high nucleic acid encapsulation efficiency and narrow particle size distribution. For example, CN110638759A also provides a preparation for in vitro transfection and in vivo delivery of mRNA, which can be less toxic and result in a better immune effect of the nucleic acid in vivo. Despite significant advances in the research of lipid-based drug delivery systems, there remains a need for lipid delivery systems that are more efficient, stable, and have a good targeting effect.

### BRIEF SUMMARY OF THE INVENTION

An aspect of the present disclosure provides a lipid composition, including a therapeutic agent or a prophylactic agent and a lipid encapsulating the therapeutic agent or the prophylactic agent, wherein the lipid encapsulating the therapeutic agent or the prophylactic agent includes a cationic lipid, a phospholipid, a steroid, and a polyethylene glycol modified lipid; and the composition further includes a cationic polymer, wherein the cationic polymer and the therapeutic agent or the prophylactic agent are associated as a complex and co-encapsulated in the lipid to form a lipopolyplex.

In an embodiment, the therapeutic agent or the prophylactic agent is a nucleic acid, such as an RNA, in particular an mRNA.

In an embodiment, the cationic lipid includes lipid compounds of formula (I), formula (II), formula (III), and formula (IV) or pharmaceutically acceptable salts thereof, as defined herein. Preferably, the cationic lipid is M5, MC3, ALC-0315, SM-102, SW-II-115, SW-II-121, SW-II-122, SW-II-134-3, SW-II-138-2, SW-II-139-2, or SW-II-140-2.

In an embodiment, the cationic lipid does not include T5′.

In an embodiment, the lipid composition includes
10-70 mol % of the cationic lipid, 10-70 mol % of the phospholipid, 10-70 mol % of the steroid, and 0.05-20 mol % of the polyethylene glycol modified lipid;
preferably includes 35-50 mol % of the cationic lipid, 10-30 mol % of the phospholipid, 24-44 mol % of the steroid, and 1-1.5 mol % of the polyethylene glycol modified lipid; and/or
the cationic lipid, DOPE, the cholesterol, and DMG-PEG; and
preferably includes 50 mol % of the cationic lipid, 10 mol % of DOPE, 38.5 mol % of the cholesterol, and 1.5 mol % of DMG-PEG, or 40 mol % of the cationic lipid, 15 mol % of DOPE, 43.5 mol % of the cholesterol, and 1.5 mol % of DMG-PEG.

In an embodiment, the therapeutic agent or the prophylactic agent is a polynucleotide, the polynucleotide includes a coding region, and the coding region encodes a modified spike protein, wherein the modified spike protein includes an amino acid sequence of SEQ ID NO: 12 or an amino acid sequence having at least 95% identity to the amino acid sequence of SEQ ID NO: 12; and wherein the polynucleotide is an RNA, wherein the coding region includes a nucleotide sequence of SEQ ID NO: 3 or a nucleotide sequence having at least 85% identity to the nucleotide sequence of SEQ ID NO: 3; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In some embodiments, the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

In an aspect, the present disclosure further provides a pharmaceutical composition, including the lipid composition of the present disclosure, and optionally a pharmaceutically acceptable excipient.

In another aspect, the present disclosure further provides an injectant, including the lipid composition of the present disclosure, and a pharmaceutically acceptable excipient for injection.

In yet another aspect, the present disclosure further provides use of the lipid composition of the present disclosure, the pharmaceutical composition of the present disclosure, or the injectant of the present disclosure in preparation of a drug for treating or preventing a disease or disorder in a subject in need thereof.

In yet another aspect, the present disclosure further provides use of the lipid composition of the present disclosure, the pharmaceutical composition of the present disclosure, or the injectant of the present disclosure in preparation of a drug for preventing and/or treating SARS-CoV-2 infection.

In yet another aspect, the present disclosure further provides a method for preventing or treating a disease or disorder in a subject in need thereof, including:

administrating to the subject in need thereof the lipid composition of the present disclosure, the pharmaceutical composition of the present disclosure, or the injectant of the present disclosure. The lipid composition and the pharmaceutical composition can be administrated by injection, preferably, by intramuscular injection.

In an aspect, the present disclosure further provides a method for delivering a therapeutic agent or a prophylactic agent to a mammalian cell of a subject, including administering to the subject the lipid composition or the pharmaceutical composition of the present disclosure, the administering including bringing the cell into contact with the lipid composition, thereby delivering the therapeutic agent and/or the prophylactic agent to the cell.

In another aspect, the present disclosure further provides a method for producing a polypeptide of interest in a mammalian cell of a subject, including bringing the cell into contact with the lipid composition or the pharmaceutical composition of the present disclosure, wherein the therapeutic agent or the prophylactic agent is an mRNA, and wherein the mRNA encodes a polypeptide of interest, whereby the mRNA is capable of being translated in the cell to produce the polypeptide of interest.

In some embodiments, the lipid composition, the pharmaceutical composition or the drug is administered parenterally, orally, transmucosally, transdermally, intramuscularly, intravenously, intradermally, subcutaneously, intraperitoneally, intraventricularly, intracranially, or intravaginally, and preferably by intramuscular injection.

### BRIEF DESCRIPTION OF THE DRAWINGS

FIG. 1 shows results of luciferase expression of LPP preparations prepared by CDC prescription and BIC prescription in livers and injection sites of mice.

FIGS. 2A-2B show results of test of in vivo luciferase expression and in vitro physicochemical properties of SW0123.351-LPP after storage for different time periods. FIG. 2A shows results of in vivo luciferase expression. FIG. 2B shows results of test of average particle size (Z-Average).

FIGS. 3A-3B show results of test of in vivo luciferase expression and in vitro physicochemical properties of an SW0123.351-LPP solution after treatment with different times of freeze thawing. FIG. 3A shows results of in vivo luciferase expression. FIG. 3B shows results of test of average particle size (Z-Average).

FIG. 4 shows results of test of immunogenicity of SW0123.351-LPP in mice before and after lyophilization.

FIGS. 5A-5C show results of test of a particle size, encapsulation efficiency, and immunogenicity of SW0123.351-LPP lyophilized powder after storage at 25° C. for different time periods. FIG. 5A shows results of test of average particle size (Z-Average). FIG. 5B shows results of test of encapsulation efficiency. FIG. 5C shows results of test of immunogenicity.

FIG. 6 shows results of luciferase expression of LNP or LPP preparations prepared by different prescriptions with a cationic lipid being M5 in injection sites of mice for 0-24 hours.

FIGS. 7A-7C show results of luciferase expression of LNP or LPP preparations prepared by different prescriptions with a cationic lipid being M5 in injection sites or livers of mice 3 hours and 6 hours after administration. FIG. 7A shows results of test 3 hours after administration. FIG. 7B shows results of test 6 hours after administration. FIG. 7C shows ratios of luciferase expression of liver/injection site of mice 6 hours after injection.

FIG. 8 shows results of luciferase expression of LNP or LPP preparations prepared by different prescriptions with a cationic lipid being SM-102, ALC-0315, SW-II-115, and SW-II-121 in injection sites of mice for 0-24 hours.

FIG. 9 shows ratios of luciferase expression of liver/injection site of mice of LNP or LPP preparations prepared by different prescriptions with a cationic lipid being M5, SM-102, ALC-0315, SW-II-121, SW-II-122, SW-II-134-3, SW-II-138-2, SW-II-139-2, SW-II-140-2, and SW-II-122.

FIG. 10 shows results of luciferase expression of LNP preparations prepared by different prescriptions with a cationic lipid being M5, MC3, and SW-II-121 in injection sites and livers of mice.

FIGS. 11A-11E show results of luciferase expression of LPP preparations prepared by different prescriptions with a cationic lipid being SW-II-121 in injection sites and livers of mice for 0-24 hours. FIG. 11A shows expression of lipid compositions in injection sites (muscular tissue). FIG. 11B shows expression of different phospholipid and PEG combinations in injection sites (muscular tissue). FIG. 11C shows expression of lipid compositions in livers. FIG. 11D shows expression of different phospholipid and PEG combinations in livers. FIG. 11E shows ratios of luciferase expression of muscle/liver of lipid compositions.

FIG. 12 shows preparation and characterization of LPP/mRNA vaccines. (A) shows prescriptions of four LPP preparations encapsulating luciferase mRNA; (B and C) show mRNA expression efficiency of four LPP preparations encapsulating luciferase mRNA in injection sites and livers 24 hours after injection; (D) shows an image of characterization of LPP-B2 by cryo-transmission electron microscope; (E) shows bioluminescence images of luciferase expression of mice 6 hours after injection of LPP/eGFP; (F) shows results of expression of eGFP in C2C12 cells and DC2.4 cells incubated with LPP/eGFP for 24 hours tested by flow cytometry; (G) shows results of expression of eGFP in mouse lymph gland cell subpopulations 24 hours after injection with LPP/eGFP tested by flow cytometry; and (H) shows results of mouse lymph gland DC maturation markers CD40, CD80, CD86, and MHC-II 24 hours after injection with blank carrier LPP or LPP/Luc for 24 hours tested by flow cytometry. The data for (F, G, and H) are represented as mean±SEM (n=5).

FIG. 13 shows results of expression of eGFP in A20 cells, Jurkat T cells, and HSkMC cells incubated with LPP/eGFP for 24 hours tested by flow cytometry.

FIG. 14 shows bioluminescence of luciferase expression in mice 24 hours after injection with LPP/eGFP. (A) shows statistical results of luciferase expression in livers and injection sites of mice 24 hours after injection with LPP/eGFP; (B) shows a bioluminescence image of luciferase expression in local injection sites and major organs (heart, liver, spleen, lung, kidney, brain, lymph gland) of mice.

FIG. 15 shows recognition of gating strategies of lymph gland cell populations.

FIG. 16 shows recognition of gating strategies of CD11c⁺ cells in a lymph gland.

FIG. 17 shows in vitro antigen characterization and in vivo immunogenicity of LPP/mRNA. (A) shows a schematic diagram of mRNA encoding a modified spike protein; (B) shows expression and deglycosylation analysis of spike glycoprotein, with arrows representing bands of glycosylated or deglycosylated spike protein respectively and triangles representing bands of glycosylated or deglycosylated S1 protein respectively; (C) shows an immune program of C57BL/6 mice (n=8); (D) shows RBD-specific IgG titers in serum of immunized mice tested by ELISA, with data represented as geometric mean, with a 95% confidence interval; (E) shows neutralization antibody titers against a wild-type strain in serum of immunized mice tested by pseudovirus neutralization assay, with data represented as geometric mean, with a 95% confidence interval; (F) shows frequencies of T cells secreting IFN-γ in immunized mouse splenocytes tested by ELISpot, with data represented as mean±SEM; (G-J) show cytokine patterns of vaccine induced T cells tested by flow cytometry, with data represented as mean±SEM; (K) shows vaccine specific IgG1 and IgG2c titers as well as IgG2c/IgG1 ratios in mouse serum collected 2 weeks after inoculation tested by ELISA; (L) shows immunization and challenge assay strategies of BALB/c aged mice; and (M) and (N) show survival rates and weight changes of mice after challenge respectively, with data represented as mean±SEM (n=8).

FIG. 18 shows expression of mRNA encoding a modified spike protein on cell surfaces of HEK-293 cells and DC2.4 cells tested by flow cytometry.

FIG. 19 shows results of IL-4 secretion induced by LPP/mRNA vaccine tested by flow cytometry.

FIG. 20 shows induction of strong immunogenicity by LPP/mRNA in rhesus monkeys. (A) shows experimental strategies for immunization, challenge, sample collection, and evaluation of rhesus monkeys; (B) shows RBD-specific IgG titers in serum of immunized rhesus monkeys tested by ELISA, with data represented as geometric mean, with a 95% confidence interval; (C) shows neutralization antibody titers against a wild-type strain in serum of immunized rhesus monkeys tested by pseudovirus neutralization assay, with data represented as geometric mean, with a 95% confidence interval; (D) shows neutralization antibody titers against Delta and Omicron (BA.1) variant strains in serum of immunized rhesus monkeys tested by pseudovirus neutralization assay, with data represented as geometric mean, with a 95% confidence interval; (E) shows neutralization antibody titers against variant strains tested by live virus cytopathic effect (CPE) assay, with data represented as geometric mean, with a 95% confidence interval; (F) shows frequencies of T cells secreting IFN-γ in PBMCs of immunized rhesus monkeys tested by ELISpot, with data represented as mean±SEM; and (G) shows frequencies of spike protein specific MBCs in rhesus monkey PBMCs tested by flow cytometry, with data represented as mean±SEM.

FIG. 21 shows LPP/mRNA protecting rhesus monkeys from SARS-CoV-2 infection vaccine and death. After challenge, RT-PCR is used to test viral RNA copies in (A) nasal swabs, (B) oropharyngeal swabs, and (C) anal swabs; (D) shows the copy number of lung tissue viral RNA tested by RT-PCR. (E) shows histopathological changes in lungs of rhesus monkeys at day 7 after the challenge.

FIGS. 22(A and B) show changes in body weight and temperature of rhesus monkeys in a vaccine group and a normal saline group 1-7 days after challenge.

FIG. 23 shows enhancement of vaccine efficacy by a homologous or heterologous LPP/mRNA booster. (A) shows experimental strategies for homologous booster immunization; (B) shows RBD-specific IgG titers in serum of immunized mice at day 84 tested by ELISA, with data represented as geometric mean, with a 95% confidence interval; (C) shows neutralization antibody titers against a wild-type strain in serum of immunized mice at day 84 tested by pseudovirus neutralization assay, with data represented as geometric mean, with a 95% confidence interval; (D) shows frequencies of T cells secreting IFN-γ in splenocytes of immunized mice at day 84 tested by ELISpot, with data represented as mean±SEM; (E-G) show neutralization antibody titers against Delta and Omicron (BA.1 and BA.4/5) variant strains in serum of immunized mice at day 84 tested by pseudovirus neutralization assay, with data represented as geometric mean, with a 95% confidence interval; (H) shows experimental strategies for heterologous booster immunization; (I) shows fold changes of RBD-specific IgG in serum of immunized mice at day 84 relative to day 28 tested by ELISA, with data represented as geometric mean, with a 95% confidence interval; (J) shows fold changes of neutralization antibody titers against a wild-type strain in serum of immunized mice at day 84 relative to day 28, tested by pseudovirus neutralization assay, with data represented as geometric mean, with a 95% confidence interval; (K) shows frequencies of T cells secreting IFN-γ in spleens and lungs of immunized mice at day 84 tested by ELISpot, with data represented as mean±SEM; and (L-N) show fold changes of neutralization antibody titers against Delta and Omicron (BA.1 and BA.4/5) variant strains in serum of immunized mice at day 84 relative to day 28 tested by pseudovirus neutralization assay, with data represented as geometric mean, with a 95% confidence interval.

FIG. 24 shows safety studies of LPP/mRNA vaccine in rhesus monkeys. (A) shows immunization, sample collection, and evaluation strategies of rhesus monkeys. (B-E) show results of test of white blood cells (WBC), lymphocyte (LYMPH), alanine aminotransferase (ALT), and aspartate aminotransferase (AST) at specific time points, respectively. (F and G) show changes of increase in body temperature and weight of rhesus monkeys throughout the entire study period, with data represented as mean±SEM, respectively; (H) shows histopathological changes of injection sites of rhesus monkeys at day 30 and day 59.

FIG. 25 shows results of test of cytokine IL-6 in rhesus monkeys before administration, 24 hours after administration at day 1 and day 29, and at day 56 after administration.

### DETAILED DESCRIPTION OF THE INVENTION

### General Definitions and Terms

All patents, patent applications, scientific publications, manufacturer's instructions and guidelines, regardless of the preceding or following text, cited herein are incorporated herein by reference in their entirety. Any content herein needs not to be construed as an admission that the present disclosure is not entitled to antedate such disclosure.

Unless otherwise indicated, scientific and technical terms used herein have the meaning commonly understood by those skilled in the art. Furthermore, the terms related to protein and nucleic acid chemistry, molecular biology, cell and tissue culture, and microbiology used herein are all widely used in the corresponding fields. Meanwhile, to better understand the present disclosure, definitions and explanations of related terms are provided below.

As used herein, the expressions “comprising”, “including”, “containing” and “having” are inclusive and mean including the listed elements, steps or components but not excluding other unlisted elements, steps or components. The expression “consisting of” does not include any element, step or component not specified. The expression “consisting substantially of” means the scope limited to the specified element, step or component, plus an optionally present element, step or component that does not significantly affect the basic and novel properties of the claimed subject matter. It needs to be understood that the expressions “consisting substantially of” and “consisting of” are included within the meaning of the expression “comprising”.

As used herein, the expression in singular form “a”, “an”, or “the” includes plural references unless the context indicates otherwise. The term “one or more” or “at least one” encompasses 1, 2, 3, 4, 5, 6, 7, 8, 9 or more.

The list of the range of values herein is solely for use as a shorthand method of referring individually to each different value falling within the range. Unless otherwise indicated herein, each individual value is incorporated into the specification as individually listed herein. Unless explicitly stated to the contrary, a numerical value or range shown herein is modified by “about”, meaning that the listed or claimed numerical value or range is ±20%, ±10%, ±5%, or ±3%.

Unless otherwise indicated, all methods described herein may be performed in any suitable order.

Herein, “nucleotide” includes deoxyribonucleotide, ribonucleotide, and derivatives thereof. As used herein, “ribonucleotide” is a constitutive substance of ribonucleic acid (RNA), consists of one molecular of base, one molecule of pentose, and one molecule of phosphoric acid, and refers to a nucleotide having a hydroxyl at a 2′-position of a β-D-ribofuranosyl group. However, “deoxyribonucleotide” is a constitutive substance of deoxyribonucleic acid (DNA), also consists of one molecular of base, one molecule of pentose, and one molecule of phosphoric acid, refers to a nucleotide having a hydroxyl substituted with hydrogen at a 2′-position of a β-D-ribofuranosyl group, and is a main chemical component of a chromosome. “Nucleotide” is generally referred to by a single letter representing the base therein: “A(a)” refers to an adenine-containing deoxyadenylic acid or adenylic acid, “C(c)” refers to a cytosine-containing deoxycytidylic acid or cytidylic acid, “G(g)” refers to a guanine-containing deoxyguanylic acid or guanylic acid, “U(u)” refers to a uracil-containing uridylic acid, and “T(t)” refers to a thymine-containing deoxythymidylic acid.

As used herein, the terms “polynucleotide” and “nucleic acid” are used interchangeably to refer to a polymer of deoxyribonucleotides (deoxyribonucleic acid, DNA) or a polymer of ribonucleotides (ribonucleic acid, RNA). “Polynucleotide sequence”, “nucleic acid sequence”, and “nucleotide sequence” are used interchangeably to denote the ordering of nucleotides in a polynucleotide. Those skilled in the art need to understand that a DNA coding strand (sense strand) and an RNA encoded thereby can be regarded as having the same nucleotide sequence, and a deoxythymidylic acid in a DNA coding strand sequence corresponds to a uridylic acid in an RNA sequence encoded thereby.

As used herein, the term “% identity” in reference to a sequence refers to the percentage of nucleotides or amino acids that are the same in an optimal alignment between the sequences to be compared. The difference between two sequences may be distributed in the local region (segment) or across the entire length of the sequences to be compared. The identity between two sequences is generally determined after the optimal alignment of segments or “comparison windows”. The optimal alignment may be performed manually, or with the aid of algorithms known in the art, including but not limited to local homology algorithms described in Smith and Waterman, 1981, Ads App.Math. 2,482 and Neddleman and Wunsch, 1970, J.Mol.Biol. 48,443, and a similarity search method described in Pearson and Lipman, 1988, Proc.Natl Acad.Sci.USA 88,2444, or performed by using computer programs, such as GAP, BESTFIT, FASTA, BLAST P, BLAST N, and TFASTA in Wisconsin Genetics Software Package, Genetics Computer Group, 575 Science Drive, Madison, Wis. For example, the percentage identity of two sequences can be determined by using the BLASTN or BLASTP algorithms publicly available at the National Center for Biotechnology Information (NCBI) website.

The % identity is obtained by determining the number of identical positions corresponding to the sequences to be compared, dividing the number by the number of positions compared (e.g., the number of positions in a reference sequence), and multiplying the result by 100. In some embodiments, the degree of identity is given to at least about 50%, at least about 55%, at least about 60%, at least about 65%, at least about 70%, at least about 75%, at least about 80%, at least about 85%, at least about 90%, at least about 95%, or about 100% of the region. In some embodiments, the degree of identity is given to the entire length of the reference sequence. The alignment for determining sequence identity can be performed by using tools known in the art, preferably by using optimal sequence alignment, e.g., by using Align, and by using standard settings, preferably EMBOSS::needle, Matrix:Blosum62, Gap Open 10.0, and Gap Extend 0.5.

As used herein, “modified” refers to unnatural. For example, an RNA may be a modified RNA. That is, the RNA may include one or more unnatural nucleobases, nucleosides, nucleotides, or linker groups. A “modified” group may also be referred to herein as an “altered” group. The group may be modified or altered chemically, structurally, or functionally. For example, a modified nucleobase may include one or more unnatural substitutions.

As used herein, the term “expression” includes transcription and/or translation of a nucleotide sequence. Thus, the expression may involve the production of a transcript and/or a polypeptide. The term “transcription” relates to the process of transcribing a genetic code in a DNA sequence into an RNA (a transcript). The term “in vitro transcription” refers to the in vitro synthesis of an RNA, in particular an mRNA, in a cell-free system (e.g., in an appropriate cell extract). A vector that can be used to produce a transcript is also referred to as “transcription vector”, which includes a regulatory sequence required for transcription. The term “transcription” encompasses “in vitro transcription”.

As used herein, the term “host cell” refers to a cell which is used to receive, maintain, replicate, and express a polynucleotide or vector.

As used herein, an “aliphatic” group is a non-aromatic group in which carbon atoms are connected into a chain, and may be saturated or unsaturated.

As used herein, the term “alkyl” refers to an optionally substituted straight or branched chain saturated hydrocarbon including one or more carbon atoms. The term “C₁-C₁₂ alkyl” or “C₁₋₁₂ alkyl” refers to an optionally substituted straight or branched chain saturated hydrocarbon including 1-12 carbon atoms. As used herein, the term “alkoxyl” refers to an alkyl described herein, which is connected to the remainder of a molecule via an oxygen atom. The term “alkylene” refers to a divalent group formed by the corresponding alkyl that loses one hydrogen atom. The term “C₁-C₁₂ alkylene” or “C₁₋₁₂ alkylene” refers to an optionally substituted straight or branched chain alkylene including 1-12 carbon atoms.

As used herein, the term “alkenyl” refers to an optionally substituted straight or branched chain hydrocarbon including two or more carbon atoms and at least one double bond. The term “C₂-C₁₂ alkenyl” or “C₂₋₁₂ alkenyl” refers to an optionally substituted straight or branched chain hydrocarbon including 2-12 carbon atoms and at least one carbon-carbon double bond. The alkenyl may include one, two, three, four or more carbon-carbon double bonds.

As used herein, the term “halogen” refers to fluorine, chlorine, bromine, and iodine.

As used herein, the term “carbocycle” refers to a monocyclic or polycyclic non-aromatic system including one or more rings composed of carbon atoms. The term “C₃₋₈ carbocycle” means a carbocycle including 3-8 carbon atoms. The carbocycle may include one or more carbon-carbon double bonds or triple bonds. Instances of carbocycle include but are not limited to cyclopropyl, cyclopentyl, cyclohexyl, etc. As used herein, when the carbocycle is saturated (i.e., includes no unsaturated bond), the carbocycle also refers to a corresponding cycloalkyl. Unless specifically indicated otherwise, the carbocycle described herein refers to unsubstituted and substituted, i.e., optionally substituted, carbocycles.

As used herein, the term “heterocycle” refers to a monocyclic or polycyclic system including one or more rings and including at least one heteroatom. The heteroatom may be, for example, a nitrogen, oxygen, phosphorus or sulfur atom. The heterocycle may include one or more double bonds or triple bonds, and may be non-aromatic. Instances of heterocycle include but are not limited to imidazolidinyl, oxazolidinyl, thiazolidinyl, pyrazolidinyl, isoxazolidinyl, isothiazolidinyl, morpholinyl, pyrrolidinyl, tetrahydrofuranyl, and piperidinyl. The heterocycle may include, for example, 3-10 atoms (non-hydrogen), i.e., 3-10 membered heterocycle (e.g., 3, 4, 5, 6, 7, 8, 9, or 10 membered), wherein one or more atoms are heteroatoms (e.g., N, O, S, or P). When the heterocycle is saturated (i.e., includes no unsaturated bond), the heterocycle also refers to a corresponding heterocyclylalkyl. Unless specifically indicated otherwise, the heterocycle described herein refers to unsubstituted and substituted heterocyclic groups, i.e., optionally substituted heterocycles.

As used herein, the term “aryl” refers to a all-carbon monocyclic or fused polycyclic aromatic ring group having a conjugated π-electron system. For example, C₆-C₁₀ alkylaryl may have 6-10 carbon atoms, e.g., 6, 7, 8, 9, 10 carbon atoms. Instances of aryl include but are not limited to phenyl, naphthyl, etc.

As used herein, the term “heteroaryl” refers to a monocyclic or fused polycyclic system including at least one ring atom selected from N, O, or S, with the remaining ring atoms being C, and having at least one aromatic ring. The heteroaryl may have 5-10 ring atoms (5-10 membered heteroaryl), including 5, 6, 7, 8, 9 or 10 membered, in particular 5 or 6 membered heteroaryl. Instances of heteroaryl include but are not limited to pyrrolyl, furanyl, thienyl, imidazolyl, oxazolyl, pyrazolyl, pyridyl, pyrimidinyl, pyrazinyl, quinolinyl, isoquinolinyl, tetrazolyl, triazolyl, triazinyl, benzofuranyl, benzothienyl, indolyl, isoindolyl, etc.

As used herein, the term “interrupted by one or more groups” means that the one or more groups are present on a carbon chain and the remainder of the carbon chain is connected to both ends of the one or more groups.

Unless specifically indicated otherwise, the groups described herein (e.g., any one of R₁-R₇, such as alkyl, alkylene, alkenyl, aryl, amino, etc.) can be optionally substituted. The optional substituent group may be selected from, but is not limited to: halogen atom (e.g., chloro, bromo, fluoro, or iodo), carboxyl (e.g., —C(O)OH), alcohol group (e.g., hydroxyl, —OH), ester group (e.g., —C(O)OR or —OC(O)R), aldehyde group (e.g., —C(O)H), carbonyl (e.g., —C(O)R, or represented by C═O), acyl halide (e.g., —C(O)X, where X is a halo selected from bromo, fluoro, chloro, and iodo), carbonate group (e.g., —OC(O)OR), alkoxyl (e.g., —OR), acetal (e.g., —C(OR)₂R″″, where each OR is the identical or different alkoxyl and R″″ is alkyl or alkenyl), phosphate radical (e.g., P(O)₄³⁻), thiol (e.g., —SH), sulfinyl (e.g., —S(O)R), sulfino (e.g., —S(O)OH), sulfo (e.g., —S(O)₂OH), thioformyl (e.g., —C(S)H), sulfate radical (e.g., S(O)₄²⁻), sulfonyl (e.g., —S(O)₂—), acylamino (e.g., —C(O)NR₂ or —N(R)C(O)R), azido (e.g., —N₃), nitro (e.g., —NO₂), cyano (e.g., —CN), isocyano (e.g., —NC), acyloxy (e.g., —OC(O)R), amino (e.g., —NR₂, NRH, or —NH₂), carbamoyl (e.g., —OC(O)NR₂, —OC(O)NRH, or —OC(O)NH₂), sulfonamido (e.g., —S(O)₂NR₂, —S(O)₂NRH, —S(O)₂NH₂, —N(R)S(O)₂R, —N(H)S(O)₂R, —N(R)S(O)₂H, —N(H)S(O)₂H), C₁-C₁₂ alkyl, C₂-C₁₂ alkenyl, C₆-C₁₀ aryl, 5-10 membered heteroaryl, or 3-10 membered heterocycle. In any of the foregoing, each R independently may be a substituent group as defined herein, such as alkyl, alkoxyl, alkylene, halogen, carbocycle, heterocycle, aryl, heteroaryl, and alkenyl. In some embodiments, the substituent group itself may be further substituted e.g., with one, two, three, four, five, or six substituent groups as defined herein. For example, the alkyl may be further substituted with one, two, three, four, five, or six substituent groups as described herein.

As used herein, the term “compound” is intended to include isotope compounds of the depicted structure. “Isotopes” refer to atoms having the same number of atoms but different mass numbers due to the different numbers of neutrons in a nucleus, e.g., deuterium isotopes. For example, isotopes of hydrogen include tritium and deuterium. In addition, the compound, salt, or complex of the present disclosure may be prepared in combination with a solvent or water molecule to form a solvate and a hydrate by a conventional method.

The term “optional” or “optionally” (e.g., optionally substituted) means that an event described subsequently may or may not occur, and the description includes instances where the event or situation occurs and instances where the event or situation does not occur. For example, “optionally substituted alkyl” means that alkyl may or may not be substituted, and the description includes substituted alkyl free radicals and unsubstituted alkyl free radicals.

It needs to be understood that when a chemical group is written in a particular order, the reverse order is also encompassed unless otherwise indicated. For example, in the general formula —(R)ᵢ-(M1)ₖ-(R)ₘ— where M₁ is defined as —C(O)NH— (i.e., —(R)ᵢ—C(O)—NH—(R)ₘ—), a compound where M₁ is —NHC(O)— (i.e., —(R)ᵢ—NHC(O)—(R)ₘ—) is also encompassed unless otherwise indicated.

As used herein, the term “contact” refers to the establishment of a physical connection between two or more entities. For example, bringing a mammalian cell into contact with a lipid composition means that the mammalian cell and the lipid nanoparticle share a physical connection. Methods for bringing a cell into contact with an external entity in vivo and in vitro are well known in the biological field. For example, bringing a lipid composition into contact with a mammalian cell in a mammalian body can be performed via different routes of administration (such as intravenous, intramuscular, intradermal, and subcutaneous), and may involve different amounts of lipid compositions. In addition, the lipid composition may make contact with more than one mammalian cell.

As used herein, the term “delivery” refers to providing an entity to a target. For example, delivering a therapeutic agent or a prophylactic agent to a subject may involve administering a composition including the therapeutic agent or the prophylactic agent to the subject.

As used herein, the term “subject” describes that an organism using the composition of the present disclosure can be provided thereto. Subjects that are expected to receive these compositions include but are not limited to humans, other primates, and other mammals such as cattles, swines, horses, sheep, cats, dogs, mice, or rats. Preferably, the subjects may be mammals, particularly humans.

As used herein, “encapsulation efficiency” refers to the ratio of the amount of a therapeutic agent or a prophylactic agent that becomes a part of a composition to the initial total amount of the therapeutic agent or the prophylactic agent for use in the preparation of the composition. For example, if 97 mg of the total 100 mg of a therapeutic agent or a prophylactic agent initially provided to a composition is encapsulated in the composition, the encapsulation efficiency can be determined to be 97%. As used herein, “encapsulation” may refer to complete, majority or partial capsulation, sealing, surrounding, or packaging.

As used herein, “lipid component” refers to a component of a composition including one or more lipids. For example, the lipid component may include one or more cationic lipids, pegylated lipids, structural lipids, or helper lipids.

The phrase “pharmaceutically acceptable” is used herein to refer to compounds, salts, materials, combinations, and/or dosage forms that are within a reasonable medical judgment range, suitable for use in contact with human and animal tissues without excessive toxicity, irritation, allergic reactions, or other issues or complications, and conform to a reasonable benefit/risk ratio.

As used herein, “pharmaceutically acceptable salt” refers to a derivative of a disclosed compound in which a parent compound is altered by converting an existing acid or base moiety into its salt form (e.g., by reacting a free basic group with a suitable organic acid). Instances of pharmaceutically acceptable salts include but are not limited to inorganic or organic acid salts of basic residues such as amine; and basic metals or organic salts of acidic residues such as carboxylic acid. Representative acid addition salts include but are not limited to acetate, adipate, alginate, ascorbate, aspartate, benzene sulfonate, benzoate, bisulfate, borate, butyrate, camphorate, camphorsulfonate, citrate, cyclopentylpropionate, digluconate, dodecyl sulfate, ethanesulphonate, fumarate, gluceptate, glycerophosphate, hemisulphate, enanthate, caproate, hydrobromide, hydrochloride, hydriodate, 2-hydroxy-ethanesulphonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectate, persulfate, 3-phenylpropionate, phosphate, picrate, pivalate, propionate, stearate, succinate, sulfate, tartrate, thiocyanate, cresylsulfonate, undecanoate, valerate, etc. Representative basic metal or alkaline earth metal salts include but are not limited to sodium, lithium, potassium, calcium, magnesium salts, etc.; and non-toxic ammonium, quaternary ammonium, and amine cations, including but not limited to ammonium, tetramethylammonium, tetraethylammonium, methylamine, dimethylamine, trimethylamine, triethylamine, ethylamine, etc. Pharmaceutically acceptable salts of the present disclosure include, for example, conventional non-toxic salts of a parent compound formed from non-toxic inorganic or organic acids. The pharmaceutically acceptable salt of the present disclosure may be synthesized by a parent compound including a basic or acidic moiety via a conventional chemical method. Generally speaking, these salts may be prepared by reacting the free acid or base form of these compounds with a stoichiometric amount of an appropriate base or acid in water or an organic solvent, or in a mixture of both; non-aqueous media such as ether, ethyl acetate, ethanol, isopropanol, or acetonitrile are generally preferred.

As used herein, “polydispersity index” or “PDI” refers to a ratio that describes the homogeneity of particle size distribution of a system. A smaller value, e.g., less than 0.3, indicates a narrower particle size distribution.

As used herein, “(potential” refers to, for example, an electrokinetic potential of a lipid in a lipid composition, and is an important indicator for characterizing the stability of a dispersed system.

As used herein, in the case of a composition, “size” or “average size” refers to the average diameter of the composition.

As used herein, the term “treating” refers to partially or completely alleviating, mitigating, ameliorating, or relieving one or more symptoms or features of a specific infection, disease, disorder, or condition, delaying its outbreak, inhibiting its progression, reducing its severity, or reducing its occurrence. “Preventing” refers to preventing potential diseases or preventing the deterioration of symptoms or the development of diseases.

The term “prophylactically or therapeutically effective amount” refers to the amount of a reagent (e.g., nucleic acid, drug, composition, therapeutic agent, diagnostic agent, prophylactic agent, etc.) that is sufficient to prevent or inhibit the occurrence of a disease or symptom and/or slow down, alleviate, and delay the development or severity of the disease or symptom. The prophylactically or therapeutically effective amount is influenced by factors including but not limited to: the development speed and severity of diseases or symptoms, the age, gender, weight, and physiological condition of a subject, the duration of treatment, and the specific route of administration. The prophylactically or therapeutically effective amount may be administered in one or more doses. The prophylactically or therapeutically effective amount may be achieved through continuous or discontinuous administration.

### Lipid Composition

Provided herein is a lipid composition. The lipid composition is a lipid delivery vector. A lipid can encapsulate a therapeutic agent or a prophylactic agent (e.g., a nucleotide) to form a nanoparticle so as to be delivered into an organism.

As used herein, the term “lipid” refers to an organic compound that includes a hydrophobic moiety and optionally also a hydrophilic moiety. The lipid is generally insoluble in water but soluble in many organic solvents. Generally, an amphiphilic lipid including a hydrophobic moiety and a hydrophilic moiety can be organized into a lipid bilayer structure in an aqueous environment, e.g., being present in the form of vesicle. Lipids can include but are not limited to: fatty acids, glycerides, phospholipids, sphingolipids, glycolipids, and steroid and cholesterol esters.

As used herein, “lipid nanoparticle” or “LNP” refers to a lipid vesicle with a uniform lipid core, which is a particle formed by lipids, in which the lipid components intermolecularly interact with each other to form a nanostructured entity. The therapeutic agent or the prophylactic agent (such as a nucleic acid, e.g., an mRNA) is encapsulated in the lipid.

Particularly preferably, the lipid composition may be, for example, a lipopolyplex (LPP) as described herein. The method for preparing such compositions may be as described herein. LPP is a particle with a core-shell structure, in which a therapeutic agent or a prophylactic agent (such as a nucleic acid, e.g., an mRNA) is included in a polymer complex, and the polymer complex itself is encapsulated in a biocompatible lipid bilayer shell to form the lipid nanoparticle of the present disclosure. In some embodiments, the lipid composition of the present disclosure is a lipopolyplex (LPP). In some embodiments, the composition of the present disclosure is a lipopolyplex (LPP) including an RNA.

In some embodiments, the lipid encapsulating a therapeutic agent or a prophylactic agent (such as a nucleic acid, e.g., an mRNA) is selected from one or more of: a cationic lipid, a phospholipid, a steroid, and/or a polyethylene glycol modified lipid. In a preferred embodiment, the cationic lipid is an ionizable cationic lipid.

In an embodiment, the lipid composition includes the cationic lipid, wherein the cationic lipid includes DOTMA, DOTAP, DDAB, DOSPA, DODAC, DODAP, DC-Chol, DMRIE, DMOBA, DLinDMA, DLenDMA, CLinDMA, DMORIE, DLDMA, DMDMA, DOGS, N4-cholesteryl-spermine, DLin-KC2-DMA, DLin-MC3-DMA, a compound of formula (I), (II), (III) or (IV) described herein, or a combination thereof. In a preferred embodiment, the cationic lipid includes M5, MC3, ALC-0315, and SM-102. In a preferred embodiment, the cationic lipid includes SW-II-115, SW-II-121, SW-II-122, SW-II-134-3, SW-II-138-2, SW-II-139-2, or SW-II-140-2. In a preferred embodiment, the cationic lipid includes M5, MC3, ALC-0315, SM-102, SW-II-115, SW-II-121, SW-II-122, SW-II-134-3, SW-II-138-2, SW-II-139-2, or SW-II-140-2.

In an embodiment, the lipid composition includes the phospholipid and/or the steroid. In an embodiment, the lipid composition includes the phospholipid as described herein, wherein the phospholipid includes 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleylphosphatidylethanolamine (POPE), distearoyl-phosphatidyl-ethanolamine (DSPE), dipalmitoylphosphatidylethanolamine (DPPE), dimyristoylphosphoethanolamine (DMPE), 1-stearoyl-2-oleyl-stearylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), or a combination thereof. In an embodiment, the lipid composition includes the steroid as described herein, wherein the steroid includes cholesterol, coprosterol, sitosterol, ergosterol, campesterol, stigmasterol, brassicasterol, tomatidine, ursolic acid, α-tocopherol, and derivatives thereof. In an embodiment, the lipid composition includes the phospholipid and the steroid as described herein. In an embodiment, the lipid composition includes DOPE. In an embodiment, the lipid composition includes DSPC. In an embodiment, the lipid composition includes the cholesterol. In an embodiment, the lipid composition includes DOPE and the cholesterol. In an embodiment, the lipid composition includes DSPC and the cholesterol.

In an embodiment, the lipid composition includes the cationic lipid M5, MC3, ALC-0315, SM-102, SW-II-115, SW-II-121, SW-II-122, SW-II-134-3, SW-II-138-2, SW-II-139-2, or SW-II-140-2, the phospholipid DOPE, and the cholesterol. In an embodiment, the lipid composition includes the cationic lipid M5, MC3, ALC-0315, SM-102, SW-II-115, SW-II-121, SW-II-122, SW-II-134-3, SW-II-138-2, SW-II-139-2, or SW-II-140-2, the phospholipid DSPC, and the cholesterol.

In some embodiments, the lipid encapsulating a polynucleotide further includes a polyethylene glycol modified lipid. In an embodiment, the polyethylene glycol modified lipid includes DMG-PEG (e.g., DMG-PEG 2000), DOG-PEG, and DSPE-PEG, or a combination thereof. In an embodiment, the polyethylene glycol modified lipid is DSPE-PEG. In an embodiment, the polyethylene glycol modified lipid is DMG-PEG (e.g., DMG-PEG 2000).

In an embodiment, the lipid composition includes the cationic lipid, DOPE, the cholesterol, and DSPE-PEG.

In an embodiment, the lipid composition includes the cationic lipid, DSPC, the cholesterol, and DSPE-PEG.

In an embodiment, the lipid composition includes the cationic lipid, DSPC, the cholesterol, and DMG-PEG.

In a preferred embodiment, the lipid composition includes the cationic lipid, DOPE, the cholesterol, and DMG-PEG.

In a preferred embodiment, the lipid composition includes the cationic lipid M5, MC3, ALC-0315, SM-102, SW-II-115, SW-II-121, SW-II-122, SW-II-134-3, SW-II-138-2, SW-II-139-2, or SW-II-140-2, DOPE, the cholesterol, and DMG-PEG.

In some embodiments, the lipid composition of the present disclosure further includes a cationic polymer, and the cationic polymer and the therapeutic agent or the prophylactic agent (such as a nucleic acid, e.g., an mRNA) are associated as a complex and co-encapsulated in the lipid.

In an embodiment, the cationic polymer comprises poly-L-lysine, protamine, polyethyleneimine (PEI), or a combination thereof. In an embodiment, the cationic polymer is the protamine. In an embodiment, the cationic polymer is PEI.

In an embodiment, the amount of the lipid in the lipid composition is calculated in mole percent (mol %), and the mole percent is determined based on the total mole of the lipid in the composition. Unless otherwise indicated, the sum of the amount (mol %) of each lipid in the composition is 100 mol %, i.e., the sum of the amount (mol %) of the cationic lipid, the phospholipid, the steroid, and the polyethylene glycol modified lipid is 100 mol %.

In an embodiment, the amount of the cationic lipid in the lipid composition is about 10-about 70 mol %. In some embodiments, the amount of the cationic lipid in the lipid composition is about 20-about 60 mol %, about 30-about 50 mol %, about 35-about 50 mol %, about 35-about 45 mol %, about 38-about 45 mol %, about 40-about 45 mol %, about 40-about 50 mol %, or about 45-about 50 mol %. For example, the amount of the cationic lipid may be about 10, 15, 20, 25, 30, 35, 38, 40, 45, 50, 55, 60, 65, or 70 mol %.

In an embodiment, the amount of the phospholipid in the lipid composition is about 10-about 70 mol %. In an embodiment, the amount of the phospholipid in the lipid composition is about 20-about 60 mol %, about 30-about 50 mol %, about 10-about 30 mol %, about 10-about 20 mol %, or about 10-about 15 mol %. For example, the amount of the phospholipid may be about 10, 15, 20, 25, 30, 33.5, 35, 40, 45, 50, 53.5, 55, 60, 65, or 70 mol %.

In an embodiment, the amount of the cholesterol in the lipid composition is about 10-about 70 mol %. In an embodiment, the amount of the cholesterol in the lipid composition is about 20-about 60 mol %, about 24-44 mol %, about 30-about 50 mol %, about 35-about 40 mol %, about 35-about 45 mol %, about 40-about 45 mol %, or about 45-about 50 mol %. For example, the amount of the cholesterol may be about 10, 13.5, 15, 18.5, 18.75, 20, 23.5, 23.75, 24, 25, 28.5, 28.75, 29, 30, 33.75, 34, 35, 38.5, 38.75, 39, 40, 43, 43.5, 44, 45, 48.5, 50, 55, 60, 65, or 70 mol %.

In an embodiment, the amount of the polyethylene glycol modified lipid in the lipid composition is about 0.05-about 20 mol %. In an embodiment, the amount of the polyethylene glycol modified lipid in the lipid composition is about 0.5-about 15 mol %, about 1-about 10 mol %, about 5-about 15 mol %, about 1-about 5 mol %, about 1-about 1.5 mol %, about 1.5-about 3 mol %, or about 2-5 mol %. For example, the amount of the polyethylene glycol modified lipid may be about 0.05, 1, 1.25, 1.5, 2, 2.5, 3, 3.5, 4, 4.5, 5, 5.5, 6, 6.5, 7, 7.5, 8, 8.5, 9, 9.5, 10, 15, or 20 mol %.

In an embodiment, the lipid composition includes 10-70 mol % of the cationic lipid, 10-70 mol % of the phospholipid, 10-70 mol % of the steroid, and 0.05-20 mol % of the polyethylene glycol modified lipid. In a preferred embodiment, the lipid composition includes 35-50 mol % of the cationic lipid, 10-30 mol % of the phospholipid, 24-44 mol % of the steroid, and 1-1.5 mol % of the polyethylene glycol modified lipid.

In an embodiment, LPP includes the therapeutic agent or the prophylactic agent (such as a nucleic acid, e.g., an mRNA) of the present disclosure, which associates with the cationic polymer as a complex; and a lipid encapsulating the complex, wherein the lipid encapsulating the complex includes a cationic lipid, a phospholipid, a steroid, and a polyethylene glycol modified lipid. In an embodiment, the phospholipid is selected from 1,2-dioleoyl-sn-glycero-3-phosphoethanolamine (DOPE), 1,2-distearoyl-sn-glycero-3-phosphocholine (DSPC), or a combination thereof. In an embodiment, the steroid is a cholesterol. In an embodiment, the cationic polymer is protamine. In an embodiment, the polyethylene glycol modified lipid is selected from 1,2-dimyristoyl-rac-glycero-3-methoxypolyethylene glycol (DMG-PEG), 1,2-distearoyl-sn-glycero-3-phosphoethanolamine-poly(ethylene glycol) (DSPE-PEG), or a combination thereof. In an embodiment, the cationic lipid is selected from M5, MC3, ALC-0315, SM-102, SW-II-115, SW-II-121, SW-II-122, SW-II-134-3, SW-II-138-2, SW-II-139-2, or SW-II-140-2.

In an embodiment, the lipid encapsulating the complex includes 50 mol % of M5, MC3, ALC-0315, SM-102, SW-II-115, SW-II-121, SW-II-122, SW-II-134-3, SW-II-138-2, SW-II-139-2, or SW-II-140-2, 10 mol % of DOPE, 38.5 mol % of the cholesterol, and 1.5 mol % of DMG-PEG. In an embodiment, the lipid encapsulating the complex includes 40 mol % of M5, MC3, ALC-0315, SM-102, SW-II-115, SW-II-121, SW-II-122, SW-II-134-3, SW-II-138-2, SW-II-139-2, or SW-II-140-2, 15 mol % of DOPE, 43.5 mol % of the cholesterol, and 1.5 mol % of DMG-PEG.

In an embodiment, the therapeutic agent or the prophylactic agent is a polynucleotide, the polynucleotide includes a coding region, and the coding region encodes a modified spike protein, wherein the modified spike protein includes an amino acid sequence of SEQ ID NO: 12 or an amino acid sequence having at least 95% identity to the amino acid sequence of SEQ ID NO: 12; and wherein the polynucleotide is an RNA, wherein the coding region includes a nucleotide sequence of SEQ ID NO: 3 or a nucleotide sequence having at least 85% identity to the nucleotide sequence of SEQ ID NO: 3; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In an embodiment, the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

In an embodiment, the lipid encapsulating the complex includes 40 mol % of M5, MC3, ALC-0315, SM-102, SW-II-115, SW-II-121, SW-II-122, SW-II-134-3, SW-II-138-2, SW-II-139-2, or SW-II-140-2, 15 mol % of DOPE, 43.5 mol % of the cholesterol, and 1.5 mol % of DMG-PEG; and the therapeutic agent or the prophylactic agent is a polynucleotide, the polynucleotide includes a coding region, and the coding region encodes a modified spike protein, wherein the modified spike protein includes an amino acid sequence of SEQ ID NO: 12 or an amino acid sequence having at least 95% identity to the amino acid sequence of SEQ ID NO: 12; and wherein the polynucleotide is an RNA, wherein the coding region includes a nucleotide sequence of SEQ ID NO: 3 or a nucleotide sequence having at least 85% identity to the nucleotide sequence of SEQ ID NO: 3; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In an embodiment, the lipid encapsulating the complex includes 40 mol % of M5, 15 mol % of DOPE, 43.5 mol % of the cholesterol, and 1.5 mol % of DMG-PEG; and the therapeutic agent or the prophylactic agent is a polynucleotide, the polynucleotide includes a coding region, and the coding region encodes a modified spike protein, wherein the modified spike protein includes an amino acid sequence of SEQ ID NO: 12 or an amino acid sequence having at least 95% identity to the amino acid sequence of SEQ ID NO: 12; and wherein the polynucleotide is an RNA, wherein the coding region includes a nucleotide sequence of SEQ ID NO: 3 or a nucleotide sequence having at least 85% identity to the nucleotide sequence of SEQ ID NO: 3; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In an embodiment, the lipid encapsulating the complex includes 40 mol % of M5, MC3, ALC-0315, SM-102, SW-II-115, SW-II-121, SW-II-122, SW-II-134-3, SW-II-138-2, SW-II-139-2, or SW-II-140-2, 15 mol % of DOPE, 43.5 mol % of the cholesterol, and 1.5 mol % of DMG-PEG; and the therapeutic agent or the prophylactic agent is a polynucleotide, and the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

In an embodiment, the lipid encapsulating the complex includes 40 mol % of M5, 15 mol % of DOPE, 43.5 mol % of the cholesterol, and 1.5 mol % of DMG-PEG; and the therapeutic agent or the prophylactic agent is a polynucleotide, and the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

### Cationic Lipid

A cationic lipid is a lipid that can carry a net positive charge at a given pH. The lipid with the net positive charge can associate with a nucleic acid via an electrostatic interaction.

Instances of cationic lipids include but are not limited to 1,2-di-O-octadecenyl-3-trimethylammonium-propane (DOTMA), 1,2-dioleoyl-3-trimethylammonium-propane (DOTAP), didecyldimethylammonium bromide (DDAB), 2,3-dioleoyloxy-N-[2(spermine carboxamide)ethyl]-N,N-dimethyl-1-propanamium trifluoroacetate (DOSPA), dioctadecyldimethyl ammonium chloride (DODAC), 1,2-dioleoyl-3-dimethylammonium-propane (DODAP), 3-(N—(N′,N′-dimethylaminoethane)-carbamoyl)cholesterol (DC-Chol), 2,3-di(tetradecoxy)propyl-(2-hydroxyethyl)-dimethylazanium (DMRIE), N,N-dimethyl-3,4-dioleyloxybenzylamine (DMOBA), 1,2-dilinoleyloxy-N,N-dimethylaminopropane (DLinDMA), 1,2-dilinolenyloxy-N,N-dimethylaminopropane (DLenDMA), 3-dimethylamino-2-(cholest-5-en-3-beta-oxybutan-4-oxy)-1-(cis,cis-9,12-octadecadienoxy)propane (CLinDMA), N-(2-aminoethyl)-N,N-dimethyl-2,3-bis(tetradecyloxy)propan-1-aminium bromide (DMORIE), N,N-dimethyl-2,3-bis(dodecyloxy)propan-1-amine (DLDMA), N,N-dimethyl-2,3-bis(tetradecyloxy)propan-1-amine (DMDMA), dioctadecylamidoglycyl spermine (DOGS), N4-cholesteryl-spermine, 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), a compound of formula (I), (II), (III) or (IV) as described herein, or a combination thereof.

In some embodiments, the cationic lipid is preferably an ionizable cationic lipid. The ionizable cationic lipid has a net positive charge, e.g., at acidic pH, and is neutral at higher pH (e.g., physiological pH). Instances of ionizable cationic lipids include but are not limited to: dioctadecylamidoglycyl spermine (DOGS), N4-cholesteryl-spermine, 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), a compound of formula (I), (II), (III) or (IV) as described herein, or a combination thereof.

In an embodiment, the cationic lipid includes a compound of formula (I), or a pharmaceutically acceptable salt thereof:

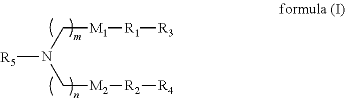

where
R₁ and R₂ are each independently selected from a bond, C₁-C₁₂ alkyl, and C₂-C₁₂ alkenyl;
R₃ and R₄ are each independently selected from C₁-C₁₂ alkyl, C₂-C₁₂ alkenyl, C₆-C₁₀ aryl, and 5-10 membered heteroaryl; and R₃ and R₄ are each independently optionally substituted with t R₆, t being an integer selected from 1-5;
R₆ is independently selected from C₁-C₁₂ alkyl and C₂-C₁₂ alkenyl; and
M₁ and M₂ are each independently selected from a bond, H, —O—, —S—, —C(O)—, —OC(O)—, —C(O)O—, —SC(S)—, —C(S)S—, 3-10 membered heterocycle, and —NR₇—, or
R₅ and one of M₁ and M₂ together with an N atom to which they are connected form 3-10 membered heterocycle, and corresponding R₁/R₃ or R₂/R₄ is absent, the heterocycle being optionally substituted with R₇;
R₅ is selected from C₃₋₈ carbocycle and —C₁₋₁₂ alkylene-Q, and Q is selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano, C₃₋₈ carbocycle, 3-10 membered heterocycle, and C₆-C₁₀ aryl, each of which is optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O);
m and n are each independently an integer selected from 0-12;
the alkyl, the alkenyl, and the alkylene are each optionally independently interrupted by one or more groups selected from —O—, —S—, —NR₇—, —C(O)—, —OC(O)—, —C(O)O—, —SC(S)—, —C(S)S—, and C₃₋₈ carbocycle, and the alkyl, the alkenyl, and the alkylene are each optionally substituted with one or more R₇; and
R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, 5-10 membered heteroaryl, 3-10 membered heterocycle, halogen, and C₃₋₈ carbocycle, each of which is optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O).

In an embodiment, R₁ and R₂ are each independently selected from C₁-C₁₂ alkyl and C₂-C₁₂ alkenyl, e.g., C₁-C₁₂ alkyl. In another embodiment, one of R₁ and R₂ is a bond, and the other is independently selected from C₁-C₁₂ alkyl and C₂-C₁₂ alkenyl, e.g., C₁-C₁₂ alkyl.

In an embodiment, R₃ and R₄ are each independently selected from C₁-C₁₂ alkyl, C₂-C₁₂ alkenyl, C₆-C₁₀ aryl, and 5-10 membered heteroaryl. In another embodiment, R₃ and R₄ are each independently selected from C₁-C₁₂ alkyl and C₂-C₁₂ alkenyl.

R₃ and R₄ may be each independently optionally substituted with t R₆, t being 1, 2, 3, 4, or 5. In an embodiment, R₆ is independently selected from C₁-C₁₂ alkyl.

In another embodiment, at least one of R₃ and R₄ is C₆-C₁₀ aryl or 5-10 membered heteroaryl, e.g., C₆-C₁₀ aryl.

In an embodiment, R₈ is selected from C₃₋₈ carbocycle and —C₁₋₁₂ alkylene-Q. Q may be selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano, C₃₋₈ carbocycle, 3-10 membered heterocycle, and C₆-C₁₀ aryl. The above groups, including the groups that encompass the option of Q, may each be optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O), where appropriate.

In another embodiment, R₅ is selected from C₃₋₈ carbocycle and —C₁₋₁₂ alkylene-Q, and Q is selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano, C₃₋₈ carbocycle, 3-10 membered heterocycle, and C₆-C₁₀ aryl. The above groups, including the groups that encompass the option of Q, may each be optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O), where appropriate.

In the compound of formula (I), R₇ may be independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, 5-10 membered heteroaryl, 3-10 membered heterocycle, halogen, and C₃₋₈ carbocycle, and preferably selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl. The above groups (e.g., H, C₁-C₁₂ alkyl, C₂-C₁₂ alkenyl, C₁-C₁₂ alkoxyl, carboxylic acid, sulfinic acid, sulfonic acid, sulfonyl, amino, carbamoyl, sulfonamido, C₆-C₁₀ aryl, 5-10 membered heteroaryl, 3-10 membered heterocycle, and C₃₋₈ carbocycle, where appropriate) are each optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O).

In an embodiment, each of the groups described above, e.g., C₃₋₈ carbocycle, —C₁₋₁₂ alkylene-Q, including —OR₇, —SR₇, —OC(O)R₇, —C(O)OR₇, —N(R₇)C(O)R₇, —N(R₇)S(O)₂R₇, —N(R₇)C(S)R₇, —N(R₇)₂, C₃₋₈ carbocycle, 3-10 membered heterocycle, and C₆-C₁₀ aryl that encompass the option of Q, C₁-C₁₂ alkyl, C₂-C₁₂ alkenyl, C₁-C₁₂ alkoxyl, carboxylic acid, sulfinic acid, sulfonic acid, sulfonyl, amino, carbamoyl, sulfonamido, C₆-C₁₀ aryl, 5-10 membered heteroaryl, 3-10 membered heterocycle, halogen, C₃₋₈ carbocycle, etc. may be optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O).

In an embodiment, the alkyl, the alkenyl, and the alkylene in the compound of formula (I) (e.g., mentioned in R₁-R₇) may each be optionally independently interrupted by one or more groups selected from —O—, —S—, —NR₇—, —C(O)—, —OC(O)—, —C(O)O—, —SC(S)—, —C(S)S—, and C₃₋₈ carbocycle, and the alkyl, the alkenyl, and the alkylene are each optionally substituted with one or more R₇. That is, chains (straight or branched chains) of the alkyl, the alkenyl, and the alkylene may each optionally include one or more groups selected from —O—, —S—, —NR₇—, —C(O)—, —OC(O)—, —C(O)O—, —SC(S)—, —C(S)S—, and C₃₋₈ carbocycle.

R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, 5-10 membered heteroaryl, 3-10 membered heterocycle, halogen, and C₃₋₈ carbocycle; and preferably, R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl. The above groups (e.g., H, C₁-C₁₂ alkyl, C₂-C₁₂ alkenyl, C₁-C₁₂ alkoxyl, carboxylic acid, sulfinic acid, sulfonic acid, sulfonyl, amino, carbamoyl, sulfonamido, C₆-C₁₀ aryl, 5-10 membered heteroaryl, 3-10 membered heterocycle, and C₃₋₈ carbocycle, where appropriate) are each optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O).

In the compound of formula (I), m and n may each be independently an integer selected from 0-12, e.g., 0, 1, 2, 3, 4, 5, 6, 7, 8, 9, 10, 11, or 12. When 0 is taken, it indicates that the corresponding group is absent.

In an embodiment, M₁ or M₂ is a bond, corresponding m or n is not 0, and a carbon chain in front of M₁ or M₂ is connected to corresponding R₁ or R₂.

In an embodiment, m or n is 0, corresponding M₁ or M₂ is not a bond, and an N atom is directly connected to M₁ or M₂.

In an embodiment, M₁ or M₂ is a bond, corresponding m or n is 0, and an N atom is directly connected to corresponding R₁ or R₂.

In an embodiment, M₁ and M₂ are each independently selected from —C(O)—, —OC(O)—, and —C(O)O—. In another embodiment, M₁ and M₂ are each independently selected from —NR₇—, R₇ being as described above.

In yet another embodiment, R₅ and one of M₁ and M₂ together with an N atom to which they are connected form 3-10 membered heterocycle, and corresponding R₁/R₃ or R₂/R₄ is absent, the heterocycle being optionally substituted with R₇, R₇ being as described above.

In an embodiment, R₅ is selected from —C₁₋₁₂ alkylene-Q, and Q is selected from H, —OR₇, —OC(O)R₇, —C(O)OR₇, —N(R₇)C(O)R₇, —N(R₇)₂, and cyano, R₇ being as described above.

In a preferred embodiment, R₁ and R₂ are each independently selected from C₁-C₁₂ alkyl and C₂-C₁₂ alkenyl,
R₃ and R₄ are each independently selected from C₁-C₁₂ alkyl and C₂-C₁₂ alkenyl; R₃ and R₄ are each independently optionally substituted with t R₆, t being an integer selected from 1-5; and R₆ is independently selected from C₁-C₁₂ alkyl and C₂-C₁₂ alkenyl;
M₁ and M₂ are each independently selected from —OC(O)—, —C(O)O—, —SC(S)—, and —C(S)S—;
R₅ is selected from —C₁₋₁₂ alkylene-Q, Q is selected from —OR₇ and —SR₇, and R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl; and
m and n are each independently an integer selected from 1-12.

In a preferred embodiment, the cationic lipid includes a lipid compound having a structure shown below, or a pharmaceutically acceptable salt thereof:

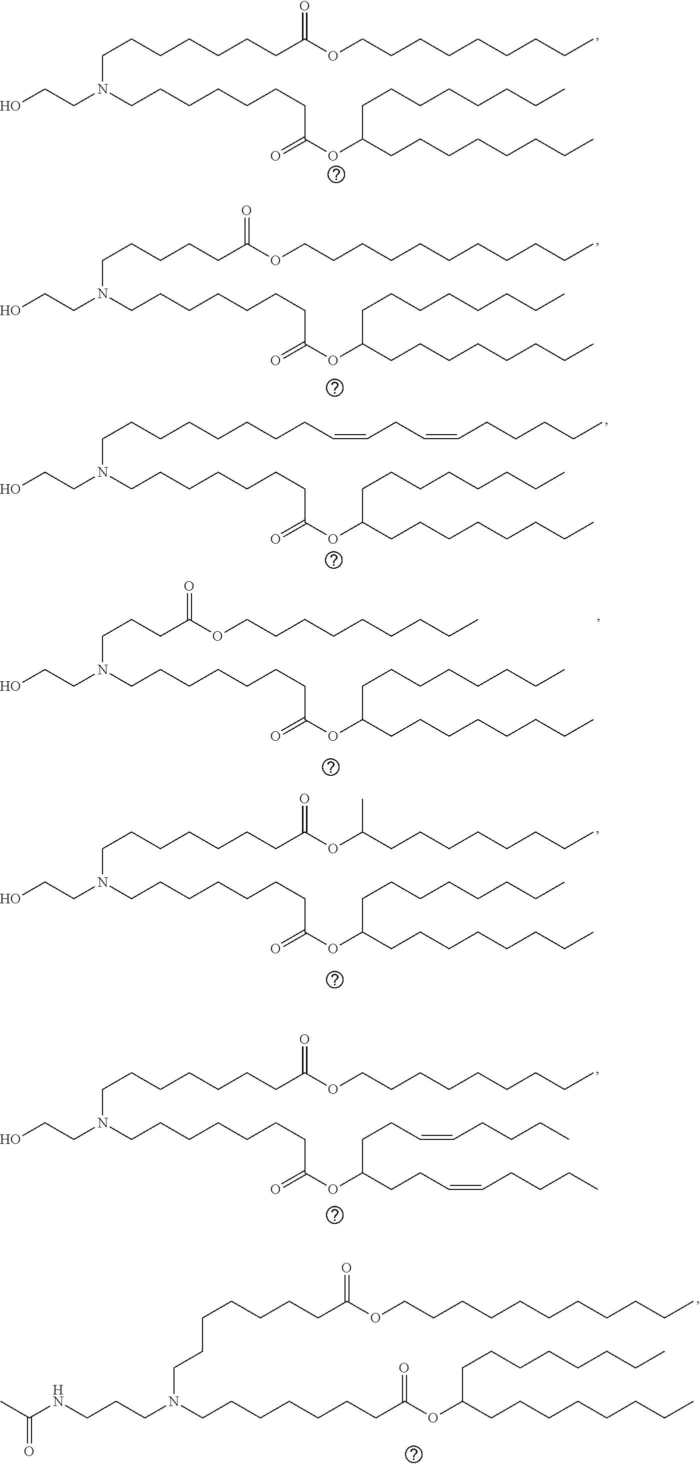

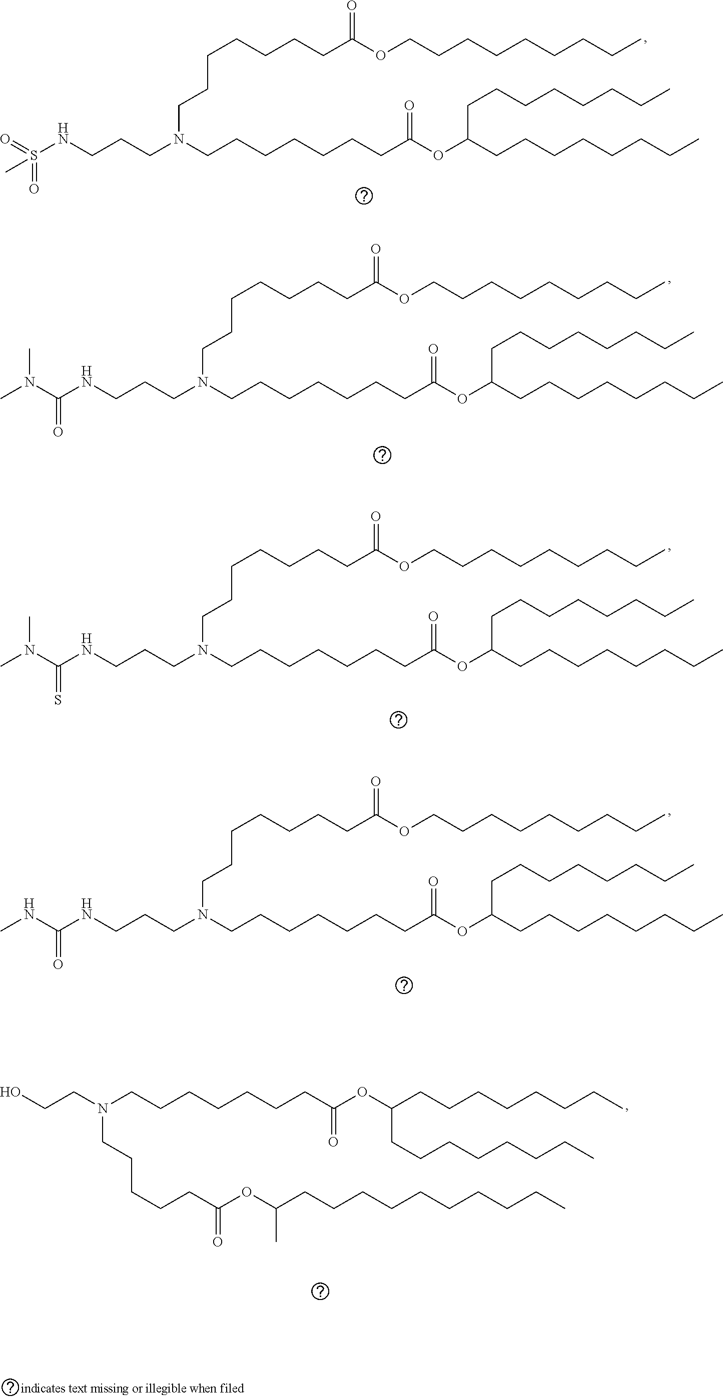

In a preferred embodiment, the cationic lipid includes M5 or SM-102.

In a preferred embodiment, the cationic lipid includes a lipid compound having a structure shown below, or a pharmaceutically acceptable salt thereof:

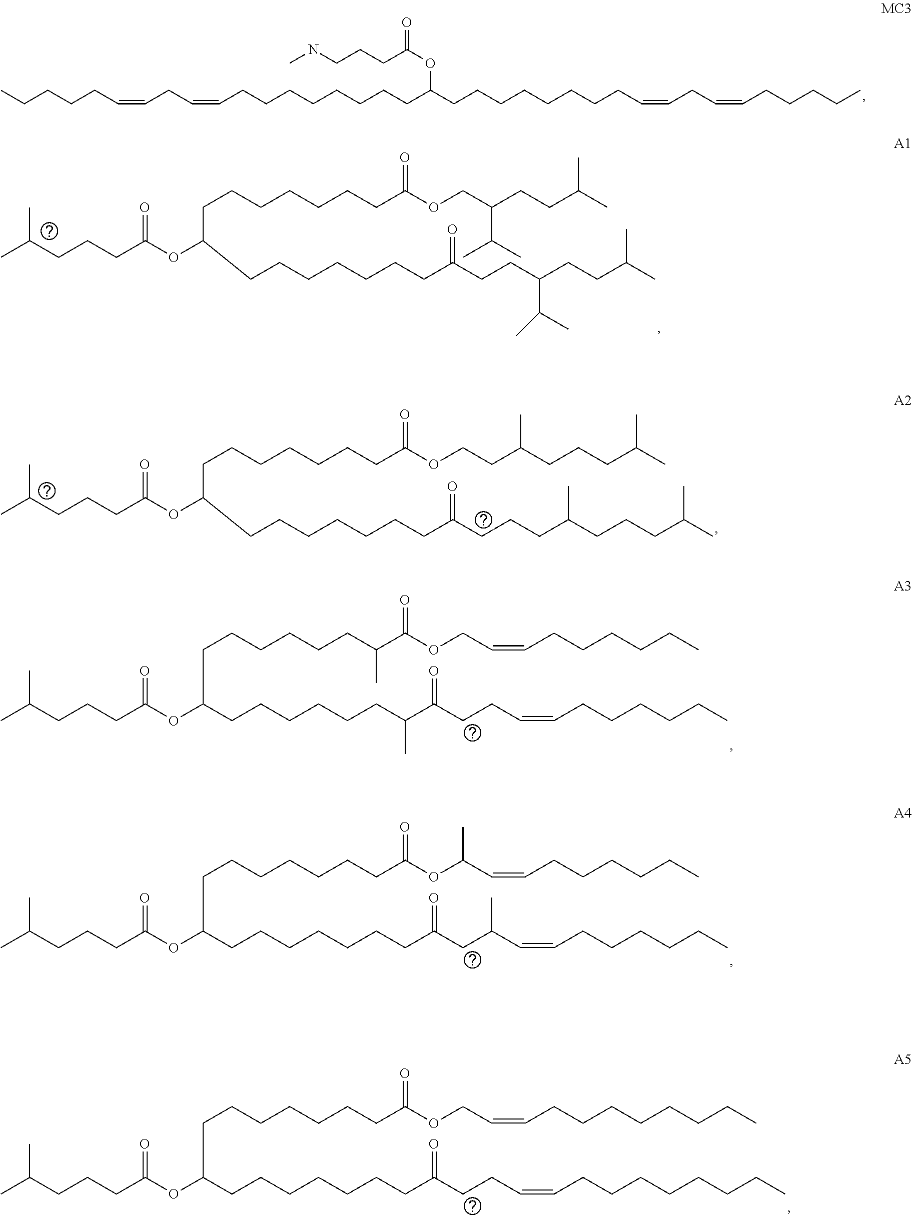

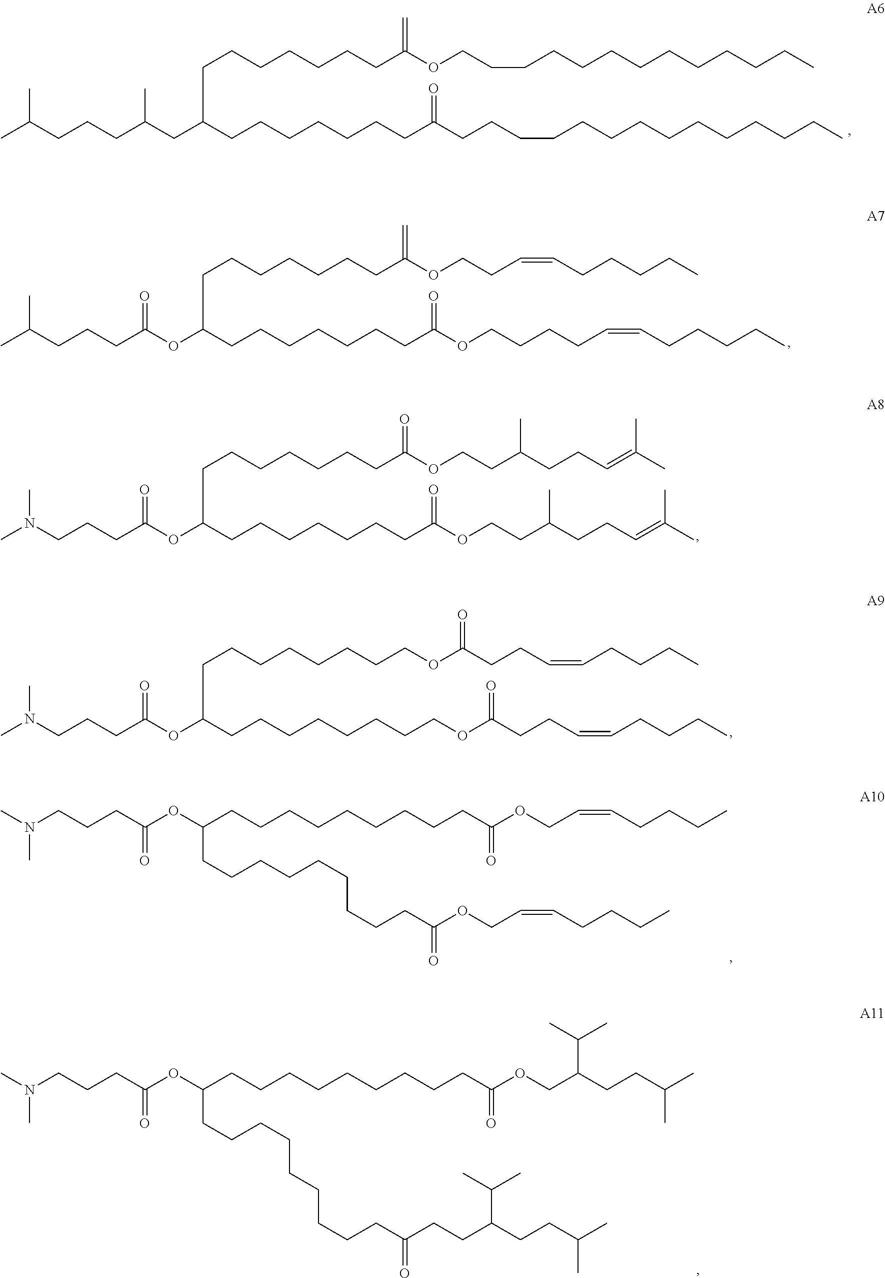

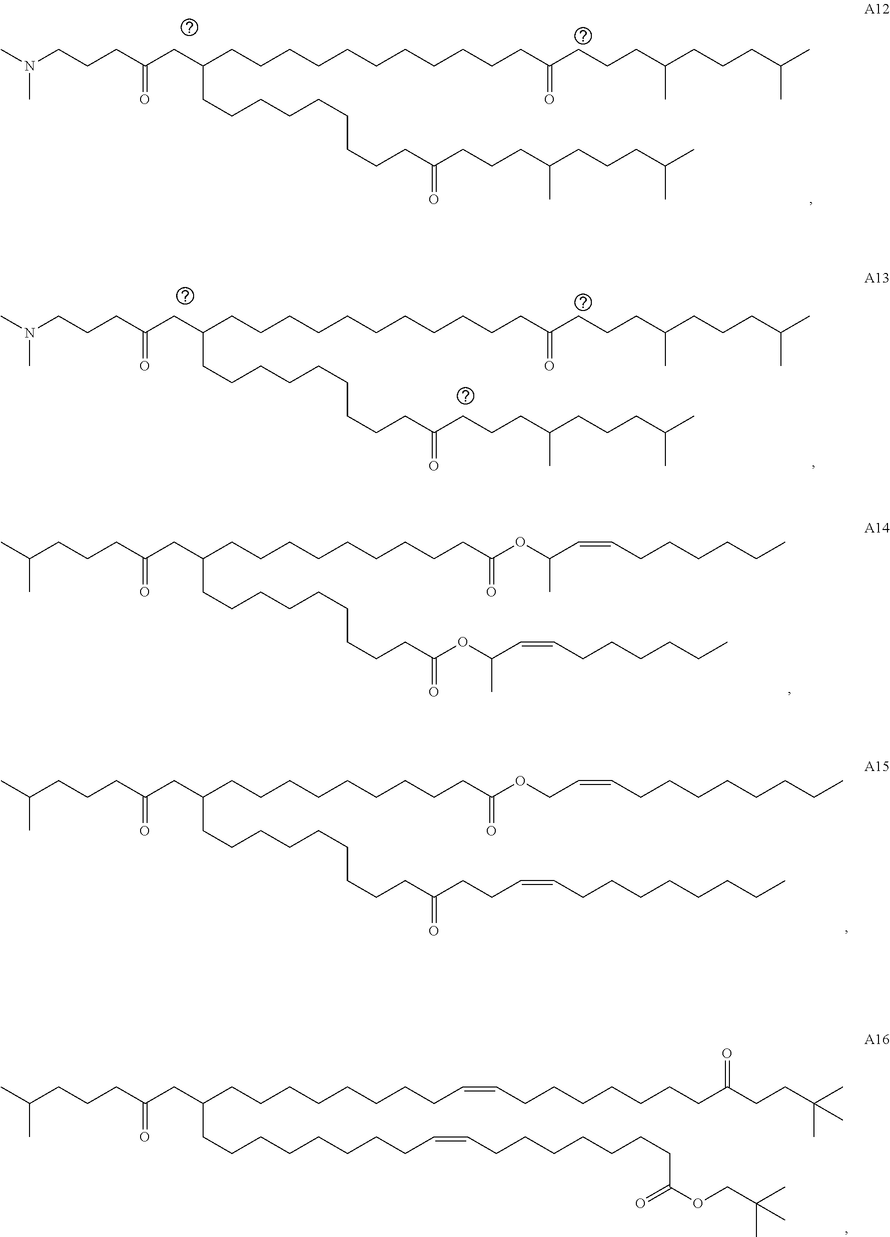

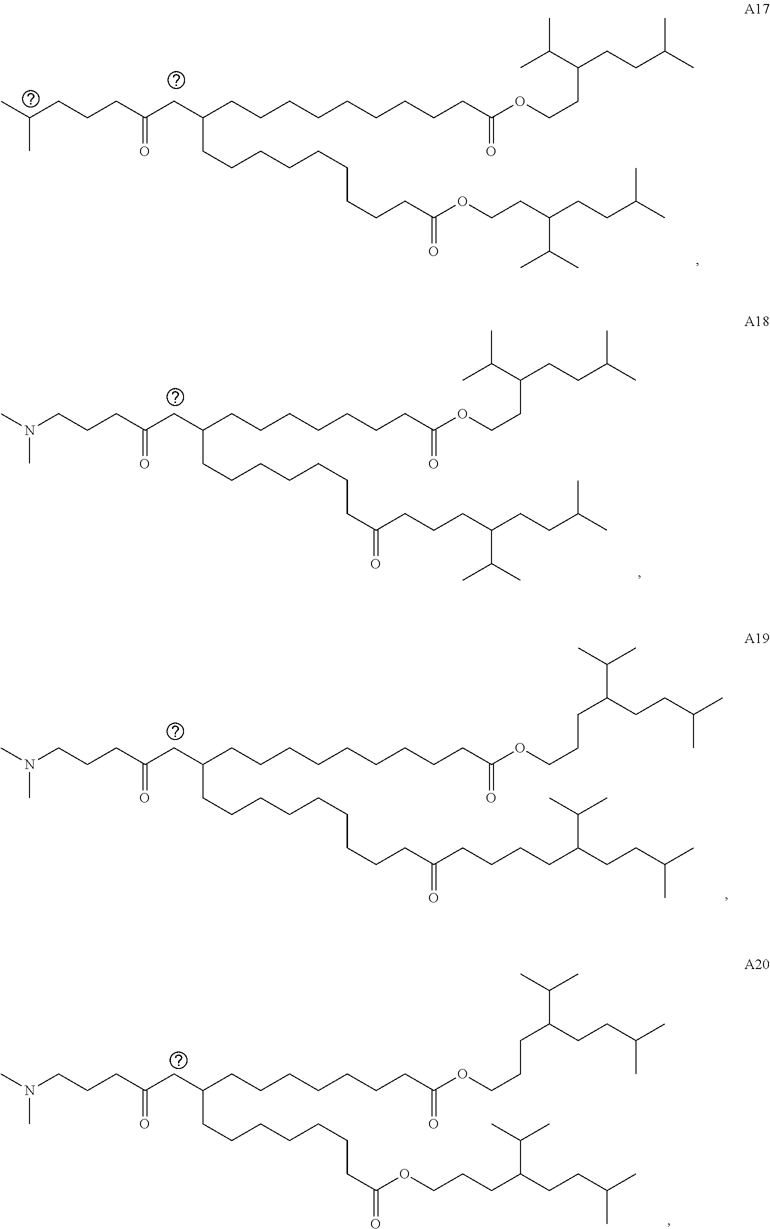

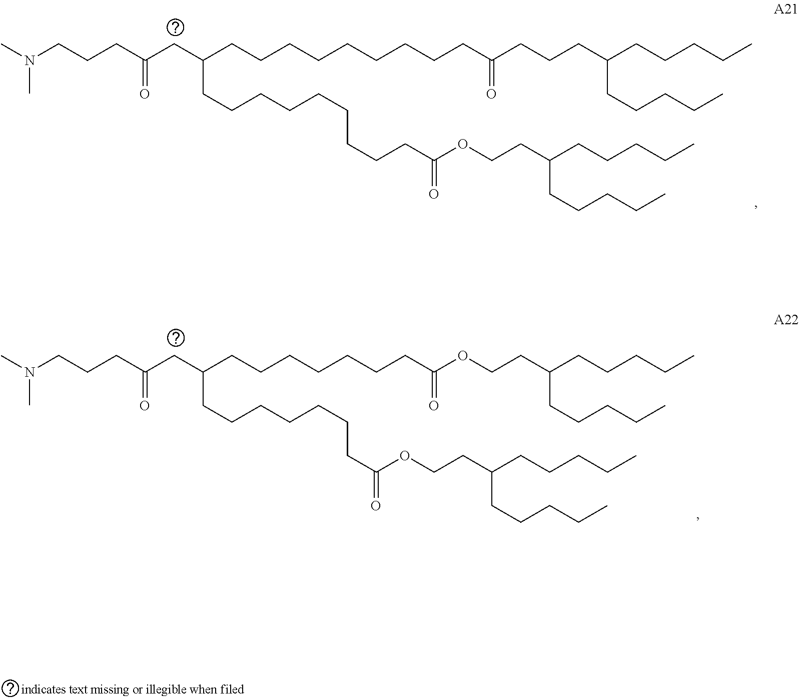

In a preferred embodiment, the cationic lipid includes MC3.

In a preferred embodiment, the cationic lipid includes a lipid compound having a structure shown below, or a pharmaceutically acceptable salt thereof:

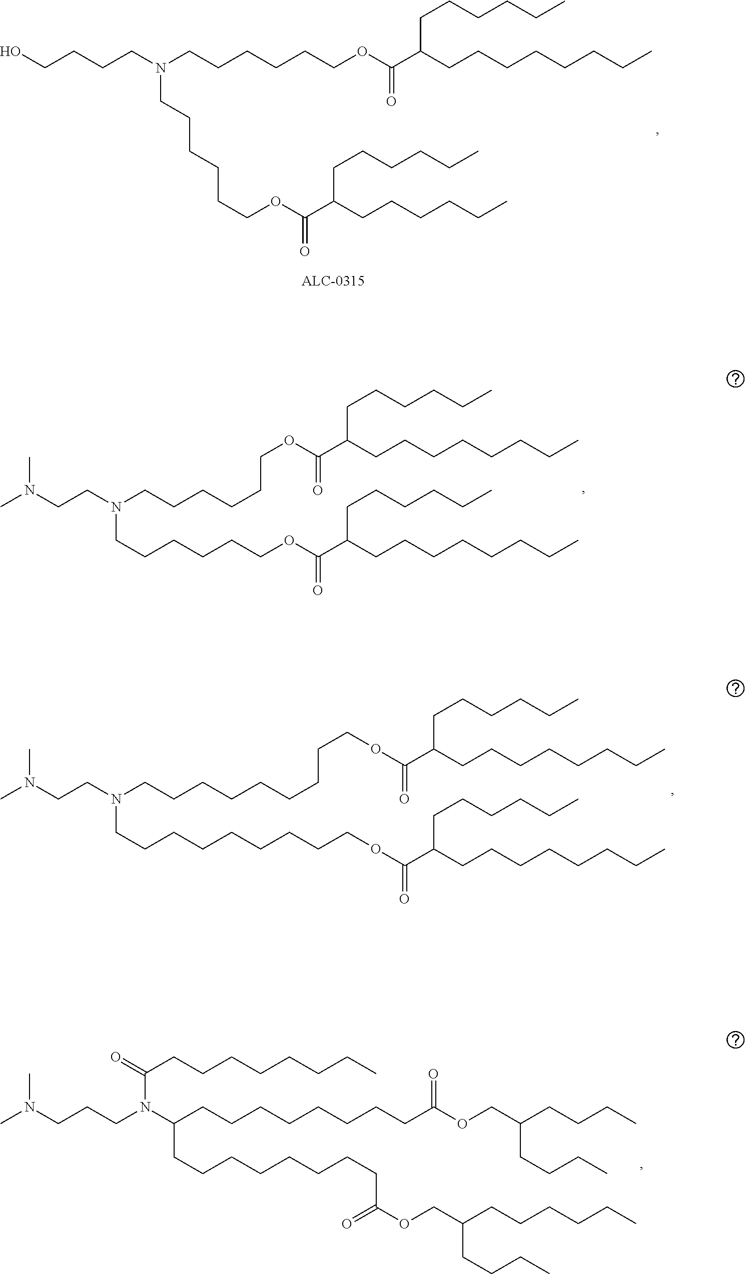

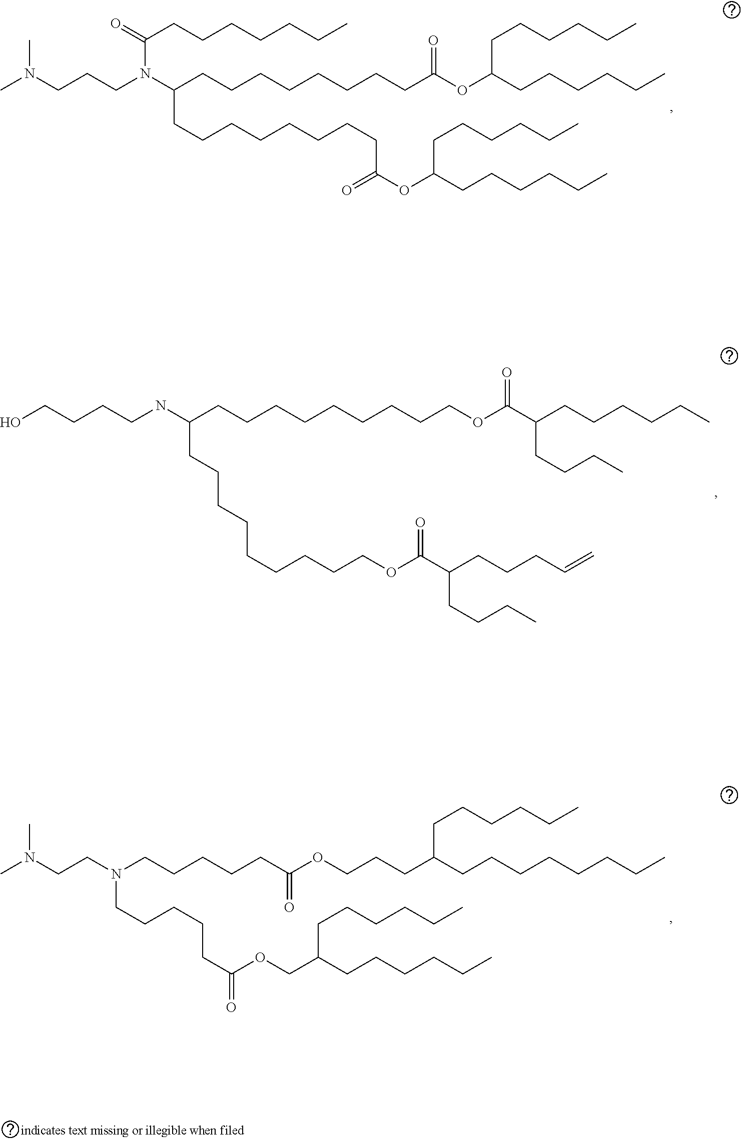

In a preferred embodiment, the cationic lipid includes ALC-0315.

In an embodiment, the cationic lipid includes a compound of formula (I), or a pharmaceutically acceptable salt thereof:

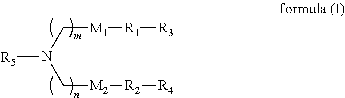

R₁ and R₂ are each independently selected from C₁-C₁₂ alkyl and C₂-C₁₂ alkenyl;
R₃ and R₄ are each independently selected from C₁-C₁₂ alkyl, C₂-C₁₂ alkenyl, C₆-C₁₀ aryl, and 5-10 membered heteroaryl,
with a proviso that at least one of R₃ and R₄ is C₆-C₁₀ aryl or 5-10 membered heteroaryl; R₃ and R₄ are each independently optionally substituted with t R₆, t being an integer selected from 1-5; and R₆ is independently selected from C₁-C₁₂ alkyl and C₂-C₁₂ alkenyl;
M₁ and M₂ are each independently selected from —OC(O)—, —C(O)O—, —SC(S)—, and —C(S)S—;
R₅ is selected from —C₁₋₁₂ alkylene-Q, Q is selected from —OR₇ and —SR₇, and R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl; and
m and n are each independently an integer selected from 1-12.

In an embodiment, R₂ is selected from C₁-C₁₂ alkyl. In another embodiment, R₂ is selected from C₁-C₆ alkyl.

In an embodiment, one of R₃ and R₄ is C₆-C₁₀ aryl or 5-10 membered heteroaryl, and the other is C₁-C₁₂ alkyl or C₂-C₁₂ alkenyl.

In a specific embodiment, R₃ and R₄ are each independently selected from C₁-C₁₂ alkyl and phenyl, with a proviso that at least one of R₃ and R₄ is phenyl. In another embodiment, one of R₃ and R₄ is phenyl, and the other is C₁-C₁₂ alkyl.

In yet another embodiment, R₃ and R₄ are each independently substituted with t R₆, t being an integer selected from 1-5, e.g., 1, 2, 3, 4, or 5. Preferably, t is an integer from 1 to 3, e.g., 1, 2, or 3, in particular 1 or 2.

In an embodiment, R₆ is independently selected from C₁-C₁₂ alkyl, e.g., C₁-C₁₀ alkyl.

In an embodiment, t is 1, and R₆ is substituted at a meta-position or a para-position on a benzene ring relative to R₁ or R₂.

In another embodiment, t is 2, and R₆ is substituted at a meta-position and a para-position on a benzene ring relative to R₁ or R₂.

In an embodiment, R₄ is substituted at a 1-position or a last position of R₂. The 1-position refers to a position of a C atom in R₂ directly connected to M₂. The last position refers to a position of a C atom in R₂ farthest away from M₂. In a specific embodiment, R₄ is selected from C₁-C₁₂ alkyl, and R₃ is phenyl.

In an embodiment, R₃ is substituted at a 1-position or a last position of R₁. The 1-position refers to a position of a C atom in R₁ directly connected to M₁. The last position refers to a position of a C atom in R₁ farthest away from M₁. In a specific embodiment, R₃ is selected from C₁-C₁₂ alkyl, and R₄ is phenyl.

In an embodiment, M₁ and M₂ are each independently selected from —OC(O)— and —C(O)O—.

In an embodiment, R₅ is selected from —C₁₋₅ alkylene-Q, e.g., C₁, C₂, C₃, C₄, or C₅ alkylene-Q. In an exemplary embodiment, R₅ is selected from —C₁₋₃ alkylene-Q, e.g., C₁, C₂, or C₃ alkylene-Q.

In another embodiment, Q is selected from —OH and —SH, in particular —OH.

In some embodiments, m and n are each independently an integer selected from 2-9, e.g., 2, 3, 4, 5, 6, 7, 8, or 9. Preferably, m and n are each independently an integer selected from 2-7, e.g., 2, 3, 4, 5, 6, or 7, and more preferably, m and n are each independently an integer selected from 5-7, e.g., 5, 6, or 7.

In certain embodiments, the compound of formula (I) includes a compound shown in formula (II):

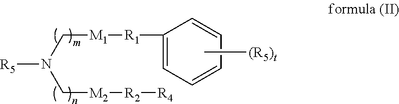

or a pharmaceutically acceptable salt thereof, wherein each group is as defined herein.

In an embodiment,
R₁ is selected from C₁-C₆ alkyl;
R₂ is selected from C₁-C₁₀ alkyl;
R₄ is selected from C₁-C₁₀ alkyl;
M₁ and M₂ are each independently selected from —OC(O)— and —C(O)O—;
R₅ is selected from —C₁₋₅ alkylene-Q, Q is selected from —OR₇ and —SR₇, and R₇ is independently selected from H, C₁-C₁₂ alkyl, and C₂-C₁₂ alkenyl;
R₆ is independently selected from C₁-C₁₂ alkyl and C₂-C₁₂ alkenyl, in particular C₁-C₁₂ alkyl;
m and n are each independently an integer selected from 2-9, e.g., 2, 3, 4, 5, 6, 7, 8, or 9; and
t is an integer selected from 1-3.

In an embodiment, R₅ is selected from —C₁₋₃ alkylene-Q, and Q is selected from —OH and —SH, in particular —OH.

In an embodiment, m and n are each independently an integer selected from 2-7, e.g., 2, 3, 4, 5, 6, or 7.

In some embodiments, t is 1 or 2.

In an embodiment, R₄ is substituted at a 1-position or a last position of R₂. The 1-position refers to a position of a C atom in R₂ directly connected to M₂. The last position refers to a position of a C atom in R₂ farthest away from M₂.

In an embodiment, t is 1, R₆ is substituted at a meta-position or a para-position on a benzene ring relative to R₁.

In another embodiment, t is 2, and R₆ is substituted at a meta-position and a para-position on a benzene ring relative to R₁.

In certain embodiments, the compound of formula (I) includes a compound shown in formula (III):

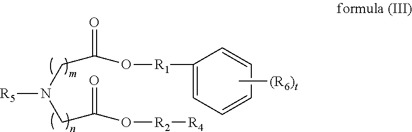

or a pharmaceutically acceptable salt thereof, wherein each group is as defined herein.

In an embodiment,
R₁ is selected from C₁-C₆ alkyl;
R₂ is selected from C₁-C₁₀ alkyl;
R₄ is selected from C₁-C₁₀ alkyl;
R₅ is selected from —C₁₋₃ alkylene-Q, and Q is selected from —OH and —SH, in particular —OH;
t is 1 or 2;
R₆ is selected from C₁-C₁₂ alkyl and C₂-C₁₂ alkenyl, in particular C₁-C₁₂ alkyl; and
m and n are each independently an integer selected from 2-7, e.g., 2, 3, 4, 5, 6, or 7.

In an embodiment, R₄ is substituted at a 1-position or a last position of R₂. The 1-position refers to a position of a C atom in R₂ directly connected to

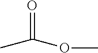

moiety. The last position refers to a position of a C atom in R₂ farthest away from

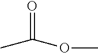

moiety.

In an embodiment, t is 1, R₆ is substituted at a meta-position or a para-position on a benzene ring relative to R₁.

In another embodiment, t is 2, and R₆ is substituted at a meta-position and a para-position on a benzene ring relative to R₁.

In certain embodiments, the compound of formula (I) includes a compound shown in formula (IV):

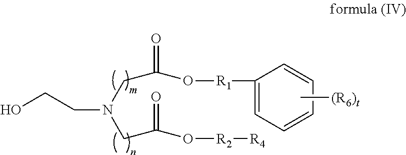

or a pharmaceutically acceptable salt thereof, wherein each group is as defined herein.

In an embodiment,
R₁ is selected from C₁-C₆ alkyl;
R₂ is selected from C₁-C₁₀ alkyl;
R₄ is selected from C₁-C₁₀ alkyl;
t is 1 or 2;
R₆ is independently selected from C₁-C₁₂ alkyl and C₂-C₁₂ alkenyl, in particular C₁-C₁₂ alkyl;
m and n are each independently an integer selected from 2-7, e.g., 2, 3, 4, 5, 6, or 7.

In an embodiment, R₄ is substituted at a 1-position or a last position of R₂. The 1-position refers to a position of a C atom in R₂ directly connected to

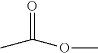

moiety. The last position refers to a position of a C atom in R₂ farthest away from

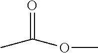

moiety.

In an embodiment, t is 1, R₆ is substituted at a meta-position or a para-position on a benzene ring relative to R₁.

In another embodiment, t is 2, and R₆ is substituted at a meta-position and a para-position on a benzene ring relative to R₁.

In a particular embodiment, the substituent groups (e.g., R₁-R₇) in the lipid compounds of the present disclosure include no alkenyl.

In a preferred embodiment, the cationic lipid includes a lipid compound having a structure shown below, or a pharmaceutically acceptable salt thereof:

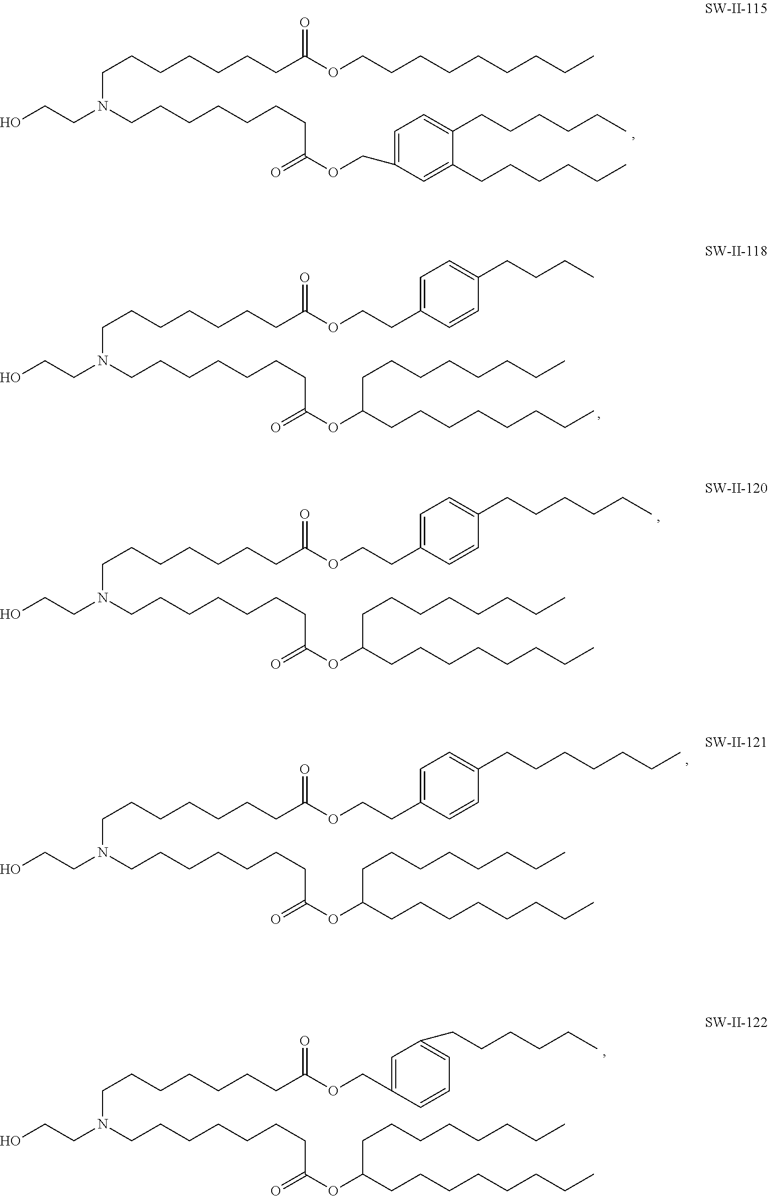 [SMILES: CCCCCCCCCOC(=O)CCCCCCCN(CCO)CCCCCCCC(=O)OCc1ccc(CCCCCC)c(CCCCCC)c1 ; CCCCCCCCC(CCCCCCCC)OC(=O)CCCCCCCN(CCO)CCCCCCCC(=O)OCCc1ccc(CCCC)cc1 ; CCCCCCCCC(CCCCCCCC)OC(=O)CCCCCCCN(CCO)CCCCCCCC(=O)OCCc1ccc(CCCCCC)cc1 ; CCCCCCCCC(CCCCCCCC)OC(=O)CCCCCCCN(CCO)CCCCCCCC(=O)OCCc1ccc(CCCCCCC)cc1 ; CCCCCCCCC(CCCCCCCC)OC(=O)CCCCCCCN(CCO)CCCCCCCC(=O)OCc1cccc(CCCCCC)c1]

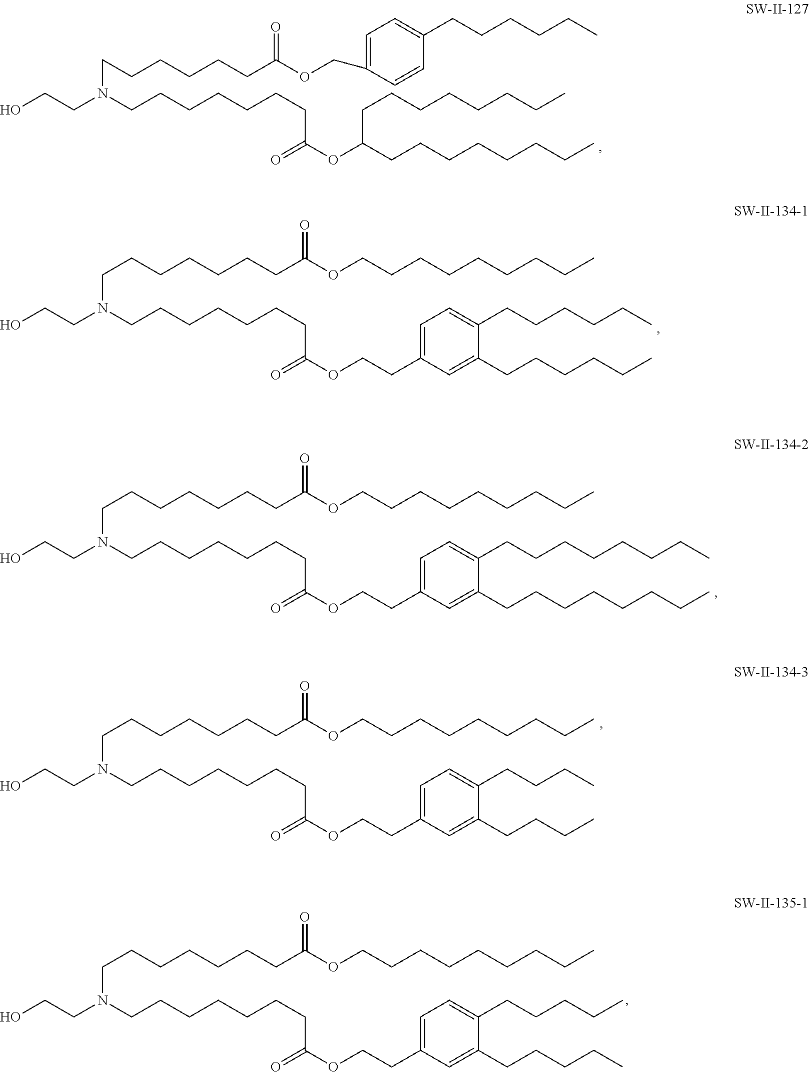 [SMILES: CCCCCCCCC(CCCCCCCC)OC(=O)CCCCCCCN(CCO)CCCCCCC(=O)OCc1ccc(CCCCCC)cc1 ; CCCCCCCCCOC(=O)CCCCCCCN(CCO)CCCCCCCC(=O)OCCc1ccc(CCCCCC)c(CCCCCC)c1 ; CCCCCCCCCOC(=O)CCCCCCCN(CCO)CCCCCCCC(=O)OCCc1ccc(CCCCCCCC)c(CCCCCCCC)c1 ; CCCCCCCCCOC(=O)CCCCCCCN(CCO)CCCCCCCC(=O)OCCc1ccc(CCCC)c(CCCC)c1 ; CCCCCCCCCOC(=O)CCCCCCCN(CCO)CCCCCCCC(=O)OCCc1ccc(CCCCC)c(CCCCC)c1]

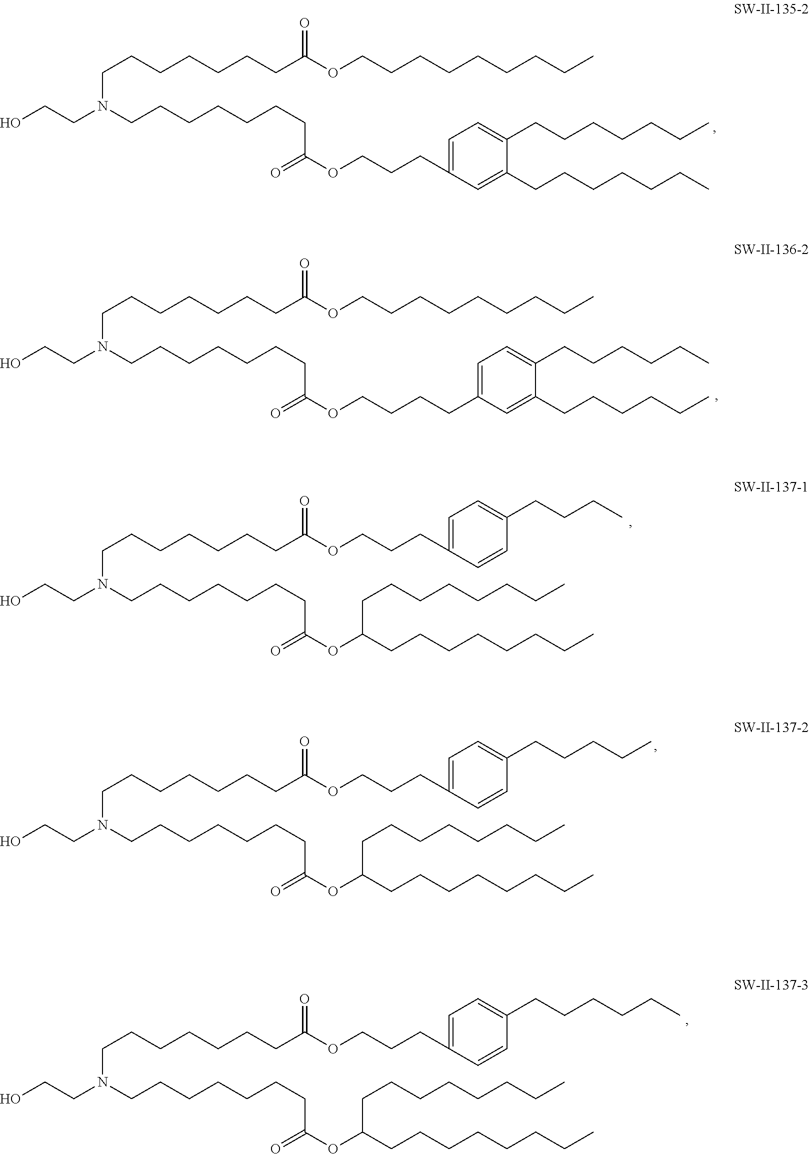 [SMILES: CCCCCCCCCOC(=O)CCCCCCCN(CCO)CCCCCCCC(=O)OCCCc1ccc(CCCCCCC)c(CCCCCCC)c1 ; CCCCCCCCCOC(=O)CCCCCCCN(CCO)CCCCCCCC(=O)OCCCCc1ccc(CCCCCC)c(CCCCCC)c1 ; CCCCCCCCC(CCCCCCCC)OC(=O)CCCCCCCN(CCO)CCCCCCCC(=O)OCCCc1ccc(CCCC)cc1 ; CCCCCCCCC(CCCCCCCC)OC(=O)CCCCCCCN(CCO)CCCCCCCC(=O)OCCCc1ccc(CCCCC)cc1 ; CCCCCCCCC(CCCCCCCC)OC(=O)CCCCCCCN(CCO)CCCCCCCC(=O)OCCCc1ccc(CCCCCC)cc1]

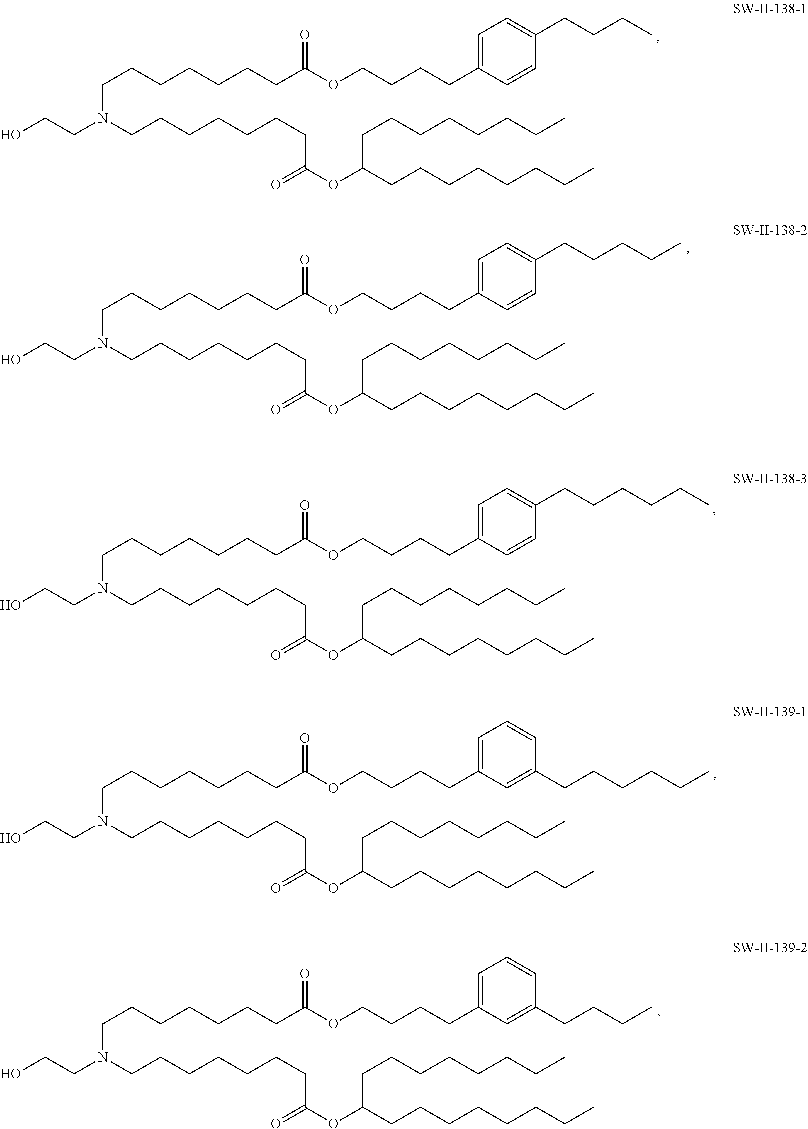 [SMILES: CCCCCCCCC(CCCCCCCC)OC(=O)CCCCCCCN(CCO)CCCCCCCC(=O)OCCCCc1ccc(CCCC)cc1 ; CCCCCCCCC(CCCCCCCC)OC(=O)CCCCCCCN(CCO)CCCCCCCC(=O)OCCCCc1ccc(CCCCC)cc1 ; CCCCCCCCC(CCCCCCCC)OC(=O)CCCCCCCN(CCO)CCCCCCCC(=O)OCCCCc1ccc(CCCCCC)cc1 ; CCCCCCCCC(CCCCCCCC)OC(=O)CCCCCCCN(CCO)CCCCCCCC(=O)OCCCCc1cccc(CCCCCC)c1 ; CCCCCCCCC(CCCCCCCC)OC(=O)CCCCCCCN(CCO)CCCCCCCC(=O)OCCCCc1cccc(CCCC)c1]

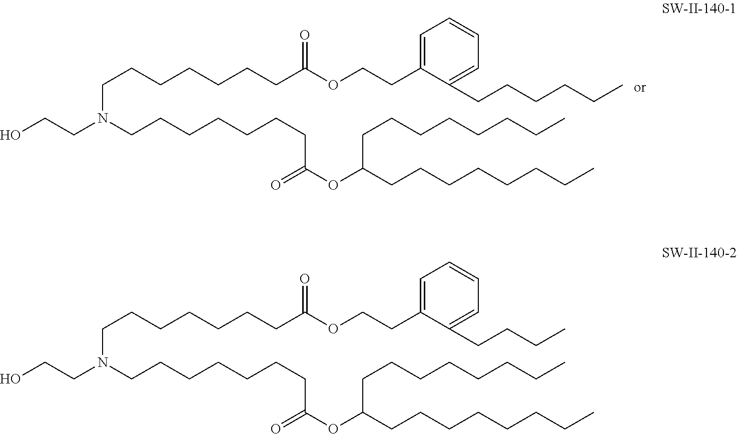 [SMILES: CCCCCCCCC(CCCCCCCC)OC(=O)CCCCCCCN(CCO)CCCCCCCC(=O)OCCc1ccccc1CCCCCC ; CCCCCCCCC(CCCCCCCC)OC(=O)CCCCCCCN(CCO)CCCCCCCC(=O)OCCc1ccccc1CCCC]

In a preferred embodiment, the cationic lipid includes the following lipid compounds: SW-II-115, SW-II-121, SW-II-122, SW-II-134-3, SW-II-138-2, SW-II-139-2, or SW-II-140-2.

In a preferred embodiment, the cationic lipid does not include T5′.

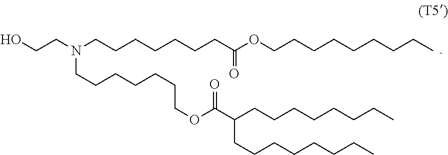 [SMILES: CCCCCCCCCOC(=O)CCCCCCCN(CCO)CCCCCCCOC(=O)C(CCCCCCCC)CCCCCCCC]

In a preferred embodiment, the cationic lipid includes the following lipid compounds: M5, MC3, ALC-0315, SM-102, SW-II-115, SW-II-121, SW-II-122, SW-II-134-3, SW-II-138-2, SW-II-139-2, or SW-II-140-2.

### Phospholipid

The lipid composition of the present disclosure includes a phospholipid, which can assist the cell permeation of the lipid composition.

Instances of phospholipids include but are not limited to: 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleylphosphatidylethanolamine (POPE), distearoyl-phosphatidyl-ethanolamine (DSPE), dipalmitoylphosphatidylethanolamine (DPPE), dimyristoylphosphoethanolamine (DMPE), 1-stearoyl-2-oleyl-stearylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), or a combination thereof.

### Steroid

The lipid composition of the present disclosure includes a steroid, which can serve as a structural component of the lipid composition.

Instances of steroids include but are not limited to, for example, cholesterol, coprosterol, sitosterol, ergosterol, campesterol, stigmasterol, brassicasterol, tomatidine, ursolic acid, α-tocopherol, and derivatives thereof.

### Polyethylene Glycol Modified Lipid

As used herein, the term “polyethylene glycol modified lipid” or “PEG modified lipid” or “PEG lipid” refers to a molecule including a polyethylene glycol moiety and a lipid moiety, and is a lipid modified with polyethylene glycol. The PEG lipid may be selected from the non-limiting groups consisting of PEG-modified phosphatidylethanolamine, PEG-modified phosphatidic acid, PEG-modified ceramide (PEG-CER), PEG-modified dialkylamine, PEG-modified diacylglycerol (PEG-DEG), PEG-modified dialkylglycerol, or a combination thereof. For example, instances of polyethylene glycol modified lipids include but are not limited to: 1,2-dimyristoyl-rac-glycero-3-methoxypolyethylene glycol (DMG-PEG), 1,2-dioleoyl-rac-glycerol,methoxypolyethylene glycol (DOGPEG), and 1,2-distearoyl-sn-glycero-3-phosphoethanolamine-poly(ethylene glycol) (DSPE-PEG).

In an embodiment, the polyethylene glycol modified lipid is DMG-PEG, e.g., DMG-PEG 2000. In an embodiment, DMG-PEG 2000 has the following structure:

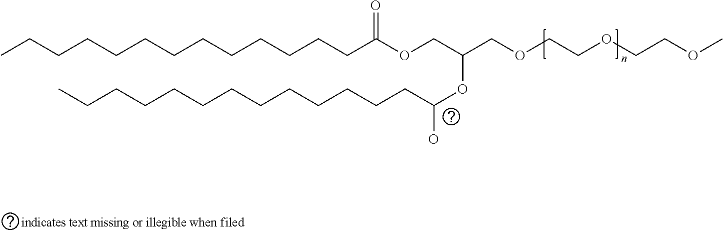

where an average value of n is 44.

### Cationic Polymer

As used herein, the term “cationic polymer” relates to any ionic polymer capable of carrying a net positive charge at a specified pH to bind electrostatically to a nucleic acid. Instances of cationic polymers include but are not limited to: poly-L-lysine, protamine, polyethyleneimine (PEI), or a combination thereof. PEI may be linear or branched PEI.

The term “protamine” refers to an arginine-rich, low molecular weight, basic protein that is present in sperm cells of various animals (particularly fish) and binds to a DNA instead of histone. In a preferred embodiment, the cationic polymer is the protamine (e.g., protamine sulfate).

### Physicochemical Properties

The physicochemical properties of a lipid composition may depend on its components. For example, a composition including cholesterol as a structural lipid may have different physicochemical properties than a composition including a different structural lipid. Similarly, the physicochemical properties of a composition may depend on the absolute or relative amount of its components. For example, a composition including a phospholipid with a higher mole fraction may have different physicochemical properties than a composition including a phospholipid with a lower mole fraction. The physicochemical properties may also vary depending on the method and conditions for preparing the composition.

The physicochemical properties of a lipid composition may be characterized by various methods. For example, microscopy (e.g., transmission electron microscopy or scanning electron microscopy) can be used to examine the morphology and size distribution of the composition. Dynamic light scattering (DLS) or potentiometry (e.g., potentiometric titration) can be used to measure the potential. DLS can also be used to determine the particle size. Instruments such as Zetasizer Nano ZS (Malvern Instruments Ltd, Malvern, Worcestershire, UK) can also be used to measure a plurality of characteristics of a composition, e.g., particle size, polydispersity index, and (potential.

As measured, for example, by DLS, the average size of a composition may be between tens and hundreds of nanometers. For example, the average size may be about 40 nm to about 250 nm, e.g., about 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, or 300 nm. In some embodiments, the average size of a composition may be about 50 nm to about 300 nm, about 50 nm to about 290 nm, about 50 nm to about 280 nm, about 50 nm to about 270 nm, about 50 nm to about 260 nm, about 60 nm to about 300 nm, about 60 nm to about 290 nm, about 60 nm to about 280 nm, about 60 nm to about 270 nm, about 70 nm to about 300 nm, about 70 nm to about 290 nm, about 70 nm to about 280 nm, about 70 nm to about 270 nm, about 70 nm to about 260 nm, about 80 nm to about 280 nm, about 80 nm to about 270 nm, about 80 nm to about 260 nm, about 80 nm to about 250 nm, about 90 nm to about 280 nm, about 90 nm to about 270 nm, or about 90 nm to about 260 nm. In some embodiments, the average size of a lipid composition may be about 90 nm to about 290 nm, or about 100 nm to about 250 nm. In a particular embodiment, the average size may be about 100 nm. In other embodiments, the average size may be about 150 nm. In other embodiments, the average size may be about 200 nm.

The lipid composition may be relatively homogeneous. The polydispersity index may be used to indicate the homogeneity of the lipid composition, e.g., the particle size distribution of the lipid composition. A smaller (e.g., less than 0.3) polydispersity index generally indicates a narrower particle size distribution. The polydispersity index of a composition may be about 0 to about 0.25, e.g., 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a lipid composition may be about 0.10 to about 0.20.

The ζ potential of a composition may be used to indicate the electrokinetic potential of the composition. For example, the ζ potential may describe the surface charge of a composition. Compositions with relatively lower charge, i.e., positively or negatively charged, are generally desirable because the compositions with higher charge may undesirably interact with cells, tissues, and other elements within the body. In some embodiments, the (potential of a composition may be about −10 mV to about +20 mV, about −10 mV to about +15 mV, about −10 mV to about +10 mV, about −10 mV to about +5 mV, about −10 mV to about 0 mV, about −10 mV to about −5 mV, about −5 mV to about +20 mV, about −5 mV to about +15 mV, about −5 mV to about +10 mV, about −5 mV to about +5 mV, about −5 mV to about 0 mV, about 0 mV to about +20 mV, about 0 mV to about +15 mV, about 0 mV to about +10 mV, about 0 mV to about +5 mV, about +5 mV to about +20 mV, about +5 mV to about +15 mV, or about +5 mV to about +10 mV.

The encapsulation efficiency of a therapeutic agent or a prophylactic agent describes the ratio of the amount of the therapeutic agent or the prophylactic agent encapsulated in or otherwise combined with the composition after preparation relative to the initial amount provided. Higher encapsulation efficiency is ideal (e.g., near 100%). The encapsulation efficiency may be measured, for example, by comparing the amount of a therapeutic agent or a prophylactic agent in a solution containing a composition before and after splitting the composition with one or more organic solvents or detergents. Fluorescence can be used to measure the amount of a therapeutic agent or a prophylactic agent (e.g., an RNA) in a solution. For the composition described herein, the encapsulation efficiency of a therapeutic agent or a prophylactic agent may be at least 50%, e.g., 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

### Pharmaceutical Composition

The present disclosure further provides a pharmaceutical composition, including the lipid composition of the present disclosure, and a pharmaceutically acceptable carrier.

Pharmaceutically acceptable carriers may include but are not limited to: diluents, binders and adhesives, lubricants, disintegrants, preservatives, vehicles, dispersing agents, glidants, sweeteners, coatings, excipients, preservatives, antioxidants (e.g., ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, propyl gallate, α-tocopherol, citric acid, ethylenediaminetetraacetic acid (EDTA), sorbitol, tartaric acid, phosphoric acid, etc.), solubilizing agents, gelling agents, softeners, solvents (e.g., water, alcohol, acetic acid, and syrup), buffering agents (e.g., phosphate buffer, histidine buffer, and acetate buffer), surfactants (e.g., non-ionic surfactants such as polysorbate 80, polysorbate 20, poloxamer, or polyethylene glycol), antibacterial agents, antifungal agents, isotonic agents (e.g., trehalose, sucrose, mannitol, sorbitol, lactose, and glucose), absorption delaying agents, chelating agents, and emulsifying agents. For the pharmaceutical composition including the lipid composition, an appropriate carrier may be selected from a buffer (e.g., citrate buffer, acetate buffer, phosphate buffer, histidine buffer, and histidine salt buffer), an isotonic agent (e.g., trehalose, sucrose, mannitol, sorbitol, lactose, and glucose), a non-ionic surfactant (e.g., polysorbate 80, polysorbate 20, and poloxamer), or a combination thereof.

The pharmaceutical composition provided herein may be in various dosage forms, including but not limited to solid, semi-solid, liquid, powder, or lyophilized forms. For the pharmaceutical composition including the lipid composition, the preferred dosage forms may generally be, for example, injection solution and lyophilized powder. The pharmaceutical composition may be prepared in various forms suitable for various routes and methods of administration. For example, the pharmaceutical composition may be prepared as liquid dosage forms (e.g., emulsions, microemulsions, nanoemulsions, solutions, suspensions, syrups, and elixirs), injectable forms, solid dosage forms (e.g., capsules, tablets, pills, powders, and granules), surface and/or transdermal administration dosage forms (e.g., ointments, pastes, creams, lotions, gels, powders, solutions, sprays, inhalants, and patches), suspensions, powders, and other forms.

The pharmaceutical composition of the present disclosure may be a pharmaceutical composition for injection, which may correspondingly include a pharmaceutically acceptable excipient for injection, and preferably, an excipient for intramuscular injection.

Therefore, the present disclosure further provides an injectant, including the lipid composition of the present disclosure, and a pharmaceutically acceptable excipient for injection. For example, a sterile injectable aqueous or oily suspension may be included. A sterile injectable preparation may be a sterile injectable solution, suspension, and/or emulsion in a non-toxic parenterally-acceptable diluent and/or solvent, e.g., a solution in 1,3-butanediol.

The above excipient for injection may include one or more of water, a saccharide solution, an electrolyte solution, and an amino acid solution. For example, the excipient for injection may include one or more of Ringer's solution, glucose solution, glucose and sodium chloride solution, isotonic sodium chloride solution, fructose solution, dextran, amino acid solution, heparin solution, mannitol solution, and sodium bicarbonate solution. Sterile fixed oil may also be employed as a solvent or suspending medium. For this purpose, any mild fixed oil may be employed, including synthetic monoglyceride or diglyceride. Fatty acids, e.g., oleic acids, may be used in preparation of an injectable preparation.

### Therapeutic Agent/Prophylactic Agent

A lipid composition may include one or more therapeutic agents or prophylactic agents. The present disclosure provides methods for delivering a therapeutic agent or a prophylactic agent to a mammalian cell or organ, producing a polypeptide of interest in the mammalian cell, and treating a disease or disorder in a mammal in need thereof, including administering to the mammal a lipid composition including the therapeutic agent or the prophylactic agent.

The therapeutic agent or the prophylactic agent includes a biologically active substance and is alternatively referred to as “active agent”. The therapeutic agent or the prophylactic agent may be a substance that causes a desired change in a cell or organ or other body tissue or system after delivery to the cell or organ. Such substances may be used to treat one or more diseases, disorders, or conditions. In some embodiments, the therapeutic agent or the prophylactic agent is a small molecule drug that can be used to treat a particular disease, disorder, or condition. Instances of drugs capable of being used for a composition include but are not limited to antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cytosine arabinoside, anthracycline, alkylating agents, platinum compounds, antimetabolites, and nucleoside analogues, e.g., methotrexate and purine and pyrimidine analogues), antiinfective agents, local anesthetics (e.g., dibucaine and chlorpromazine), β-adrenergic blocking agents (e.g., propranolol, timolol and labetalol), antihypertensive agents (e.g., clonidine and hydralazine), antidepressant agents (e.g., imipramine, amitriptyline and doxepin), antispasmodic agents (e.g., phenytoin), antihistamines (e.g., diphenhydramine, chlorpheniramine, and promethazine), antibiotics/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma drugs, vitamins, sedatives, and imaging agents.

In some embodiments, the therapeutic agent or the prophylactic agent is a cytotoxin, a radioactive ion, a chemotherapeutic agent, a vaccine, a compound that elicits an immune response, or another therapeutic agent or prophylactic agent. Cytotoxins or cytotoxic agents include any agent that is detrimental to cells. Instances include but are not limited to taxol, cytochalasin B, gramicidin D, ethidium bromide, emetine, mitomycin, etoposide, teniposide, vincristine, vinblastine, colchicine, doxorubicin, daunorubicin, dihydroxy anthracin dione, mitoxantrone, mithramycin, actinomycin D, 1-dehydrotestosterone, glucocorticoid, procaine, tetracaine, lidocaine, propranolol, puromycin, maytansinoid such as maytansinol, rachelmycin (CC-1065), and analogues or homologues thereof. Radioactive ions include but are not limited to iodine (e.g., iodine 125 or iodine 131), strontium 89, phosphorus, palladium, cesium, iridium, phosphate radical, cobalt, yttrium 90, samarium 153, and praseodymium. Vaccines include compounds and preparations that are capable of providing immunity against one or more conditions associated with infectious diseases such as influenza, measles, human papilloma virus (HPV), rabies, meningitis, whooping cough, tetanus, pestilence, hepatitis, and tuberculosis, and may include an mRNA encoding an infectious disease derived antigen and/or epitope. Vaccines may also include compounds and preparations that direct the immune response against cancer cells, and may include an mRNA encoding a tumor cell derived antigen, epitope, and/or neoepitope. Compounds that elicit an immune response may include vaccines, corticosteroids (e.g., dexamethasone), and other substances. In some embodiments, a vaccine and/or compound capable of eliciting an immune response is administered intramuscularly via a composition including a compound according to formula (I), (II), (III) or (IV). Other therapeutic agents or prophylactic agents include but are not limited to antimetabolites (e.g., methotrexate, 6-mercaptopurine, 6-thioguanine, cytarabine and 5-fluorouracil dacarbazine), alkylating agents (e.g., mechlorethamine, thiotepa, chlorambucil, rachelmycin (CC-1065), melphalan, carmustine, (BSNU), lomustine (CCNU), cyclophosphamide, busulfan, dibromannitol, streptozotocin, mitomycin C, and cis-diamminedichloroplatinum (II)(DDP), cis-platinum), anthracycline (e.g., daunorubicin (formerly referred to as daunomycin), and doxorubicin), antibiotics (e.g., dactinomycin (formerly referred to as actinomycin), bleomycin, mithramycin, and anthramycin (AMC)), and antimitotic agents (e.g., vincristine, vinblastine, taxol, and maytansinoid).

In other embodiments, the therapeutic agent or the prophylactic agent is a protein. Therapeutic proteins capable of being used in a nanoparticle of the present disclosure include but are not limited to gentamycin, amikacin, insulin, erythropoietin (EPO), granulocyte colony-stimulating factor (G-CSF), granulocyte-macrophage colony-stimulating factor (GM-CSF), factor VIR, luteinizing hormone-releasing hormone (LHRH) analogue, interferon, heparin, hepatitis B surface antigen, typhoid vaccine, and cholera vaccine.

### Polynucleotide

In some embodiments, the therapeutic agent or the prophylactic agent is a polynucleotide or a nucleic acid (e.g., a ribonucleic acid or a deoxyribonucleic acid).

In some embodiments, the therapeutic agent or the prophylactic agent of the present disclosure is an RNA. As used herein, the definition of “RNA” encompasses single-stranded, double-stranded, linear, and circular RNAs. The RNA of the present disclosure may be chemically synthesized, recombinantly produced, and in vitro transcribed RNAs. In an embodiment, the RNA of the present disclosure is used for expression of a polypeptide in a host cell.

In an embodiment, the therapeutic agent or the prophylactic agent of the present disclosure is a single-stranded RNA. In an embodiment, the RNA of the present disclosure is an in vitro transcribed RNA (IVT-RNA). IVT-RNA may be obtained by in vitro transcription via an RNA polymerase using a DNA template.

In some embodiments, the therapeutic agent or the prophylactic agent of the present disclosure is a messenger RNA (mRNA). Generally speaking, the mRNA may include a 5′-UTR sequence, a coding sequence for polypeptides, a 3′-UTR sequence, and an optionally present poly(A) sequence. mRNA may be produced, for example, via in vitro transcription or chemical synthesis. In an embodiment, the mRNA of the present disclosure includes (1) 5′-UTR, (2) a coding sequence, (3) 3′-UTR, and (4) an optionally present poly(A) sequence. In an embodiment, the RNA of the present disclosure is a nucleoside modified mRNA. In an embodiment, the mRNA of the present disclosure includes an optionally present 5′ cap.

As used herein, the term “untranslated region (UTR)” generally refers to a region (non-coding region) in an RNA (e.g., an mRNA) that is not translated into an amino acid sequence, or a corresponding region in a DNA. Generally, UTR located at a 5′ end (upstream) of an open reading frame (initiation codon) may be referred to as 5′ untranslated region 5′-UTR; and UTR located at a 3′ end (downstream) of an open reading frame (termination codon) may be referred to as 3′-UTR. In the presence of a 5′ cap, the 5′-UTR is located downstream the 5′ cap, for example, directly adjacent to the 5′ cap. In a particular embodiment, an optimized “Kozak sequence” may be included in the 5′-UTR, for example, near the initiation codon, to improve translation efficiency. In the presence of a poly(A) sequence, the 3′-UTR is located upstream the poly(A) sequence, for example, directly adjacent to the poly(A) sequence.

As used herein, the term “poly(A) sequence” or “poly(A) tail” refers to a nucleotide sequence including consecutive or inconsecutive adenosines. The poly(A) sequence is generally located at a 3′ end of RNA, for example, 3′ end (downstream) of 3′-UTR. In some embodiments, the poly(A) sequence does not include any nucleotides other than adenosine at its 3′ end. The poly(A) sequence may be produced by transcription by a DNA-dependent RNA polymerase based on a coding sequence of a DNA template during the preparation of IVT-RNA, or connected to a free 3′ end of IVT-RNA, e.g., 3′ end of 3′-UTR, via a DNA-independent RNA polymerase (poly(A) polymerase).

As used herein, the term “5′ cap” generally involves an N7-methylguanosine structure (also referred to as “m7G cap”, and “m7Gppp-”) connected to a 5′ end of mRNA via a 5′ to 5′ triphosphate bond. The 5′ cap may be co-transcribed and added to an RNA during in vitro transcription (e.g., using an anti-reverse cap analog “ARCA”), or may be connected to an RNA after transcription using a capping enzyme.

In some embodiments, the therapeutic agent or the prophylactic agent of the present disclosure is a DNA. Such DNA may be, for example, a DNA template for in vitro transcription of the RNA of the present disclosure, or a DNA vaccine for expression of a polypeptide antigen in a host cell. The DNA may be double-stranded, single-stranded, linear, and circular DNA.

The DNA template may be provided in an appropriate transcription vector. Generally speaking, the DNA template may be a double-stranded complex including a nucleotide sequence (coding strand) that is identical to the coding sequence described herein, and a nucleotide sequence (template strand) that is complementary to the coding sequence described herein. As is known to those skilled in the art, the DNA template may include a promoter, 5′-UTR, a coding sequence, 3′-UTR, and an optionally present poly(A) sequence. The promoter may be a promoter available to an appropriate RNA polymerase (in particular a DNA-dependent RNA polymerase) known to those skilled in the art, including but not limited to promoters of SP6, T3 and T7 RNA polymerases. The 5′-UTR, coding sequence, 3′-UTR, and poly(A) sequence in the DNA template are corresponding or complementary to those included in the RNA described herein. The polynucleotide as a DNA vaccine may be provided in a plasmid vector (e.g., a circular plasmid vector).

In a preferred embodiment, the therapeutic agent or the prophylactic agent of the present disclosure is an mRNA of a code-modified SARS-CoV-2 spike protein. For exemplary nucleic acid sequences thereof, reference may be made to SEQ ID NOs: 3, 5, 6 or 7.

### Coronavirus

As used herein, “severe acute respiratory syndrome coronavirus 2”, “novel coronavirus”, and “SARS-CoV-2” are used interchangeably. SARS-CoV-2 is known to be the pathogen responsible for “coronavirus disease 2019 (COVID-19)”.

SARS-CoV-2 is a sense single stranded RNA ((+)ssRNA) enveloped virus, belonging to the β genus of coronaviridae. SARS-CoV-2 encodes four structural proteins: spike protein (S), envelope protein (E), membrane protein (M), and nucleocapsid protein (N). The spike protein (S protein) mediates the specific binding of a virus to a host cell and the fusion of a viral envelope to a host cell membrane, and thus is a key molecule for virus infection of a host cell.

As used herein, “SARS-CoV-2 spike protein”, “SARS-CoV-2S protein”, or “S protein” refers to a spike protein of SARS-CoV-2. SARS-CoV-2 includes but is not limited to a prototype strain and variant strains. At present, a plurality of SARS-CoV-2 variant strains have been discovered, including but not limited to Alpha (B.1.1.7 and Q lineage), Beta (B.1.351 and descendant lineage), Gamma (P.1 and descendant lineage), Delta (B.1.617.2 and AY lineage), and Omicron (lineage B.1.1.529 and BA lineage) variant strains. The SARS-CoV-2 variant strains may have a mutant SARS-CoV-2S protein. The SARS-CoV-2 variant strains may be identified, for example, based on the sequence of spike proteins. For example, a mutant SARS-CoV-2S protein may include a mutation, e.g., amino acid deletion and/or substitution, as compared to a prototype SARS-CoV-2S protein (e.g., SEQ ID NO: 11).

The SARS-CoV-2S protein is synthesized as a glycoprotein having approximately 1,273-1,300 amino acids (an exemplary amino acid sequence is shown in SEQ ID NO: 11), including an N-terminal signal peptide, an S1 subunit, and an S2 subunit. The S1 subunit includes an N-terminal domain, a receptor binding domain (RBD), and subdomains 1 and 2 (SD1/2). The S2 subunit includes a fusion peptide (FP), heptad repeat HR1, HR2, a transmembrane domain, and a cytoplasmic domain. For the description related to the SARS-CoV-2S protein, reference may also be made to, e.g., Huang Y et al., Acta Pharmacol Sin. 2020; 41(9):1141-1149.

Studies have shown that RBD of the S1 subunit recognizes a target host cell via a specific receptor, angiotensin converting enzyme 2 (ACE2), while the S2 subunit is responsible for membrane fusion. In a native state, the S protein is present on a viral surface in a metastable pre-fusion trimer conformation. During infection, RBD binds to a host cell receptor, and a host protease (e.g., Furin) cleaves an S1/S2 cleavage site of the S protein, which disrupts the stability of a pre-fusion trimer, resulting in the shedding of the S1 subunit and the conversion of the S2 subunit into a post-fusion stable conformation. A Furin cleavage site is an exposed ring structure including a plurality of arginine residues, and includes an amino acid motif Arg-Xₐₐ-X_{bb}-Arg (where Xₐₐ is any amino acid; X_{bb} is any amino acid, preferably Arg or Lys). In an embodiment, the amino acid sequence of the Furin cleavage site is Arg-Arg-Ala-Arg (“RRAR”), corresponding to amino acids 682-685 in SEQ ID NO: 11.

The modified spike protein encoded by the therapeutic agent or the prophylactic agent of the present disclosure may include an inactivated Furin cleavage site. Specifically, the polypeptide antigen of the present disclosure includes an inactivated Furin cleavage site Gln-Ser-Ala-Gln (QSAQ), thereby having a higher expression level in a host cell and/or inducing a stronger immune response in a subject. As used herein, “inactivated Furin cleavage site” refers to an amino acid sequence that cannot be recognized and cleaved by the Furin. As used herein, “active Furin cleavage site” or “Furin cleavage site” refers to an amino acid sequence that can be recognized and cleaved by the Furin.

In an aspect, the therapeutic agent or the prophylactic agent of the present disclosure is a polynucleotide that encodes a modified spike protein, wherein the modified spike protein includes an inactivated Furin cleavage site, and wherein the inactivated Furin cleavage site has an amino acid sequence of QSAQ, and the polypeptide includes:

the following amino acid substitutions numbered according to SEQ ID NO: 11: D614G, K986P, and V987P (“K986P/V987P”, also referred to as “2P mutation”).

In some embodiments, the polypeptide encoded by the polynucleotide of the present disclosure includes an amino acid sequence of SEQ ID NO: 12 or has at least 90%, 91%, 92%, 93%, 94%, 95%, 96%, 97%, 98%, or 99% identity to the amino acid sequence of SEQ ID NO: 12.

In some embodiments, the polynucleotide includes a coding region, and the coding region encodes a modified spike protein.

As used herein, “coding sequence” refers to being capable of serving as a template in a polynucleotide for the synthesis of a nucleotide sequence with a defined nucleotide sequence (e.g., a tRNA and an mRNA) or a defined amino acid sequence in a biological process. The coding sequence may be a DNA sequence or an RNA sequence. If the mRNA corresponding to the DNA sequence (including the same coding strand as the mRNA sequence and a template strand of a strand complementary thereto) is translated into a polypeptide in a biological process, the DNA sequence or the mRNA sequence may be considered to encode the polypeptide.

As used herein, “codon” refers to three consecutive nucleotide sequences (also referred to as triplet code) in a polynucleotide that encode a particular amino acid. Synonymous codons (codons encoding the same amino acid) are used at different frequencies in different species, referred to as “codon bias”. It is generally believed that for a given species, coding sequences using their biased codons may have higher translation efficiency and accuracy in an expression system of the species. Thus, “codon optimization” may be carried out on the polynucleotide, i.e., the codons in the polynucleotide are altered to reflect codons preferred by a host cell, and preferably, the amino acid sequences encoded thereby are not altered. Those skilled in the art will understand that due to degeneracy of codons, the polynucleotide of the present disclosure may include such a coding sequence that differs from the coding sequence described herein (e.g., having about 70%, 75%, 80%, 85%, 90%, 95%, 96%, 97%, 98%, or 99% identity to the coding sequence described herein) but encodes the same amino acid sequence. In a particular embodiment, the RNA of the present disclosure includes codons optimized for cells of a host (e.g., a subject, in particular a human), such that the polypeptide of the present disclosure is optimally expressed in the host (e.g., a subject, in particular a human).

In an embodiment, the polynucleotide of the present disclosure includes the coding sequence of the polypeptide as described herein. In an embodiment, the polynucleotide of the present disclosure includes a nucleotide sequence complementary to the coding sequence of the polypeptide as described herein. In an embodiment, the coding sequence includes an initiation codon at a 5′ end thereof and a termination codon at a 3′ end thereof. In an embodiment, the coding sequence includes the open reading frame (ORF) described herein.

In an embodiment, the coding sequence of the present disclosure encodes a polypeptide, the polypeptide including:
(1) an amino acid sequence of SEQ ID NO: 12; or (2) an amino acid sequence having at least 90%, 91%, 92%, 93%, 94%, 95%, 96%, 97%, 98%, or 99% identity to the amino acid sequence of SEQ ID NO: 12.

In an embodiment, the coding sequence of the polypeptide described herein includes a nucleotide sequence, the nucleotide sequence including: (1) a nucleotide sequence of SEQ ID NO: 3; (2) a nucleotide sequence having at least 70%, 75%, 80%, 85%, 90%, 95%, 96%, 97%, 98%, or 99% identity to the nucleotide sequence of SEQ ID NO: 3; (3) a nucleotide sequence of SEQ ID NO: 5; or (4) a nucleotide sequence having at least 70%, 75%, 80%, 85%, 90%, 95%, 96%, 97%, 98%, or 99% identity to the nucleotide sequence of SEQ ID NO: 5.

In some embodiments, the polynucleotide of the present disclosure is an RNA. In some embodiments, the RNA of the present disclosure further includes a structural element, including but not limited to a 5′ cap, 5′-UTR, 3′-UTR, and a poly(A) sequence, that helps to improve the stability and/or translation efficiency of the RNA.

In some embodiments, the RNA of the present disclosure includes the 5′-UTR. In a preferred embodiment, the 5′-UTR includes a nucleotide sequence of SEQ ID NO: 13. In a preferred embodiment, the 3′-UTR includes a nucleotide sequence of SEQ ID NO: 14. In some embodiments, the RNA of the present disclosure includes the 5′-UTR and the 3′-UTR. In a specific embodiment, the 5′-UTR includes a nucleotide sequence of SEQ ID NO: 13, and the 3′-UTR includes a nucleotide sequence of SEQ ID NO: 14.

In some embodiments, the RNA of the present disclosure includes the poly(A) sequence. In an embodiment, the poly(A) sequence includes consecutive adenosines. In an embodiment, the poly(A) sequence may include at least 20, 30, 40, 50, 60, 70, 75, 80, 85, 95, or 100 and up to 120, 150, 180, 200, or 300 adenosines. In an embodiment, the poly(A) sequence includes at least 50 nucleotides. In an embodiment, the poly(A) sequence includes at least 80 nucleotides. In an embodiment, the poly(A) sequence includes at least 100 nucleotides. In some embodiments, the poly(A) sequence includes about 70, 80, 90, 100, 120, or 150 nucleotides. In an embodiment, a consecutive adenosine sequence in the poly(A) sequence is interrupted by a sequence including a U, C, or G nucleotide. In an embodiment, the poly(A) sequence includes a nucleotide sequence of SEQ ID NO: 17.

In an embodiment, the RNA of the present disclosure includes a nucleotide sequence of SEQ ID NO: 3. In an embodiment, the RNA of the present disclosure includes a nucleotide sequence of SEQ ID NO: 6.

In an embodiment, the RNA of the present disclosure (a) includes a nucleotide sequence having at least 70%, 75%, 80%, 85%, 90%, 95%, 96%, 97%, 98%, or 99% identity to the nucleotide sequence of SEQ ID NO: 3 or 6; and (b) encodes an amino acid sequence having at least 90%, 91%, 92%, 93%, 94%, 95%, 96%, 97%, 98%, or 99% identity to the amino acid sequence of SEQ ID NO: 12.

In some embodiments, the polynucleotide of the present disclosure is a DNA. In some embodiments, the DNA of the present disclosure includes the coding sequence of the polypeptide as described herein. In some embodiments, the DNA of the present disclosure includes, from a 5′ end to a 3′ end, (1) a T7 promoter, (2) 5′-UTR, (3) a coding sequence, (4) 3′-UTR, and (5) an optionally present poly(A) sequence as described herein.

In some embodiments, the T7 promoter includes a nucleotide sequence of SEQ ID NO: 19.

In some embodiments, the DNA of the present disclosure includes the 5′-UTR. In a preferred embodiment, the 5′-UTR includes a nucleotide sequence of SEQ ID NO: 15. In a preferred embodiment, the 3′-UTR includes a nucleotide sequence of SEQ ID NO: 16. In some embodiments, the RNA of the present disclosure includes the 5′-UTR and the 3′-UTR. In a specific embodiment, the 5′-UTR includes a nucleotide sequence of SEQ ID NO: 15, and the 3′-UTR includes a nucleotide sequence of SEQ ID NO: 16.

In some embodiments, the DNA of the present disclosure includes the poly(A) sequence. In an embodiment, the poly(A) sequence includes consecutive deoxyadenosines. In an embodiment, the poly(A) sequence may include at least 20, 30, 40, 50, 60, 70, 75, 80, 85, 95, or 100 and up to 120, 150, 180, 200, or 300 deoxyadenosine. In an embodiment, a consecutive adenosine sequence in the poly(A) sequence is interrupted by a sequence including a T, C, or G nucleotide. In an embodiment, the poly(A) sequence includes a nucleotide sequence of SEQ ID NO: 18.

In an embodiment, the DNA of the present disclosure includes a nucleotide sequence of SEQ ID NO: 5. In an embodiment, the DNA of the present disclosure includes a nucleotide sequence of SEQ ID NO: 7.

In an embodiment, the DNA of the present disclosure (a) includes a nucleotide sequence having at least 70%, 75%, 80%, 85%, 90%, 95%, 96%, 97%, 98%, or 99% identity to the nucleotide sequence of SEQ ID NO: 5 or 7; and (b) encodes an amino acid sequence having at least 90%, 91%, 92%, 93%, 94%, 95%, 96%, 97%, 98%, or 99% identity to the amino acid sequence of SEQ ID NO: 12.

### Modified Nucleotide

In some embodiment, the mRNA herein includes a modified nucleotide, wherein the modified nucleotide is selected from one or more of the following nucleotides: 2-aminoadenosine, 2-thiothymidine, inosine, pyrrolopyrimidine, 3-methyladenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, N-1-methyl-pseudouridine, 2-thiouridine, and 2-thiocytidine; methylated bases; insertion bases; 2′-fluororibose, ribose, 2′-deoxyribose, arabinose, and hexose; thiophosphoryl and 5′-N-phosphoramidite bond; Moreover, the modified nucleotides described in PCT/CN2020/074825 and PCT/CN2020/106696 are modified.

In an embodiment, the RNA (e.g., an mRNA) of the present disclosure is modified by including one or more modified nucleobases. In an embodiment, the modified nucleobase includes a modified cytosine, a modified uracil, or a combination thereof. In an embodiment, the modified uracil is independently selected from pseudouracil, 1-methyl-pseudouracil, 5-methyl-uracil, or a combination thereof. In an embodiment, the modified cytosine is independently selected from 5-methylcytosine, 5-hydroxymethylcytosine, or a combination thereof. In an embodiment, the proportion of modified nucleobases in the RNA of the present disclosure is 10%-100%, that is, the RNA of the present disclosure may be modified by replacing 10%-100% of the nucleobases therein with modified nucleobases.

In some embodiments, the RNA (e.g., an mRNA) of the present disclosure is modified by replacing one or more uracils with the modified uracil. In an embodiment, the modified uracil includes 1-methyl-pseudouracil, pseudouracil, 5-methyl-uracil, or a combination thereof. In an embodiment, the modified uracil includes pseudouracil. In an embodiment, the modified uracil includes 5-methyl-uracil. In an embodiment, the modified uracil includes 1-methyl-pseudouracil.

In an embodiment, the RNA is modified by replacing at least one uracil with the modified uracil. In an embodiment, the RNA is modified by replacing all uracils with the modified uracil. In an embodiment, the proportion of modified uracils in the RNA is 10%-100%, e.g., 10%, 20%, 30%, 40%, 50%, 60%, 70%, 80%, 90%, or 100%. In an embodiment, the proportion of modified uracils in the RNA is 20%-100%. In an embodiment, 20%-100% of uracils in the RNA are replaced with 1-methyl-pseudouracil. In a preferred embodiment, 100% of uracils in the RNA are replaced with 1-methyl-pseudouracil.

### 1-Methyl-Pseudouridine has the Following Structure:

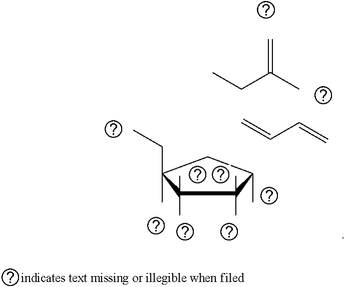

In a specific embodiment, the mRNA of the present disclosure includes a nucleotide sequence of SEQ ID NO: 6, and 100% of uracils therein are replaced with 1-methyl-pseudouracil.

### Use of Lipid Composition and Pharmaceutical Composition

Lipid compositions, pharmaceutical compositions, and injectants may be used for treating diseases, disorders, or conditions. To be precise, these lipid compositions, pharmaceutical compositions, and injectants may be used for treating diseases, disorders, or conditions that feature loss or abnormal protein or polypeptide activity. For example, a lipid composition and a pharmaceutical composition including an mRNA with code loss or abnormal polypeptide may be administered or delivered to a cell. The polypeptide can be produced in the subsequent translation of the mRNA, thereby reducing or eliminating problems due to the absence or aberrant activity of the polypeptide. Since the translation can occur quickly, these methods and the lipid compositions, pharmaceutical compositions, and injectants may be used for treating acute diseases, disorders, or conditions, e.g., sepsis, stroke, and myocardial infarction. The therapeutic agent or the prophylactic agent included in the lipid composition can also alter the rate of transcription of a given mRNA, thereby affecting gene expression.

The diseases, disorders, or conditions for which the lipid compositions, pharmaceutical compositions, and injectants can be administrated and that feature dysfunction or abnormal protein or polypeptide activity include but are not limited to rare diseases, infectious diseases (in a form of vaccines and therapeutic agents), cancer and proliferative diseases, genetic diseases (e.g., cystic fibrosis), autoimmune diseases, diabetes, neurodegenerative diseases, cardiovascular and renal vascular diseases, and metabolic diseases. Various diseases, disorders or conditions may be characterized by the loss of protein activity (or substantial reduction such that appropriate protein functions do not occur). These proteins may be absent, or they may be substantially nonfunctional. Specific instances of dysfunctional proteins are missense mutant variants of a cystic fibrosis transmembrane conductance regulator (CFTR) gene, and these mutant variants produce dysfunctional protein variants of a CFTR protein, thereby causing cystic fibrosis. The present disclosure provides a method of treating such diseases, disorders or conditions in a subject by administering a lipid composition, a pharmaceutical composition or an injectant, the lipid composition includes an RNA and a lipid component, and the lipid component includes a cationic lipid, a phospholipid, a PEG lipid, and a structural lipid, wherein the RNA may be an mRNA encoding a polypeptide that antagonizes or otherwise overcomes abnormal protein activity present in a subject cell.

The method provided by the present disclosure relates to administrating the lipid composition including one or more therapeutic agents or prophylactic agents, and a pharmaceutical composition or injectant including these compositions. For the features and embodiments of the present disclosure, the terms, therapeutic agent and prophylactic agent, are used interchangeably herein. The lipid compositions and pharmaceutical compositions may be administered to a subject using any reasonable amount and any route of administration, and the reasonable amount and the route of administration can effectively achieve the prevention, treatment, and diagnosis of diseases, disorders, or conditions, or be for any other purpose. The specific amount administered to a given subject may vary depending on the species, age and general condition of the subject; the purpose of administration; the specific composition; the mode of application, etc.

In some embodiments, the lipid composition and the pharmaceutical composition including a therapeutic agent or a prophylactic agent of the present disclosure may be administrated to a subject via any method known to those skilled in the art, e.g., parenterally, orally, transmucosally, transdermally, intramuscularly, intravenously, intradermally, subcutaneously, or intraperitoneally. Preferably, the lipid composition of the present disclosure is injected intramuscularly.

In an aspect, the lipid composition, the pharmaceutical composition, or the injectant provided by the present disclosure is used for preventing and/or treating SARS-CoV-2 infection.

In an aspect, the present disclosure further provides use of the lipid composition, the pharmaceutical composition, or the injectant of the present disclosure in preparation of a drug for preventing and/or treating SARS-CoV-2 infection.

In an aspect, the present disclosure further provides a method for preventing and/or treating SARS-CoV-2 infection in a subject, including administrating a therapeutically effective amount of the lipid composition, the pharmaceutical composition, or the injectant of the present disclosure. In an embodiment, the method includes administrating a therapeutically effective amount of a pharmaceutical composition including the mRNA of the present disclosure, in particuar a pharmaceutical composition including the LNP or LPP as described herein.

In an embodiment, the SARS-CoV-2 infection includes at least one infection caused by SARS-CoV-2. In an embodiment, the SARS-CoV-2 includes a SARS-CoV-2 prototype strain and BA.1, BA.2, Beta and Delta variant strains.

In a specific embodiment, the present disclosure provides a method for preventing and/or treating at least one infection caused by SARS-CoV-2 in a subject, including administrating a prophylactically or therapeutically effective amount of the lipid composition, the pharmaceutical composition, or the injectant of the present disclosure. In an embodiment, the SARS-CoV-2 is selected from a SARS-CoV-2 prototype strain and Alpha, Beta, Omicron(BA.1), Omicron(BA.4/5) and Delta variant strains.

In some embodiments, the prophylactically or therapeutically effective amount is provided in one or more administrations. In some embodiments, the prophylactically or therapeutically effective amount is provided in two administrations. In some embodiments, the prophylactically or therapeutically effective amount is provided in three administrations.

In some embodiments, the lipid composition, the pharmaceutical composition, or the injectant of the present disclosure may be administrated in a homologous booster strategy or a heterologous booster strategy. The term “homologous booster” refers to interval inoculation of vaccines using the same technical route. The term “heterologous booster” refers to interval inoculation of vaccines using different technical routes. In an embodiment, the lipid composition, the pharmaceutical composition, or the injectant of the present disclosure is administrated three times to provide the prophylactically or therapeutically effective amount. In an embodiment, the lipid composition, the pharmaceutical composition, or the injectant of the present disclosure is administrated at an interval after one administration of an inactivated vaccine to provide the prophylactically or therapeutically effective amount. In an embodiment, the lipid composition, the pharmaceutical composition, or the injectant of the present disclosure is administrated at an interval after two administrations of an inactivated vaccine to provide the prophylactically or therapeutically effective amount.

### Beneficial Effects

The lipid composition, the lipid composition, the pharmaceutical composition, or the injectant provided by the present disclosure can exhibit excellent effects, for example, but not limited to: (1) improving the expression efficiency of an included mRNA; (2) having high stability under different storage conditions, and keeping the expression efficiency of the included mRNA and the in vitro physicochemical properties of the lipid composition stable; (3) still having high stability after repeated freeze-thawing; (4) still having high stability after lyophilization; (5) good targeting effect and low hepatotoxicity; and (6) resulting in efficient APC uptake and DC maturation.

Further, the lipid composition, the pharmaceutical composition, or the injectant including an mRNA encoding a modified spike protein provided by the present disclosure can exhibit excellent effects, for example, but not limited to: (1) inducing a high level of humoral immune response and a cellular immune response in a subject body; (2) protecting the subject from death and corresponding pathological changes caused by SARS-CoV-2 infection, and having a significant prophylactic effect on the replication of SARS-CoV-2 in a lung; (3) homologous or heterologous booster immunization that can enhance an immune response to variant strains; and (4) having good safety.

### EXAMPLES

The present disclosure is further described by reference to the examples below. It needs to be understood that these examples are only exemplary and do not constitute a limitation to the present disclosure. The following materials and instruments are all commercially available or prepared according to methods well known in the art. The following experiments are performed according to the manufacturer's instructions or according to the methods and steps well known in the art.

### Experimental Materials

The cationic lipid according to formula (I) was synthesized by Stemirna Therapeutics or prepared by reference to, for example, CN110520409A, WO2018081480A1, or US11,246,933B1; phospholipid (DOPE) was purchased from CordenPharma; cholesterol was purchased from Sigma-Aldrich; mPEG2000-DMG (i.e., DMG-PEG 2000) was purchased from Avanti Polar Lipids, Inc.; PBS was purchased from Invitrogen; protamine sulfate was purchased from Beijing Scrianen Pharmaceutical Co., Ltd.; mPEG2000-DSPE was purchased from lipoid GmbH; DSPC was purchased from Avanti Polar Lipids, Inc.

### Example 1 Synthesis of the Compound According to Formula (I)

### General Consideration

Unless otherwise indicated, all solvents and reagents used were commercially available and used as received. ¹H NMR spectra were recorded in CDCl₃ using Bruker Ultrashield 300 MHz instrument at 300 K. Chemical shifts were reported in parts per million (ppm) for ¹H relative to TMS (0.00). Silica gel column chromatography was performed on ISCO CombiFlash Rf+Lumen instrument using ISCO RediSep Rf Gold flash column (particle size: 20-40 microns).

The procedures described below may be used for synthesis of compounds SW-II-115 to SW-II-140-2.

The following abbreviations are used herein:
THF: Tetrahydrofuran
MeCN: Methyl cyanide
LAH: Lithium aluminium hydride
DCM: Dichloromethane
DMAP: 4-Dimethylaminopyridine
LDA: Lithium diisopropylamide
rt: Room temperature
DME: 1,2-Dimethoxyethane
n-BuLi: n-Butyllithium
CPME: Cyclopentyl methyl ether
EDCI N-(3-Dimethylaminopropyl)-N′-ethylcarbodiimide
DIEA: N,N-iisopropylethylamine
PE: Petroleum ether
EA: Ethyl acetate

### A. Compound SW-II-115

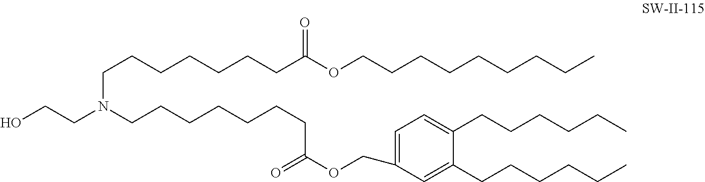 [SMILES: CCCCCCCCCOC(=O)CCCCCCCN(CCO)CCCCCCCC(=O)OCc1ccc(CCCCCC)c(CCCCCC)c1]

### 1. Synthesis of Intermediate 3

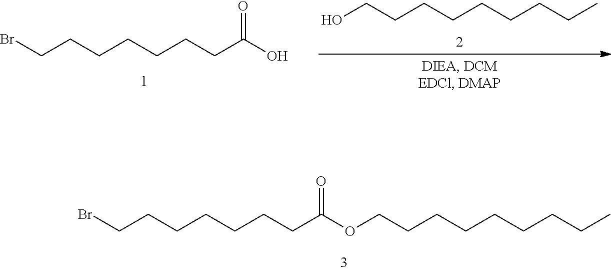

EDCI (17.3 g, 90 mmol, 2 eq.) and DMAP (2.2 g, 18 mmol, 0.4 eq.) were added into a DCM solution (100 mL) containing compound 1 (10 g, 45 mmol, 1 eq.) and compound 2 (7.8 g, 54 mmol, 1.2 eq.), and then DIEA (23.2 g, 180 mmol, 4 eq.) was added. The reaction mixture was stirred at room temperature for 16 hours under N₂ protection. TLC (petroleum ether:ethyl acetate=30:1) showed that compound 1 was consumed and the desired product was formed. The reaction mixture was diluted with DCM (20 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-20:1) to obtain colorless oily compound 3 (4.365 g, 28%).

### 2. Synthesis of Intermediate 5

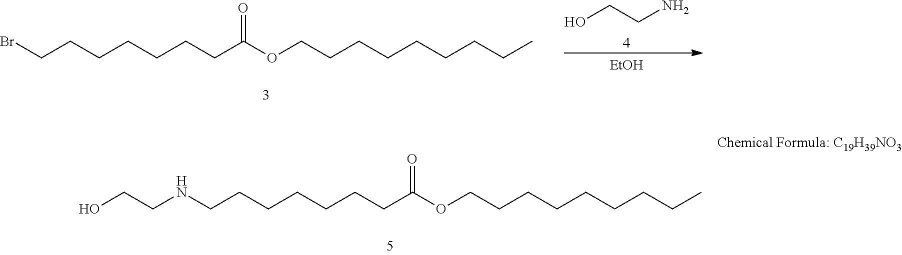

An EtOH solution of compound 3 (500 mg, 1.437 mmol, 1 eq.) and compound 4 (2.63 g, 43.103 mmol, 30 eq.) was stirred at 60° C. for 16 hours under N₂ protection. TLC (DCM:MeOH=10:1) showed that compound 3 was consumed. TLC (DCM/MeOH=10/1) showed that a new major spot was observed. The reaction mixture was concentrated under reduced pressure. The residue was diluted with EtOAc (50 mL) and washed with H₂O (3×50 mL). The organic layer was dried with anhydrous Na₂SO₄, filtered, and concentrated under reduced pressure. The residue was purified by silica gel column chromatography, and eluted with DCM/MeOH (1:0-10:1, v/v) to obtain yellow oily compound 5 (264 mg, 56%).

### 3. Synthesis of Intermediate 8

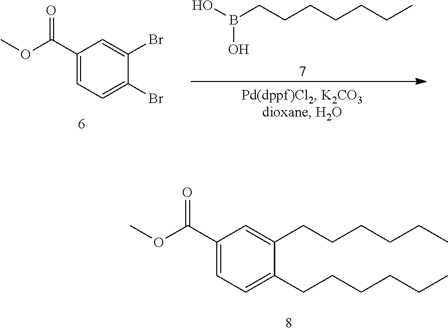

Pd(dppf)Cl₂ (112 mg, 0.171 mmol, 0.1 eq.) and potassium carbonate (709 mg, 5.136 mmol, 3 eq.) were added into a dioxane/water (5 mL/0.5 mL) mixed solvent of compound 6 (500 mg, 1.712 mmol, 1 eq.) and compound 7 (1.113 g, 8.562 mmol, 5 eq.). The mixture was stirred overnight at 100° C. under N₂. TLC (PE:EA=15:1) showed that the reaction was complete and a new major spot was observed. The mixture was extracted with EA and washed with water. The organic layer was dried with anhydrous Na₂SO₄, filtered, and concentrated under vacuum. The residue was purified by silica gel column chromatography, and eluted with PE:EA (1:0-10:1) to obtain colorless oily compound 8 (455 mg, 88%).

### 4. Synthesis of Intermediate 9

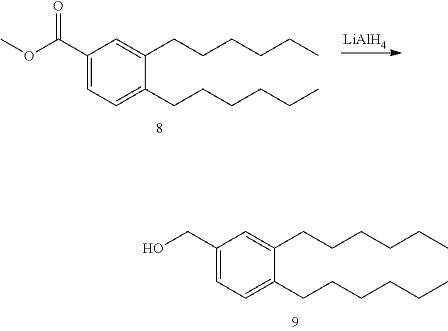 [SMILES: CCCCCCc1ccc(C(=O)OC)cc1CCCCCC ; "LiAlH4" ; CCCCCCc1ccc(CO)cc1CCCCCC]

At 0° C. and under N₂ protection, LiAlH₄(1.5 mL, 1.497 mmol, 1 M, in THF, 1 eq.) was added into a THF (5 mL) solution of compound 8 (455 mg, 1.497 mmol, 1 eq.). The mixture was stirred at room temperature for 2 hours under N₂. TLC (PE:EtOAc=5:1) showed that the reaction was complete and a new major spot was observed. The mixture was quenched with water (1.5 mL) and treated with 2 N HCl to regulate the pH between 6 and 7, extracted with EA and washed with brine. The organic layer was dried with anhydrous Na₂SO₄, filtered, and concentrated under vacuum to obtain crude compound 9 (419 mg, >100%), which was colorless and oily and did not need to be further purified.

### 5. Synthesis of Intermediate 10

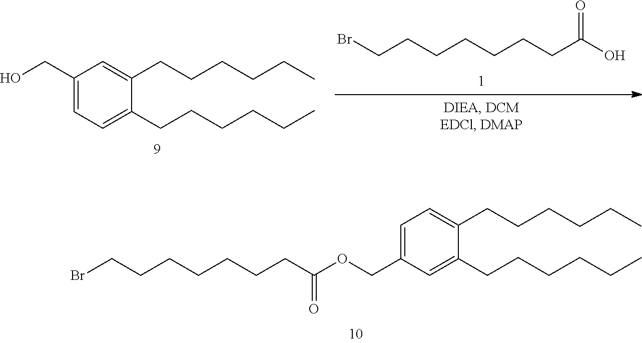 [SMILES: O=C(O)CCCCCCCBr ; CCCCCCc1ccc(CO)cc1CCCCCC ; CCCCCCc1ccc(COC(=O)CCCCCCCBr)cc1CCCCCC]

EDCI (583 mg, 3.036 mmol, 2 eq.) and DMAP (74 mg, 0.607 mmol, 0.4 eq.) were added into a DCM (4 mL) solution containing compound 1 (339 mg, 1.518 mmol, 1 eq.) and compound 9 (419 mg, 1.518 mmol, 1 eq.), and then DIEA (783 mg, 6.072 mmol, 4 eq.) was added. The reaction mixture was stirred at room temperature for 16 hours under N₂ protection. TLC (petroleum ether:ethyl acetate=10:1) showed that the desired product was formed. The reaction mixture was extracted with EA and washed with water. The organic layer was dried with anhydrous Na₂SO₄, filtered, and concentrated under vacuum. The residue was purified by silica gel column chromatography, and eluted with petroleum ether:ethyl acetate (1:0-10:1) to obtain compound 10 (443 mg, 60.7%), which was colorless oil.

### 6. Synthesis of Final Product SW-II-115

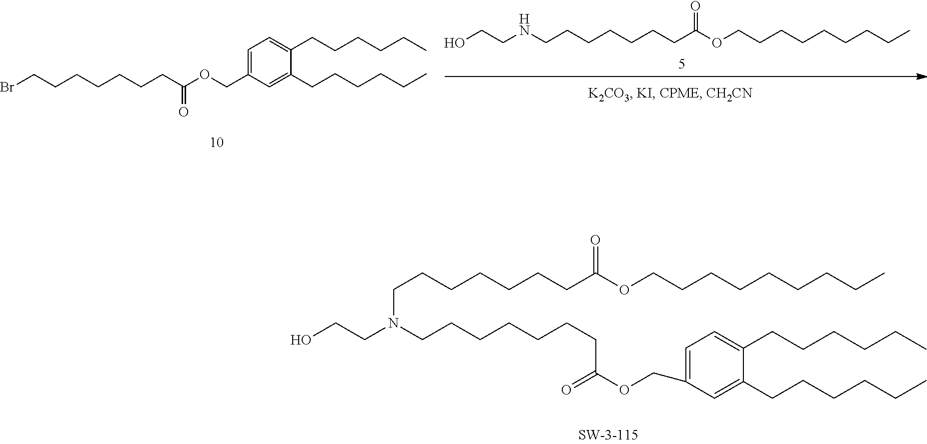

K₂CO₃ (530 mg, 3.84 mmol, 6 eq.) and KI (212 mg, 1.28 mmol, 2 eq.) were added into a mixed solvent CPME/CH₃CN (3 mL/3 mL) containing compound 10 (307 mg, 0.64 mmol, 1 eq.) and compound 5 (210 mg, 0.64 mmol, 1 eq.). After the addition was completed, the mixture was stirred overnight at 90° C. under N₂. TLC (DCM:MeOH=10:1) showed that the reaction was complete and a new major spot was observed. The mixture was extracted with EA and washed with water. The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily compound SW-II-115 (266 mg, 57%).

LCMS: Rt: 1.293 min; MS m/z(ELSD): 730.5[M+H]⁺;

HPLC: 99.472% purity, ELSD; RT=4.895 min.

¹H NMR (400 MHz, CDCl₃) δ7.21-6.99 (m, 3H), 5.05 (s, 2H), 4.05 (t, J=6.8 Hz, 2H), 3.58 (t, J=5.3 Hz, 2H), 2.69-2.46 (m, 10H), 2.31 (dt, J=20.0, 7.5 Hz, 4H), 1.69-1.18 (m, 51H), 0.89 (dt, J=12.4, 6.3 Hz, 9H).

¹³C NMR (101 MHz, CDCl₃) δ173.90(s), 173.68(s), 140.80 (d, J=13.0 Hz), 133.31(s), 129.25 (d, J=16.2 Hz), 128.30(s), 125.75(s), 77.30 (d, J=11.5 Hz), 77.04(s), 76.72(s), 66.22(s), 64.43(s), 58.12(s), 55.72(s), 53.90(s), 34.32 (d, J=1.9 Hz), 32.69(s), 32.48(s), 31.81 (d, J=11.2 Hz), 31.25(s), 29.59-28.91(m), 28.66(s), 27.17(s), 26.64(s), 25.94(s), 24.91 (d, J=5.1 Hz), 22.65 (d, J=3.3 Hz) 14.10(s).

### B. Compound SW-II-118

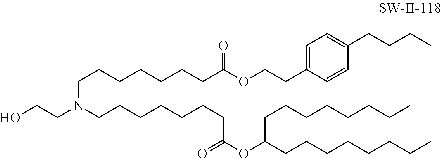 [SMILES: CCCCCCCCC(CCCCCCCC)OC(=O)CCCCCCCN(CCO)CCCCCCCC(=O)OCCc1ccc(CCCC)cc1]

### 1. Synthesis of Intermediate 3

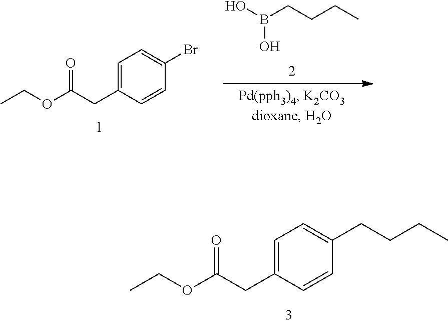

A toluene (10 ml) and H₂O (1 ml) solution of compound 1 (1.22 g, 5.0 mmol, 1.0 eq.), compound 2 (765 mg, 7.5 mmol, 1.5 eq.), Pd(PPh₃)₄ (tetrakis(triphenylphosphine)palladium, 289 mg, 0.25 mmol, 0.05 eq.) and K₂CO₃ (1.38 g, 10.0 mmol, 2.0 eq.) was stirred at 110° C. for 1 hour under N₂ protection. TLC (petroleum ether:ethyl acetate=19:1) showed that compound 1 was consumed and a new spot was observed. The reaction mixture was diluted with DCM (50 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-10:1) to obtain colorless oily compound 3 (0.5 g, 45%).

¹H NMR (400 MHz, CDCl₃) δ7.16 (dd, J=23.5, 8.1 Hz, 4H), 4.14 (q, J=7.1 Hz, 2H), 3.57 (s, 2H), 2.64-2.48 (m, 2H), 1.66-1.51 (m, 2H), 1.35 (dd, J=15.0, 7.4 Hz, 2H), 1.25 (t, J=7.1 Hz, 3H), 0.92 (t, J=7.3 Hz, 3H).

### 2. Synthesis of Intermediate 4

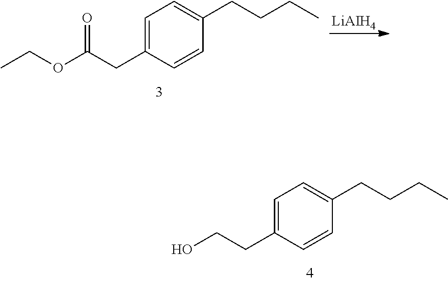

LiAlH₄ (193 mg, 5.09 mmol, 4.0 eq.) was added into a THF (10 mL) solution containing compound 3 (280 mg, 1.27 mmol, 1.0 eq.) at −78° C., and then the reaction mixture reacted at 10° C. for 3 hours. TLC showed that the reaction was very good. The reaction mixture was concentrated, diluted with Na₂SO₄ (20 mL), and extracted with EA (30 mL×2). The organic phase was dried with anhydrous Na₂SO₄, filtered, and concentrated under reduced pressure to obtain yellow oily compound 4 (3.12 g, crude product).

### 3. Synthesis of Intermediate 6

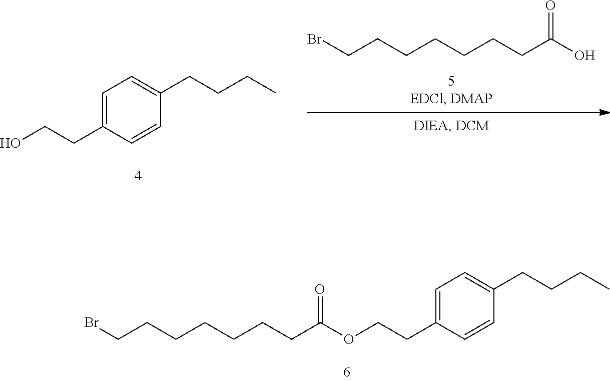 [SMILES: O=C(O)CCCCCCCBr ; CCCCc1ccc(CCO)cc1 ; CCCCc1ccc(CCOC(=O)CCCCCCCBr)cc1]

A DCM (5 mL) solution containing compound 4 (215 mg, 1.2 mmol, 1.0 eq.), compound 5 (404 mg, 1.8 mmol, 1.5 eq.), EDCI (1.15 g, 6.0 mmol, 5.0 eq.), DMAP (732 mg, 1.8 eq.), DIEA (1.29 g, 12.0 mmol, 10.0 eq.) and DIEA (1.29 g, 12.0 mmol, 10.0 eq.) was stirred at 10° C. for 16 hours under N₂ protection. TLC (DCM:MeOH=10:1) showed that the reaction was complete and a new major spot was observed. The mixture was concentrated under reduced pressure. The residue was purified by silica gel column chromatography, and eluted with PE:EA (1:0-10:1, v/v) to obtain colorless oily compound 6 (145 mg, 31%).

¹H NMR (400 MHz, CDCl₃) δ7.12 (s, 4H), 4.27 (t, J=7.1 Hz, 2H), 3.52 (t, J=6.7 Hz, 1H), 3.40 (t, J=6.8 Hz, 1H), 2.90(t, J=7.1 Hz, 2H), 2.65-2.50 (m, 2H), 2.28 (t, J=7.5 Hz, 2H), 1.93-1.70 (m, 2H), 1.64-1.56 (m, 4H), 1.44-1.27 (m, 8H), 0.92 (t, J=7.3 Hz, 3H).

### 4. Synthesis of Final Product SW-II-118

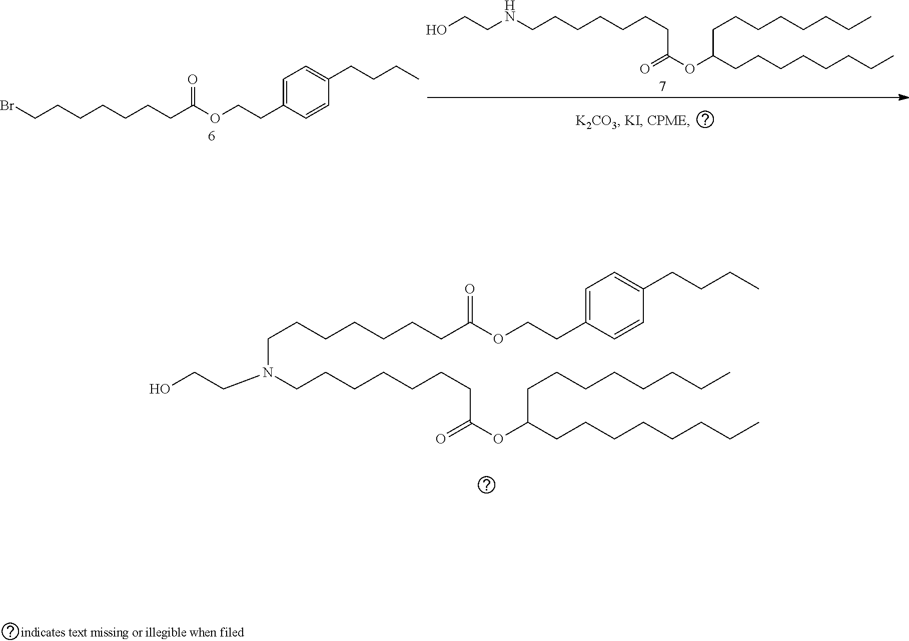 [SMILES: CCCCc1ccc(CCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccc(CCCC)cc1]

A CPME (1 mL) and CH₃CN (1 mL) mixed solvent containing a mixture of compound 6 (140 mg, 0.37 mmol, 1.0 eq.), compound 7 (243 mg, 0.55 mmol, 1.5 eq.), K₂CO₃ (153 mg, 1.11 mmol, 3.0 eq.) and KI (123 mg, 0.74 mmol, 2.0 eq.) was stirred at 90° C. for 16 hours under N₂. The reaction mixture was concentrated under reduced pressure. The residue was diluted with EtOAc (50 mL) and washed with NaHCO₃ (30 mL). The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily SW-II-118 (105 mg, 61%).

LCMS: Rt: 1.946 min; MS m/z(ELSD): 744.4[M+H]⁺;

HPLC: 99.64% purity, ELSD; RT=5.875 min.

¹H NMR (400 MHz, CDCl₃) δ7.11 (s, 4H), 4.91-4.79 (m, 1H), 4.26 (t, J=7.2 Hz, 2H), 3.80-3.68 (m, 2H), 2.90(t, J=7.1 Hz, 4H), 2.81-2.67 (m, 4H), 2.62-2.52 (m, 2H), 2.28(td, J=7.5, 2.6 Hz, 4H), 1.64-1.51 (m, 11H), 1.38-1.17 (m, 42H), 0.93-0.82 (m, 9H).

¹³C NMR (101 MHz, CDCl₃) δ 173.61(d, J=11.7 Hz), 141.11(s), 134.90(s), 128.74(s), 128.51(s), 77.40(s), 77.08(s), 76.77(s), 74.17(s), 64.90(s), 57.48(s), 56.24(s), 53.98(s), 35.25(s), 34.66(d, J=14.4 Hz), 34.16(d, J=5.1 Hz), 33.67(s), 31.86(s), 29.52(d, J=2.4 Hz), 29.24(s), 29.21-28.74(m), 26.90(d, J=4.9 Hz), 25.42-24.92(m), 24.92-24.88(m) 24.74(s), 22.67(s), 22.37(s), 14.04(d, J=15.7 Hz).

### C. Compound SW-II-120

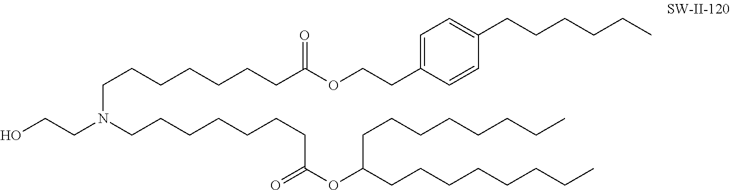 [SMILES: CCCCCCCCC(CCCCCCCC)OC(=O)CCCCCCCN(CCO)CCCCCCCC(=O)OCCc1ccc(CCCCCC)cc1]

### 1. Synthesis of Intermediate 3

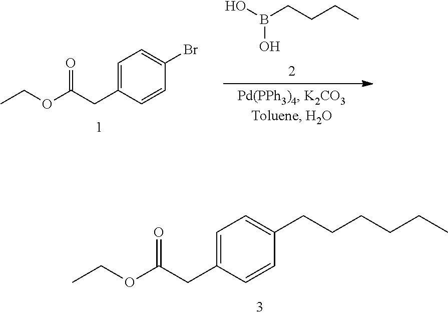

A toluene (10 ml) and H₂O (1 ml) mixed solution containing compound 1 (1.22 g, 5.0 mmol, 1.0 eq.), compound 2 (1.30 mg, 10.0 mmol, 2.0 eq.), Pd(PPh₃)₄ (289 mg, 0.25 mmol, 0.05 eq.) and K₂CO₃ (1.38 g, 10.0 mmol, 2.0 eq.) was stirred at 110° C. for 1 hour under N₂ protection. TLC (petroleum ether:ethyl acetate=19:1) showed that compound 1 was consumed and a new spot was observed. The reaction mixture was diluted with DCM (50 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-10:1) to obtain colorless oily compound 3 (0.78 g, 62%).

¹H NMR (400 MHz, CDCl₃) δ7.19 (d, J=8.1 Hz, 2H), 7.13 (d, J=8.1 Hz, 2H), 4.14(q, J=7.1 Hz, 2H), 3.57 (s, 2H), 2.62-2.51 (m, 2H), 1.58(d, J=11.1 Hz, 2H), 1.35-1.21 (m, 9H), 0.88 (t, J=6.7 Hz, 3H).

### 2. Synthesis of Intermediate 4

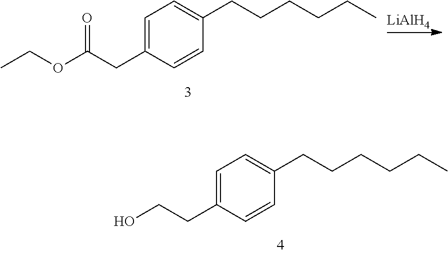

LiAlH₄ (477 mg, 12.56 mmol, 4.0 eq.) was added into a THF (10 mL) solution containing compound 3 (780 mg, 3.14 mmol, 1.0 eq.) at −78° C., and then the reaction mixture was stirred at 10° C. for 3 hours. Thin layer chromatography showed that the reaction proceeded well. The reaction mixture was concentrated, diluted with Na₂SO₄ (20 mL), and extracted with EA (30 mL*2). The organic phase was dried with anhydrous Na₂SO₄, filtered, and concentrated under reduced pressure to obtain colorless oily compound 4 (640 mg, crude product).

### 3. Synthesis of Intermediate 6

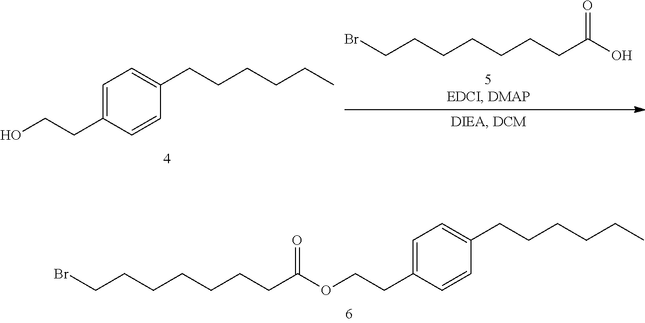 [SMILES: O=C(O)CCCCCCCBr ; CCCCCCc1ccc(CCO)cc1 ; CCCCCCc1ccc(CCOC(=O)CCCCCCCBr)cc1]

A DCM (10 mL) solution containing compound 4 (640 mg, 3.10 mmol, 1.0 eq.), compound 5 (1.06 g, 4.70 mmol, 1.5 eq.), EDCI (2.98 g, 15.5 mmol, 5.0 eq.), DMAP (1.85 g, 15.0 eq.) and DIEA (4.0 g, 31.0 mmol, 10.0 eq.) was stirred at 10° C. for 16 hours under N₂ protection. TLC (DCM:MeOH=10:1) showed that the reaction was complete and a new major spot was observed. The mixture was concentrated under reduced pressure. The residue was purified by silica gel column chromatography, and eluted with PE:EA (1:0-10:1, v/v) to obtain colorless oily compound 6 (465 mg, 36%).

### 4. Synthesis of Final Product SW-II-120

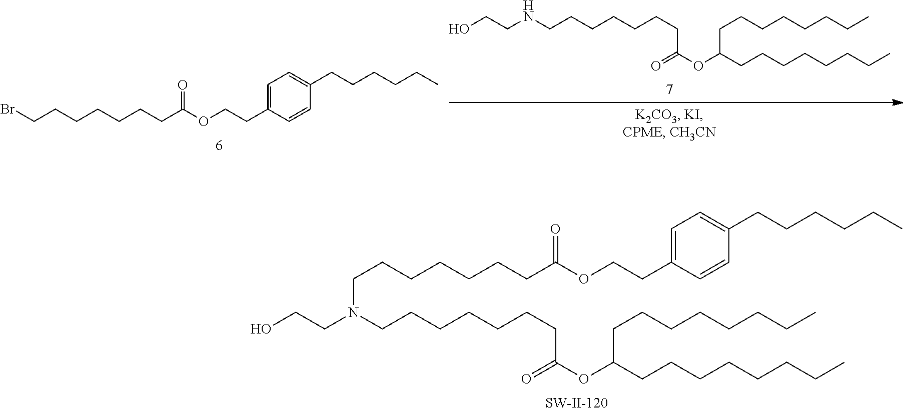 [SMILES: CCCCCCCCC(CCCCCCCC)OC(=O)CCCCCCCNCCO ; CCCCCCc1ccc(CCOC(=O)CCCCCCCBr)cc1 ; CCCCCCCCC(CCCCCCCC)OC(=O)CCCCCCCN(CCO)CCCCCCCC(=O)OCCc1ccc(CCCCCC)cc1]

A CPME(1 mL) and CH₃CN(1 mL) mixed solvent containing a mixture of compound 6 (100 mg, 0.25 mmol, 1.0 eq.), compound 7 (161 mg, 0.36 mmol, 1.5 eq.), K₂CO₃ (104 mg, 0.75 mmol, 3.0 eq.) and KI (83 mg, 0.50 mmol, 2.0 eq.) was stirred at 90° C. for 16 hours under N₂. The reaction mixture was concentrated under reduced pressure. The residue was diluted with EtOAc (50 mL) and washed with NaHCO₃ (30 mL). The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily SW-II-120 (100 mg, 52%).

LCMS: Rt: 2.500 min; MS m/z(ELSD): 772.4[M+H]⁺;

HPLC: 99.70% purity, ELSD; RT=8.675 min.

¹H NMR (400 MHz, CDCl₃) δ7.07 (d, J=8.9 Hz, 4H), 4.89-4.73 (m, 1H), 4.23 (t, J=7.2 Hz, 2H), 3.83-3.65 (m, 2H), 2.87 (t, J=7.2 Hz, 4H), 2.82-2.67 (m, 4H), 2.61-2.45 (m, 2H), 2.25(td, J=7.5, 2.5 Hz, 4H), 1.65-1.44 (m, 15H), 1.27 (dd, J=13.2, 11.3 Hz, 42H), 0.85 (t, J=6.8 Hz, 9H).

¹³C NMR (101 MHz, CDCl₃) δ 173.57(d, J=11.5 Hz), 141.13(s), 134.88(s), 128.73(s), 128.48(s), 77.45(s), 77.13(s), 76.81(s), 74.14(s), 64.89(s), 57.34(s), 56.17(s), 53.92(s), 35.57(s), 34.64(d, J=16.1 Hz), 34.14(d, J=3.3 Hz), 31.79(d, J=13.4 Hz), 31.49(s), 29.50(d, J=2.2 Hz), 29.23(s), 29.10-28.71(m), 26.85(d, J=5.0 Hz), 25.49-25.38(m), 25.13(d, J=35.4 Hz), 24.72(s), 22.63(d, J=5.8 Hz), 14.11(s).

### D. Compound SW-II-121

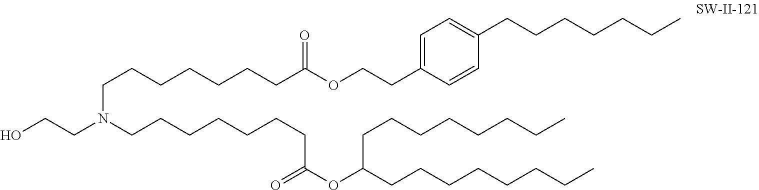 [SMILES: CCCCCCCCC(CCCCCCCC)OC(=O)CCCCCCCN(CCO)CCCCCCCC(=O)OCCc1ccc(CCCCCCC)cc1]

### 1. Synthesis of Intermediate 3

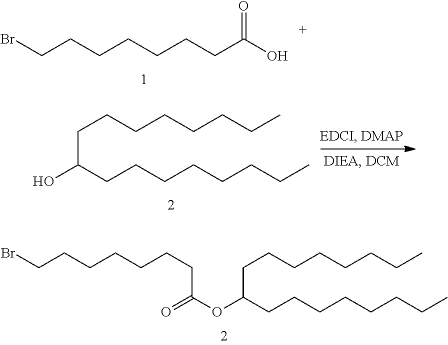 [SMILES: O=C(O)CCCCCCCBr ; CCCCCCCCC(O)CCCCCCCC ; CCCCCCCCC(CCCCCCCC)OC(=O)CCCCCCCBr]

EDCI (1.495 g, 7.8 mmol, 2.0 eq.), DMAP (0.19 g, 1.56 mmol, 0.4 eq.) and DIEA (2.57 mL, 15.6 mmol, 4.0 eq.) were added into a DCM (20 mL) solution containing compound 1 (1.3 g, 5.86 mmol, 1.5 eq.) and compound 2 (1 g, 3.9 mmol, 1.0 eq.). The reaction mixture was stirred at room temperature for 16 hours under N₂. TLC (petroleum ether:ethyl acetate=19:1) showed that compound 2 was consumed and the desired product was formed. The reaction mixture was diluted with DCM (20 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-10:1) to obtain yellow oily compound 3 (1.2 g, 66.9%).

¹H NMR (400 MHz, CDCl₃) δ4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Intermediate 5

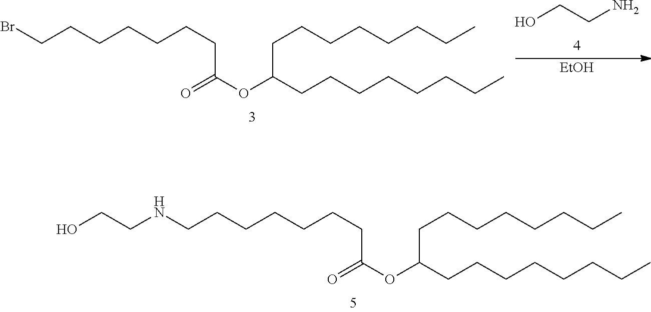

An EtOH (5 mL) solution containing compound 3 (5.2 g, 11.30 mmol, 1.0 eq.) and compound 4 (20.6 g, 339 mmol, 30 eq.) was stirred at 60° C. for 16 hours under N₂ protection. TLC (petroleum ether:ethyl acetate=19:1) showed that compound 3 was consumed. Moreover, TLC (DCM/MeOH=10/1) showed that a new major spot was observed. The reaction mixture was concentrated under reduced pressure. The residue was diluted with EtOAc (50 mL) and washed with H₂O (3×50 mL). The organic layer was dried with anhydrous Na₂SO₄, filtered, and concentrated under vacuum. The residue was purified by silica gel column chromatography, and eluted with DCM:MeOH (1:0-10:1, v/v) to obtain yellow oily compound 5 (3 g, 60%).

### 3. Synthesis of Intermediate 8

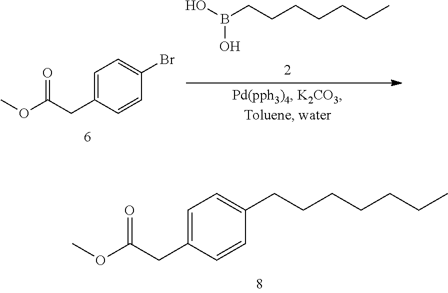

Pd(pph₃)₄ (238 mg, 0.206 mmol, 0.05 eq.) and K₂CO₃ (1.7 g, 12.35 mmol, 3 eq.) were added into a toluene/water (10 mL/1 mL) mixed solution containing compound 6 (1 g, 4.115 mmol, 1 eq.) and compound 7 (889 mg, 6.173 mmol, 1.5 eq.). The mixture was stirred at 110° C. for 2 hours under N₂. TLC (PE:EA=10:1) showed that the reaction was complete and a new major spot was observed. The mixture was extracted with EA and washed with water. The organic layer was dried with anhydrous Na₂SO₄, filtered, and concentrated under vacuum. The residue was purified by silica gel column chromatography, and eluted with PE:EA (1:0-10:1) to obtain colorless oily compound 8 (714 mg, 66%).

### 4. Synthesis of Intermediate 9

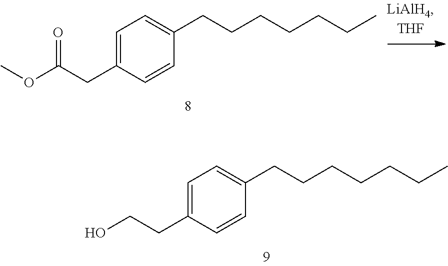 [SMILES: CCCCCCCc1ccc(CC(=O)OC)cc1 ; CCCCCCCc1ccc(CCO)cc1]

LiAlH₄ (2.7 mL, 2.725 mmol, 1 M, in THF, 1 eq.) was added into a mixture of compound 8 (714 mg, 2.725 mmol, 1 eq.) in a THF (7 mL) solution at 0° C. under N₂ protection. The mixture was stirred at room temperature for 2 hours. TLC (PE:EtOAc=10:1) showed that the reaction was complete and a new major spot was observed. The mixture was quenched with water (2.7 mL) and treated with 2 N HCl to regulate the pH between 6 and 7, extracted with EA and washed with brine. The organic layer was dried with anhydrous Na₂SO₄, filtered, and concentrated under vacuum. The residue was purified by silica gel column chromatography, and eluted with PE:EA (1:0-10:1) to obtain colorless oily compound 9 (103 mg, 63%).

### 5. Synthesis of Intermediate 11

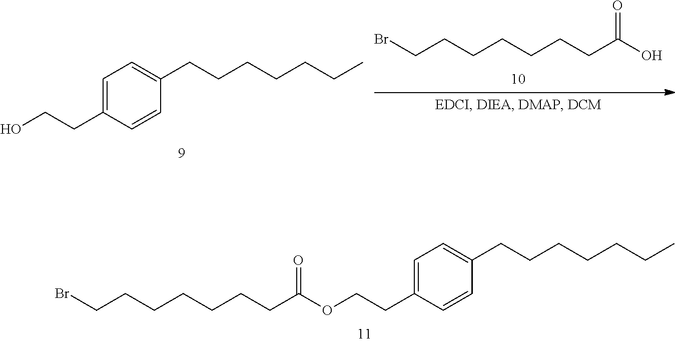

EDCI (524 mg, 2.728 mmol, 2 eq.), DMAP (67 mg, 0.546 mmol, 0.4 eq.), and DIEA (704 mg, 5.456 mmol, 4 eq.) were added into DCM (3 mL) containing compound 9 (300 mg, 1.364 mmol, 1 eq.) and compound 10 (363 mg, 1.64 mmol, 1.2 eq.). The reaction mixture was stirred at room temperature for 16 hours under N₂. TLC (petroleum ether:ethyl acetate=10:1) showed that the desired product was formed. The reaction mixture was extracted with EA and washed with water. The organic layer was dried with anhydrous Na₂SO₄, filtered, and concentrated under vacuum. The residue was purified by silica gel column chromatography, and eluted with petroleum ether:ethyl acetate (1:0-10:1) to obtain colorless oily compound 11 (169 mg, 29%).

### 6. Synthesis of Final Product SW-II-121

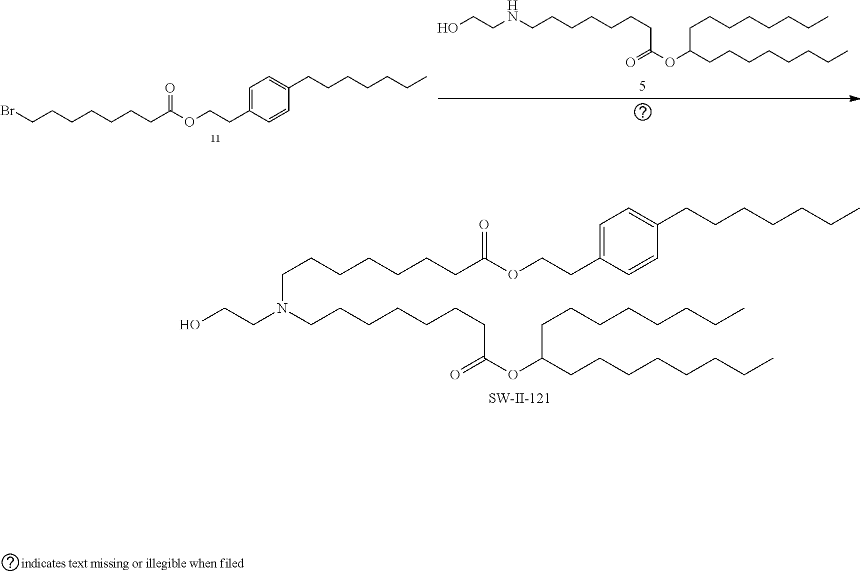 [SMILES: CCCCCCCCC(CCCCCCCC)OC(=O)CCCCCCCNCCO ; CCCCCCCc1ccc(CCOC(=O)CCCCCCCBr)cc1 ; CCCCCCCCC(CCCCCCCC)OC(=O)CCCCCCCN(CCO)CCCCCCCC(=O)OCCc1ccc(CCCCCCC)cc1]

K₂CO₃ (330 mg, 2.394 mmol, 6 eq.) and KI (132 mg, 0.798 mmol, 2 eq.) were added into a CPME/CH₃CN (2 mL/2 mL) mixed solvent containing compound 11 (169 mg, 0.399 mmol, 1 eq.) and compound 5 (176 mg, 0.399 mmol, 1 eq.). After the addition was completed, the mixture was stirred overnight at 90° C. under N₂. TLC (DCM:MeOH=10:1) showed that the reaction was complete and a new major spot was observed. The mixture was extracted with EA and washed with water. The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily compound SW-II-121 (145 mg, 46%).

LCMS: Rt: 1.493 min; MS m/z(ELSD): 786.5[M+H]⁺;

HPLC: 99.869% purity, ELSD; RT=10.655 min.

¹H NMR (400 MHz, CDCl₃) δ 7.11 (s, 4H), 4.92-4.80 (m, 1H), 4.26 (t, J=7.2 Hz, 2H), 3.80 (s, 2H), 2.87 (dd, J=26.6, 19.4 Hz, 7H), 2.62-2.51 (m, 2H), 2.28(td, J=7.2, 3.6 Hz, 4H), 1.75-1.45 (m, 14H), 1.42-1.09 (m, 45H), 0.88 (t, J=6.8 Hz, 9H).

¹³C NMR (101 MHz, CDCl₃) δ 173.61(d, J=12.3 Hz), 141.20(s), 134.90(s), 128.75(s), 128.51(s), 77.35(s), 77.03(s), 76.72(s), 74.21(s), 64.93(s), 54.15(s), 35.59(s), 34.66(d, J=16.6 Hz), 34.16(d, J=3.0 Hz), 31.85(d, J=4.4 Hz), 31.55(s), 29.64-29.15(m), 29.15-28.78(m), 26.85(d, J=4.5 Hz), 25.33(s), 24.95(s), 24.72(s), 22.68(s), 14.12(s).

### E. Compound SW-II-122

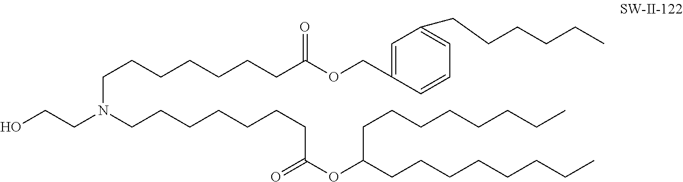 [SMILES: CCCCCCCCC(CCCCCCCC)OC(=O)CCCCCCCN(CCO)CCCCCCCC(=O)OCc1cccc(CCCCCC)c1]

### 1. Synthesis of Compound 3

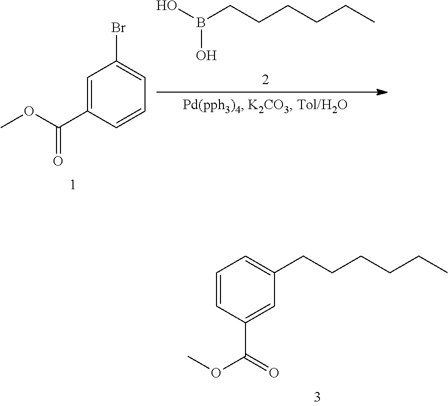

Compound 1 (1 g, 4.65 mmol, 1 eq.) and compound 2 (726 mg 5.58 mmol, 1.2 eq.) were dissolved in toluene/water (10/1, 20 mL), and then K₂CO₃ (1.92 g, 13.9 mmol, 3 eq.) and Pd(pph₃)₄ (269 mg, 0.23 mmol, 0.05 eq.) were added into the mixture. The reaction mixture was placed in N₂, heated to 110° C., and stirred for 2 hours. TLC (petroleum ether/ethyl acetate=19/1) showed that compound 1 was consumed and a new spot was observed. The reaction mixture was quenched with H₂O (80 mL) and extracted with ethyl acetate (60 mL×3). The organic layer was dried with anhydrous sodium sulfate, filtered, and concentrated under reduced pressure. The residue was purified by silica gel column chromatography, and eluted with petroleum ether/ethyl acetate (1/0-10/1) to obtain yellow oily compound 3 (800 mg, 78%).

### 2. Synthesis of Compound 4

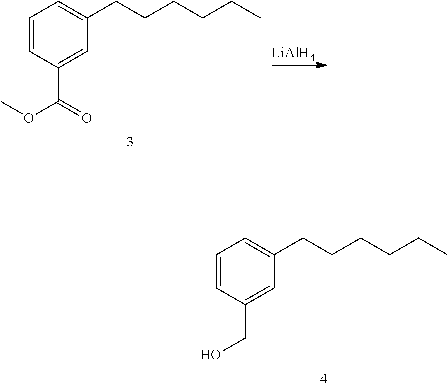

LiAlH₄ (3.2 mL 3.18 mmol, 1 eq.) was added into compound 3 (700 mg, 3.18 mmol, 1.0 eq.) dissolved in THF (14 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with water (3.2 mL) and 1 M HCl (3.2 mL) respectively. Water (6 mL) was then added into the mixture. The mixture was extracted with ethyl acetate (60 mL×3). The organic layer was washed with brine (30 mL×2), dried with anhydrous sodium sulfate, filtered, and concentrated under reduced pressure. The residue was purified by silica gel column chromatography, and eluted with ethyl acetate/petroleum ether=1/10 to obtain yellow oily compound 4 (600 mg, 98%).

### 3. Synthesis of Compound 6

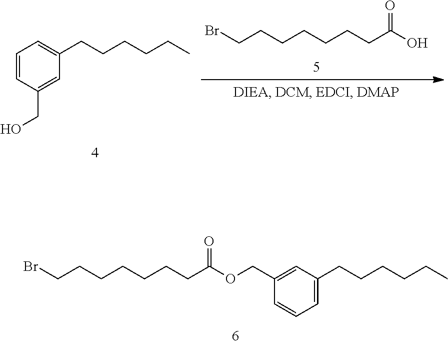 [SMILES: CCCCCCc1cccc(CO)c1 ; O=C(O)CCCCCCCBr ; CCCCCCc1cccc(COC(=O)CCCCCCCBr)c1]

Compound 4 (680 mg, 3.5 mmol, 1.0 eq.) and compound 5 (1.13 g, 5.1 mmol, 1.5 eq.) were dissolved in DCM (10 mL). EDCI (1.20 g, 6.25 mmol, 2.0 eq.), DMAP (166 mg, 1.36 mmol, 0.4 eq.), and DIEA (1.78 g, 13.8 mmol, 4.0 eq.) were added into the mixture. After the addition was completed, the reaction mixture was stirred overnight at room temperature under nitrogen protection. TLC (DCM/MeOH=30/1) showed that a starting material was consumed and a new spot was formed. The mixture was quenched with water (70 mL) and extracted with DCM (80 mL×3). The combined organic layer was washed with brine (2×20 mL), dried with anhydrous sodium sulfate, filtered, and concentrated under reduced pressure. The residue was purified by silica gel column chromatography, and eluted with a solution with ethyl acetate/petroleum ether=3/97 to obtain yellow oily compound 6 (680 mg, 48.5%).

### 4. Synthesis of SW-II-122

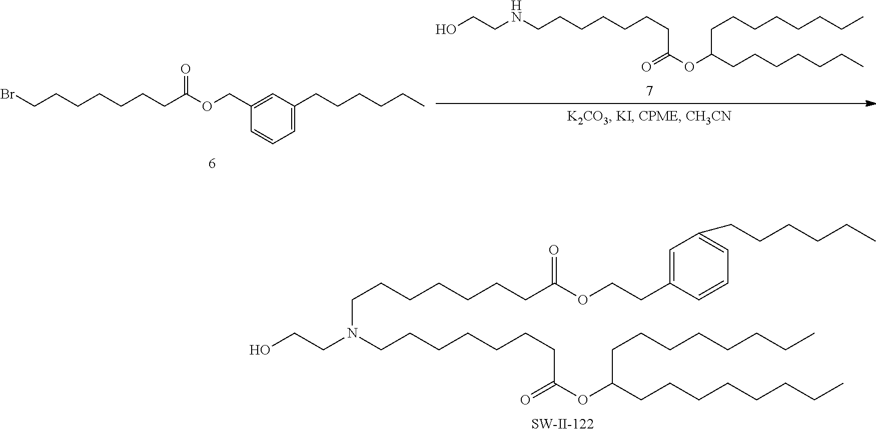 [SMILES: CCCCCCCCC(CCCCCCC)OC(=O)CCCCCCCNCCO ; CCCCCCc1cccc(COC(=O)CCCCCCCBr)c1 ; CCCCCCCCC(CCCCCCCC)OC(=O)CCCCCCCN(CCO)CCCCCCCC(=O)OCCc1cccc(CCCCCC)c1]

Compound 6 (108 mg, 0.27 mmol, 1.2 eq.) and compound 7 (100 mg, 0.23 mmol, 1 eq.) were dissolved in a CPME (2 mL) and CH₃CN (2 mL) mixed solvent. Potassium carbonate (157 mg, 1.14 mmol, 5.0 eq.) and potassium iodide (75 mg, 0.45 mmol, 2.0 eq.) were added into the mixture. After the addition was completed, the reaction mixture was stirred at 90° C. for 16 hours under nitrogen protection. TLC (DCM/MeOH=10/1) showed that the reaction was complete. The reaction mixture was concentrated under reduced pressure. The residue was purified by silica gel column chromatography, and eluted with DCM/MeOH (1/0-10:1, v/v) to obtain SW-II-122 (68 mg, 40%), which was colorless and oily.

LCMS: Rt: 1.487 min; MS m/z(ELSD): 758.5[M+H]⁺;

HPLC: 97.3% purity, ELSD; RT=7.622 min.

¹H NMR (400 MHz, CDCl₃) δ7.32 (d, J=26.4 Hz, 1H), 7.17(dd, J=27.2, 21.1 Hz, 3H), 5.09 (s, 2H), 4.91-4.79 (m, 1H), 3.85 (s, 2H), 2.98 (s, 2H), 2.87 (s, 4H), 2.65-2.54 (m, 2H), 2.35 (t, J=7.6 Hz, 2H), 2.28 (t, J=7.6 Hz, 2H), 1.74-1.57 (m, 9H), 1.50 (d, J=5.6 Hz, 4H), 1.37-1.15 (m, 43H), 0.94-0.80 (m, 9H).

¹³C NMR (101 MHz, CDCl₃) δ 173.55(d, J=2.4 Hz), 143.35(s), 135.92(s), 128.67-128.19(m), 125.47(s), 77.36(s), 77.04(s), 76.73(s), 74.22(s), 66.27(s), 57.15(s), 56.74(s), 54.14(s), 35.88(s), 34.55(s), 34.15(d, J=3.6 Hz), 31.79(d, J=15.2 Hz), 31.43(s), 29.52(d, J=2.8 Hz), 29.25(s), 28.92(dd, J=14.2, 5.8 Hz), 26.77(d, J=4.8 Hz), 25.33(s), 24.92(s), 24.71(s), 24.48(s), 22.64(d, J=6.8 Hz), 14.12(s).

### F. Compound SW-II-127

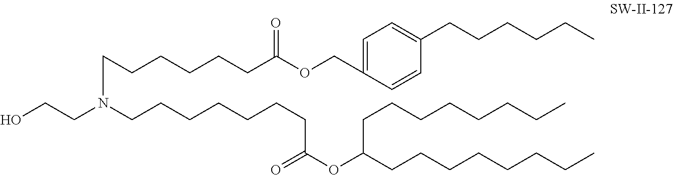 [SMILES: CCCCCCCCC(CCCCCCCC)OC(=O)CCCCCCCN(CCO)CCCCCCC(=O)OCc1ccc(CCCCCC)cc1]

### 1. Synthesis of Compound 3

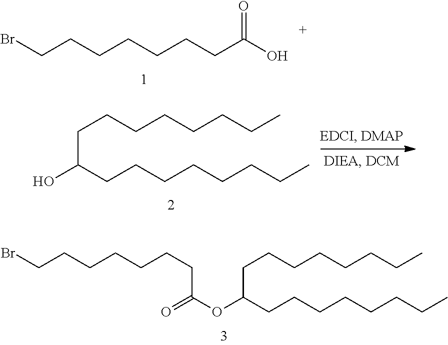 [SMILES: O=C(O)CCCCCCCBr ; CCCCCCCCC(O)CCCCCCCC ; CCCCCCCCC(CCCCCCCC)OC(=O)CCCCCCCBr]

Compound 1 (1.3 g, 5.86 mmol, 1.5 eq.) and compound 2 (1 g, 3.9 mmol, 1.0 eq.) were dissolved in DCM (20 mL). EDCI (1.495 g, 7.8 mmol, 2.0 eq.) and DMAP (0.19 g, 1.56 mmol, 0.4 eq.) were added into the mixture. Then, DIEA (2.57 mL, 15.6 mmol, 4.0 eq.) was added. The reaction mixture was stirred at room temperature for 16 hours under nitrogen protection. TLC (petroleum ether/ethyl acetate=19/1) showed that compound 2 was consumed and the desired product was formed. The reaction mixture was diluted with DCM (20 mL), washed with H₂O (40 mL), dried with anhydrous Na₂SO₄, filtered, and concentrated under reduced pressure. The residue was purified by silica gel column chromatography, and eluted with petroleum ether/ethyl acetate (1/0-10/1) to obtain yellow oily compound 3 (1.2 g, 66.9%).

¹H NMR (400 MHz, CDCl₃) δ4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Compound 5

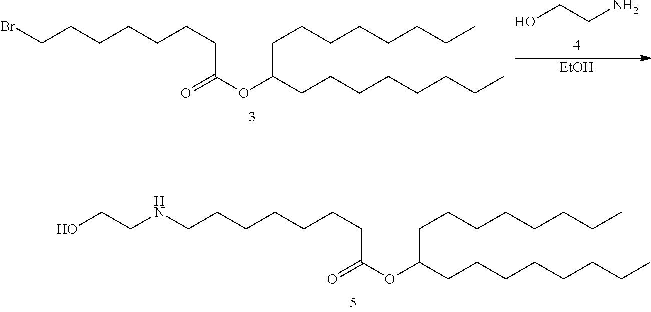

Compound 3 (5.2 g, 11.30 mmol, 1.0 eq.) and compound 4 (20.6 g, 339 mmol, 30 eq.) were added into EtOH (5 mL). Then, the mixture was stirred at 60° C. for 16 hours under nitrogen protection. TLC (petroleum ether/ethyl acetate=19/1) showed that compound 3 was consumed. Moreover, TLC (DCM/MeOH=10/1) showed that a new major spot was observed. The reaction mixture was concentrated under reduced pressure. The residue was diluted with EtOAc (50 mL) and washed with H₂O (3×50 mL). The organic layer was dried with anhydrous sodium sulfate, filtered, and concentrated under reduced pressure. The residue was purified by silica gel column chromatography, and eluted with DCM/MeOH (1/0-10:1, v/v) to obtain yellow oily compound 5 (3 g, 60%).

¹H NMR(400 MHz, CDCl₃) δ4.95-4.75 (m, 1H), 3.74-3.58 (m, 2H), 2.87-2.74 (m, 2H), 2.69-2.56 (m, 2H), 2.36 (s, 2H), 2.28 (t, J=7.5 Hz, 2H), 1.65-1.42 (m, 8H), 1.38-1.17 (m, 30H), 0.88 (t, J=6.8 Hz, 6H).

### 3. Synthesis of Compound 8

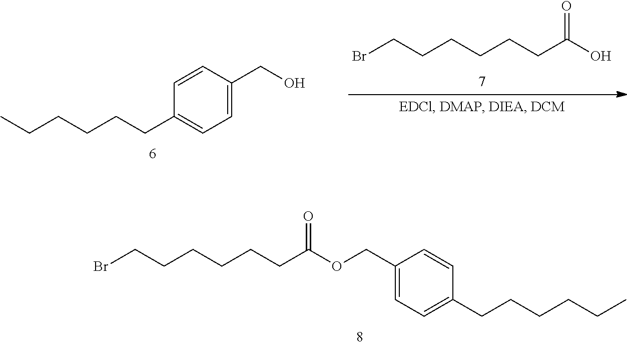 [SMILES: O=C(O)CCCCCCBr ; CCCCCCc1ccc(CO)cc1 ; CCCCCCc1ccc(COC(=O)CCCCCCBr)cc1]

Compound 7 (522 mg, 2.5 mmol, 1.2 eq.) and compound 6 (400 mg, 2.083 mmol, 1 eq.) were dissolved in DCM (4 mL). EDCI (800 mg, 4.166 mmol, 2 eq.), DMAP (102 mg, 0.833 mmol, 0.4 eq.), and DIEA (1.075 mg, 8.332 mmol, 4 eq.) were added into the mixture. After the addition was completed, the reaction mixture was stirred overnight at room temperature under nitrogen protection. TLC (PE:EA=10:1) showed that a starting material was consumed and a new spot was formed. The reaction mixture was concentrated under reduced pressure. The residue was purified by silica gel column chromatography, and eluted with PE/EA (1/0-10/1) to obtain colorless oily compound 8 (454 mg, 57%).

### 4. Synthesis of SW-II-127

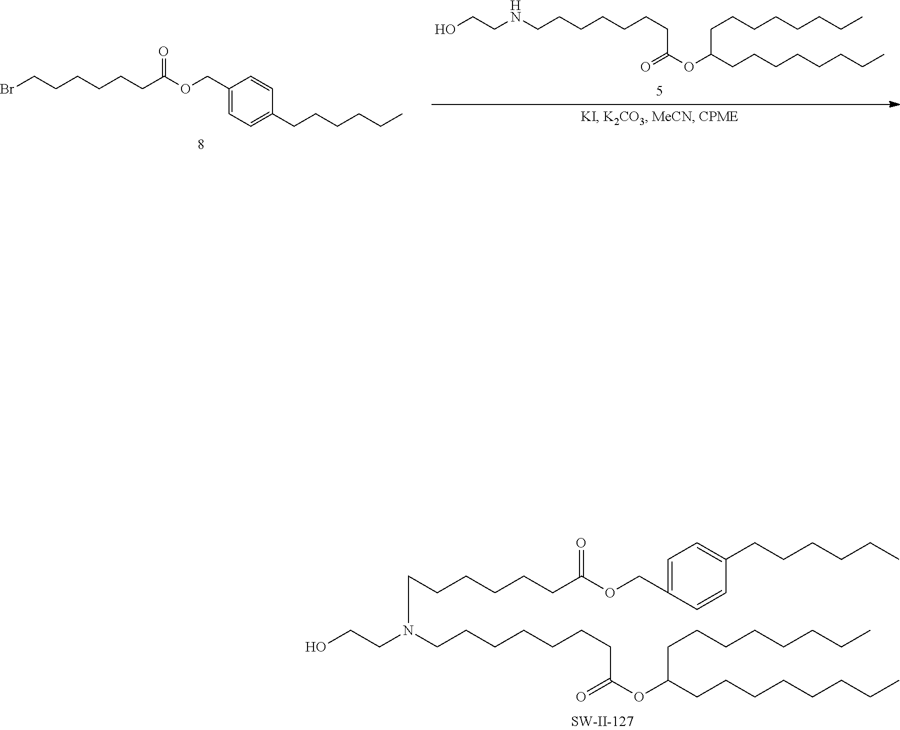 [SMILES: CCCCCCc1ccc(COC(=O)CCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCC(=O)OCc1ccc(CCCCCC)cc1]

Compound 8 (100 mg, 0.262 mmol, 1 eq.) and compound 5 (139 mg, 0.314 mmol, 1.2 eq.) were dissolved in CPME/CH3CN (1 mL/1 mL). Potassium carbonate (217 mg, 1.572 mmol, 6 eq.) and potassium iodide (87 mg, 0.524 mmol, 2 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10:1, v/v) to obtain yellow oily compound SW-II-127 (42.49 mg, 22%).

LCMS: Rt: 1.323 min; MS m/z(ELSD): 744.5[M+H]⁺;

HPLC: 99.742% purity, ELSD; RT=7.339 min.

¹H NMR (400 MHz, CDCl₃) δ7.25 (s, 2H), 7.17(d, J=8.0 Hz, 2H), 5.07 (s, 2H), 4.91-4.82 (m, 1H), 3.83 (s, 2H), 2.90 (d, J=44.8 Hz, 5H), 2.64-2.55 (m, 2H), 2.35 (t, J=7.4 Hz, 2H), 2.28 (t, J=7.5 Hz, 2H), 1.76-1.46 (m, 14H), 1.42-1.19 (m, 41H), 0.88 (t, J=6.8 Hz, 9H).

¹³C NMR (101 MHz, CDCl₃) δ 173.50(d, J=8.5 Hz), 133.17(s), 128.61(s), 128.34(s), 77.29(d, J=11.4 Hz), 77.03(s), 76.71(s), 74.23(s), 66.19(s), 54.20(s), 35.71(s), 34.56(s), 34.10(d, J=8.8 Hz), 31.80(d, J=15.4 Hz), 31.43(s), 29.53(d, J=2.5 Hz), 29.25(s), 28.95(d, J=10.5 Hz), 28.63(s), 26.71(d, J=18.2 Hz), 25.33(s), 24.93(s), 24.62(s), 22.65(d, J=6.6 Hz), 14.13(s).

### G. Compound SW-II-134-1

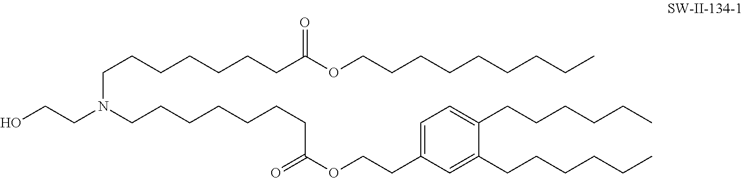 [SMILES: CCCCCCCCCOC(=O)CCCCCCCN(CCO)CCCCCCCC(=O)OCCc1ccc(CCCCCC)c(CCCCCC)c1]

### 1. Synthesis of Compound 3

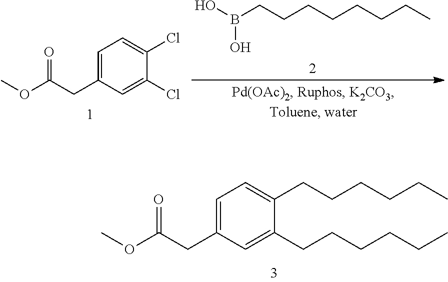 [SMILES: COC(=O)Cc1ccc(Cl)c(Cl)c1 ; CCCCCCCCB(O)O ; CCCCCCc1ccc(CC(=O)OC)cc1CCCCCC]

Palladium acetate (51 mg, 0.228 mmol, 0.1 eq.), Ruphos (213 mg, 0.457 mmol, 0.2 eq.), and potassium carbonate (945 mg, 6.849 mmol, 3 eq.) were added into a mixture of compound 1 (500 mg, 2.283 mmol, 1 eq.) and compound 2 (890 mg, 6.849 mmol, 3 eq.) in toluene/water (5 mL/1 mL). The mixture was stirred overnight at 110° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (723 mg, 99.6%).

### 2. Synthesis of Compound 4

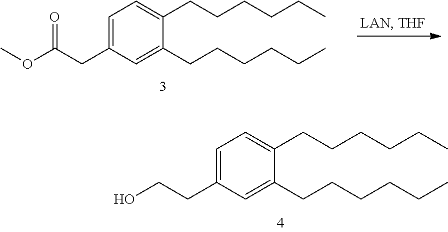 [SMILES: CCCCCCc1ccc(CC(=O)OC)cc1CCCCCC ; CCCCCCc1ccc(CCO)cc1CCCCCC]

At 0° C. and under nitrogen environment, lithium aluminium hydride (2.3 mL, 2.27 mmol, 1 M, in THF, 1 eq.) was added into a mixture of compound 3 (723 mg, 2.27 mmol, 1 eq.) in THF (8 mL). The mixture was stirred at room temperature for 3 hours. TLC (PE/EA=5/1) showed that the reaction was complete and a new major spot was observed. The mixture was quenched with water (2.3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (381 mg, >58%), which did not need to be further purified.

### 3. Synthesis of Compound 6

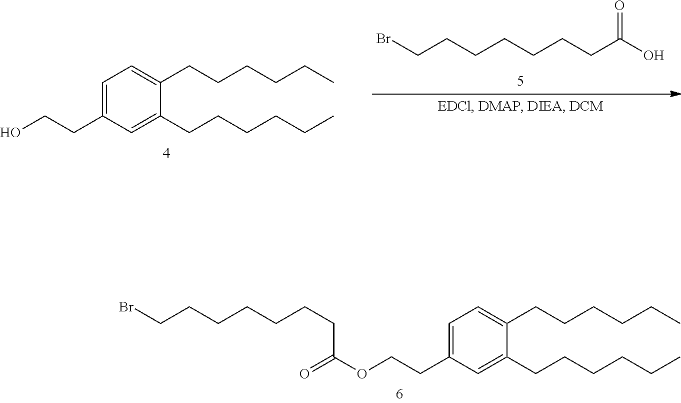 [SMILES: O=C(O)CCCCCCCBr ; CCCCCCc1ccc(CCO)cc1CCCCCC ; CCCCCCc1ccc(CCOC(=O)CCCCCCCBr)cc1CCCCCC]

EDCI (499 mg, 2.6 mmol, 2 eq.) and DMAP (63 mg, 0.52 mmol, 0.4 eq.) were added into a mixture of compound 4 (381 mg, 1.3 mmol, 1 eq.) and compound 5 (352 mg, 1.6 mmol, 1.2 eq.) in DCM (4 mL), and then DIEA (671 mg, 5.2 mmol, 4 eq.) was added. The reaction mixture was stirred at room temperature for 16 hours under nitrogen. TLC (petroleum ether/ethyl acetate=20/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (272 mg, 44%).

### 4. Synthesis of SW-II-134-1

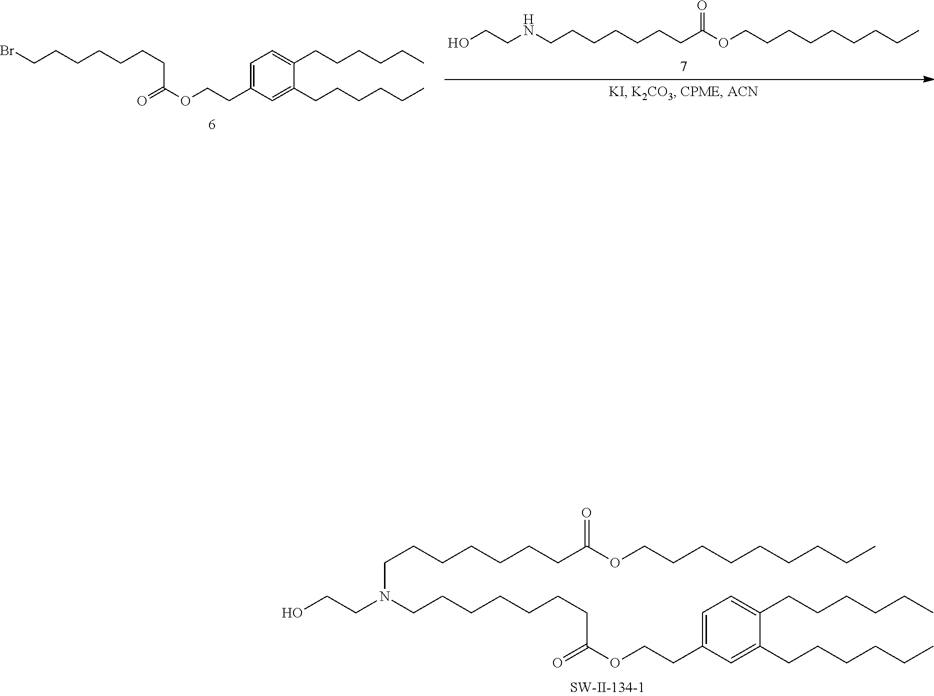 [SMILES: CCCCCCCCCOC(=O)CCCCCCCNCCO ; CCCCCCc1ccc(CCOC(=O)CCCCCCCBr)cc1CCCCCC ; CCCCCCCCCOC(=O)CCCCCCCN(CCO)CCCCCCCC(=O)OCCc1ccc(CCCCCC)c(CCCCCC)c1]

Potassium carbonate (251 mg, 1.818 mmol, 6 eq.) and potassium iodide (101 mg, 0.61 mmol, 2 eq.) were added into a mixture of compound 6 (150 mg, 0.303 mmol, 1 eq.) and compound 7 (110 mg, 0.333 mmol, 1.1 eq.) in CPME/CH₃CN (2 mL/2 mL). After the addition, the mixture was stirred overnight at 90° C. under nitrogen. TLC (DCM/MeOH=15/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10:1, v/v) to obtain yellow oily compound SW-II-134-1 (168 mg, 75%).

LCMS: Rt: 1.276 min; MS m/z(ELSD): 744.4[M+H]⁺;

HPLC: 98.481% purity, ELSD; RT=10.724 min.

¹H NMR (400 MHz, CDCl₃) δ7.06 (d, J=7.6 Hz, 1H), 7.01-6.93 (m, 2H), 4.25 (t, J=7.3 Hz, 2H), 4.05 (t, J=6.8 Hz, 2H), 3.85-3.72 (m, 2H), 2.98-2.69 (m, 8H), 2.62-2.48 (m, 4H), 2.29 (t, J=7.5 Hz, 4H), 1.72-1.48 (m, 14H), 1.45-1.17 (m, 36H), 0.89(dt, J=11.9, 6.0 Hz, 9H).

¹³CNMR(101 MHz, CDCl₃) δ 173.78(d, J=16.7 Hz), 140.72(s), 138.81(s), 134.91(s), 129.70(s), 129.22(s), 126.19(s), 77.30(d, J=11.4 Hz), 77.03(s), 76.72(s), 65.02(s), 64.49(s), 57.42(s), 56.36(s), 54.08(s), 34.76(s), 34.22(d, J=4.2 Hz), 32.74(s), 32.36(s), 31.81(d, J=9.1 Hz), 31.35(d, J=5.3 Hz), 29.49(d, J=2.8 Hz), 29.24(d, J=2.2 Hz), 28.92(s), 28.66(s), 26.86(s), 25.93(s), 25.04(s), 24.78(d, J=6.6 Hz), 22.65(d, J=2.6 Hz), 14.10(s).

### H. Compound SW-II-134-2

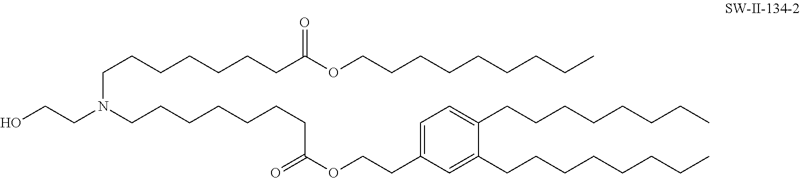 [SMILES: CCCCCCCCCOC(=O)CCCCCCCN(CCO)CCCCCCCC(=O)OCCc1ccc(CCCCCCCC)c(CCCCCCCC)c1]

### 1. Synthesis of Compound 3

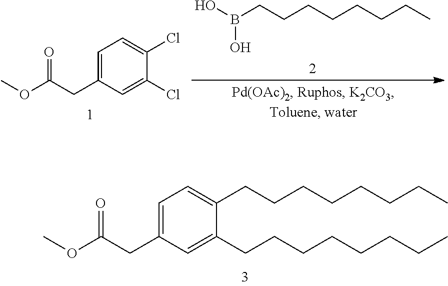 [SMILES: COC(=O)Cc1ccc(Cl)c(Cl)c1 ; CCCCCCCCB(O)O ; CCCCCCCCc1ccc(CC(=O)OC)cc1CCCCCCCC]

Palladium acetate (51 mg, 0.228 mmol, 0.1 eq.), Ruphos (213 mg, 0.457 mmol, 0.2 eq.), and potassium carbonate (945 mg, 6.849 mmol, 3 eq.) were added into a mixture of compound 1 (500 mg, 2.283 mmol, 1 eq.) and compound 2 (1.08 g, 6.849 mmol, 3 eq.) in toluene/water (5 mL/1 mL). The mixture was stirred overnight at 110° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (854 mg, 100%).

### 2. Synthesis of Compound 4

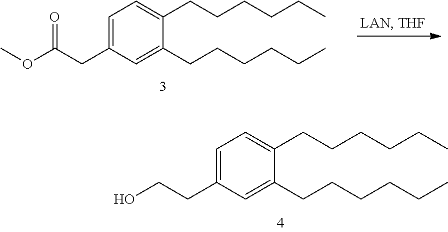 [SMILES: CCCCCCc1ccc(CC(=O)OC)cc1CCCCCC ; CCCCCCc1ccc(CCO)cc1CCCCCC]

At 0° C. and under nitrogen environment, lithium aluminium hydride (2.3 mL, 2.28 mmol, 1 M, in THF, 1 eq.) was added into a mixture of compound 3 (854 mg, 2.28 mmol, 1 eq.) in THF (9 mL). The mixture was stirred at room temperature for 3 hours. TLC (PE/EA=5/1) showed that the reaction was complete and a new major spot was observed. The mixture was quenched with water (2.3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (724 mg, 92%), which did not need to be further purified.

### 3. Synthesis of Compound 6

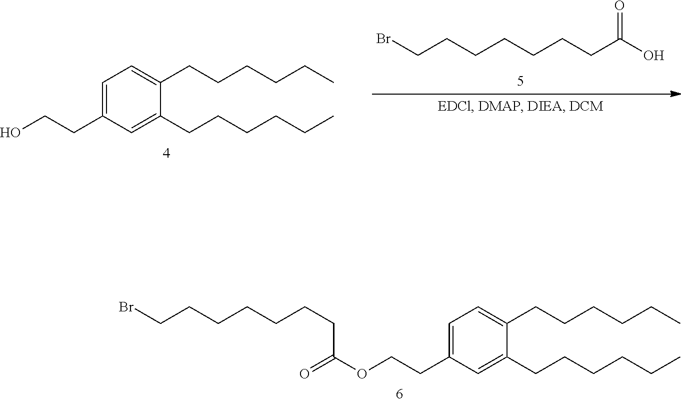 [SMILES: O=C(O)CCCCCCCBr ; CCCCCCc1ccc(CCO)cc1CCCCCC ; CCCCCCc1ccc(CCOC(=O)CCCCCCCBr)cc1CCCCCC]

EDCI (803 mg, 4.18 mmol, 2 eq.) and DMAP (102 mg, 0.84 mmol, 0.4 eq.) were added into a mixture of compound 4 (724 mg, 2.09 mmol, 1 eq.) and compound 5 (560 mg, 2.51 mmol, 1.2 eq.) in DCM (8 mL), and then DIEA (1.078 g, 8.36 mmol, 4 eq.) was added. The reaction mixture was stirred at room temperature for 16 hours under nitrogen. TLC (petroleum ether/ethyl acetate=20/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (473 mg, 41%).

### 4. Synthesis of SW-II-134-2

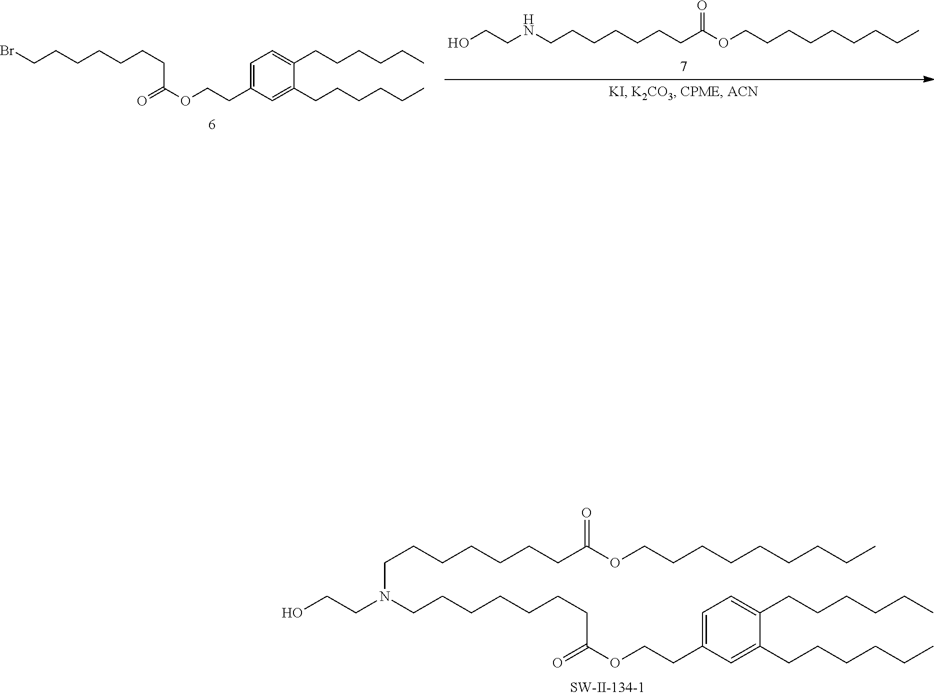 [SMILES: CCCCCCCCCOC(=O)CCCCCCCNCCO ; CCCCCCc1ccc(CCOC(=O)CCCCCCCBr)cc1CCCCCC ; CCCCCCCCCOC(=O)CCCCCCCN(CCO)CCCCCCCC(=O)OCCc1ccc(CCCCCC)c(CCCCCC)c1]

Potassium carbonate (225 mg, 1.63 mmol, 6 eq.) and potassium iodide (90 mg, 0.54 mmol, 2 eq.) were added into a mixture of compound 6 (150 mg, 0.27 mmol, 1 eq.) and compound 7 (108 mg, 0.33 mmol, 1.1 eq.) in CPME/CH₃CN (2 mL/2 mL). After the addition, the mixture was stirred overnight at 90° C. under nitrogen. TLC (DCM/MeOH=15/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10:1, v/v) to obtain yellow oily compound SW-II-134-2 (71.77 mg, 33%).

LCMS: Rt: 1.527 min; MS m/z(ELSD): 800.4[M+H]⁺;

HPLC: 97.311% purity, ELSD; RT=9.025 min.

¹H NMR (400 MHz, CDCl₃) δ7.06 (d, J=7.6 Hz, 1H), 6.96 (d, J=9.6 Hz, 2H), 4.25 (t, J=7.3 Hz, 2H), 4.05 (t, J=6.8 Hz, 2H), 3.80-3.66 (m, 2H), 2.86 (dd, J=12.8, 5.6 Hz, 4H), 2.78-2.67 (m, 4H), 2.60-2.52 (m, 4H), 2.29 (t, J=7.5 Hz, 4H), 1.57(dt, J=15.8, 7.3 Hz, 14H), 1.30 (d, J=20.3 Hz, 45H), 0.88 (t, J=6.7 Hz, 9H).

¹³C NMR (101 MHz, CDCl₃) δ 173.82(d, J=16.9 Hz), 140.73(s), 138.82(s), 134.91(s), 129.71(s), 129.23(s), 126.19(s), 77.36(s), 77.14(d, J=20.4 Hz), 76.72(s), 65.03(s), 64.49(s), 57.57(s), 56.13(s), 54.02(s), 34.76(s), 34.25(d, J=4.2 Hz), 32.76(s), 32.37(s), 31.89(d, J=5.3 Hz), 31.40(d, J=6.0 Hz), 29.84(d, J=3.7 Hz), 29.63-29.14(m), 28.97(s), 28.65(s), 26.93(s), 25.66(d, J=54.4 Hz), 24.80(d, J=6.6 Hz), 22.68(d, J=1.8 Hz), 14.12(s).

### 1. Compound SW-II-134-3

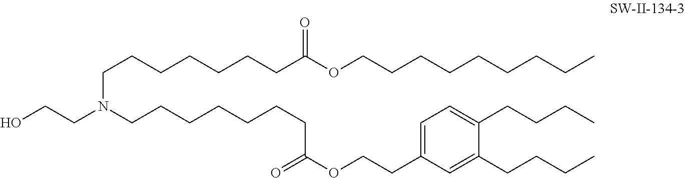 [SMILES: CCCCCCCCCOC(=O)CCCCCCCN(CCO)CCCCCCCC(=O)OCCc1ccc(CCCC)c(CCCC)c1]

### 1. Synthesis of Compound 3

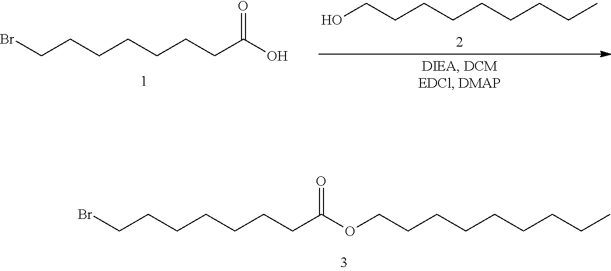

EDCI (17.3 g, 90 mmol, 2 eq.) and DMAP (2.2 g, 18 mmol, 0.4 eq.) were added into a mixture of compound 1 (10 g, 45 mmol, 1 eq.) and compound 2 (7.8 g, 54 mmol, 1.2 eq.) in DCM (100 mL), and then DIEA (23.2 g, 180 mmol, 4 eq.) was added. The reaction mixture was stirred at room temperature for 16 hours under nitrogen. TLC (petroleum ether/ethyl acetate=30/1) showed that compound 1 was consumed and the desired product was formed. The reaction mixture was extracted with ethyl acetate (20 mL), washed with water (40 mL×3), dried with anhydrous sodium sulfate, filtered, and concentrated under reduced pressure. The residue was purified by silica gel column chromatography, and eluted with petroleum ether/ethyl acetate (1/0-20/1) to obtain colorless oily compound 3 (4.365 g, 28%).

### 2. Synthesis of Compound 5

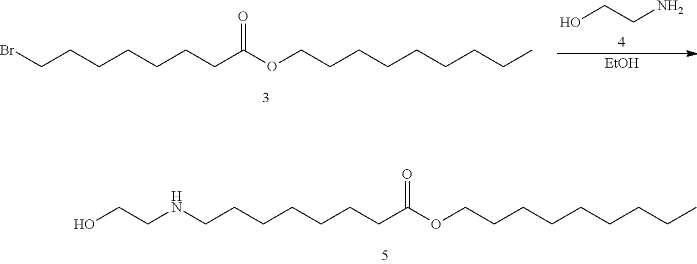

A mixture of compound 3 (5 g, 14.38 mmol, 1 eq.) and compound 4 (8.8 g, 143.7 mmol, 10 eq.) in ethanol (2 mL) was stirred at 55° C. for 16 hours under nitrogen. TLC (DCM/MeOH=10/1) showed that a new major spot was observed. The reaction mixture was extracted with ethyl acetate (50 mL) and washed with water (3×50 mL). The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound 5 (1.008 g, 21%).

### 3. Synthesis of Compound 8

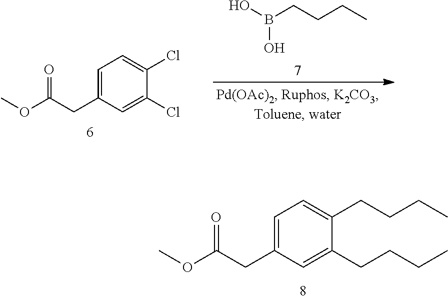

Palladium acetate (51 mg, 0.228 mmol, 0.1 eq.), Ruphos (213 mg, 0.457 mmol, 0.2 eq.), and potassium carbonate (945 mg, 6.849 mmol, 3 eq.) were added into a mixture of compound 6 (500 mg, 2.283 mmol, 1 eq.) and compound 7 (699 mg, 6.849 mmol, 3 eq.) in toluene/water (5 mL/1 mL). The mixture was stirred overnight at 110° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 8 (507 mg, 85%).

### 4. Synthesis of Compound 9

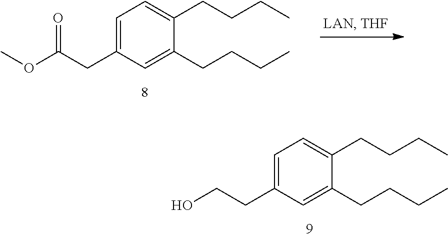 [SMILES: CCCCc1ccc(CC(=O)OC)cc1CCCC ; CCCCc1ccc(CCO)cc1CCCC]

At 0° C. and under nitrogen environment, lithium aluminium hydride (2 mL, 1.935 mmol, 1 M, in THF, 1 eq.) was added into a mixture of compound 8 (507 mg, 1.935 mmol, 1 eq.) in THF (5 mL). The mixture was stirred at room temperature for 3 hours. TLC (PE/EA=5/1) showed that the reaction was complete and a new major spot was observed. The mixture was quenched with water (2 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 9 (492 mg, >100%), which did not need to be further purified.

### 5. Synthesis of Compound 10

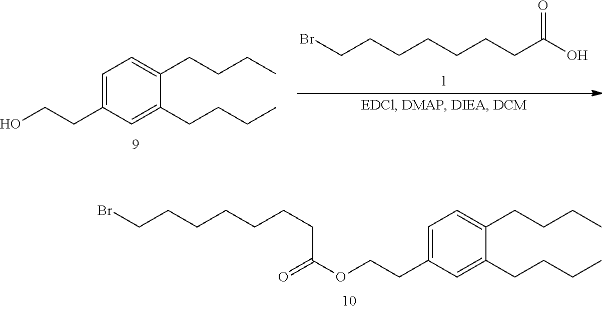 [SMILES: O=C(O)CCCCCCCBr ; CCCCc1ccc(CCO)cc1CCCC ; CCCCc1ccc(CCOC(=O)CCCCCCCBr)cc1CCCC]

EDCI (808 mg, 4.206 mmol, 2 eq.) and DMAP (103 mg, 0.84 mmol, 0.4 eq.) were added into a mixture of compound 9 (492 mg, 2.103 mmol, 1 eq.) and compound 1 (563 mg, 2.523 mmol, 1.2 eq.) in DCM (5 mL), and then DIEA (1.085 g, 8.412 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-10/1) to obtain colorless oily compound 10 (329 mg, 36%).

### 6. Synthesis of SW-II-134-3

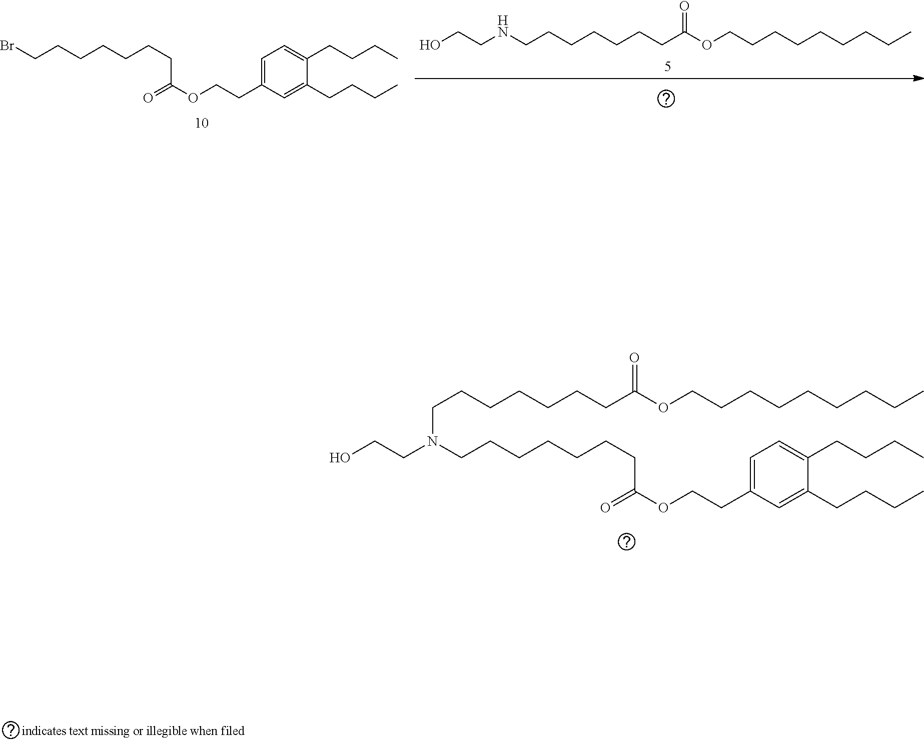

Potassium carbonate (282 mg, 2.04 mmol, 6 eq.) and potassium iodide (113 mg, 0.68 mmol, 2 eq.) were added into a mixture of compound 10 (150 mg, 0.34 mmol, 1 eq.) and compound 5 (134 mg, 0.41 mmol, 1.2 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10/1, v/v) to obtain yellow oily compound SW-II-134-3 (63.59 mg, 25%).

LCMS: Rt: 1.247 min; MS m/z(ELSD): 688.3[M+H]⁺;

HPLC: 95.945% purity, ELSD; RT=6.186 min.

¹H NMR (400 MHz, CDCl₃) δ7.07 (d, J=7.6 Hz, 1H), 6.97 (dd, J=9.9, 2.2 Hz, 2H), 4.26 (t, J=7.2 Hz, 2H), 4.05(t, J=6.8 Hz, 2H), 2.88 (dd, J=14.8, 7.6 Hz, 4H), 2.78-2.74 (m, 2H), 2.67-2.54 (m, 8H), 2.29 (t, J=7.5 Hz, 4H), 1.68-1.47 (m, 15H), 1.37-1.22 (m, 27H), 0.98-0.86 (m, 9H).

¹³C NMR (101 MHz, CDCl₃) δ 173.86(d, J=17.1 Hz), 140.66(s), 138.76(s), 134.93(s), 129.74(s), 129.24(s), 126.19(s), 77.36(s), 77.04(s), 76.72(s), 65.01(s), 64.48(s), 57.73(s), 55.73(s), 53.93(s), 34.76(s), 34.28(d, J=3.9 Hz), 33.54(d, J=4.5 Hz), 32.41(s), 31.95(d, J=16.5 Hz), 29.49(s), 29.15(dd, J=21.1, 2.4 Hz), 28.66(s), 27.04(s), 25.95(d, J=3.3 Hz), 24.85(d, J=6.6 Hz), 22.98-22.58(m), 14.08(d, J=7.5 Hz).

### J. SW-II-135-1

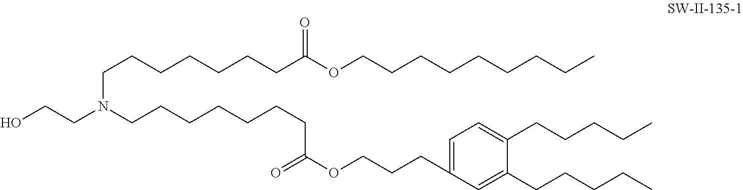 [SMILES: CCCCCCCCCOC(=O)CCCCCCCN(CCO)CCCCCCCC(=O)OCCCc1ccc(CCCCC)c(CCCCC)c1]

### 1. Synthesis of Compound 3

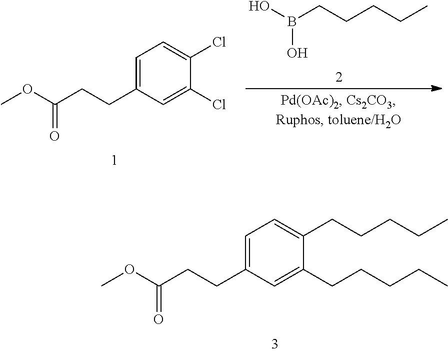

Compound 1 (500 mg, 2.16 mmol, 1.0 eq.) and compound 2 (750 mg, 6.46 mmol, 3.0 eq.) were dissolved in toluene/H₂O (5 mL/1 mL). Ruphos (201 mg, 0.43 mmol, 0.2 eq.), Pd(OAc)₂ (48.5 mg, 0.22 mmol, 0.1 eq.), and Cs₂CO₃ (2.10 g, 6.46 mmol, 3.0 eq.) were added into the mixture. The reaction mixture was subjected to heating reflux at 110° C. for 16 hours under nitrogen protection. TLC (petroleum ether/ethyl acetate=10/1) showed that the reaction was complete and the desired product was formed. The reaction mixture was washed with H₂O (40 mL) and extracted with three times with EA (50 mL). The obtained organic phase was washed twice with brine (20 mL), dried with anhydrous Na₂SO₄, filtered, and concentrated under reduced pressure. The residue was purified by silica gel column chromatography, and eluted with petroleum ether/ethyl acetate (1/0-30/1) to obtain yellow oily compound 3 (540 mg, 82.44%).

### 2. Synthesis of Compound 4

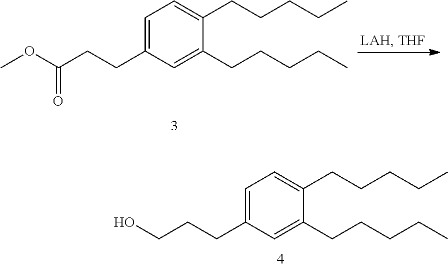 [SMILES: CCCCCc1ccc(CCC(=O)OC)cc1CCCCC ; CCCCCc1ccc(CCCO)cc1CCCCC]

LiAlH₄ (3.55 mL, 3.55 mmol, 1 M in THF, 2 eq.) was added into compound 3 (540 mg, 1.78 mmol, 1.0 eq.) dissolved in THF (5 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with water (10 mL), then regulated with 1 M hydrochloric acid to pH=6-7, and extracted three times with ethyl acetate (50 mL). The organic layer was washed with brine, dried with anhydrous sodium sulfate, filtered, and concentrated under reduced pressure. The residue was purified by silica gel column chromatography, and eluted with PE/EA (1/0-10/1) to obtain colorless oily compound 4 (442 mg, 90.2%).

### 3. Synthesis of Compound 6

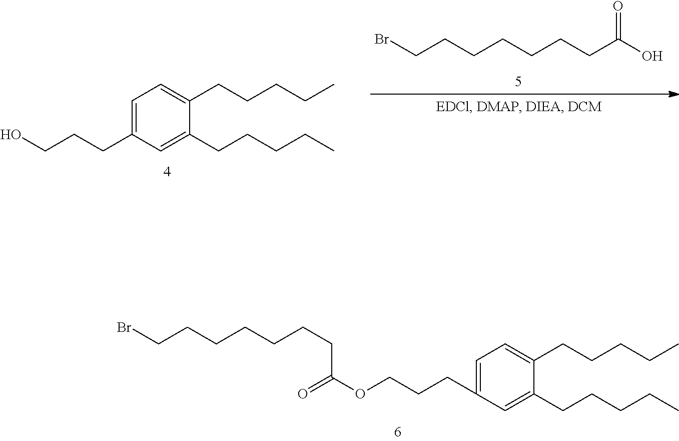 [SMILES: O=C(O)CCCCCCCBr ; CCCCCc1ccc(CCCO)cc1CCCCC ; CCCCCc1ccc(CCCOC(=O)CCCCCCCBr)cc1CCCCC]

Compound 4 (442 mg, 1.60 mmol, 1.0 eq.) and compound 5 (428.5 mg, 1.92 mmol, 1.2 eq.) were dissolved in DCM (5 mL). EDCI (612 mg, 3.2 mmol, 2.0 eq.) and DMAP (78.2 mg, 0.64 mmol, 0.4 eq.) were added into the mixture, and then DIEA (826 mg, 6.4 mmol, 4.0 eq.) was added. The reaction mixture was stirred at room temperature for 16 hours under nitrogen protection. TLC (petroleum ether/ethyl acetate=10/1) showed that compound 4 was consumed and the desired product was formed. The reaction mixture was washed with H₂O (40 mL) and extracted with three times with EA (50 mL). The obtained organic phase was washed twice with brine (20 mL), dried with anhydrous Na₂SO₄, filtered, and concentrated under reduced pressure. The residue was purified by silica gel column chromatography, and eluted with petroleum ether/ethyl acetate (1/0-10/1) to obtain yellow oily compound 3 (342 mg, 44.5%).

### 4. Synthesis of SW-II-135-1

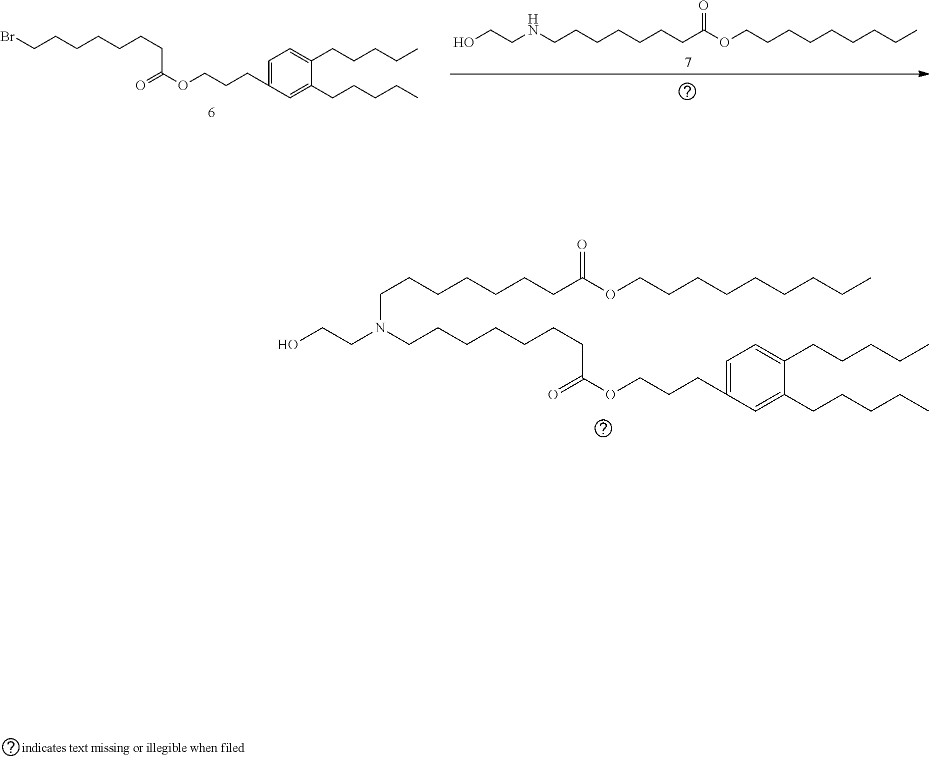 [SMILES: CCCCCc1ccc(CCCOC(=O)CCCCCCCBr)cc1CCCCC ; CCCCCCCCCOC(=O)CCCCCCCNCCO ; CCCCCCCCCOC(=O)CCCCCCCN(CCO)CCCCCCCC(=O)OCCCc1ccc(CCCCC)c(CCCCC)c1]

Compound 6 (175 mg, 0.365 mmol, 1.2 eq.) and compound 7 (100 mg, 0.304 mmol, 1.0 eq.) were dissolved in CPME/CH₃CN (1 mL/1 mL). Potassium carbonate (210 mg, 1.52 mmol, 5.0 eq.) and potassium iodide (101 mg, 0.61 mmol, 2.0 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10/1, v/v) to obtain yellow oily compound SW-II-135-1 (83.89 mg, 55.6%).

LCMS: Rt: 1.356 min; MS m/z(ELSD): 730.5[M+H]⁺;

HPLC: 100% purity at ELSD; RT=12.614 min.

¹H NMR (400 MHz, CDCl3) δ 6.97 (d, J=7.6 Hz, 1H), 6.91-6.74 (m, 2H), 4.76 (s, 1H), 3.99(dt, J=13.6, 6.4 Hz, 4H), 3.72-3.58 (m, 2H), 2.85-2.73 (m, 2H), 2.72-2.61 (m, 4H), 2.59-2.41 (m, 6H), 2.22 (dd, J=13.2, 7.2 Hz, 4H), 1.93-1.79 (m, 2H), 1.62-1.41 (m, 14H), 1.23 (d, J=24.4 Hz, 32H), 0.82(ddd, J=13.6, 8.0, 5.6 Hz, 9H).

¹³C NMR (101 MHz, CDCl3) δ 172.81(d, J=6.4 Hz), 139.55(s), 137.38(s), 137.14(s), 128.16(d, J=2.4 Hz), 124.69(s), 76.51(s), 76.19(s), 75.88(s), 63.43(s), 62.78(s), 56.53(s), 54.90(s), 52.84(s), 33.23(d, J=2.4 Hz), 31.73(s), 31.28(s), 30.91(dd, J=20.0, 6.4 Hz), 30.10(d, J=3.2 Hz), 29.29(s), 28.36(d, J=22.8 Hz), 28.23(s), 27.97(s), 27.64(s), 25.92(s), 24.92(s), 24.34(s), 23.84(s), 21.62(d, J=7.6 Hz), 13.08(d, J=4.7 Hz).

### K. Compound SW-II-135-2

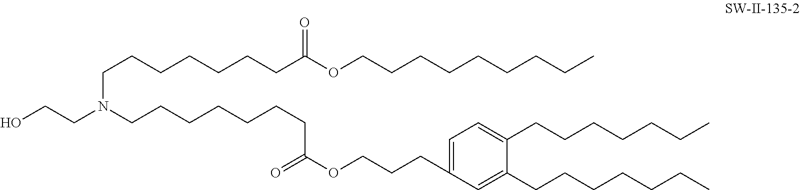 [SMILES: CCCCCCCCCOC(=O)CCCCCCCN(CCO)CCCCCCCC(=O)OCCCc1ccc(CCCCCCC)c(CCCCCCC)c1]

### 1. Synthesis of Compound 3

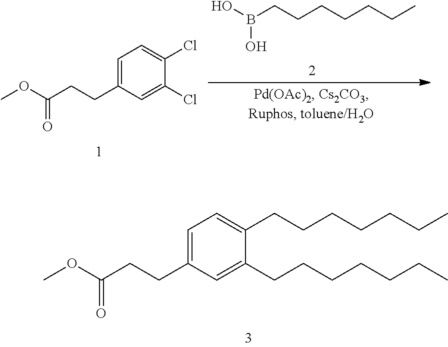

Compound 1 (500 mg, 2.16 mmol, 1.0 eq.) and compound 2 (931 mg, 6.46 mmol, 3.0 eq.) were dissolved in toluene/H₂O (5 mL/1 mL). Ruphos (201 mg, 0.43 mmol, 0.2 eq.), Pd(OAc)₂ (48.5 mg, 0.22 mmol, 0.1 eq.), and Cs₂CO₃ (2.10 g, 6.46 mmol, 3.0 eq.) were added into the mixture. The reaction mixture was subjected to heating reflux at 110° C. for 16 hours under nitrogen protection. TLC (petroleum ether/ethyl acetate=10/1) showed that the reaction was complete and the desired product was formed. The reaction mixture was washed with H₂O (40 mL) and extracted with three times with EA (50 mL). The obtained organic phase was washed twice with brine (20 mL), dried with anhydrous Na₂SO₄, filtered, and concentrated under reduced pressure. The residue was purified by silica gel column chromatography, and eluted with petroleum ether/ethyl acetate (1/0-30/1) to obtain yellow oily compound 3 (651 mg, 84%).

### 2. Synthesis of Compound 4

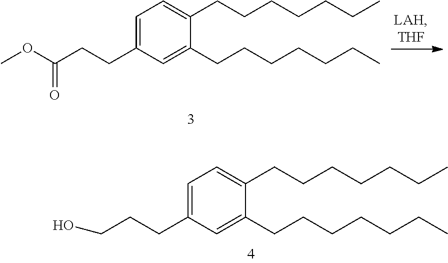 [SMILES: CCCCCCCc1ccc(CCC(=O)OC)cc1CCCCCCC ; CCCCCCCc1ccc(CCCO)cc1CCCCCCC]

LiAlH₄ (3.62 mL, 3.62 mmol, 1 M in THF, 2 eq.) was added into compound 3 (651 mg, 1.81 mmol, 1.0 eq.) dissolved in THF (7 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with water (10 mL), then regulated with 1 M hydrochloric acid to pH=6-7, and extracted three times with ethyl acetate (50 mL). The organic layer was washed with brine, dried with anhydrous sodium sulfate, filtered, and concentrated under reduced pressure. The residue was purified by silica gel column chromatography, and eluted with PE/EA (1/0-10/1) to obtain colorless oily compound 4 (571 mg, 95.2%).

### 3. Synthesis of Compound 6

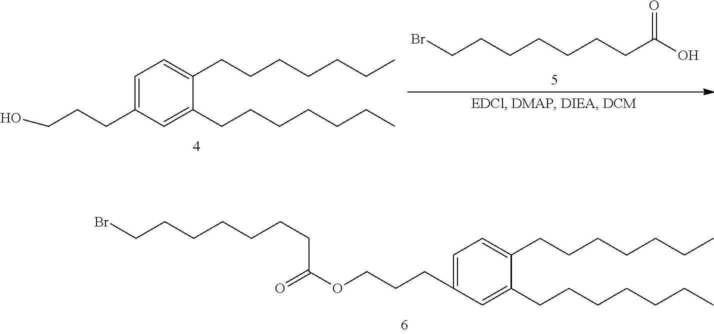

Compound 4 (571 mg, 1.72 mmol, 1.0 eq.) and compound 5 (459 mg, 2.06 mmol, 1.2 eq.) were dissolved in DCM (6 mL). EDCI (657 mg, 3.44 mmol, 2.0 eq.) and DMAP (84 mg, 0.68 mmol, 0.4 eq.) were added into the mixture, and then DIEA (887.5 mg, 6.88 mmol, 4.0 eq.) was added. The reaction mixture was stirred at room temperature for 16 hours under nitrogen protection. TLC (petroleum ether/ethyl acetate=10/1) showed that compound 4 was consumed and the desired product was formed. The reaction mixture was washed with H₂O (50 mL) and extracted with three times with EA (60 mL). The obtained organic phase was washed twice with brine (25 mL), dried with anhydrous Na₂SO₄, filtered, and concentrated under reduced pressure. The residue was purified by silica gel column chromatography, and eluted with petroleum ether/ethyl acetate (1/0-10/1) to obtain yellow oily compound 3 (245 mg, 26.5%).

### 4. Synthesis of SW-II-135-2

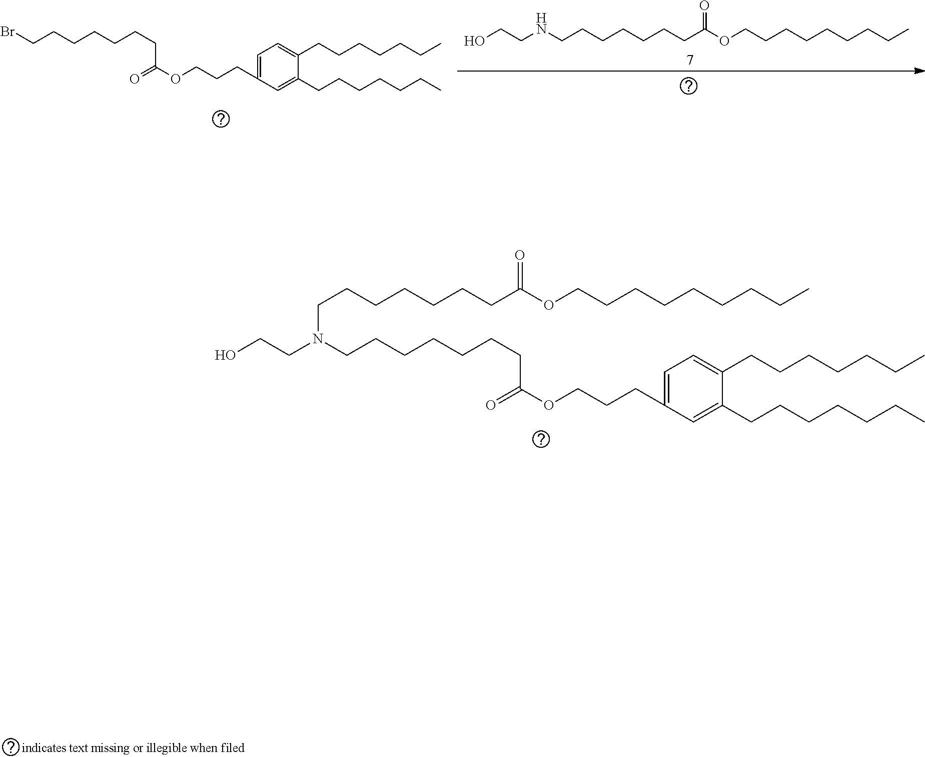

Compound 6 (245 mg, 0.456 mmol, 1.5 eq.) and compound 7 (100 mg, 0.3 mmol, 1.0 eq.) were dissolved in CPME/CH₃CN (1 mL/1 mL). Potassium carbonate (210 mg, 1.52 mmol, 5.0 eq.) and potassium iodide (101 mg, 0.61 mmol, 2.0 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10/1, v/v) to obtain yellow oily compound SW-II-135-2 (31.41 mg, 21.9%).

LCMS: Rt: 1.608 min; MS m/z(ELSD): 786.4[M+H]⁺;

HPLC: 95.16% purity, ELSD; RT=7.919 min.

¹H NMR (400 MHz, CDCl₃) δ6.98 (d, J=7.6 Hz, 1H), 6.87 (d, J=2.4 Hz, 2H), 4.28-4.13 (m, 1H), 4.04-3.95 (m, 4H), 3.94-3.84 (m, 2H), 3.14-2.89 (m, 6H), 2.59-2.43 (m, 6H), 2.23 (dd, J=13.8, 7.2 Hz, 4H), 1.88-1.82 (m, 2H), 1.70 (s, 4H), 1.57-1.46(m, 10H), 1.33-1.16 (m, 40H), 0.90-0.72 (m, 9H).

¹³C NMR (100 MHz, CDCl₃) δ 172.82(d, J=6.8 Hz), 139.61(s), 137.29(d, J=16.4 Hz), 128.15(s), 124.67(s), 76.41(s), 76.09(s), 75.77(s), 63.50(s), 62.87(s), 55.49(s), 54.92(s), 52.98(s), 33.16(d, J=2.4 Hz), 31.77(s), 31.33(s), 30.80(d, J=6.5 Hz), 30.42(d, J=3.6 Hz), 29.29(s), 28.99-28.66(m), 28.47(s), 28.23(d, J=2.8 Hz), 28.06-27.45(m), 25.58(s), 24.91(s), 23.71(s), 22.79(s), 21.66(s), 13.10(s).

### L. Compound SW-II-136-2

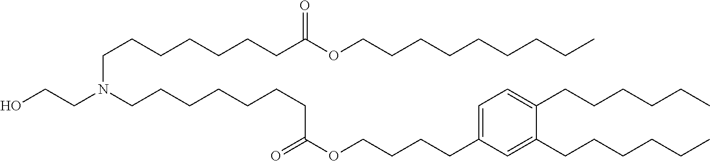 [SMILES: CCCCCCCCCOC(=O)CCCCCCCN(CCO)CCCCCCCC(=O)OCCCCc1ccc(CCCCCC)c(CCCCCC)c1]

### 1. Synthesis of Compound 3

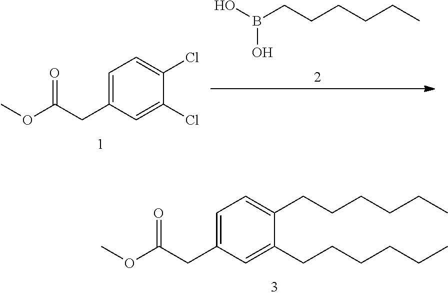

Compound 1 (3 g, 13.70 mmol, 1.0 eq.) and compound 2 (5.34 g, 41.09 mmol, 3.0 eq.) were dissolved in toluene/H₂O (30 mL/3 mL). Ruphos (1.28 g, 2.74 mmol, 0.2 eq.), Pd(OAc)₂ (308.3 mg, 1.37 mmol, 0.1 eq.), and K₂CO₃ (5.67 g, 41.10 mmol, 3.0 eq.) were added into the mixture. The reaction mixture was subjected to heating reflux at 110° C. for 16 hours under nitrogen protection. TLC (PE/EA=10/1) showed that the reaction was complete and the desired product was formed. The reaction mixture was washed with H₂O (90 mL) and extracted with three times with EA (110 mL). The obtained organic phase was washed twice with brine (40 mL), dried with anhydrous Na₂SO₄, filtered, and concentrated under reduced pressure. The residue was purified by silica gel column chromatography, and eluted with PE/EA (1/0-30/1) to obtain yellow oily compound 3 (1.98 g, 45.5%).

### 2. Synthesis of Compound 4

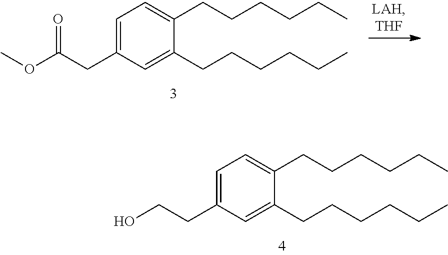 [SMILES: CCCCCCc1ccc(CC(=O)OC)cc1CCCCCC ; CCCCCCc1ccc(CCO)cc1CCCCCC]

LiAlH₄ (1 M, 12.45 mL, 2.0 eq.) was added into compound 3 (1.98 g, 6.23 mL, 1.0 eq.) dissolved in THF (20 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with H₂O (70 mL), then regulated with 1 M hydrochloric acid to pH=6-7, and extracted three times with EA (80 mL). The organic layer was washed with brine, dried with anhydrous Na₂SO₄, filtered, and concentrated under reduced pressure. The residue was purified by silica gel column chromatography, and eluted with PE/EA (1/0-10/1) to obtain colorless oily compound 4 (1.28 g, 71.1%).

### 3. Synthesis of Compound 7

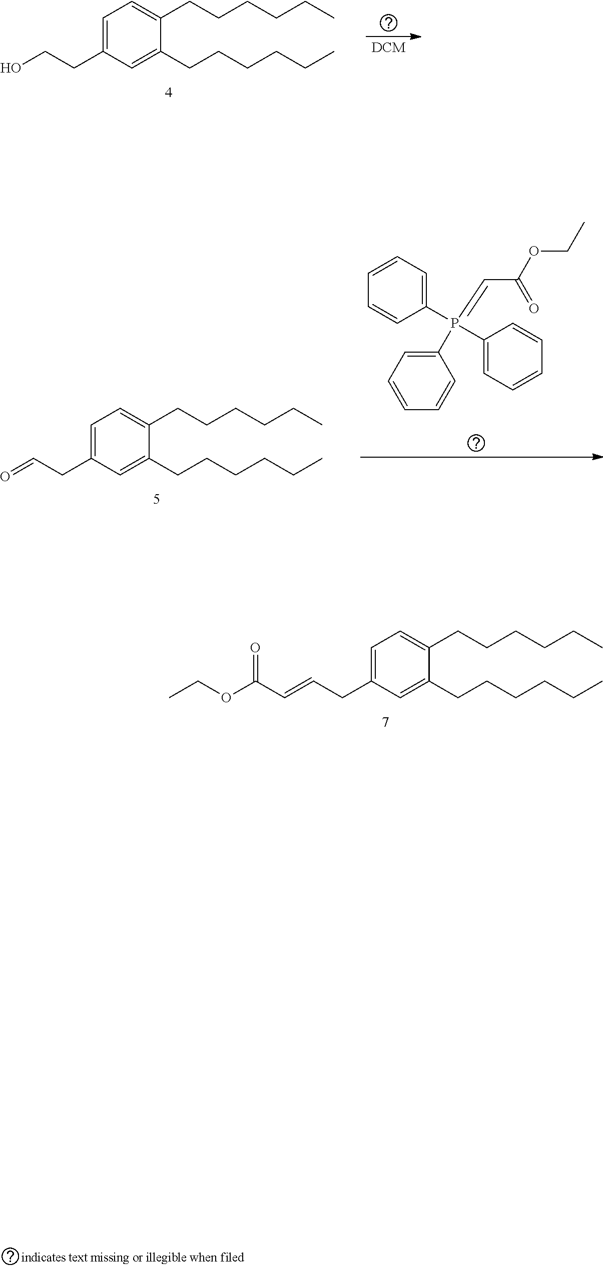 [SMILES: CCCCCCc1ccc(CCO)cc1CCCCCC ; CCCCCCc1ccc(CC=O)cc1CCCCCC ; CCOC(=O)C=P(c1ccccc1)(c1ccccc1)c1ccccc1 ; CCCCCCc1ccc(C/C=C/C(=O)OCC)cc1CCCCCC]

DMSO (3.63 g, 51.72 mmol, 15 eq.), TEA (1.25 g, 12.4 mmol, 4.0 eq.), and PySO₃ (1.27 g, 7.97 mmol, 2.57 eq.) were added into compound 4 (900 g, 3.1 mmol, 1.0 eq.) dissolved in DCM (9 mL) at 0° C. under nitrogen protection. The mixture was stirred at 0° C. for 30 minutes, then heated to room temperature and stirred for 90 minutes under nitrogen protection. Then, compound 6 (4.74 g, 13.62 mmol, 3.0 eq.) was added into the mixture. The reaction mixture reacted at 25° C. for 2 hours under nitrogen protection. TLC (PE/EA=10/1) showed that the reaction was complete and the desired product was formed. The reaction mixture was washed with H₂O (60 mL) and extracted with three times with EA (70 mL). The obtained organic phase was washed twice with brine (40 mL), dried with anhydrous Na₂SO₄, filtered, and concentrated under reduced pressure. The residue was purified by silica gel column chromatography, and eluted with PE/EA (1/0-10/1) to obtain yellow oily compound 7 (345 mg, 27.9%).

### 4. Synthesis of Compound 8

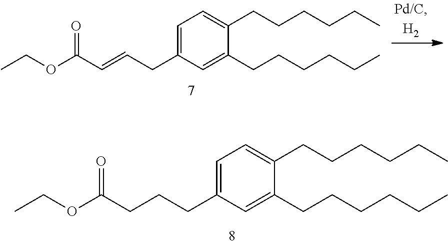

Compound 7 (340 mg, 0.95 mmol, 1.0 eq.) and Pd/C (100 mg) were added into MeOH (4 ml). The reaction mixture was stirred at room temperature for 16 hours under hydrogen protection. TLC (PE/EA=10/1) showed that the raw materials were consumed completely and the desired product was produced. The reaction mixture was filtered with diatomite, washed with MeOH (40 mL×2), dried with anhydrous Na₂SO₄, and concentrated under reduced pressure to obtain light-yellow oily compound 8 (298 g, 88.2%).

### 5. Synthesis of Compound 9

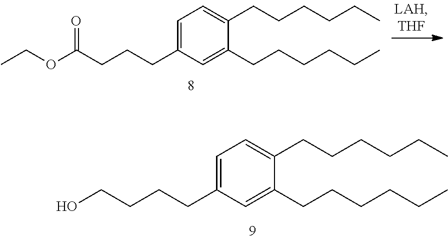 [SMILES: CCCCCCc1ccc(CCCC(=O)OCC)cc1CCCCCC ; CCCCCCc1ccc(CCCCO)cc1CCCCCC]

LiAlH₄ (1 M, 1.66 mL, 2.0 eq.) was added into compound 8 (298 mg, 0.83 mmol, 1.0 eq.) dissolved in THF (3 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with H₂O (20 mL), then regulated with 1 M hydrochloric acid to pH=6-7, and extracted three times with EA (30 mL). The organic layer was washed with brine, dried with anhydrous Na₂SO₄, filtered, and concentrated under reduced pressure. The residue was purified by silica gel column chromatography, and eluted with PE/EA (1/0-10/1) to obtain colorless oily compound 9 (254 mg, 98.3%).

### 6. Synthesis of Compound 11

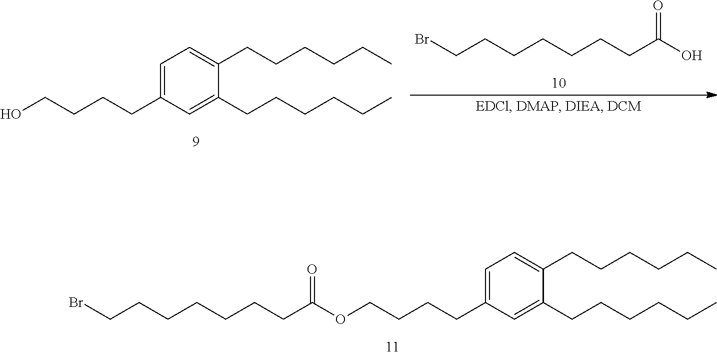

Compound 9 (254 mg, 0.80 mmol, 1.0 eq.) and compound 10 (214 mg, 0.96 mmol, 1.2 eq.) were dissolved in DCM (3 mL). EDCI (305.6 mg, 1.6 mmol, 2.0 eq.) and DMAP (39 mg, 0.32 mmol, 0.4 eq.) were added into the mixture, and then DIEA (412.8 mg, 3.2 mmol, 4.0 eq.) was added. The reaction mixture was stirred at room temperature for 16 hours under nitrogen protection. TLC (PE/EA=10/1) showed that compound 9 was consumed and the desired product was formed. The reaction mixture was regulated with 1 M hydrochloric acid to pH=4-6, and extracted with three times with EA (30 mL). The obtained organic phase was washed twice with brine (15 mL), dried with anhydrous Na₂SO₄, filtered, and concentrated under reduced pressure. The residue was purified by silica gel column chromatography, and eluted with PE/EA (1/0-7/1) to obtain yellow oily compound 11 (210 mg, 50.5%).

### 7. Synthesis of SW-II-136-2

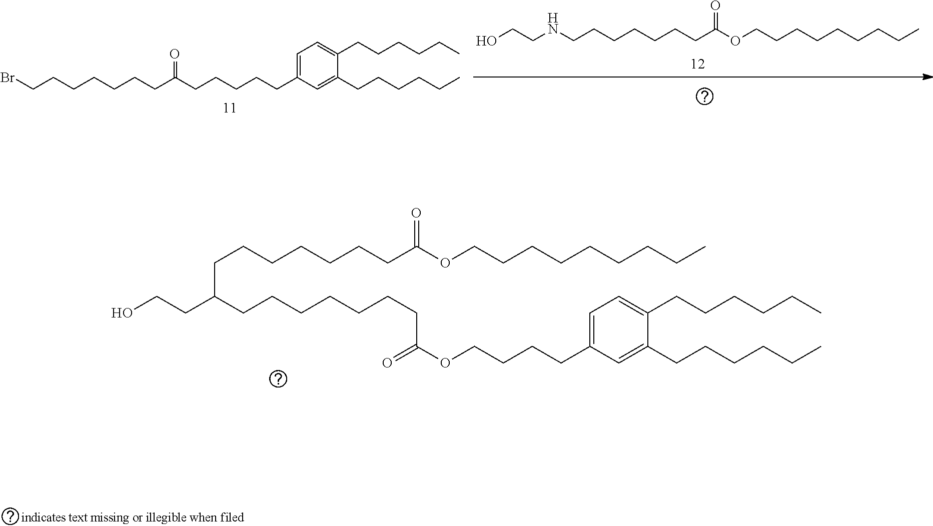

Compound 11 (200 mg, 0.38 mmol, 1.2 eq.) and compound 12 (105 mg, 0.32 mmol, 1.0 eq.) were dissolved in CPME/CH₃CN (1.5 mL/1.5 mL). K₂CO₃ (220.2 mg, 1.60 mmol, 5.0 eq.) and KI (106 mg, 0.64 mmol, 2.0 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with EA and washed with water. The organic layer was dried with anhydrous Na₂SO₄, filtered, and concentrated under reduced pressure. The residue was purified by silica gel column chromatography, and eluted with DCM/MeOH(1/0-10/1, v/v) to obtain yellow oily compound SW-II-136-2 (208 mg, 90.4%).

LCMS: Rt: 2.146 min; MS m/z(ELSD): 773.3[M+H]⁺;

HPLC: 99.49% purity, ELSD; RT=8.055 min.

¹H NMR (400 MHz, CDCl₃) δ7.04 (d, J=7.6 Hz, 1H), 6.92 (d, J=9.6 Hz, 2H), 4.45 (s, 1H), 4.06 (dd, J=12.0, 5.2 Hz, 4H), 3.64 (t, J=5.2 Hz, 2H), 2.72 (t, J=5.2 Hz, 2H), 2.65-2.50(m, 10H), 2.29 (t, J=7.6 Hz, 4H), 1.69-1.48 (m, 18H), 1.41-1.24 (m, 36H), 0.95-0.78 (m, 9H).

¹³C NMR (101 MHz, CDCl₃) δ 173.86(d, J=2.8 Hz), 140.48(s), 139.24(s), 138.01(s), 129.13(d, J=14.8 Hz), 125.67(s), 77.37(s), 77.05(s), 76.73(s), 64.45(s), 64.23(s), 57.88(s), 55.91(s), 53.94(s), 35.07(s), 34.29(d, J=3.2 Hz), 32.79(s), 32.35(s), 31.82(d, J=8.4 Hz), 31.38(s), 29.50(d, J=2.4 Hz), 29.16(dd, J=18.0, 2.0 Hz), 28.66(s), 28.35(s), 27.78(s), 27.08(s), 26.02(d, J=17.2 Hz), 24.89(d, J=1.6 Hz), 22.65(s), 14.10(s).

### M. Compound SW-II-137-1

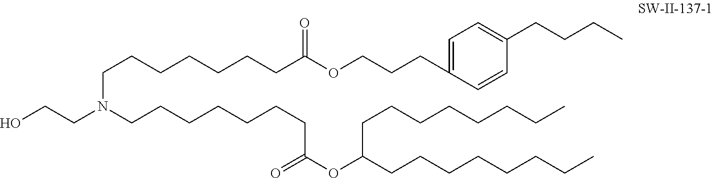 [SMILES: CCCCCCCCC(CCCCCCCC)OC(=O)CCCCCCCN(CCO)CCCCCCCC(=O)OCCCc1ccc(CCCC)cc1]

### 1. Synthesis of Compound 3

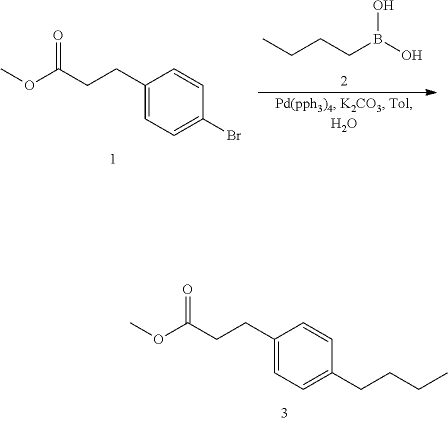

Compound 1 (500 mg, 1.95 mmol, 1.0 eq.) was dissolved in toluene (5.0 mL). Then, compound 2 (239 mg, 2.34 mmol, 1.2 eq.), Pd(PPh₃)₄ (225 mg, 0.19 mmol, 0.1 eq.), water (1 mL), and K₂CO₃ (808 g, 5.85 mmol, 3.0 eq.) were added. The reaction was carried out at 110° C. for 3 hours under nitrogen protection. TLC (PE/EA=5/1) showed that the raw materials had reacted completely and the desired product was formed. H₂O (70 mL) was added into the reaction. Extraction was carried out with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-5:1, v/v) to obtain colorless oily compound (320 mg, 70%).

### 2. Synthesis of Compound 4

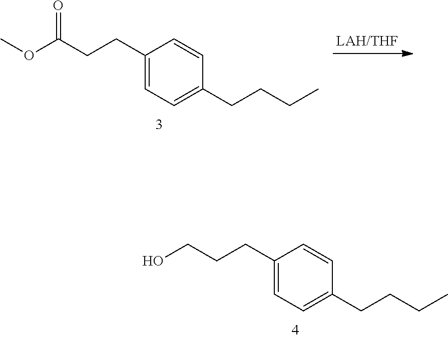 [SMILES: CCCCc1ccc(CCC(=O)OC)cc1 ; CCCCc1ccc(CCCO)cc1]

Compound 3 (300 mg, 1.28 mmol, 1.0 eq.) was dissolved in THF (4.0 mL). LAH (97 mg, 2.56 mmol, 2.0 eq.) was added at 0° C. under nitrogen protection. Then, the reaction was carried out at room temperature for 2 hours. TLC (PE/EA=10/1) showed that the raw materials reacted completely and the desired product was produced. A HCl (1 M, 4 mL) solution and H₂O (10 mL) were added for quenching. Extraction was carried out with EA (50 mL×3). The organic phase was washed with saturated salt solution (2×30 mL), dried with anhydrous Na₂SO₄, filtered, and spin-dried under reduced pressure. The residue was purified by silica gel column, and eluted with PE/EA (1/0-10/1, v/v) to obtain yellow oily compound 4 (224 mg, 84.8%).

### 3. Synthesis of Compound 6

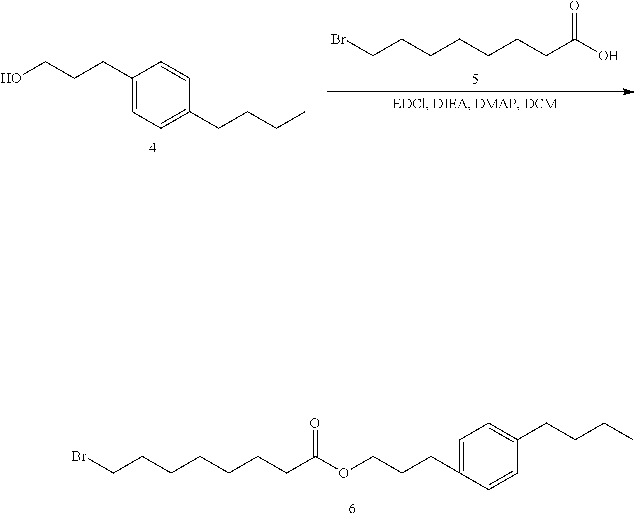 [SMILES: O=C(O)CCCCCCCBr ; CCCCc1ccc(CCCO)cc1 ; CCCCc1ccc(CCCOC(=O)CCCCCCCBr)cc1]

Compound 4 (90 mg, 0.47 mmol, 1.0 eq.) was dissolved in DCM (3.0 mL). Compound 5 (127 mg, 0.56 mmol, 1.2 eq.), EDCI (180 mg, 0.94 mmol, 2.0 eq.), DIEA (242 mg, 1.88 mmol, 4.0 eq.), and DMAP (23 mg, 0.18 mmol, 0.4 eq.) were added. Then, the reaction was carried out overnight at room temperature under nitrogen protection. TLC (PE/EA=20/1) showed that the raw materials had reacted completely and the desired product was formed. The reaction mixture was quenched with a HCl (1 M) solution, regulated to PH=4-6, and extracted with EA (40 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-20/1, v/v) to obtain colorless oily compound 6 (90 mg, 48.6%).

### 4. Synthesis of SW-II-137-1

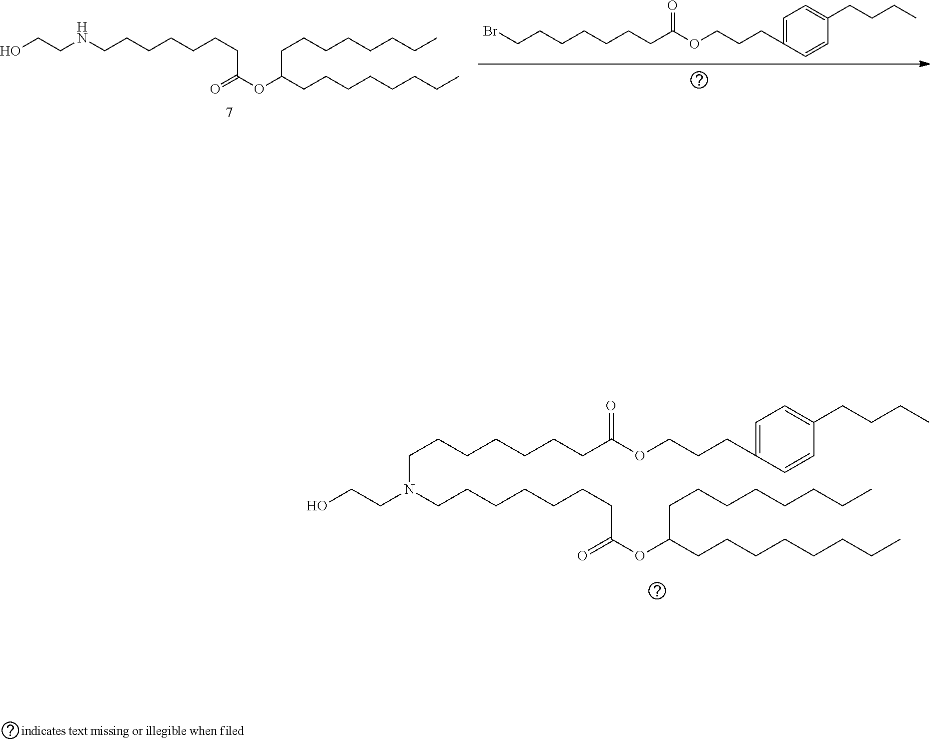 [SMILES: CCCCCCCCC(CCCCCCCC)OC(=O)CCCCCCCNCCO ; CCCCc1ccc(CCCOC(=O)CCCCCCCBr)cc1 ; CCCCCCCCC(CCCCCCCC)OC(=O)CCCCCCCN(CCO)CCCCCCCC(=O)OCCCc1ccc(CCCC)cc1]

Compound 6 (90 mg, 0.25 mmol, 1.0 eq.) was dissolved in MeCN (2 mL). Compound 7 (110 mg, 0.25 mmol, 1.0 eq.), KI (76 mg, 0.50 mmol, 2.0 eq.), CPME (2 mL), and K₂CO₃ (157 mg, 1.25 mmol, 5.0 eq.) were added. The reaction was carried out overnight at 90° C. under nitrogen protection. TLC (DCM/MeOH=10/1) showed that the raw materials reacted completely and the desired product was formed. Quenching was carried out with water (50 mL). Extraction was carried out with EA (40 mL×3). The organic phases were combined, washed with saturated salt solution (2×30 mL), dried with anhydrous Na₂SO₄, filtered, and spin-dried under reduced pressure. The residue was purified by silica gel column, and eluted with DCM/MeOH (1/0-10/1, v/v) to obtain yellow oily compound (98 mg, 52.12%, SW-II-137-1).

LCMS: Rt: 1.596 min; MS m/z(ELSD): 758.4[M+H]⁺;

HPLC: 98.02% purity, ELSD; RT=5.993 min.

¹H NMR (400 MHz, CDCl₃) δ7.02 (d, J=8.8 Hz, 4H), 4.92-4.71 (m, 1H), 4.01 (t, J=6.4 Hz, 2H), 3.78 (s, 1H), 3.55 (t, J=5.2 Hz, 2H), 2.76-2.40(m, 10H), 2.21 (dd, J=15.6, 7.7 Hz, 4H), 1.95-1.80 (m, 2H), 1.49(ddd, J=24.4, 15.8, 6.2 Hz, 15H), 1.34-1.13 (m, 37H), 0.82(dt, J=13.6, 7.2 Hz, 9H).

¹³C NMR (101 MHz, CDCl₃) δ 173.79(s), 173.57(s), 140.49(s), 138.30(s), 128.43(s), 128.22(s), 77.43(s), 77.11(s), 76.79(s), 74.11(s), 63.66(s), 57.96(s), 55.75(s), 53.90(s), 35.22(s), 34.63(s), 34.20 (d, J=11.6 Hz), 33.70(s), 31.80 (d, J=11.2 Hz), 30.30(s), 29.51 (d, J=2.8 Hz), 29.13 (dd, J=9.6, 6.8 Hz), 27.12 (d, J=2.8 Hz), 26.29(s), 25.31(s), 24.97 (d, J=15.6 Hz), 22.66(s), 22.37(s), 14.02(d, J=15.2 Hz)

### N. Compound SW-II-137-2

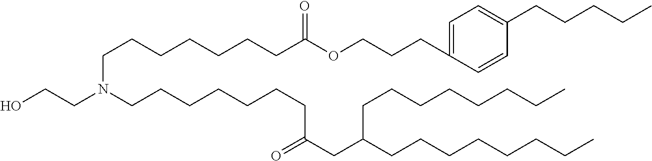 [SMILES: CCCCCCCCC(CCCCCCCC)CC(=O)CCCCCCCN(CCO)CCCCCCCC(=O)OCCCc1ccc(CCCCC)cc1]

### 1. Synthesis of Compound 3

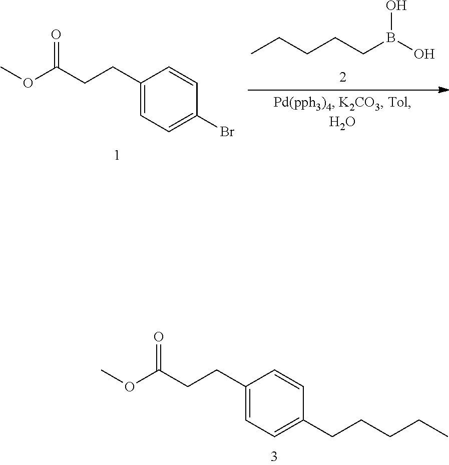

Compound 1 (500 mg, 2.06 mmol, 1.0 eq.), compound 2 (286 mg, 2.47 mmol, 1.2 eq.), Pd(PPh₃)₄ (119 mg, 0.1 mmol, 0.1 eq.), and K₂CO₃ (851 mg, 6.21 mmol, 3.0 eq.) were dissolved in toluene (5.0 mL). Water (0.5 mL) was added. Then, the reaction was carried out at 110° C. for 3 hours under nitrogen protection. TLC (PE/EA=5/1) showed that the raw materials reacted completely and the desired compound was formed. The reaction mixture was quenched with H₂O (70 mL), and extracted with EA (80 mL×3). The organic phase was washed with saturated salt solution (2×30 mL), dried with anhydrous Na₂SO₄, filtered, and spin-dried under reduced pressure. The residue was purified by silica gel column, and eluted with (PE/EA=5/1, v/v) to obtain colorless oily compound 3 (420 mg, 87.5%).

### 2. Synthesis of Compound 4

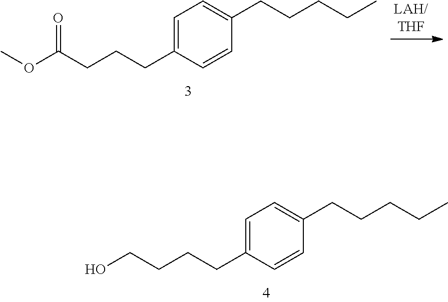 [SMILES: CCCCCc1ccc(CCCC(=O)OC)cc1 ; CCCCCc1ccc(CCCCO)cc1]

Compound 3 (420 mg, 1.78 mmol, 1.0 eq.) was dissolved in THF (3.0 mL). LAH (1 M, 7 mL, 2.0 eq.) was added dropwise at 0° C. under nitrogen protection. Then, the reaction was carried out at room temperature for 2 hours. TLC (PE/EA=5/1) showed that the raw materials reacted completely and the desired product was formed. Quenching was carried out with a HCl (1 M, 4 mL) solution and H₂O (10 mL). Extraction was carried out with EA (50 mL×3). The organic phase was washed with saturated salt solution (2×30 mL), dried with anhydrous Na₂SO₄, filtered, and spin-dried under reduced pressure. The residue was purified by silica gel column, and eluted with (PE/EA=5/1, v/v) to obtain colorless oily compound 4 (320 mg, 94%).

### 3. Synthesis of Compound 6

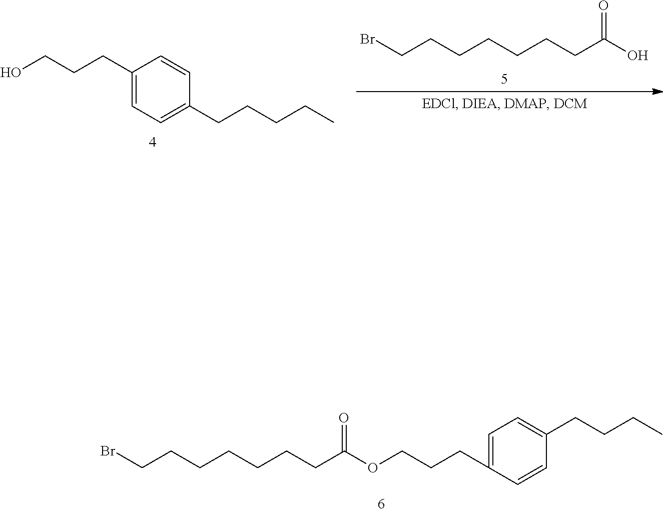 [SMILES: O=C(O)CCCCCCCBr ; CCCCCc1ccc(CCCO)cc1 ; CCCCc1ccc(CCCOC(=O)CCCCCCCBr)cc1]

Compound 4 (320 mg, 1.55 mmol, 1.0 eq.) was dissolved in DCM (4.0 mL). Compound 5 (416 mg, 1.86 mmol, 1.2 eq.), EDCI (594 mg, 3.11 mmol, 2.0 eq.), DIEA (802 mg, 6.21 mmol, 4.0 eq.), and DMAP (76 mg, 0.62 mmol, 0.4 eq.) were added. Then, the reaction was carried out overnight at room temperature under nitrogen protection. TLC (PE/EA=20/1) showed that the raw materials reacted completely and the desired product was formed. The reaction mixture was quenched with a HCl (1 M) solution, regulated to PH=4-6, and extracted with DCM (60 mL×3). The organic phase was washed with saturated salt solution (2×35 mL), dried with anhydrous Na₂SO₄, filtered, and spin-dried under reduced pressure. The residue was purified by silica gel column, and eluted with (PE/EA=5/1, v/v) to obtain colorless oily compound 6 (300 mg, 47.17%).

### 4. Synthesis of SW-II-137-2

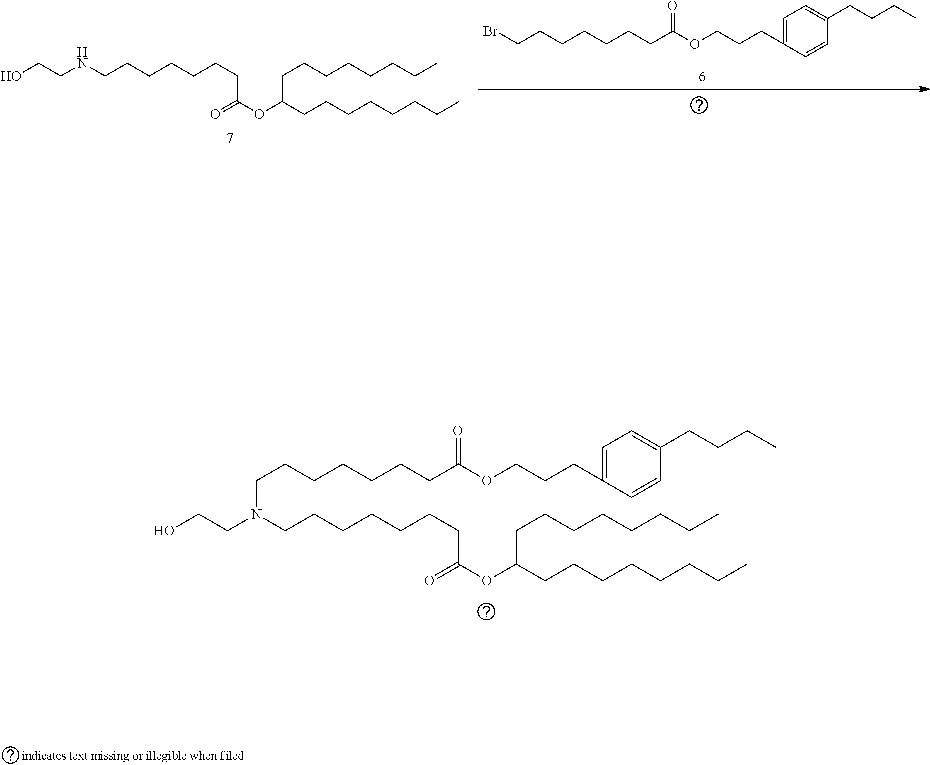 [SMILES: CCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCC)cc1]

Compound 6 (167 mg, 0.41 mmol, 1.2 eq.), compound 7 (150 mg, 0.34 mmol, 1.0 eq.), KI (113 mg, 0.68 mmol, 2.0 eq.), and CPME (2 mL) were dissolved in MeCN (2 mL). K₂CO₃ (235 mg, 1.70 mmol, 5.0 eq.) was added. The reaction was carried out overnight at 90° C. under nitrogen protection. TLC (DCM/MeOH=10/1) showed that the raw materials reacted completely and the desired product was produced. The reaction mixture was quenched with water (50 mL). Extraction was carried out with EA (60 ml×3). The organic phase was dried with anhydrous Na₂SO₄, filtered, and spin-dried under reduced pressure. The residue was purified by silica gel column, and eluted with DCM/MeOH (1/0-10/1, v/v) to obtain light-yellow oily compound (105 mg, 40.3%, SW-II-137-2).

LCMS: Rt: 1.660 min; MS m/z(ELSD): 772.4[M+H]⁺;

HPLC: 98.38% purity, ELSD; RT=8.743 min.

¹H NMR (400 MHz, CDCl₃) δ7.10 (d, J=8.8 Hz, 4H), 5.04-4.74 (m, 1H), 4.08 (t, J=6.4 Hz, 2H), 3.58 (t, J=5.2 Hz, 2H), 2.65 (dd, J=9.6, 5.6 Hz, 4H), 2.60-2.44 (m, 6H), 2.29 (dd, J=16.4, 7.6 Hz, 4H), 2.01-1.88 (m, 2H), 1.59(dt, J=9.2, 7.2 Hz, 6H), 1.54-1.42 (m, 8H), 1.39-1.11 (m, 41H), 0.88(dt, J=11.8, 6.0 Hz, 9H).

¹³C NMR (101 MHz, CDCl₃) δ 173.86(s), 173.63(s), 140.59(s), 138.34(s), 128.45(s), 128.24(s), 77.36(s), 77.04(s), 76.72(s), 74.14(s), 63.69(s), 58.11(s), 55.71(s), 53.90(s), 35.53(s), 34.68(s), 34.23(d, J=14.8 Hz), 31.82(d, J=11.6 Hz), 31.56(s), 31.26(s), 30.32(s), 29.53(d, J=2.8 Hz), 29.19(dd, J=8.0, 4.4 Hz), 27.20(d, J=2.4 Hz), 26.64(s), 25.33(s), 25.02(d, J=15.6 Hz), 22.62(d, J=11.6 Hz), 14.08(d, J=8.0 Hz).

### O. Compound SW-II-137-3

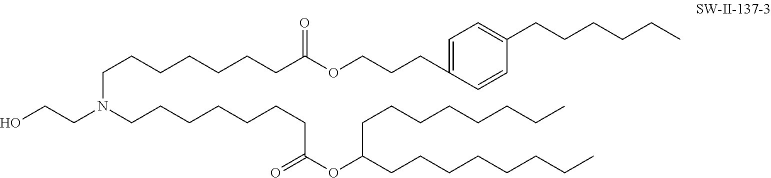 [SMILES: CCCCCCCCC(CCCCCCCC)OC(=O)CCCCCCCN(CCO)CCCCCCCC(=O)OCCCc1ccc(CCCCCC)cc1]

### 1. Synthesis of Compound 3

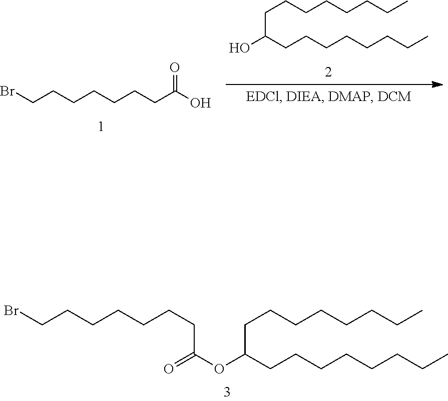 [SMILES: CCCCCCCCC(O)CCCCCCCC ; O=C(O)CCCCCCCBr ; CCCCCCCCC(CCCCCCCC)OC(=O)CCCCCCCBr]

EDCI (16.9 g, 88 mmol, 2 eq.) and DMAP (2.1 g, 18 mmol, 0.4 eq.) were added into a mixture of compound 1 (11.8 g, 53 mmol, 1.2 eq.) and compound 2 (11.2 g, 44 mmol, 1 eq.) in DCM (110 mL), and then DIEA (22.7 g, 176 mmol, 4 eq.) was added. The reaction mixture was stirred at room temperature for 16 hours under nitrogen. TLC (petroleum ether/ethyl acetate=30/1) showed that compound 1 was consumed and the desired product was formed. The reaction mixture was extracted with ethyl acetate (200 mL), washed with water (200 mL×3), dried with anhydrous sodium sulfate, filtered, and concentrated under reduced pressure. The residue was purified by silica gel column chromatography, and eluted with petroleum ether/ethyl acetate (1/0-20/1) to obtain colorless oily compound 3 (7.391 g, 37%).

### 2. Synthesis of Compound 5

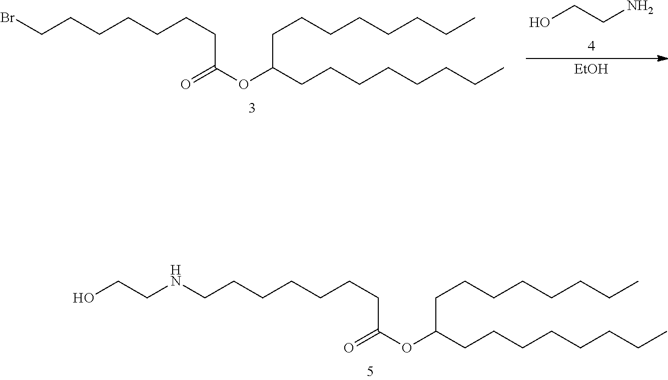

A mixture of compound 3 (7.391 mg, 16.07 mmol, 1 eq.) and compound 4 (29.4 g, 482.02 mmol, 30 eq.) in ethanol (2 mL) was stirred at 55° C. for 16 hours under nitrogen. TLC (DCM/MeOH=10/1) showed that a new major spot was observed. The reaction mixture was extracted with ethyl acetate (100 mL) and washed with water (3×100 mL). The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound 5 (3.695 g, 52%).

### 3. Synthesis of Compound 8

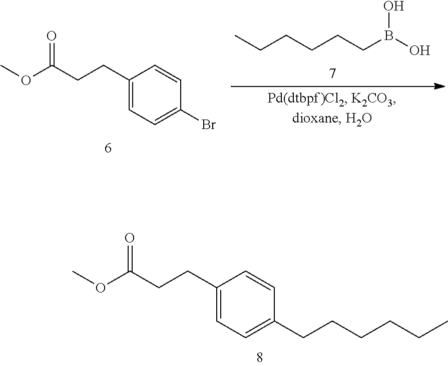

Pd(dtbpf)Cl₂ (269 mg, 0.41 mmol, 0.1 eq.) and potassium carbonate (1.7 g, 12.36 mmol, 3 eq.) were added into a mixture of compound 6 (1 g, 4.12 mmol, 1 eq.) and compound 7 (803 g, 6.17 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 8 (568 mg, 56%).

### 4. Synthesis of Compound 9

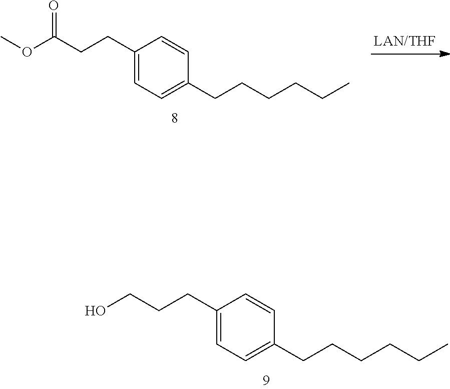 [SMILES: CCCCCCc1ccc(CCC(=O)OC)cc1 ; CCCCCCc1ccc(CCCO)cc1]

At 0° C. and under nitrogen environment, lithium aluminium hydride (2.3 mL, 2.29 mmol, 1 M, in THF, 1 eq.) was added into a mixture of compound 8 (568 mg, 2.29 mmol, 1 eq.) in THF (6 mL). The mixture was stirred at room temperature for 3 hours. TLC (PE/EA=5/1) showed that the reaction was complete and a new major spot was observed. The mixture was quenched with water (2.3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 9 (541 mg, >100%), which did not need to be further purified.

### 5. Synthesis of Compound 10

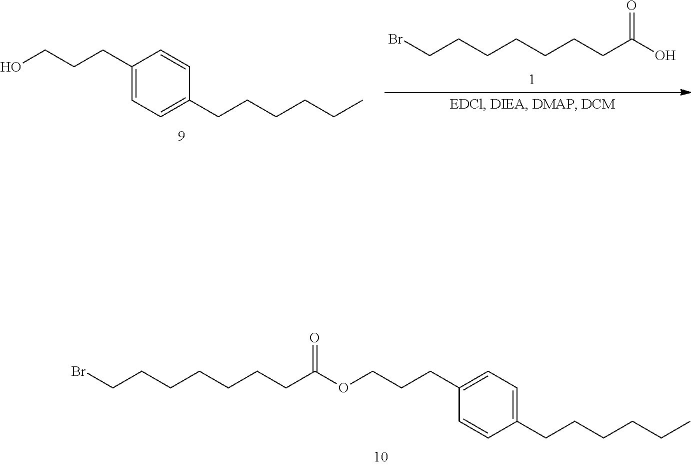 [SMILES: CCCCCCc1ccc(CCCO)cc1 ; O=C(O)CCCCCCCBr ; CCCCCCc1ccc(CCCOC(=O)CCCCCCCBr)cc1]

EDCI (768 mg, 4 mmol, 2 eq.) and DMAP (98 mg, 0.8 mmol, 0.4 eq.) were added into a mixture of compound 9 (441 mg, 2 mmol, 1 eq.) and compound 1 (536 mg, 2.4 mmol, 1.2 eq.) in DCM (5 mL), and then DIEA (1.032 g, 8 mmol, 4 eq.) was added. The reaction mixture was stirred at room temperature for 16 hours under nitrogen. TLC (petroleum ether/ethyl acetate=10/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-10/1) to obtain colorless oily compound 10 (372 mg, 44%).

### 6. Synthesis of SW-II-137-3

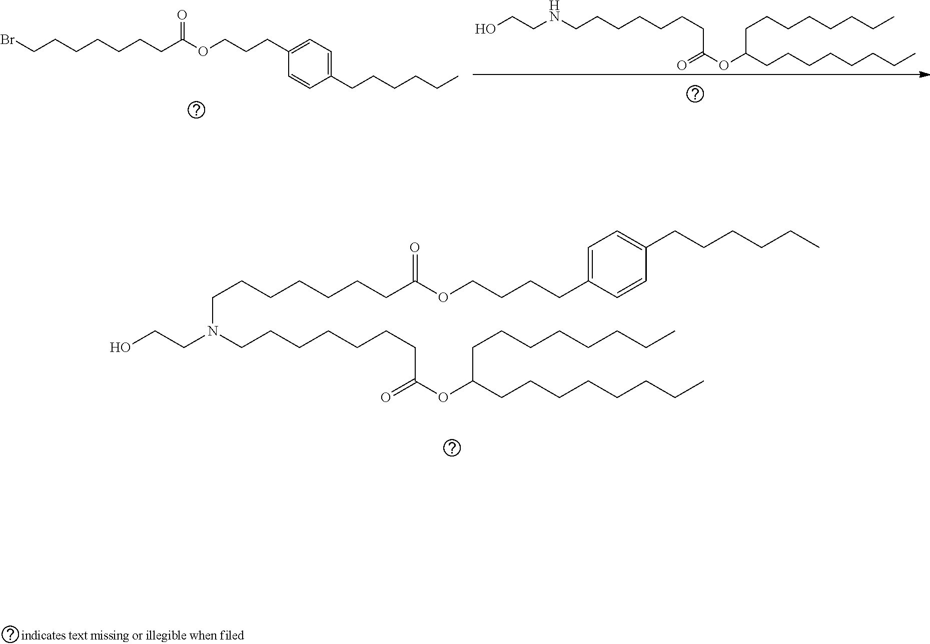 [SMILES: CCCCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCCc1ccc(CCCCCC)cc1]

Potassium carbonate (244 mg, 1.765 mmol, 6 eq.) and potassium iodide (117 mg, 0.706 mmol, 2 eq.) were added into a mixture of compound 10 (150 mg, 0.353 mmol, 1 eq.) and compound 5 (156 mg, 0.353 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH(1/0-10/1, v/v) to obtain yellow oily compound SW-II-137-3 (56.17 mg, 20%).

LCMS: Rt: 1.550 min; MS m/z(ELSD): 786.4[M+H]⁺;

HPLC: 98.597% purity, ELSD; RT=13.153 min.

¹H NMR (400 MHz, CDCl₃) δ7.09 (s, 4H), 4.92-4.78 (m, 1H), 4.08 (t, J=6.6 Hz, 2H), 3.62 (t, J=5.2 Hz, 2H), 2.78-2.50 (m, 10H), 2.35-2.22 (m, 4H), 2.00-1.88 (m, 2H), 1.57(ddd, J=28.9, 13.5, 4.5 Hz, 14H), 1.38-1.20 (m, 42H), 0.88 (t, J=6.8 Hz, 9H).

¹³C NMR (101 MHz, CDCl₃) δ 173.83(s), 173.60(s), 140.60(s), 138.33(s), 128.44(s), 128.24(s), 77.36(s), 77.04(s), 76.72(s), 74.15(s), 63.69(s), 57.95(s), 55.83(s), 53.95(s), 35.56(s), 34.65(s), 34.21(d, J=13.0 Hz), 31.81(d, J=12.3 Hz), 31.54(s), 30.32(s), 29.52(d, J=3.1 Hz), 29.34-28.94(m), 27.13(d, J=2.5 Hz), 26.31(s), 25.33(s), 24.99(d, J=15.7 Hz), 22.64(d, J=5.7 Hz), 14.11(s).

### P. Compound SW-II-138-1

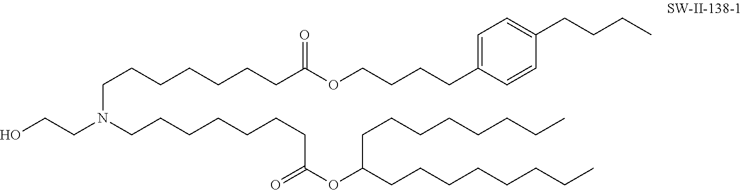 [SMILES: CCCCCCCCC(CCCCCCCC)OC(=O)CCCCCCCN(CCO)CCCCCCCC(=O)OCCCCc1ccc(CCCC)cc1]

### 1. Synthesis of Compound 2

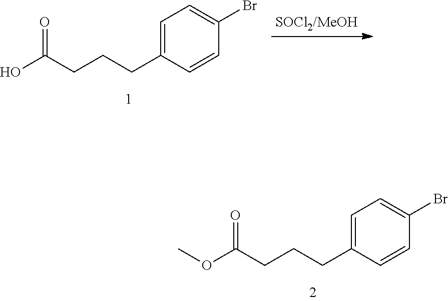

Compound 1 (4 g, 16.46 mmol, 1.0 eq.) was dissolved in MeOH (40 mL). Cooling was carried out to 0° C. SOCl₂ (3.9 g, 32.92 mmol, 2.0 eq.) was added dropwise. Then the reaction was carried out at room temperature for 1 hour. TLC (PE/EA=5/1) showed that the raw materials were consumed completely and the desired product was formed. The system was directly spin-dried under reduced pressure. A NaHCO₃ (70 mL) solution was added into the residue. Extraction was carried out with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain yellow oily compound 2 (4.1 mg, 95%).

### 2. Synthesis of Compound 4

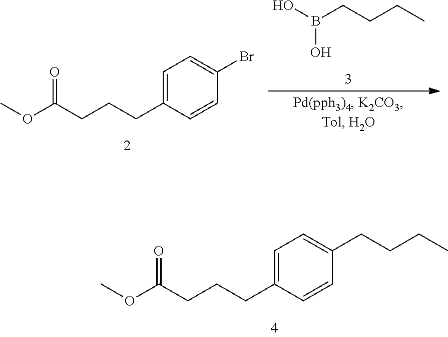

Compound 2 (500 mg, 1.95 mmol, 1.0 eq.), compound 3 (239 mg, 2.34 mmol, 1.2 eq.), Pd(PPh₃)₄ (225 mg, 0.19 mmol, 0.1 eq.), and K₂CO₃ (808 g, 5.85 mmol, 3.0 eq.) were dissolved in toluene (5.0 mL). Water (1 mL) was added. Then the reaction was carried out at 110° C. for 3 hours under nitrogen protection. TLC (PE/EA=5/1) showed that the raw materials were consumed completely and the desired product was formed. H₂O (70 mL) was added into the reaction for quenching. Extraction was carried out with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain yellow oily compound 4 (320 mg, 70%).

### 3. Synthesis of Compound 5

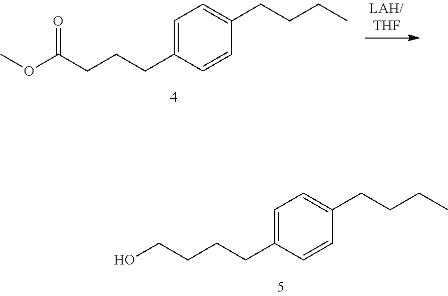 [SMILES: CCCCc1ccc(CCCC(=O)OC)cc1 ; CCCCc1ccc(CCCCO)cc1]

Compound 4 (300 mg, 1.28 mmol, 1.0 eq.) was dissolved in THF (4.0 mL). LAH (97 mg, 2.56 mmol, 2.0 eq.) was added at 0° C. Then the reaction was carried out at room temperature for 2 hours under nitrogen protection. TLC (PE/EA=5/1) showed that the raw materials were consumed completely and the desired product was formed. A HCl (1 M, 4 mL) solution and H₂O (10 mL) were added for quenching reaction. Extraction was carried out with EA (50 mL×3). The organic phase was washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain yellow oily compound 5 (224 mg, 84.8%).

### 4. Synthesis of Compound 7

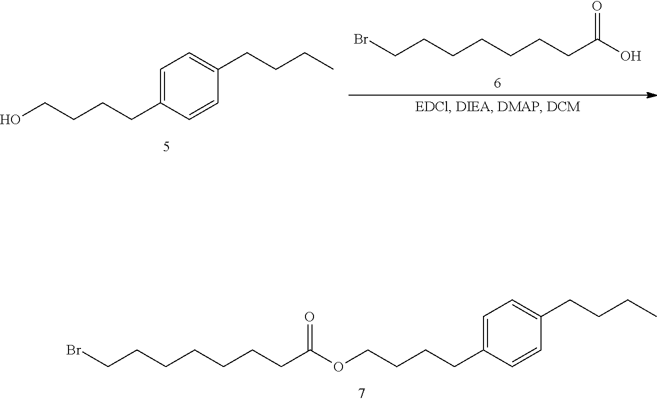

Compound 7 (224 mg, 1.09 mmol, 1.0 eq.) was dissolved in DCM (3.0 mL). Compound 6 (290 mg, 1.30 mmol, 1.2 eq.), EDCI (415 mg, 2.17 mmol, 2.0 eq.), DIEA (561 mg, 4.35 mmol, 4.0 eq.), and DMAP (53 mg, 0.43 mmol, 0.4 eq.) were added. Then, the reaction was carried out overnight at room temperature under nitrogen protection. TLC (PE/EA=30/1) showed that the raw materials were consumed completely and the desired product was formed. The reaction mixture was quenched with a HCl (1 M) solution, regulated to PH=4-6, and extracted with DCM (80 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 7 (208 mg, 46.7%).

### 5. Synthesis of SW-II-138-1

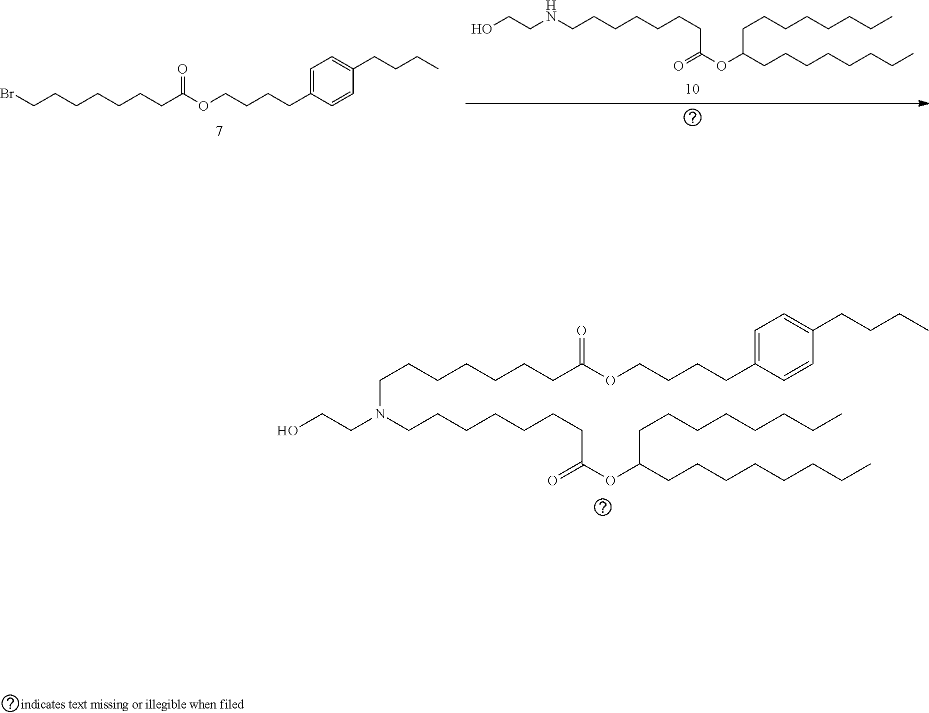 [SMILES: CCCCc1ccc(CCCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCCc1ccc(CCCC)cc1]

Compound 10 (110 mg, 0.25 mmol, 1 eq.), compound 7 (153 mg, 0.37 mmol, 1.5 eq.), KI (83 mg, 0.50 mmol, 2.0 eq.), and CPME (2 mL) were dissolved in MeCN (2 mL). K₂CO₃ (172 mg, 1.25 mmol, 5.0 eq.) was added. The reaction was carried out overnight at 90° C. under nitrogen protection. TLC (DCM/MeOH=10/1) showed that the raw materials were consumed completely and the desired product was formed. The reaction mixture was directly spin-dried under reduced pressure. The residue was purified by silica gel column, and eluted with DCM/MeOH (1/0-10/1, v/v) to obtain light-yellow oily compound (65 mg, 32%, SW-II-138-1).

LCMS: Rt: 1.684 min; MS m/z(ELSD): 772.4[M+H]⁺;

HPLC: 96.56% purity, ELSD; RT=6.346 min.

¹H NMR (400 MHz, CDCl₃) δ7.09 (s, 4H), 4.86 (s, 1H), 4.09(d, J=6.0 Hz, 2H), 3.97 (s, 2H), 3.07 (d, J=38.8 Hz, 6H), 2.69-2.51 (m, 4H), 2.28(td, J=7.3, 3.6 Hz, 4H), 1.79 (s, 4H), 1.70-1.46 (m, 16H), 1.42-1.17 (m, 37H), 0.90(dt, J=13.6, 7.2 Hz, 9H).

¹³C NMR (101 MHz, CDCl₃) δ 173.80(s), 173.53(s), 140.32(s), 139.13(s), 128.28(d, J=13.6 Hz), 77.43(s), 77.11(s), 76.80(s), 74.21(s), 64.22(s), 56.85(s), 55.98(s), 53.93(s), 35.22(s), 35.01(s), 34.54(s), 34.14(d, J=5.6 Hz), 33.71(s), 31.85(s), 29.50(d, J=2.8 Hz), 29.22(s), 29.12-28.60(m), 28.26(s), 27.78(s), 26.70(d, J=4.4 Hz), 25.31(s), 24.82(d, J=17.6 Hz), 24.28(s), 22.65(s), 22.37(s), 14.03(d, J=15.2 Hz).

### Q. SW-II-138-2

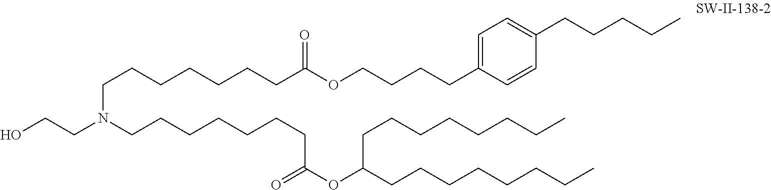 [SMILES: CCCCCCCCC(CCCCCCCC)OC(=O)CCCCCCCN(CCO)CCCCCCCC(=O)OCCCCc1ccc(CCCCC)cc1]

### 1. Synthesis of Compound 3

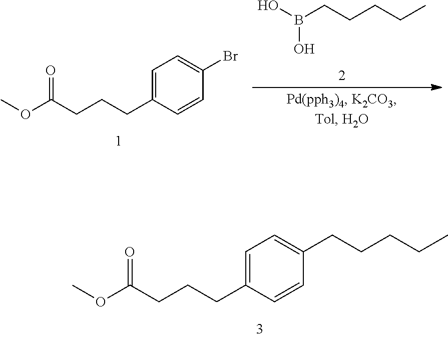

Compound 1 (500 mg, 1.95 mmol, 1.0 eq.), compound 2 (271 mg, 2.34 mmol, 1.2 eq.), Pd(PPh₃)₄ (225 mg, 0.20 mmol, 0.1 eq.), and K₂CO₃ (809 g, 5.86 mmol, 3.0 eq.) were dissolved in toluene (5.0 mL). Water (1 mL) was added. Then the reaction was carried out at 110° C. for 3 hours under nitrogen protection. TLC (PE/EA=5/1) showed that the raw materials were consumed completely and the desired product was formed. The reaction mixture was quenched with water (70 mL), and extracted with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 3 (320 mg, 70%).

### 2. Synthesis of Compound 4

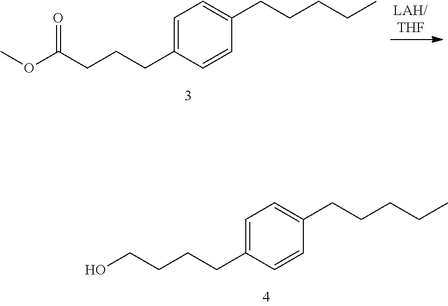 [SMILES: CCCCCc1ccc(CCCC(=O)OC)cc1 ; CCCCCc1ccc(CCCCO)cc1]

Compound 3 (320 mg, 1.29 mmol, 1.0 eq.) was dissolved in THF (3.0 mL). LAH (67 mg, 1.77 mmol, 2.0 eq.) was added at 0° C. Then, the reaction was carried out at room temperature for 2 hours under nitrogen protection. TLC (PE/EA=5/1) showed that the raw materials were consumed completely and the desired product was formed. The reaction mixture was quenched with a HCl (1 M, 2 mL) solution and H₂O (10 mL), and extracted with EA (50 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 4 (180 mg, 64%).

### 3. Synthesis of Compound 6

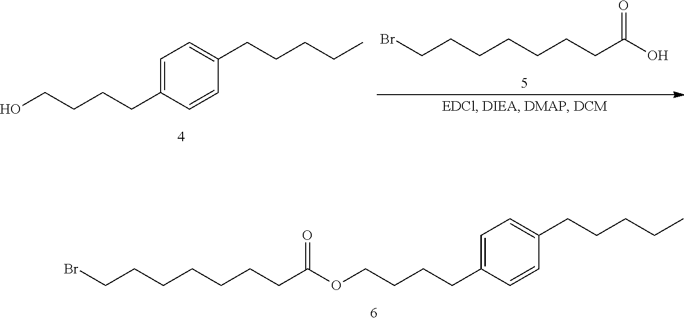

Compound 4 (180 mg, 0.82 mmol, 1.0 eq.) was dissolved in DCM (3.0 mL). Compound 5 (245 mg, 1.10 mmol, 1.2 eq.), EDCI (347 mg, 1.82 mmol, 2.0 eq.), DIEA (470 mg, 3.63 mmol, 4.0 eq.), and DMAP (45 mg, 0.36 mmol, 0.4 eq.) were added. Then, the reaction was carried out overnight at room temperature under nitrogen protection. TLC (PE/EA=30/1) showed that the raw materials were consumed completely and the desired product was formed. The reaction mixture was quenched with a HCl (1 M) solution, regulated to PH=5-6, and extracted with DCM (80 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 6 (220 mg, 63.6%).

### 4. Synthesis of SW-II-138-2

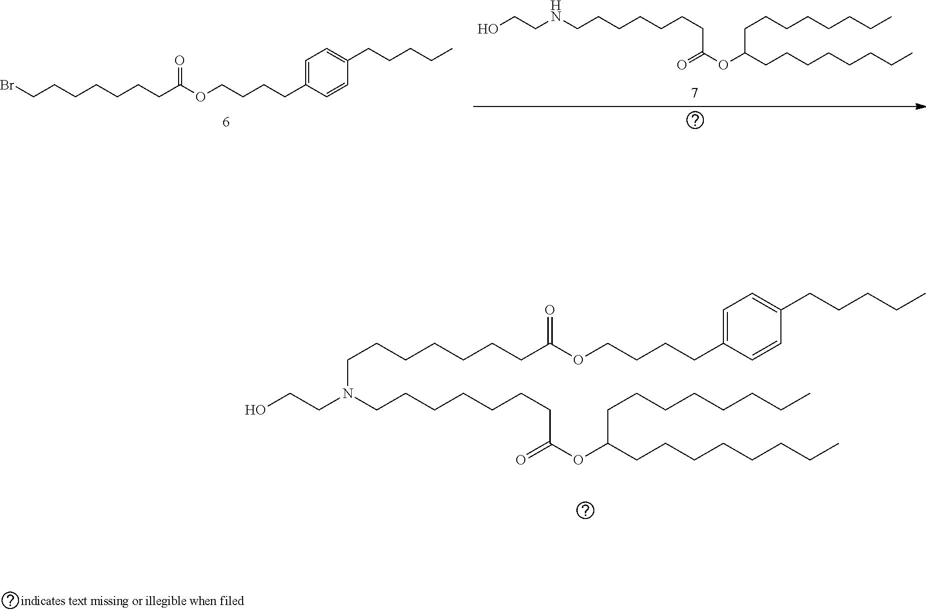 [SMILES: CCCCCc1ccc(CCCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCCc1ccc(CCCCC)cc1]

Compound 6 (158 mg, 0.37 mmol, 1.5 eq.), compound 7 (110 mg, 0.25 mmol, 1.0 eq.), KI (83 mg, 0.50 mmol, 2.0 eq.), and CPME (2 mL) were dissolved in MeCN (2 mL). K₂CO₃ (172 mg, 1.25 mmol, 5.0 eq.) was added. Then, the reaction was carried out overnight at 90° C. under nitrogen protection. TLC (DCM/MeOH=10/1) showed that the raw materials were consumed completely and the desired product was formed. The reaction mixture was directly spin-dried under reduced pressure. The residue was purified by silica gel column, and eluted with DCM/MeOH (1/0-10/1, v/v) to obtain colorless oily target product (100 mg, 51%, SW-II-138-2).

LCMS: Rt: 1.834 min; MS m/z(ELSD): 786.4[M+H]⁺;

HPLC: 99.20% purity, ELSD; RT=7.990 min.

¹H NMR(400 MHz, CDCl₃) δ7.00 (s, 4H), 4.88-4.73 (m, 2H), 4.00 (t, J=5.6 Hz, 2H), 3.81-3.54 (m, 2H), 3.00-2.81 (m, 2H), 2.81-2.65 (m, 4H), 2.50 (dd, J=16.4, 8.4 Hz, 4H), 2.20(td, J=7.6, 3.2 Hz, 4H), 1.56(ddd, J=18.4, 10.4, 5.2 Hz, 13H), 1.43 (d, J=5.6 Hz, 4H), 1.34-1.07 (m, 40H), 0.81(dt, J=11.2, 5.6 Hz, 9H).

¹³C NMR (101 MHz, CDCl₃) δ 173.78(s), 173.52(s), 140.32(s), 139.11(s), 128.25(d, J=11.6 Hz), 77.49(s), 77.17(s), 76.85(s), 74.14(s), 64.17(s), 57.25(s), 55.82(s), 53.85(s), 35.50(s), 35.01(s), 34.56(s), 34.14(d, J=7.2 Hz), 31.84(s), 31.53(s), 31.23(s), 29.49(d, J=2.8 Hz), 29.21(s), 28.94(dd, J=6.4, 4.4 Hz), 28.25(s), 27.77(s), 26.84(d, J=4.4 Hz), 25.30(s), 25.25-24.59(m), 22.59(d, J=11.2 Hz), 14.05(d, J=7.6 Hz).

### R. SW-II-138-3

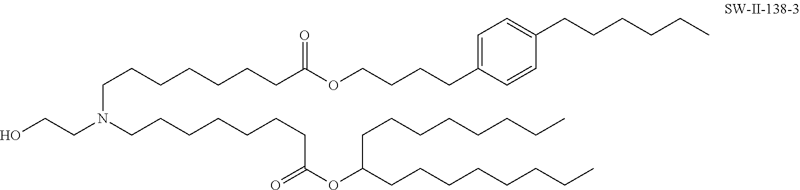 [SMILES: CCCCCCCCC(CCCCCCCC)OC(=O)CCCCCCCN(CCO)CCCCCCCC(=O)OCCCCc1ccc(CCCCCC)cc1]

### 1. Synthesis of Compound 3

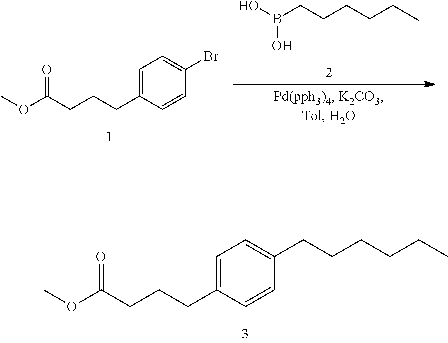

Compound 1 (500 mg, 1.95 mmol, 1.0 eq.), compound 2 (305 mg, 2.34 mmol, 1.2 eq.), Pd(PPh₃)₄ (225 mg, 0.20 mmol, 0.1 eq.), and K₂CO₃ (809 g, 5.86 mmol, 3.0 eq.) were dissolved in toluene (5.0 mL). Water (1 mL) was added. Then, the reaction was carried out at 110° C. for 3 hours under nitrogen protection. TLC (PE/EA=5/1) showed that the raw materials were consumed completely and the desired compound was formed. The reaction mixture was quenched with water (80 mL), and extracted with EA (80 mL×3). The organic phases were combined, washed with saturated salt solution (2×40 mL), dried with anhydrous Na₂SO₄, filtered, and spin-dried under reduced pressure. The residue was purified by silica gel column, and eluted with PE/EA (1/0-5/1, v/v) to obtain colorless oily compound 3 (260 mg, 51.3%).

### 2. Synthesis of Compound 4

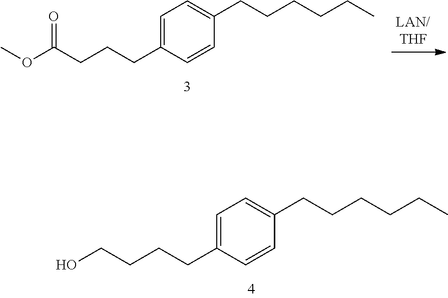 [SMILES: CCCCCCc1ccc(CCCC(=O)OC)cc1 ; CCCCCCc1ccc(CCCCO)cc1]

Compound 3 (260 mg, 0.99 mmol, 1.0 eq.) was dissolved in THF (4.0 mL). LAH (75 mg, 1.98 mmol, 2.0 eq.) was added at 0° C. Then, the reaction was carried out at room temperature for 2 hours under nitrogen protection. TLC (PE/EA=5/1) showed that the raw materials reacted completely and the desired compound was formed. The reaction mixture was quenched with a HCl (1 M, 4 mL) solution and H₂O (20 mL), and extracted with EA (50 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain colorless oily compound 4 (230 mg, 98%).

### 3. Synthesis of Compound 6

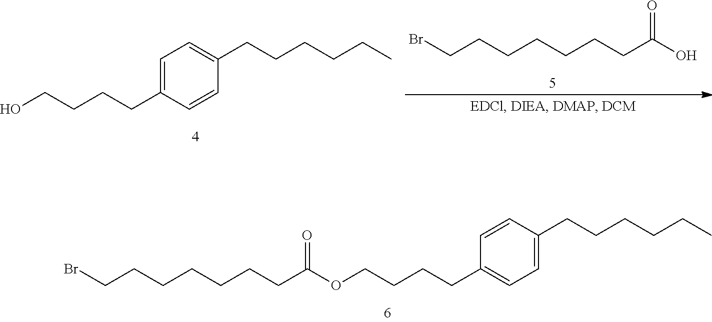

Compound 4 (240 mg, 1.03 mmol, 1.0 eq.) was dissolved in DCM (4.0 mL). Compound 5 (275 mg, 1.23 mmol, 1.2 eq.), EDCI (392 mg, 2.07 mmol, 2.0 eq.), DIEA (530 mg, 4.10 mmol, 4.0 eq.), and DMAP (50 mg, 0.41 mmol, 0.4 eq.) were added in sequence. Then the reaction was carried out overnight at room temperature under nitrogen protection. TLC (PE/EA=20/1) showed that the raw materials were consumed completely and the desired compound was formed. The reaction mixture was quenched with HCl (1 M), regulated to PH=5-6, and extracted with DCM (80 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-20/1, v/v) to obtain colorless oily compound 6 (180 mg, 40.9%).

### 4. Synthesis of SW-II-138-3

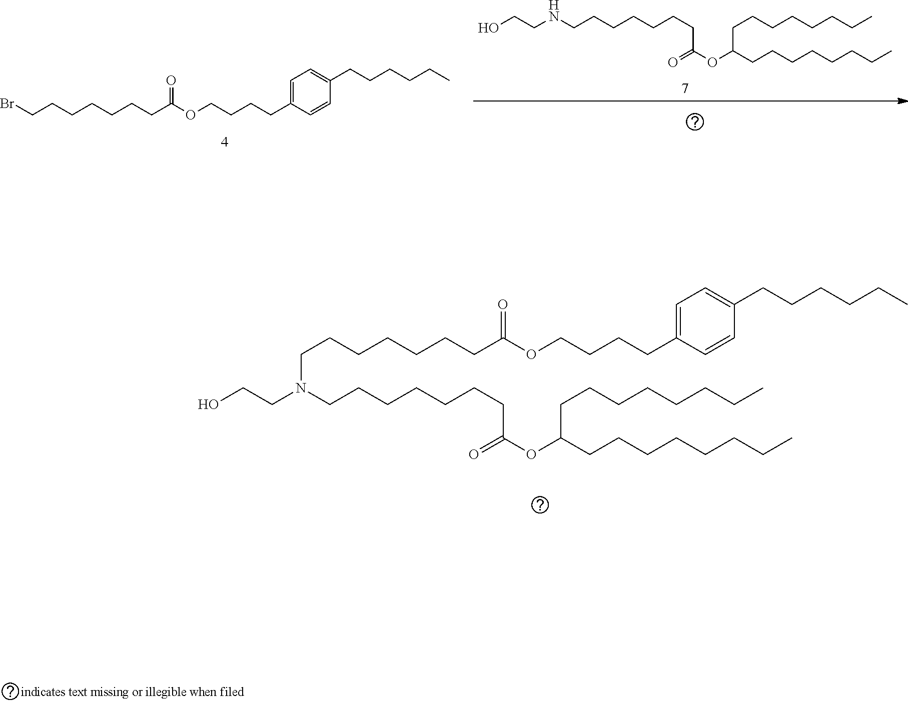 [SMILES: CCCCCCCCC(CCCCCCCC)OC(=O)CCCCCCCNCCO ; CCCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCCC)cc1]

Compound 6 (164 mg, 0.37 mmol, 1 eq.), compound 7 (110 mg, 0.24 mmol, 1.0 eq.), KI (83 mg, 0.49 mmol, 2.0 eq.), and CPME (2 mL) were dissolved in MeCN (2 mL). K₂CO₃ (172 mg, 1.24 mmol, 5.0 eq.) was added. Then, the reaction was carried out overnight at 90° C. under nitrogen protection. TLC (DCM/MeOH=10/1) showed that the raw materials were consumed completely and the desired product was formed. The reaction mixture was directly spin-dried under reduced pressure. The residue was purified by silica gel column, and eluted with DCM/MeOH (1/0-10/1, v/v) to obtain colorless oily target product (108 mg, 52.76%, SW-II-138-3).

LCMS: Rt: 2.007 min; MS m/z(ELSD): 800.4[M+H]⁺;

HPLC: 97.95% purity, ELSD; RT=9.455 min.

¹H NMR (400 MHz, CDCl₃) δ7.08 (s, 4H), 4.86(p, J=6.4 Hz, 1H), 4.08 (s, 2H), 3.60 (t, J=5.2 Hz, 3H), 2.76-2.42(m, 10H), 2.28(td, J=7.6, 2.8 Hz, 4H), 1.70-1.42 (m, 18H), 1.28(d, J=20.0 Hz, 41H), 0.88 (t, J=6.8 Hz, 9H).

¹³C NMR (101 MHz, CDCl₃) δ 173.85(s), 173.59(s), 140.39(s), 139.15(s), 128.27(d, J=12.0 Hz), 77.38(s), 77.07(s), 76.75(s), 74.13(s), 64.17(s), 58.06(s), 55.75(s), 53.92(s), 35.57(s), 35.03(s), 34.66(s), 34.22(d, J=13.2 Hz), 31.81(d, J=12.4 Hz), 31.54(s), 29.52(d, J=2.9 Hz), 29.34-28.95(m), 28.29(s), 27.79(s), 27.16(d, J=3.6 Hz), 26.50(s), 25.32(s), 24.99(d, J=17.6 Hz), 22.64(d, J=5.6 Hz), 14.10(s).

### S. Compound SW-II-139-1

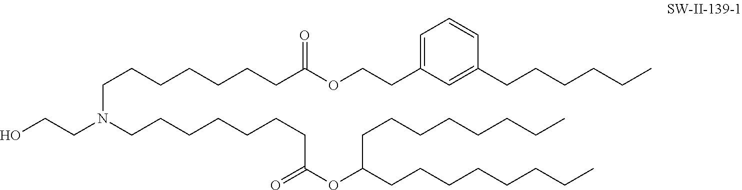 [SMILES: CCCCCCCCC(CCCCCCCC)OC(=O)CCCCCCCN(CCO)CCCCCCCC(=O)OCCc1cccc(CCCCCC)c1]

### 1. Synthesis of Compound 3

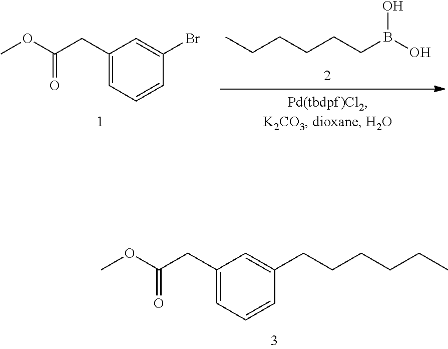

Pd(dtbpf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (852 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (691 mg, 68%).

### 2. Synthesis of Compound 4

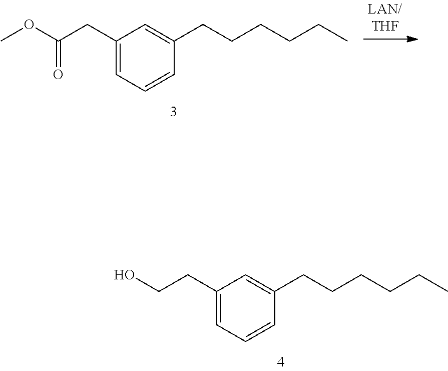 [SMILES: CCCCCCc1cccc(CC(=O)OC)c1 ; CCCCCCc1cccc(CCO)c1]

At 0° C. and under nitrogen environment, lithium aluminium hydride (3 mL, 2.95 mmol, 1 M, in THF, 1 eq.) was added into a mixture of compound 3 (691 mg, 2.95 mmol, 1 eq.) in THF (7 mL). The mixture was stirred at room temperature for 3 hours. TLC (PE/EA=5/1) showed that the reaction was complete and a new major spot was observed. The mixture was quenched with water (3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (547 mg, 90%), which did not need to be further purified.

### 3. Synthesis of Compound 6

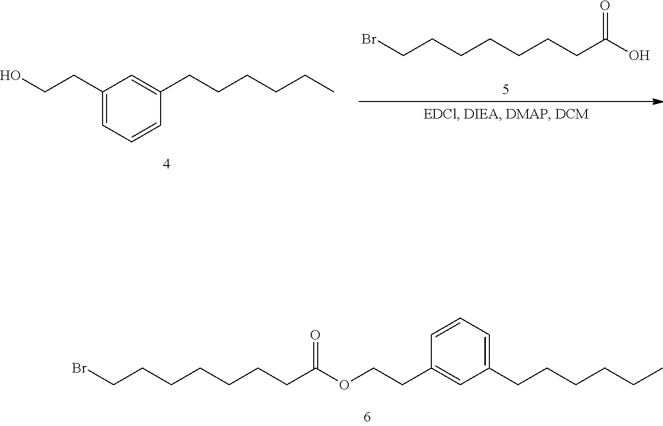 [SMILES: O=C(O)CCCCCCCBr ; CCCCCCc1cccc(CCO)c1 ; CCCCCCc1cccc(CCOC(=O)CCCCCCCBr)c1]

EDCI (833 mg, 4.34 mmol, 2 eq.) and DMAP (106 mg, 0.87 mmol, 0.4 eq.) were added into a mixture of compound 4 (447 mg, 2.17 mmol, 1 eq.) and compound 5 (581 mg, 2.6 mmol, 1.2 eq.) in DCM (5 mL), and then DIEA (1.12 g, 8.68 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (455 mg, 51%).

### 4. Synthesis of SW-II-139-1

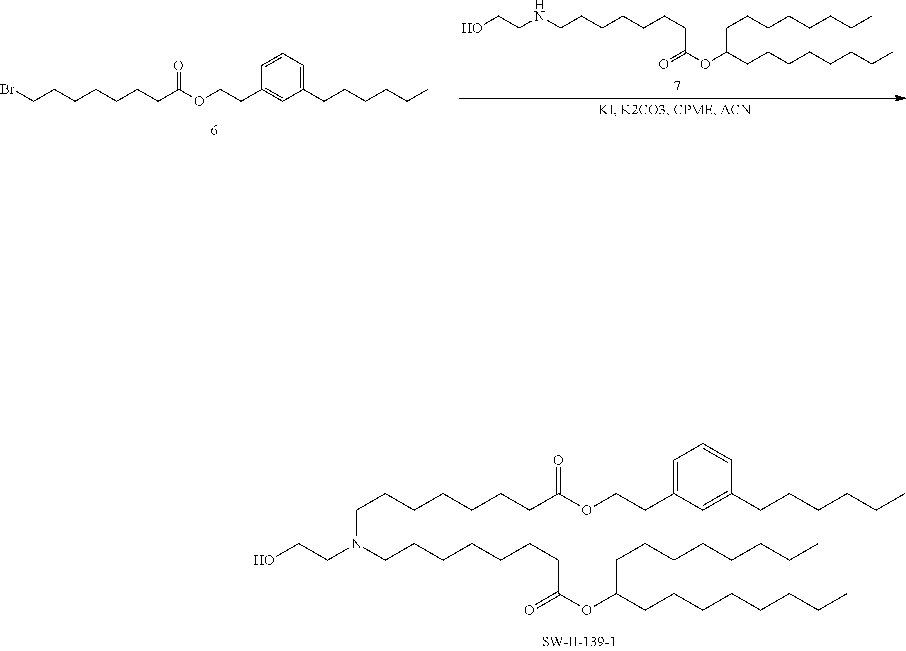 [SMILES: CCCCCCc1cccc(CCOC(=O)CCCCCCCBr)c1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1cccc(CCCCCC)c1]

Potassium carbonate (252 mg, 1.825 mmol, 6 eq.) and potassium iodide (121 mg, 0.73 mmol, 2 eq.) were added into a mixture of compound 6 (150 mg, 0.365 mmol, 1 eq.) and compound 7 (161 mg, 0.365 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-139-1 (54.53 mg, 19%).

LCMS: Rt: 1.521 min; MS m/z(ELSD): 772.4[M+H]⁺;

HPLC: 99.637% purity, ELSD; RT=12.347 min.

¹H NMR (400 MHz, CDCl₃) δ7.20 (t, J=7.7 Hz, 1H), 7.03 (t, J=6.8 Hz, 3H), 4.94-4.78 (m, 1H), 4.27 (t, J=7.2 Hz, 2H), 3.65 (t, J=5.1 Hz, 2H), 2.90 (t, J=7.2 Hz, 2H), 2.73 (t, J=4.9 Hz, 2H), 2.67-2.41 (m, 6H), 2.28(td, J=7.5, 2.7 Hz, 4H), 1.67-1.45 (m, 14H), 1.41-1.19 (m, 42H), 0.88 (dd, J=7.9, 5.7 Hz, 9H).

¹³C NMR (101 MHz, CDCl₃) δ 173.65(d, J=11.3 Hz), 143.17(s), 137.67(s), 129.04(s), 128.34(s), 126.61(s), 126.11(s), 77.30(d, J=11.6 Hz), 77.04(s), 76.72(s), 74.16(s), 64.85(s), 57.88(s), 55.93(s), 53.97(s), 35.94(s), 35.13(s), 34.64(s), 34.20(d, J=10.5 Hz), 31.80(d, J=13.7 Hz), 31.50(s), 29.52(d, J=2.9 Hz), 29.34-28.92(m), 27.08(d, J=3.9 Hz), 26.10(s), 25.33(s), 25.05(s), 24.82(s), 22.64(d, J=6.5 Hz), 14.11(s).

### T. Compound SW-II-139-2

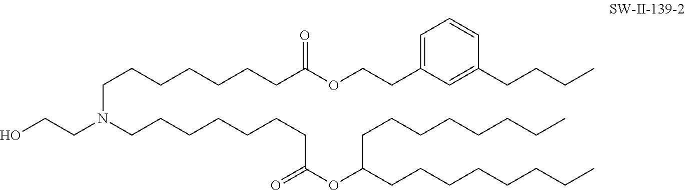 [SMILES: CCCCCCCCC(CCCCCCCC)OC(=O)CCCCCCCN(CCO)CCCCCCCC(=O)OCCc1cccc(CCCC)c1]

### 1. Synthesis of Compound 3

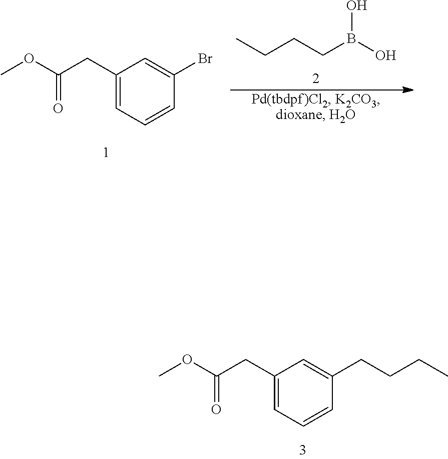

Pd(dtbpf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (668 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (605 mg, 67%).

### 2. Synthesis of Compound 4

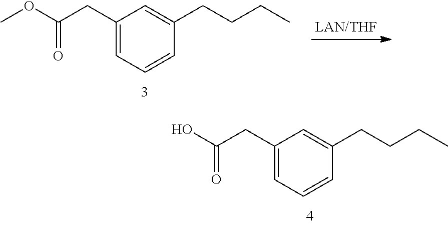

At 0° C. and under nitrogen environment, lithium aluminium hydride (3 mL, 2.94 mmol, 1 M, in THF, 1 eq.) was added into a mixture of compound 3 (605 mg, 2.94 mmol, 1 eq.) in THF (7 mL). The mixture was stirred at room temperature for 3 hours. TLC (PE/EA=5/1) showed that the reaction was complete and a new major spot was observed. The mixture was quenched with water (3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (534 mg, >100%), which did not need to be further purified.

### 3. Synthesis of Compound 6

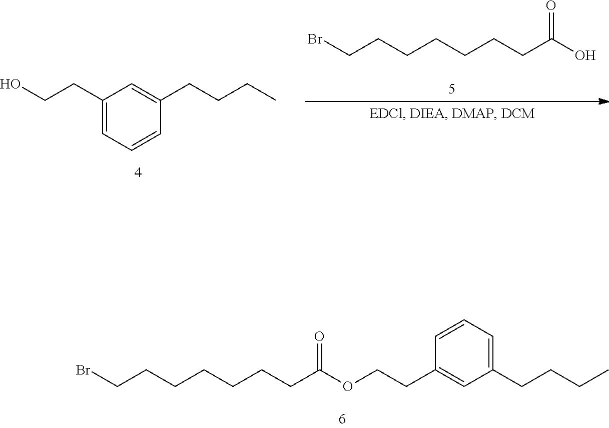 [SMILES: O=C(O)CCCCCCCBr ; CCCCc1cccc(CCO)c1 ; CCCCc1cccc(CCOC(=O)CCCCCCCBr)c1]

EDCI (937 mg, 4.88 mmol, 2 eq.) and DMAP (119 mg, 0.976 mmol, 0.4 eq.) were added into a mixture of compound 4 (434 mg, 2.44 mmol, 1 eq.) and compound 5 (652 mg, 2.93 mmol, 1.2 eq.) in DCM (5 mL), and then DIEA (1.259 g, 9.76 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (355 mg, 38%).

### 4. Synthesis of SW-II-139-2

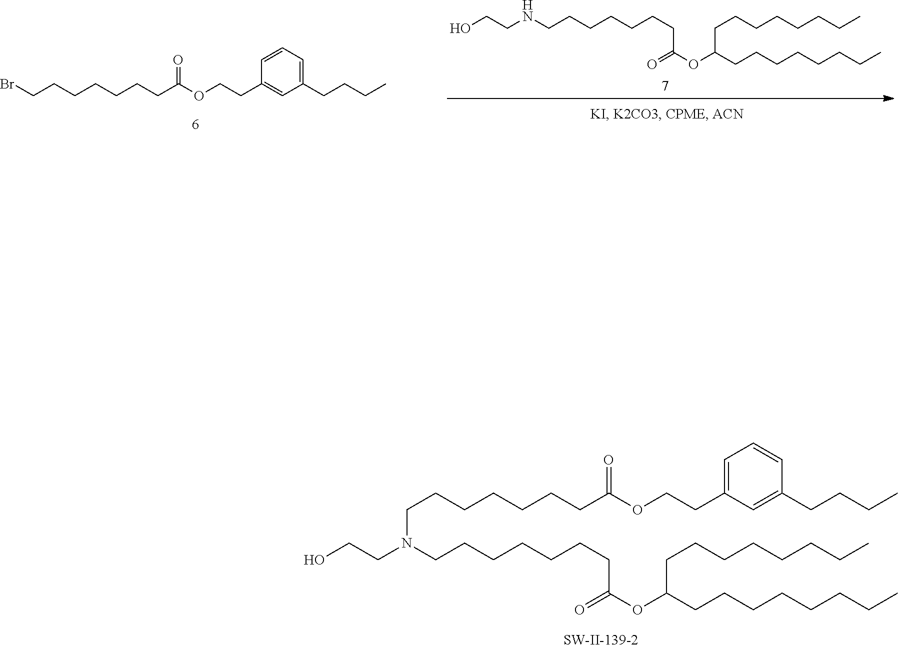 [SMILES: CCCCc1cccc(CCOC(=O)CCCCCCCBr)c1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1cccc(CCCC)c1]

Potassium carbonate (220 mg, 1.595 mmol, 5 eq.) and potassium iodide (106 mg, 0.638 mmol, 2 eq.) were added into a mixture of compound 6 (122 mg, 0.319 mmol, 1 eq.) and compound 7 (140 mg, 0.319 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-139-2 (45.48 mg, 19%).

LCMS: Rt: 1.346 min; MS m/z(ELSD): 744.3[M+H]⁺;

HPLC: 97.994% purity, ELSD; RT=11.235 min.

¹H NMR (400 MHz, CDCl₃) δ7.20 (t, J=7.8 Hz, 1H), 7.03 (t, J=7.6 Hz, 3H), 4.91-4.81 (m, 1H), 4.27 (t, J=7.2 Hz, 2H), 3.89-3.75 (m, 2H), 2.99-2.79 (m, 7H), 2.64-2.48 (m, 2H), 2.28(td, J=7.5, 3.1 Hz, 4H), 1.74-1.08 (m, 53H), 0.90(dt, J=13.6, 7.2 Hz, 9H).

¹³C NMR (101 MHz, CDCl₃) δ 173.60(d, J=11.7 Hz), 143.13(s), 137.65(s), 129.06(s), 128.34(s), 126.64(s), 126.11(s), 77.30(d, J=11.4 Hz), 77.04(s), 76.72(s), 74.22(s), 64.88(s), 57.28(s), 56.55(s), 54.11(s), 35.60(s), 35.12(s), 34.56(s), 34.15(d, J=4.0 Hz), 33.68(s), 31.86(s), 29.52(d, J=2.8 Hz), 29.24(s), 28.91(dd, J=7.0, 4.2 Hz), 26.81(d, J=3.9 Hz), 25.33(s), 25.12-24.98(m), 24.83(d, J=22.2 Hz), 22.67(s), 22.40(s), 14.04(d, J=14.4 Hz).

### U. Compound SW-II-140-1

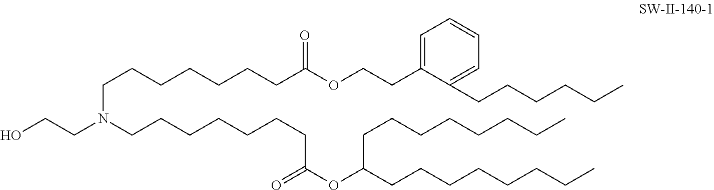 [SMILES: CCCCCCCCC(CCCCCCCC)OC(=O)CCCCCCCN(CCO)CCCCCCCC(=O)OCCc1ccccc1CCCCCC]

### 1. Synthesis of Compound 3

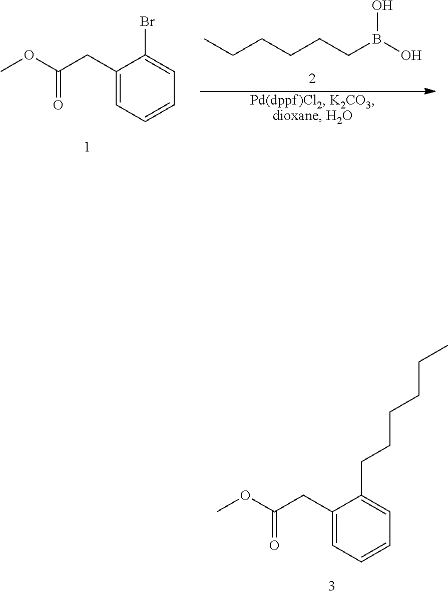

Pd(dppf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (852 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (748 mg, 73%).

### 2. Synthesis of Compound 4

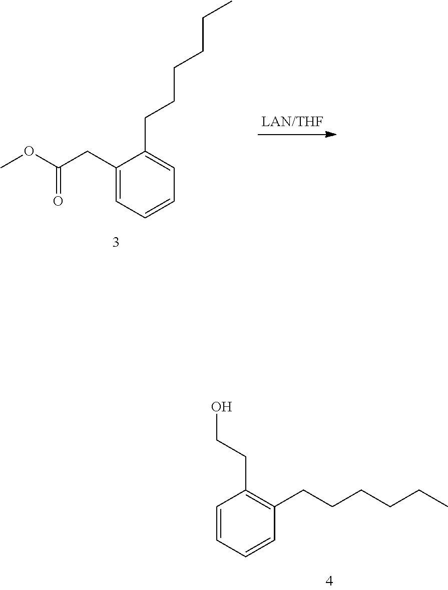 [SMILES: CCCCCCc1ccccc1CC(=O)OC ; CCCCCCc1ccccc1CCO]

At 0° C. and under nitrogen environment, lithium aluminium hydride (3.2 mL, 3.2 mmol, 1 M, in THF, 1 eq.) was added into a mixture of compound 3 (748 mg, 3.2 mmol, 1 eq.) in THF (8 mL). The mixture was stirred at room temperature for 3 hours. TLC (PE/EA=5/1) showed that the reaction was complete and a new major spot was observed. The mixture was quenched with water (3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (493 mg, 75%), which did not need to be further purified.

### 3. Synthesis of Compound 6

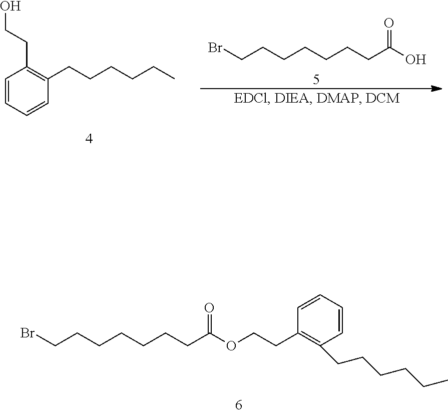

EDCI (733 mg, 3.82 mmol, 2 eq.) and DMAP (93 mg, 0.76 mmol, 0.4 eq.) were added into a mixture of compound 4 (393 mg, 1.91 mmol, 1 eq.) and compound 5 (511 mg, 2.29 mmol, 1.2 eq.) in DCM (5 mL), and then DIEA (986 mg, 7.64 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain compound 6 (327 mg, 42%), which was colorless oil.

### 4. Synthesis of SW-II-140-1

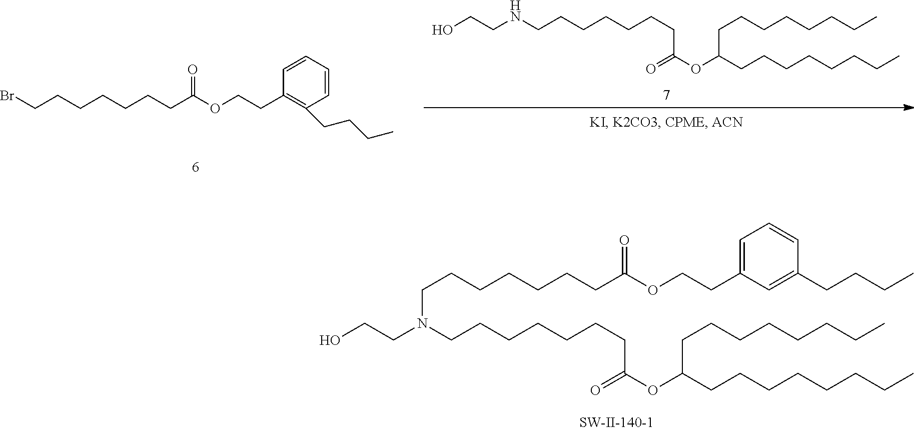 [SMILES: CCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1cccc(CCCC)c1]

Potassium carbonate (302 mg, 2.19 mmol, 6 eq.) and potassium iodide (121 mg, 0.73 mmol, 2 eq.) were added into a mixture of compound 6 (150 mg, 0.365 mmol, 1 eq.) and compound 7 (161 mg, 0.365 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-140-1 (180 mg, 64%).

LCMS: Rt: 1.568 min; MS m/z(ELSD): 772.4[M+H]⁺;

HPLC: 98.053% purity, ELSD; RT=8.702 min.

¹H NMR (400 MHz, CDCl₃) δ7.23-7.05 (m, 4H), 4.95-4.79 (m, 1H), 4.25 (t, J=7.4 Hz, 2H), 3.62 (t, J=4.8 Hz, 2H), 2.96 (dd, J=15.4, 8.0 Hz, 2H), 2.74-2.49 (m, 8H), 2.28 (dd, J=14.2, 7.2 Hz, 4H), 1.67-1.44 (m, 14H), 1.41-1.20 (m, 42H), 0.90(dt, J=13.2, 7.1 Hz, 9H).

¹³C NMR (101 MHz, CDCl₃) δ 173.68(d, J=10.2 Hz), 141.26(s), 135.23(s), 129.73(s), 129.37(s), 126.72(s), 125.92(s), 77.35(s), 77.03(s), 76.71(s), 74.17(s), 64.52(s), 57.99(s), 55.87(s), 53.94(s), 34.66(s), 34.21(d, J=11.5 Hz), 32.75(s), 31.83(d, J=9.8 Hz), 31.32(s), 29.65-28.88(m), 27.15(d, J=3.7 Hz), 26.35(s), 25.33(s), 25.07(s), 24.83(s), 22.66(d, J=3.4 Hz), 14.12(s).

### V. SW-II-140-2

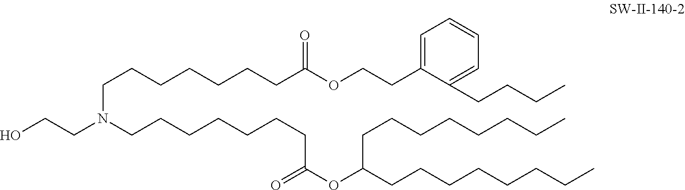 [SMILES: CCCCCCCCC(CCCCCCCC)OC(=O)CCCCCCCN(CCO)CCCCCCCC(=O)OCCc1ccccc1CCCC]

### 1. Synthesis of Compound 3

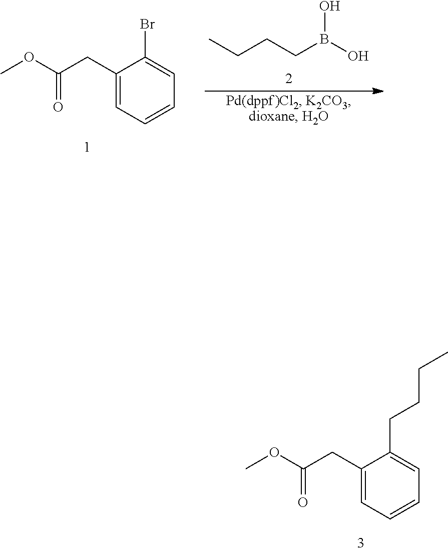

Pd(dppf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (668 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (406 mg, 45%).

### 2. Synthesis of Compound 4

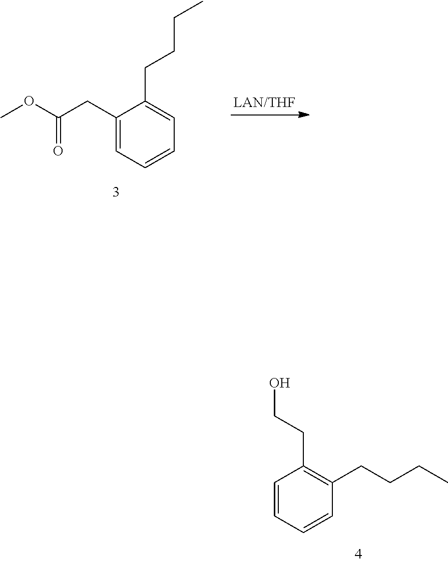

At 0° C. and under nitrogen environment, lithium aluminium hydride (2 mL, 1.97 mmol, 1 M, in THF, 1 eq.) was added into a mixture of compound 3 (406 mg, 1.97 mmol, 1 eq.) in THF (5 mL). The mixture was stirred at room temperature for 3 hours. TLC (PE/EA=5/1) showed that the reaction was complete and a new major spot was observed. The mixture was quenched with water (2 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (341 mg, 97%), which did not need to be further purified.

### 3. Synthesis of Compound 6

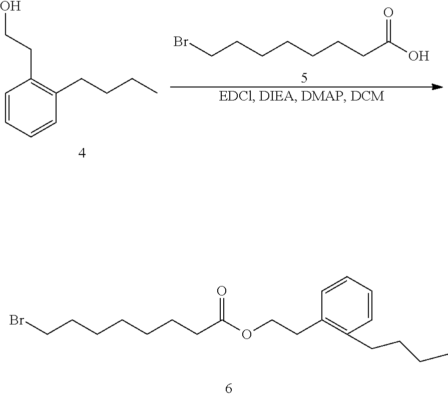

EDCI (518 mg, 2.7 mmol, 2 eq.) and DMAP (66 mg, 0.54 mmol, 0.4 eq.) were added into a mixture of compound 4 (241 mg, 1.35 mmol, 1 eq.) and compound 5 (361 mg, 1.62 mmol, 1.2 eq.) in DCM (3 mL), and then DIEA (697 mg, 5.4 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (185 mg, 32%).

### 4. Synthesis of SW-II-140-2

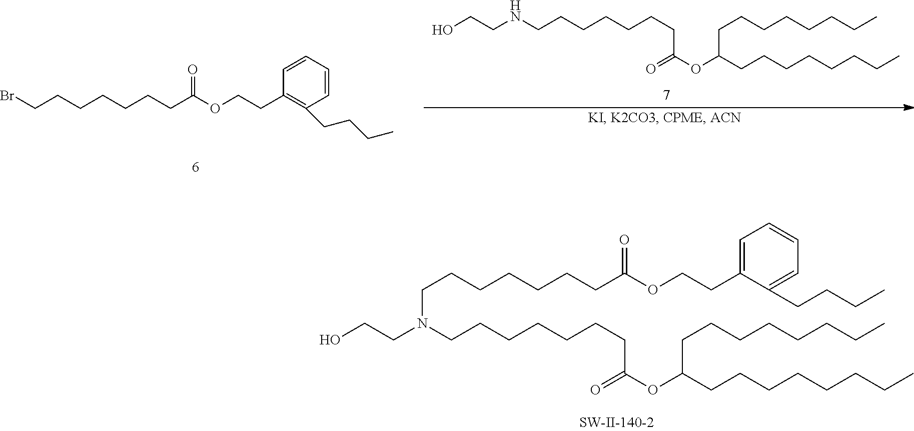 [SMILES: CCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccccc1CCCC]

Potassium carbonate (400 mg, 2.898 mmol, 6 eq.) and potassium iodide (160 mg, 0.966 mmol, 2 eq.) were added into a mixture of compound 6 (185 mg, 0.483 mmol, 1 eq.) and compound 7 (213 mg, 0.483 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-140-2 (161 mg, 45%).

LCMS: Rt: 1.696 min; MS m/z(ELSD): 744.3[M+H]⁺;

HPLC: 94.658% purity, ELSD; RT=5.938 min.

¹H NMR (400 MHz, CDCl₃) δ7.22-7.03 (m, 4H), 4.94-4.78 (m, 1H), 4.25 (t, J=7.3 Hz, 2H), 3.70-3.54 (m, 2H), 2.96 (t, J=7.4 Hz, 2H), 2.77-2.41 (m, 8H), 2.28(dd, J=14.3, 7.1 Hz, 4H), 1.65-1.18 (m, 52H), 0.91(dt, J=13.3, 7.1 Hz, 9H).

¹³C NMR (101 MHz, CDCl₃) δ 173.67(d, J=10.8 Hz), 141.22(s), 135.23(s), 129.73(s), 129.39(s), 126.72(s), 125.92(s), 77.36(s), 77.04(s), 76.72(s), 74.17(s), 64.52(s), 57.92(s), 55.92(s), 53.96(s), 34.66(s), 34.21(d, J=11.2 Hz), 33.51(s), 32.44(s), 31.83(d, J=9.3 Hz), 29.53(d, J=2.9 Hz), 29.14(dd, J=11.3, 8.5 Hz), 27.12(d, J=4.1 Hz), 26.23(s), 25.33(s), 25.06(s), 24.82(s), 22.73(d, J=9.9 Hz), 14.08(d, J=8.8 Hz).

### Example 2 In Vivo Expression of LPP Preparations Prepared by CDC Prescription and BIC Prescription

This example used the CDC prescription (see, for example, Yang R et al. A core-shell structured COVID-19 mRNA vaccine with favorable biodistribution pattern and promising immunity. Signal Transduct Target Ther. 2021 May 31; 6(1): 213) and the BIC prescription (see below) to prepare LPP preparations including a luciferase mRNA (Trilink Biotechnologies, SEQ ID NO: 1).

The process for preparation of the LPP preparation by the CDC prescription was as follows: preparation of a lipid mixed solution: M5:DOPE:mPEG2000-DSPE=49:49:2 was dissolved in an ethanol solution in a weight ratio.

Preparation of an aqueous mRNA solution: a luciferase mRNA was diluted into 0.35 mg/mL aqueous mRNA solution with 50 mM citric acid-sodium citrate buffer (pH of 3-4).

Preparation of a protamine sulfate solution: protamine sulfate was dissolved in nuclease-free water to prepare a protamine sulfate solution with a working concentration of 0.2 mg/mL.

Preparation of a core nanoparticle solution: by using a microfluidic technology, the protamine sulfate solution was mixed with the mRNA solution under the following conditions: volume=4.0 mL; flow rate ratio=3(mRNA):1(protamine solution), total flow rate=12 mL/min, pre-waste=0.35 mL, post-waste=0.1 mL, and room temperature, to obtain a core nanoparticle solution formed by protamine and mRNA.

Preparation of LPP: the core nanoparticle solution and the lipid solution were secondarily mixed under the following conditions: volume=4.0 mL, flow rate ratio=3(core nanoparticle solution): 1(lipid solution), total flow rate=12 mL/min, pre-waste=0.35 mL, post-waste=0.1 mL, and room temperature, to obtain an LPP-mRNA solution.

Centrifugal ultrafiltration: the LPP-mRNA solution was subjected to ultrafiltration and centrifugation to remove ethanol (centrifuge force of 3,400 g, centrifuge time of 60 minutes, and temperature of 4° C.), to prepare the LPP preparation prepared by the CDC prescription.

The process for preparation of the LPP preparation by the BIC prescription was as follows: preparation of a lipid mixed solution: M5:DOPE:cholesterol:mPEG2000-DMG=40:15:43.5:1.5 was dissolved in an ethanol solution in a mole ratio.

Preparation of an aqueous mRNA solution: a luciferase mRNA was diluted into 0.35 mg/mL aqueous mRNA solution with 50 mM citric acid-sodium citrate buffer (pH of 3-4).

Preparation of a protamine sulfate solution: protamine sulfate was dissolved in nuclease-free water to prepare a protamine sulfate solution with a working concentration of 0.2 mg/mL.

Preparation of a core nanoparticle solution: by using a microfluidic technology, the protamine sulfate solution was mixed with the mRNA solution under the following conditions: volume=4.0 mL; flow rate ratio=3(mRNA):1(protamine solution), total flow rate=12 mL/min, pre-waste=0.35 mL, post-waste=0.1 mL, and room temperature, to obtain a core nanoparticle solution formed by protamine and mRNA.

Preparation of LPP: the core nanoparticle solution and the lipid solution were secondarily mixed under the following conditions: volume=4.0 mL, flow rate ratio=3(core nanoparticle solution): 1(lipid solution), total flow rate=12 mL/min, pre-waste=0.35 mL, post-waste=0.1 mL, and room temperature, to obtain an LPP-mRNA solution.

Centrifugal ultrafiltration: the LPP-mRNA solution was subjected to ultrafiltration and centrifugation to remove ethanol (centrifuge force of 3,400 g, centrifuge time of 60 minutes, and temperature of 4° C.), to prepare the LPP preparation prepared by the BIC prescription.

Female BALB/c mice (Charles River Laboratories) of 6-8 weeks of age were divided into three groups (CDC prescription group, BIC prescription group, and PBS group, with 3 mice per group). An LPP solution containing 10 μg of luciferase mRNA prepared by the CDC prescription, an LPP solution prepared by the BIC prescription containing 10 μg of luciferase mRNA, and control PBS were administered by intramuscular injection (hind limbs of mice), respectively. 6 Hours after the administration, mice were injected intraperitoneally with 30 mg of a D-fluorescein substrate (Maokang Biotechnology). The bioluminescence was measured in a Xenogen IVIS-200 imaging system 15 minutes after substrate injection to evaluate the in vivo expression of the luciferase mRNA in the injection sites and livers of the mice.

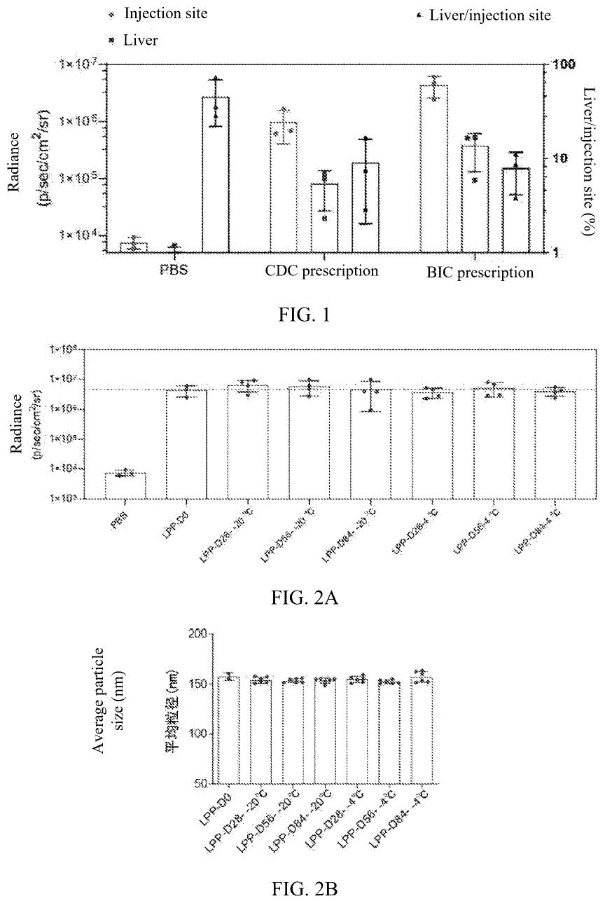

The experimental results are shown in FIG. 1, where the three columns in each group are the luciferase expression of injection site, the luciferase expression in the liver, and the ratio of luciferase expression of liver/injection site, from left to right respectively. It is observed that the LPP prepared by the BIC prescription has higher fluorescence at the injection site than the LPP prepared by the CDC prescription, while the liver/injection site (%) has a lower ratio of luciferase expression than the LPP prepared by the CDC prescription. It is shown that the LPP prepared by the BIC prescription has higher expression efficiency than the LPP prepared by the CDC prescription, and has less expression in the liver, a good targeting effect, and low hepatotoxicity. Considering that the LPP in the BIC prescription is a four-lipid composition, while the LPP in the CDC prescription is a three-lipid composition, the following examples further study the four-lipid composition, and optimizes the delivery system of the lipid composition.

### Example 3 Comparison of Expression Amounts of LPP Preparations with Different Lipid Ratios

This example used the formulation in Table 1 to prepare four-lipid LPP preparations SW0123.351-1LPP to SW0123.351-23LPP containing eGFP mRNA (Trilink Biotechnologies, SEQ ID NO: 2).

The process for preparation of SW0123.351-1LPP was as follows: preparation of a lipid mixed solution: M5:DOPE:cholesterol:mPEG2000-DMG=40:15:43.5:1.5 was dissolved in an ethanol solution in a ratio.

Preparation of an aqueous mRNA solution: a luciferase mRNA was diluted into 0.35 mg/mL aqueous mRNA solution with 50 mM citric acid-sodium citrate buffer (pH of 3-4).

Preparation of a protamine sulfate solution: protamine sulfate was dissolved in nuclease-free water to prepare a protamine sulfate solution with a working concentration of 0.2 mg/mL.

Preparation of a core nanoparticle solution: by using a microfluidic technology, the protamine sulfate solution was mixed with the mRNA solution under the following conditions: volume=4.0 mL; flow rate ratio=3(mRNA):1(protamine solution), total flow rate=12 mL/min, pre-waste=0.35 mL, post-waste=0.1 mL, and room temperature, to obtain a core nanoparticle solution formed by protamine and mRNA.

Preparation of LPP: the core nanoparticle solution and the lipid solution were secondarily mixed under the following conditions: volume=4.0 mL, flow rate ratio=3(core nanoparticle solution): 1(lipid solution), total flow rate=12 mL/min, pre-waste=0.35 mL, post-waste=0.1 mL, and room temperature, to obtain an LPP-mRNA solution.

Centrifugal ultrafiltration: the LPP-mRNA solution was subjected to ultrafiltration and centrifugation to remove ethanol (centrifuge force of 3,400 g, centrifuge time of 60 minutes, and temperature of 4° C.), to prepare the LPP preparation numbered SW0123.351-1LPP.

The preparation method of the LPP preparations numbered SW0123.351-2LPP to SW0123.351-23LPP was consistent with that of SW0123.351-1LPP, with the difference in the lipid ratio (as shown in Table 1). 100 ng of SW0123.351-1LPP to SW0123.351-23LPP solutions containing eGFP mRNA were taken respectively, to transfect dendritic cells (DC2.4 cells) with PBS as a control. 24 Hours after the administration, the cells were lysed, and the expression amount of a GFP protein was tested. The results are shown in Table 1, where SW0123.351-1, SW0123.351-5, SW0123.351-6, SW0123.351-9, SW0123.351-10, SW0123.351-12, SW0123.351-13, SW0123.351-18, SW0123.351-19, SW0123.351-20, and SW0123.351-21 all show a good in vitro transfection effect, while SW0123.351-2, SW0123.351-3, and SW0123.351-4 have no expression substantially. It is shown that the proportional relationship of M5, DOPE, cholesterol and mPEG2000-DMG influences the expression effect. When the molar ratio of lipids is M5:DOPE:cholesterol:mPEG2000-DMG=35-50:15-30:24-44:1-1.5, the effect is an increase in expression amount (the increase in expression amount means that the expression amount is greater than the average value of GFP expression).

The following examples make further study with SW0123.351-13LPP as SW0123.351-LPP.

TABLE 1
Influence of different lipid ratios on expression amount

| | | | | | GFP |
|---|---|---|---|---|---|
| | | | mPEG2000- | Choles- | expression |
| NO. | M5% | DOPE % | DMG % | terol % | (pg/ul) |
| SW0123.351-1 | 40 | 15 | 1.5 | 43.5 | 1906.1 |
| SW0123.351-2 | 10 | 40 | 1.5 | 48.5 | 152.7 |
| SW0123.351-3 | 20 | 30 | 1.5 | 48.5 | 134.8 |
| SW0123.351-4 | 70 | 10 | 1.5 | 18.5 | 792.5 |
| SW0123.351-5 | 50 | 15 | 1.25 | 33.75 | 2165.1 |
| SW0123.351-6 | 45 | 15 | 1.25 | 38.75 | 2109.2 |
| SW0123.351-7 | 35 | 15 | 1.5 | 48.5 | 784.0 |
| SW0123.351-8 | 55 | 30 | 1.5 | 13.5 | 1225.5 |
| SW0123.351-9 | 40 | 25 | 1 | 34 | 2489.5 |
| SW0123.351-10 | 40 | 20 | 1 | 39 | 2767.1 |
| SW0123.351-11 | 50 | 30 | 1 | 19 | 1349.3 |
| SW0123.351-12 | 40 | 15 | 1.5 | 43.5 | 1906.1 |
| SW0123.351-13 | 35 | 20 | 1 | 44 | 2940.8 |
| SW0123.351-14 | 55 | 25 | 1.25 | 18.75 | 1346.5 |
| SW0123.351-15 | 55 | 15 | 1 | 29 | 433.8 |
| SW0123.351-16 | 35 | 25 | 1.25 | 38.75 | 1679.3 |
| SW0123.351-17 | 55 | 15 | 1 | 29 | 1219.3 |
| SW0123.351-18 | 50 | 15 | 1 | 34 | 2108.7 |
| SW0123.351-19 | 50 | 20 | 1.25 | 28.75 | 2082.9 |
| SW0123.351-20 | 45 | 15 | 1.25 | 38.75 | 2181.9 |
| SW0123.351-21 | 45 | 30 | 1 | 24 | 1952.4 |
| SW0123.351-22 | 55 | 20 | 1.25 | 23.75 | 674.3 |
| SW0123.351-23 | 50 | 25 | 1.5 | 23.5 | 891.6 |

### Example 4 Comparison of In Vivo Expression Amount and In Vitro Physicochemical Property of SW0123.351-LPP in Different Storage Environments

This example used the preparation method as provided in Example 3 to prepare an SW0123.351-LPP solution containing luciferase mRNA (SEQ ID NO: 1, 0.1 mg/ml).

The appropriate amount of an SW0123.351-LPP solution containing luciferase mRNA was taken and stored in a 4° C. environment, and the appropriate amount of an SW0123.351-LPP solution containing luciferase mRNA was taken and stored in a −20° C. environment, recorded as day 0. Sampling was carried out at weeks 4, 8, and 12, i.e., days 28, 56, and 84, after storage, respectively, to test the in vivo luciferase signal expression and the in vitro physicochemical property. The specific test methods were as follows:

Test of in vivo luciferase signal expression: female BALB/c mice (Charles River Laboratories) of 6-8 weeks of age were divided into eight groups (3 mice in each of PBS and LPP-DO group, and 4 mice in each of LPP-D28—20° C., LPP-D56—20° C., LPP-D84—20° C., LPP-D28—4° C., LPP-D56—4° C., and LPP-D84—4° C. groups). The control PBS, the SW0123.351-LPP solution containing 10 g of luciferase mRNA at day 0, the SW0123.351-LPP solutions stored in −20° C. environment at days 28, 56, and 84, and the SW0123.351-LPP solutions stored in a 4° C. environment at days 28, 56, and 84 were administered by intramuscular injection (hind limbs of mice), respectively. 6 Hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the mice.

Test of particle size: 50 ul of LPP samples (2 replicates of the SW0123.351-LPP solution on day 0, 6 replicates of SW0123.351-LPP solutions stored in a −20° C. environment at days 28, 56, and 84, and 6 replicates of the SW0123.351-LPP solutions stored in a 4° C. environment at days 28, 56, and 84) were taken and diluted with 950 uL of purified water to obtain diluted LPP samples. The samples were placed in a dynamic light scattering laser particle size analyzer (Malvern, ZS-90) for test.

The results of in vivo luciferase signal expression in different storage environments are shown in FIG. 2A, where after SW0123.351-LPP is stored in a 4° C. environment and at −20° C. for 12 weeks, respectively, the in vivo luciferase expression has no significant change, and is very stable.

The in vitro physicochemical property i.e., the average particle size (Z-Average), in different storage environments, are as shown in FIG. 2B, where after the SW0123.351-LPP is stored in a 4° C. environment and at −20° C. for 12 weeks, the particle size of the lipid nanoparticles is very stable, and is always maintained around 150 nm, without significant change, indicating the stability thereof.

The above experimental results show that the in vivo expression level and the in vitro physicochemical property of SW0123.351-LPP provided herein in a 4° C. refrigeration environment and in a −20° C. freezing environment can both be maintained in a stable state for a long time, indicating the high stability of the four-lipid composition in different storage environments.

### Example 5 Comparison of In Vivo Expression Amount and In Vitro Physicochemical Property of SW0123.351-LPP after Treatment with Different Times of Freeze Thawing

This example used the preparation method as provided in Example 3 to prepare an SW0123.351-LPP solution containing luciferase mRNA (SEQ ID NO: 1, 0.1 mg/ml).

The appropriate amount of SW0123.351-LPP solutions containing luciferase mRNA were taken respectively and stored in a −20° C. environment, and treated with 2 times, 4 times, 6 times, 8 times, and 10 times of freeze thawing, respectively, to test the in vivo luciferase signal expression and the in vitro physicochemical property. The samples without freeze thawing treatment were recorded as LPP-FTO. The LPP with treatment with 2 times, 4 times, 6 times, 8 times and 10 times of freeze thawing was recorded as LPP-FT2, LPP-FT4, LPP-FT6, LPP-FT8 and LPP-FT10, respectively. The specific test methods were as follows:

Test of in vivo luciferase signal expression: female BALB/c mice (Charles River Laboratories) of 6-8 weeks of age were divided into seven groups (3 mice in each of PBS and LPP-FTO group, and 4 mice in each of LPP-FT2, LPP-FT4, LPP-FT6, LPP-FT8, and LPP-FT10 groups). The control PBS, and the SW0123.351-LPP solutions containing 10 g of luciferase mRNA stored in −20° C. environment and respectively treated with 0 time, 2 times, 4 times, 6 times, 8 times, and 10 times of freeze thawing were administered by intramuscular injection (hind limbs of mice), respectively. 6 Hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the mice.

Test of particle size: 50 ul of LPP samples (2 replicates of LPP-FTO, 6 replicates of LPP-FT2, 6 replicates of LPP-FT4, 6 replicates of LPP-FT6, 6 replicates of LPP-FT8, and 6 replicates of LPP-FT10) were taken and diluted with 950 uL of purified water to obtain diluted LPP samples. The samples were placed in a dynamic light scattering laser particle size analyzer (Malvern, ZS-90) for test.

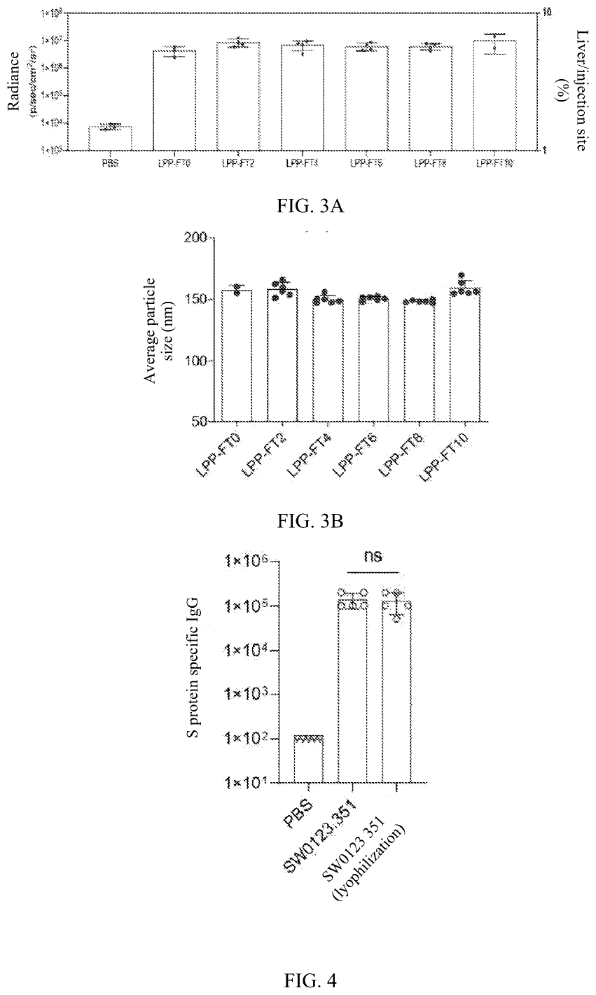

The results of in vivo luciferase signal expression of the SW0123.351-LPP solutions after treatment with different times of freeze thawing are as shown in FIG. 3A, where the in vivo luciferase signal expression of SW0123.351-LPP after treatment with 2 times, 4 times, 6 times, 8 times, and 10 times of freeze thawing has always no significant change, and maintains stable.

The in vitro physicochemical property i.e., the average particle size (Z-Average), of the SW0123.351-LPP solutions after treatment with different times of freeze thawing are as shown in FIG. 3B, where after SW0123.351-LPP is treated with 2 times, 4 times, 6 times, 8 times, and 10 times of freeze thawing, the particle size of the lipid nanoparticles has also no significant change, and is always maintained around 150 nm.

The above experimental results show that the in vivo expression level and the in vitro physicochemical property of SW0123.351-LPP provided herein after multiple times of freeze thawing can both be maintained in a stable state, indicating the high stability of the four-lipid composition under repeated freeze thawing.

### Example 6 Immunogenicity of SW0123.351 in Mice Before and After Lyophilization

This example used the preparation method as provided in Example 3 to respectively prepare SW0123.351-LPP containing COVID-19 mRNA (SEQ ID NO: 3) for lyophilization, to prepare SW0123.351-LPP lyophilized powder containing COVID-19 mRNA (0.1 mg/ml).

Female C57BL/6 mice (Shanghai Lingchang Biotechnology Co., Ltd.) of 6-8 weeks of age were divided into three groups (PBS group, freshly prepared SW0123.351-LPP solution group, and post-reconstitution SW0123.351-LPP solution group, with 5 mice per group). The appropriate amount of SW0123.351-LPP lyophilized powder containing COVID-19 mRNA was taken for reconstitution with purified water. C57BL/6 mice were immunized by intramuscular injection (hind limbs of mice) at days 1 and 14. 10 μg of each of a post-reconstitution SW0123.351-LPP solution containing COVID-19 mRNA and a freshly prepared SW0123.351-LPP solution containing COVID-19 mRNA was administrated respectively. Blood was taken from the orbits of the mice at day 14 after secondary immunization, and the S protein specific binding antibodies in serum were tested, with PBS as a control. The specific test methods were as follows:

A recombinant SARS-CoV-2 spike protein antigen (50 ng, Sino Biological) was diluted with carbonate buffer (0.1 M, pH 9.6) and coated onto EIA/RIA plates (Corning) overnight at 4° C. The plates were washed in PBST (0.05% Tween-20) and blocked with 10% goat serum (in PBS) at 37° C. for 2 hours. The serum samples were serially diluted using 2% goat serum with an initial dilution titer of 50, and then the serum samples to be tested were diluted at a 2-fold ratio, with a negative serum control set in each plate. The diluted serum samples were added, with 100 μl per well, and incubated at 37° C. (constant temperature incubator) for 2 hours. The serum samples were discarded, and 250 μl of a PBST washing solution was added to each well using a multichannel pipette, for washing 3 times. 100 μl of goat anti-mouse IgG HRP (abeam) diluted 10,000 fold was added to each well, and incubated at 37° C. (incubator) for 1 hour. The secondary antibodies were discarded, and 300 μl of PBST washing solution was added to each well using a multichannel pipette, for washing 5 times. After washing, 100 μl of 3,3′,5,5′-tetramethylbenzidine (TMB) (ebioscience) color developing solution was added to each well for color developing for 5 minutes in dark. 100 μl of 2 N H2SO4 was added to each well to terminate the reaction, and the test was carried out. The OD values were read at 450 nm wavelength using a microplate reader (BioTek). The endpoint titer was determined as the reciprocal of the last diluted serum. The OD450 nm of the samples that was greater than 2.1 times the OD450 nm of negative control group was determined as a positive value.

The results of immunogenicity are shown in FIG. 4, where the S protein specific binding antibodies of SW0123.351-LPP after lyophilization are comparable, without a significant difference. It shows the stability of the immunogenicity of the four-lipid composition after lyophilization.

### Example 7 Study on Stability of SW0123.351 Lyophilized Powder

This example used the preparation method as provided in Example 3 to respectively prepare SW0123.351-LPP containing COVID-19 mRNA (SEQ ID NO: 3) for lyophilization, to prepare SW0123.351-LPP lyophilized powder containing COVID-19 mRNA (0.1 mg/ml).

The appropriate amount of SW0123.351-LPP lyophilized powder was taken and stored in a 25° C. environment, recorded as day 0. Sampling and reconstitution were carried out at months 1, 2, and 3 after storage, respectively. The particle size, encapsulation efficiency and immunogenicity of the LPP solutions after reconstitution were tested. The specific test methods were as follows:

Test of particle size: 50 ul of solutions after sampling and reconstitution at day 0 and months 1, 2, and 3 after the SW0123.351 lyophilized powder was stored in a 25° C. environment were taken, and diluted with 950 uL of purified water to obtain diluted LPP samples. The samples were placed in a dynamic light scattering laser particle size analyzer (Malvern, ZS-90) for test.

Test of encapsulation efficiency: the mRNA contents in the LPP solutions after reconstitution at day 0 and months 1, 2, and 3 after the SW0123.351 lyophilized powder was stored in a 25° C. environment were tested using Quant-iT™RiboGreen™RNA reagent (Thermo Scientific). Firstly, the LPP solutions were diluted with nuclease-free water and 1×TE buffer (10 mM Tris-HCl, 0.1 mM EDTA, pH 8.00), and then mixed with an equal volume of 2% Triton X-100, and incubated at room temperature for 1 hour to release the encapsulated mRNA. Then, 100 μL of each sample was taken and transferred to a 96-well plate to which 100 μL of RiboGreen™RNA reagent diluted 200 fold was added. The plate was shaken and incubated at room temperature for 10 minutes. Then, the fluorescence values were read by a Bio-Tek Synergy I plate reader (BioTek). The standard sample was treated in the same way. The calibration curves of fluorescence and mRNA concentration were plotted by linear regression from which the mRNA concentrations of the samples were calculated. The encapsulation efficiency of the LPP solution was defined as the percentage of the amount of encapsulated mRNA in the amount of total mRNA in the test sample.

Test of immunogenicity: female C57BL/6 mice (Shanghai Lingchang Biotechnology Co., Ltd.) of 6-8 weeks of age were divided into four groups (group at day 0, group at month 1, group at month 2, and group at month 3, after storage of SW0123.351 lyophilized powder in a 25° C. environment, with 5 mice per group). C57BL/6 mice were immunized by intramuscular injection (hind limbs of mice) at days 1 and 14. The SW0123.351-LPP solutions containing 10 μg of COVID-19 mRNA after reconstitution at day 0 and months, 1, 2, and 3 after storage in a 25° C. environment were administrated respectively. Blood was taken from the orbits of the mice at day 14 after secondary immunization, and the S protein specific binding antibodies in serum were tested as described in Example 6, with PBS as a control.

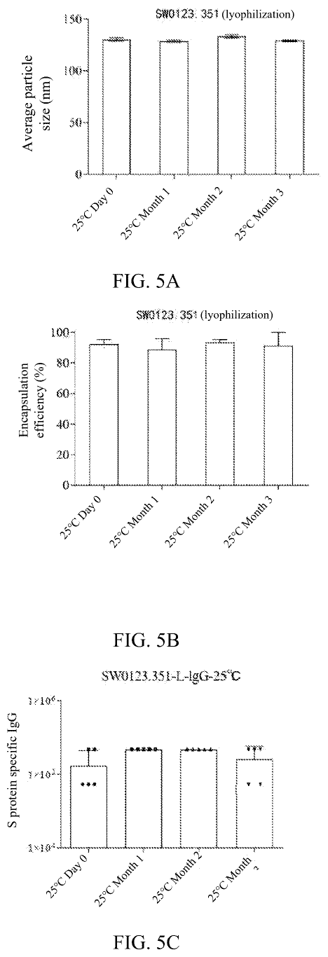

The results of test of particle size and the results of test of encapsulation efficiency of the lyophilized powder at day 0 and months, 1, 2, and 3 after storage in a 25° C. environment are shown in FIG. 5A and FIG. 5B, respectively, where after the SW0123.351-LPP lyophilized powder was stored for 3 months in a 25° C. environment, the in vitro physicochemical property has no significant change, and is very stable. It is further shown that the SW0123.351LPP lyophilized powder provided herein stored in a 25° C. environment has the performance in all aspects that can be maintained in a stable state, indicating the stability of the in vitro physicochemical property of the lyophilized powder of the four-lipid composition.

The results of immunogenicity of the lyophilized powder at day 0 and months, 1, 2, and 3 after storage in a 25° C. environment are shown in FIG. 5C, where after the SW0123.351-LPP lyophilized powder was stored for 3 months in a 25° C. environment, the in vivo expression has no significant change, indicating the stability of the immunogenicity thereof, and further indicating the stability of the immunogenicity of the lyophilized powder of the four-lipid composition.

### Example 8 Comparison of LNP and LPP Preparations Prepared by Different Prescriptions with Cationic Lipid being M5

### 8.1. Preparation of LNP-mRNA Preparations and LPP-mRNA Preparations

8.1.1 Preparation of lipid nanoparticle (LNP-mRNA) preparations:

Preparation of an aqueous mRNA solution: a luciferase mRNA was diluted into 0.35 mg/mL aqueous mRNA solution with 50 mM citric acid-sodium citrate buffer (pH of 3-4).

Preparation of a lipid solution: M5(or MC3):phospholipid:cholesterol:PEG was dissolved in an ethanol solution in a molar ratio of 50:10:38.5:1.5 according to the lipid types listed in Table 2, to prepare a 10 mg/mL lipid solution.

Preparation of LNP: by using a microfluidic technology (Micro & Nano (Shanghai) Biologics Co., Ltd., Model: Inano D), the lipid solution and the aqueous mRNA solution were mixed under the following conditions: volume=4.0 mL; flow rate ratio=3(aqueous mRNA solution):1(lipid solution), and total flow rate=12 mL/min, to obtain an LNP-mRNA solution.

Centrifugal ultrafiltration: the LNP-mRNA solution was added into an ultrafiltration tube for centrifugal ultrafiltration and concentration (centrifuge force of 3,400 g, centrifuge time of 60 minutes, and temperature of 4° C.), to obtain LNP-mRNA preparations numbered M5-A, M5-B, M5-C, M5-D, and MC3.

### 8.1.2 Preparation of Lipopolyplex (LPP-mRNA) Preparations:

Preparation of an aqueous mRNA solution: a luciferase mRNA was diluted into 0.35 mg/mL aqueous mRNA solution with 50 mM citric acid-sodium citrate buffer (pH of 3-4).

Preparation of a lipid solution: M5(or MC3):phospholipid:cholesterol:PEG was dissolved in an ethanol solution in a molar ratio of 50:10:38.5:1.5 according to the lipid types listed in Table 2, to prepare a 10 mg/mL lipid solution.

Preparation of a protamine sulfate solution: protamine sulfate was dissolved in nuclease-free water to prepare a protamine sulfate solution with a working concentration of 0.2 mg/mL.

Preparation of a core nanoparticle solution: by using a microfluidic technology, the protamine sulfate solution was mixed with the mRNA solution under the following conditions: volume=4.0 mL; flow rate ratio=3(mRNA):1(protamine solution), total flow rate=12 mL/min, pre-waste=0.35 mL, post-waste=0.1 mL, and room temperature, to obtain a core nanoparticle solution formed by protamine and mRNA.

Preparation of LPP: the core nanoparticle solution and the lipid solution were secondarily mixed under the following conditions: volume=4.0 mL, flow rate ratio=3(aqueous mRNA solution):1(lipid solution), total flow rate=12 mL/min, pre-waste=0.35 mL, post-waste=0.1 mL, and room temperature, to obtain an LPP-mRNA solution.

Centrifugal ultrafiltration: the LPP-mRNA solution was subjected to ultrafiltration and centrifugation to remove ethanol (centrifuge force of 3,400 g, centrifuge time of 60 minutes, and temperature of 4° C.), to obtain LPP-mRNA preparations numbered M5-A, M5-B, M5-C, M5-D, and MC3.

8.2. Physicochemical Properties of Different Phospholipids and PEGs after Combination

This example used the LNP preparations and LPP preparations of M5-A, M5-B, M5-C, M5-D, and MC3 as prepared in Examples 8.1.1 and 8.1.2 to test the physicochemical properties of different phospholipids and PEGs after combination. The specific test methods were as follows:

Test of particle size and zeta potential: 50 ul of LPP or LNP samples were taken and diluted with 950 uL of purified water to obtain diluted LPP or LNP samples. The samples were placed in a dynamic light scattering laser particle size analyzer (Malvern, ZS-90) for test.

Test of encapsulation efficiency: as described in Example 7, the encapsulation efficiency of the LPP solutions or the LNP solutions was tested using Quant-iT™RiboGreen™RNA reagent (Thermo Scientific).

Test of polydispersity index (PDI): the polydispersity index (PDI) of the LPP or LNP solutions was tested using Zetasizer Nano ZS (Malvern Instruments Ltd, Malvern, Worcestershire, UK).

The experimental results are shown in Table 2, where the prepared LPP nanoparticles have a larger particle size than the LNP nanoparticles, and the LPP preparations of M5-B, M5-C, M5-D, and M5-E have higher encapsulation efficiency than the LNP preparations. LNP has a smaller PDI, but the PDI of both LNP and LPP is less than 0.3, indicating that both have a narrower particle size distribution. The Zeta potentials of the two are close.

TABLE 2
Table for physicochemical properties of different phospholipids and PEGs after combination

| | | | | | | | | Encap- | | Poly- | |
|---|---|---|---|---|---|---|---|---|---|---|---|
| | Phospho- | | | Cationic | Phospho- | Choles- | | sulation | Particle | dispersity | Zeta |
| | lipid | PEG | | lipid | lipid | terol | PEG | efficiency | size | index | potential |
| | type | type | Type | (%) | (%) | (%) | (%) | (%) | (mm) | (PDI) | (mV) |
| M5-A | DOPE | PEG- | LNP | 50 | 10 | 38.5 | 1.5 | 96.6% | 89.4 | 0.07 | 16.7 |
| | | DSPE | LPP | | | | | 94.4% | 96.2 | 0.09 | 24.2 |
| M5-B | | PEG- | LNP | | | | | 82.9% | 76.7 | 0.16 | 21.6 |
| | | DMG | LPP | | | | | 95.8% | 112.6 | 0.16 | 19.7 |
| M5-C | DSPC | PEG- | LNP | | | | | 89.6% | 92.5 | 0.25 | 24.5 |
| | | DMG | LPP | | | | | 94.2% | 111.0 | 0.16 | 26.8 |
| M5-D | | PEG- | LNP | | | | | 93.6% | 83.3 | 0.20 | 19.7 |
| | | DSPE | LPP | | | | | 96.1% | 102.5 | 0.12 | 18.6 |
| MC3 | DSPC | PEG- | LNP | | | | | 94.3% | 86.5 | 0.17 | 24.7 |
| | | DMG | LPP | | | | | 97.4% | 96.3 | 0.27 | 20.7 |

### 8.3. Comparison of In Vivo Expression Amounts of Preparations Prepared by Different Prescriptions

This example used the LNP preparations and LPP preparations of M5-A, M5-B, M5-C, M5-D, and MC3 as prepared in Examples 8.1.1 and 8.1.2 to test the luciferase signal expression of the preparations prepared by different prescriptions in mice. The specific test methods were as follows:

Test of in vivo luciferase signal expression: female Balb/c mice (Beijing Vital River Laboratory Animal Technology Co., Ltd.) of 6-8 weeks of age were divided into 10 groups (MC3-LNP, MC3-LPP, A-LNP, A-LPP, B-LNP, B-LPP, C-LNP, C-LPP, D-LNP, and D-LPP groups, with 3 mice per group). MC3-LNP, MC3-LPP, M5-A-LNP, MS-A-LPP, MS-B-LNP, MS-B-LPP, MS-C-LNP, MS-C-LPP, MS-D-LNP, and MS-D-LPP solutions containing 10 μg of luciferase mRNA were administered by intramuscular injection (hind limbs of mice), respectively. 3 Hours, 6 hours, and 24 hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the injection sites and livers of the mice.

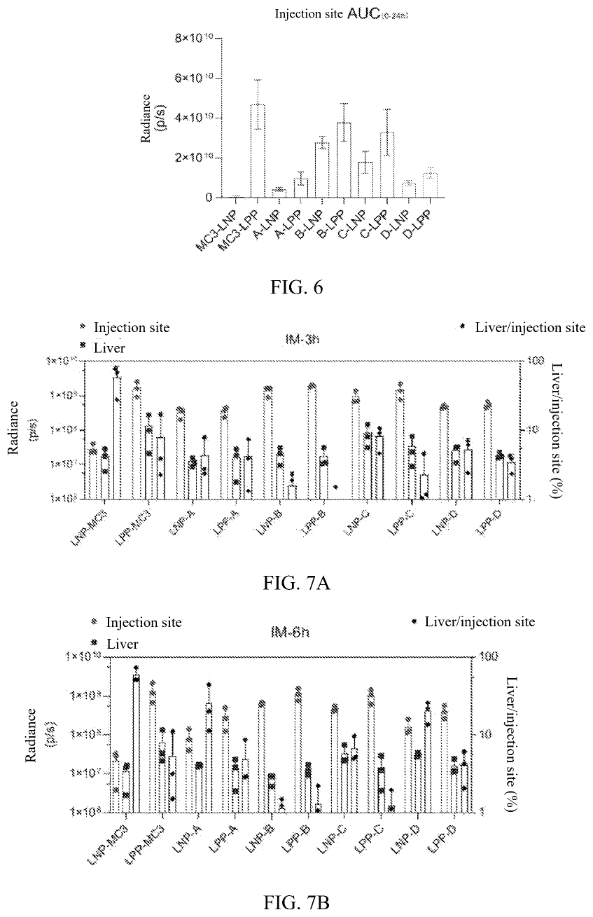

The results of total luciferase signal expression of the preparations prepared by different prescriptions at the injection sites of the mice within 0-24 hours are shown in FIG. 6, where the results for all lipid compositions show that under the same prescription, LPP has a higher fluorescent expression signal in the mice than LNP, indicating that under the same prescription, LPP has a higher expression amount than LNP; moreover, lipid composition B in the LPP delivery system has the highest expression amount, indicating that when the phospholipid is DOPE and the PEG type is PEG-DMG, the optimal expression is achieved.

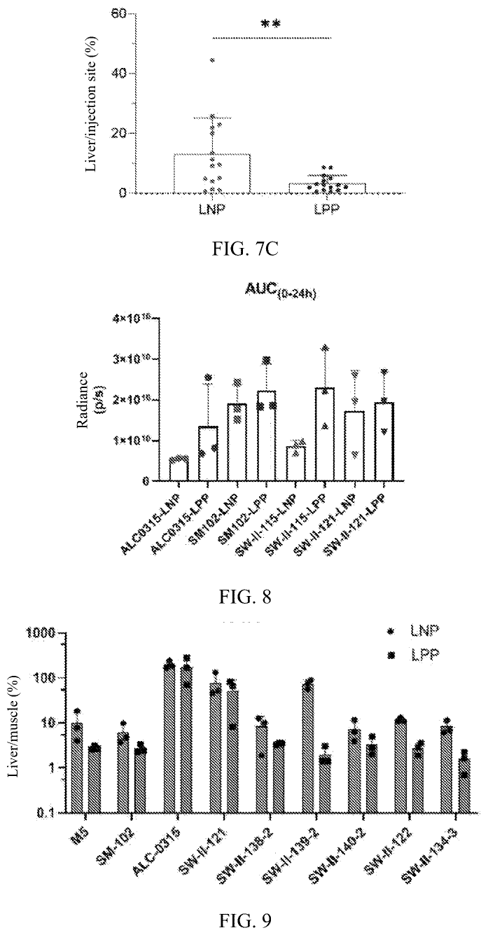

The results of luciferase signal expression of the preparations prepared by different prescriptions in the livers and injection sites of the mice 3 hours after the administration are shown in FIG. 7A and Table 3, where the three columns in each group are the luciferase expression of injection site, the luciferase expression in the liver, and the ratio of luciferase expression of liver/injection site, from left to right respectively. It is observed that the expression of the LPP preparations is close to the expression of the LNP preparations in the muscle tissue of the injection site, M5-A/B/C/D-LPP has higher expression than M5-A/B/C/D-LNP group, and MC3-LPP also has higher expression than MC3-LNP group; meanwhile, the LPP groups all have a lower ratio of luciferase expression of liver/injection site than the LNP groups. It is shown that 3 hours after the intramuscular injection, LPP has a higher expression amount in the injection site than LNP, and has a good targeting effect, a small ratio of expression into the liver, a small influence on the liver, and small hepatotoxicity.

The results of luciferase signal expression of the preparations prepared by different prescriptions in the livers and injection sites of the mice 6 hours after the administration are shown in FIG. 7B and Table 4, where the three columns in each group are the luciferase expression of injection site, the luciferase expression in the liver, and the ratio of luciferase expression of liver/injection site, from left to right respectively. It is observed that M5-A/B/C/D-LPP has 2-4 fold higher expression in the injection site than M5-A/B/C/D-LNP group, and MC3-LPP also has higher expression than MC3-LNP group; meanwhile, the LPP groups still have a lower ratio of luciferase expression of liver/injection site than the LNP groups. It is shown that 6 hours after the intramuscular injection, the difference in expression between the LPP preparations and the LNP preparations further increases, and the LPP preparations have a significantly higher expression amount in the injection site than the LNP preparations, and still have an excellent targeting effect. It is observed that under the same prescription, both M5-B-LNP and M5-B-LPP have a lower ratio of luciferase expression of liver/injection site, mainly express in the injection site and less express in the liver, further indicating that when the phospholipid is DOPE and the PEG type is PEG-DMG, the preparations will have better in vivo distribution and a stronger targeting effect.

The ratios of luciferase expression of liver/injection site 6 hours after the administration are shown in FIG. 7C, where LPP has a significantly higher ratio of luciferase expression of liver/injection site than LNP. It is further shown that compared with the LNP preparations, the LPP preparations not only have higher in vivo expression efficiency, but also have a good targeting effect, a small influence on the liver, and small hepatotoxicity.

TABLE 3
Luciferase expression of each preparation in muscle
and liver 3 hours after intramuscular administration

| | Intramuscular injection- | Intramuscular injection- |
|---|---|---|
| Sample | muscle (3 hours) | liver (3 hours) |
| M5-A-LNP | 3.34E ± 08 | 1.21E ± 07 |
| M5-A-LPP | 3.61E ± 08 | 1.65E ± 07 |
| M5-B-LNP | 1.41E ± 09 | 2.04E ± 07 |
| M5-B-LPP | 1.97E ± 09 | 1.78E ± 07 |
| M5-C-LNP | 9.34E ± 08 | 8.16E ± 07 |
| M5-C-LPP | 1.49E ± 09 | 3.29E ± 07 |
| M5-D-LNP | 4.85E ± 08 | 2.50E ± 07 |
| M5-D-LPP | 5.61E ± 08 | 1.87E ± 07 |
| MC3-LNP | 2.90E ± 07 | 1.75E ± 07 |
| MC3-LPP | 1.72E ± 09 | 1.35E ± 08 |

TABLE 4
Luciferase expression of each preparation in muscle
and liver 6 hours after intramuscular administration

| | Intramuscular injection- | Intramuscular injection- |
|---|---|---|
| Sample | muscle (3 hours) | liver (3 hours) |
| M5-A-LNP | 8.57E ± 07 | 1.62E ± 07 |
| M5-A-LPP | 2.98E ± 08 | 1.37E ± 07 |
| M5-B-LNP | 6.54E ± 08 | 7.31E ± 06 |
| M5-B-LPP | 1.16E ± 09 | 1.27E ± 07 |
| M5-C-LNP | 4.74E ± 08 | 3.26E ± 07 |
| M5-C-LPP | 1.06E ± 09 | 1.49E ± 07 |
| M5-D-LNP | 1.65E ± 08 | 3.06E ± 07 |
| M5-D-LPP | 4.10E ± 08 | 1.56E ± 07 |
| MC3-LNP | 2.13E ± 07 | 1.12E ± 07 |
| MC3-LPP | 1.35E ± 09 | 6.28E ± 07 |

### Example 9 Comparison of LNP and LPP Preparations Prepared by Different Prescriptions with Cationic Lipids being SM102, ALC0315, SW-II-115, and SW-II-121

This example used the preparation method as provided in Example 2 to prepare LPP and LNP preparations containing luciferase mRNA (0.1 mg/ml) with cationic lipids being SM-102, ALC-0315, SW-II-115, and SW-II-121 respectively as described in Table 5, so as to test the luciferase signal expression of the LPP and LNP preparations prepared by different prescriptions in the mice. The specific test methods were as follows:

Test of in vivo luciferase signal expression: female Balb/c mice (Beijing Vital River Laboratory Animal Technology Co., Ltd.) of 6-8 weeks of age were divided into 8 groups (ALC0315-LNP, ALC0315LPP, SM102-LNP, SM102-LPP, SW-II-115-LNP, SW-II-115-LPP, SW-II-121-LNP, and SW-II-121-LPP groups, with 3 mice per group). ALC0315-LNP, ALC0315LPP, SM102-LNP, SM102-LPP, SW-II-115-LNP, SW-II-115-LPP, SW-II-121-LNP, and SW-II-121-LPP solutions containing 10 g of luciferase mRNA were administered by intramuscular injection (hind limbs of mice), respectively. 3 Hours, 6 hours, and 24 hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the mice.

The results of total luciferase signal expression of each group within 0-24 hours are shown in FIG. 8, where the results for all lipid compositions show that under the same prescription, the LPP preparations have a higher fluorescent expression signal in the mice than the LNP preparations, indicating that under the same prescription, although the cationic lipids are different, all the LPP preparations have a higher expression amount than the LNP preparations, and have better expression efficiency.

TABLE 5
Different prescriptions with cationic lipids being
SM102, ALC0315, SW-II-115, and SW-II-121

| | Phospho- | | | Cationic | Phospho- | Choles- | |
|---|---|---|---|---|---|---|---|
| | lipid | | | lipid | lipid | terol | PEG |
| | type | PEG type | Sample | (%) | (%) | (%) | (%) |
| SM102 | DSPC | PEG-DMG | LNP | 50 | 10 | 38.5 | 1.5 |
| | | | LPP | | | | |
| ALC0315 | | | LNP | | | | |
| | | | LPP | | | | |
| SW-II-115 | | | LNP | | | | |
| | | | LPP | | | | |
| SW-II-121 | | | LNP | | | | |
| | | | LPP | | | | |

### Example 10 Comparison of LNP and LPP Preparations Prepared by Different Prescriptions with Cationic Lipids being M5, SM102, ALC0315, SW-II-121, SW-II-122, SW-II-134-3, SW-II-138-2, SW-II-139-2, and SW-II-140-2

This example used the preparation methods as provided in Examples 8.1.1 and 8.1.2 to prepare LPP and LNP preparations containing luciferase mRNA (0.1 mg/ml) with cationic lipids being M5, SM-102, ALC-0315, SW-II-121, SW-II-122, SW-II-134-3, SW-II-138-2, SW-II-139-2, SW-II-140-2 respectively, so as to test the luciferase signal expression of the lipid compositions prepared by different prescriptions in the mice. The specific test methods were as follows:

Test of in vivo luciferase signal expression: female Balb/c mice (Beijing Vital River Laboratory Animal Technology Co., Ltd.) of 6-8 weeks of age were divided into 18 groups (M5-LNP, M5-LPP, SM102-LNP, SM102-LPP, ALC0315-LNP, ALC0315LPP, SW-II-121-LNP, SW-II-121-LPP, SW-II-122-LNP, SW-II-122-LPP, SW-II-134-3-LNP, SW-II-134-3-LPP, SW-II-138-2-LNP, SW-II-138-2-LPP, SW-II-139-2-LNP, SW-II-139-2-LPP, SW-II-140-2-LNP, and SW-II-140-2-LPP groups, with 3 mice per group). M5-LNP, M5-LPP, SM102-LNP, SM102-LPP, ALC0315-LNP, ALC0315LPP, SW-II-121-LNP, SW-II-121-LPP, SW-II-122-LNP, SW-II-122-LPP, SW-II-134-3-LNP, SW-II-134-3-LPP, SW-II-138-2-LNP, SW-II-138-2-LPP, SW-II-139-2-LNP, SW-II-139-2-LPP, SW-II-140-2-LNP, and SW-II-140-2-LPP solutions containing 10 μg of luciferase mRNA were administered by intramuscular injection (hind limbs of mice), respectively. 6 Hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the livers and injection sites (muscles) of the mice.

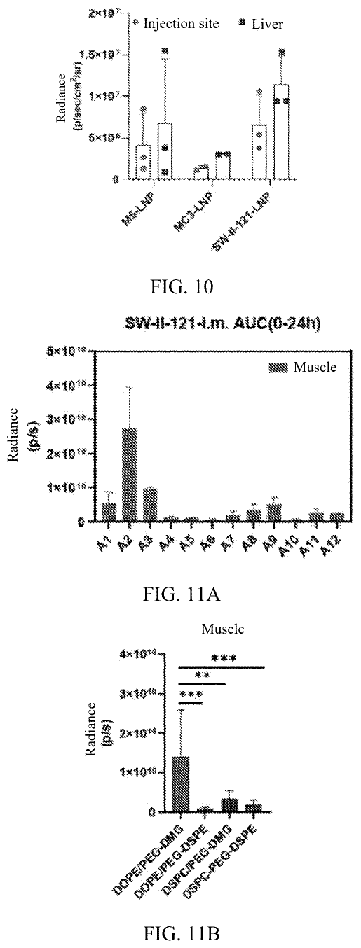

The results of ratios of luciferase signal expression of liver/muscle of the LNP and LPP preparations prepared by the prescriptions with different cationic lipids 6 hours after injection are shown in FIG. 9 and Table 6, where the two columns in each group are the ratio of luciferase expression of liver/injection site of the LNP preparations and the ratio of luciferase expression of liver/injection site of the LPP preparations, from left to right respectively. It is observed that the LPP preparations have a higher fluorescent expression signal in the mice than the LNP preparations, indicating again that under the same prescription, although the cationic lipids are different, all the LPP preparations have a higher expression amount than the LNP preparations, and have better expression efficiency.

TABLE 6
Ratios of luciferase expression of liver/muscle
of LNP and LPP preparations prepared by prescriptions
with different cationic lipids

| | Liver/muscle | Liver/muscle |
|---|---|---|
| Lipid type | (%, LNP) | (%, LPP) |
| M5 | 10.1 ± 7.5 | 2.8 ± 0.3 |
| SM-102 | 6.2 ± 3.3 | 2.7 ± 0.6 |
| ALC-0315 | 199.8 ± 35.8 | 173.2 ± 103.9 |
| SW-II-121 | 77.8 ± 48.9 | 52.2 ± 38.9 |
| SW-II-138-2 | 8.2 ± 5.7 | 3.5 ± 0.2 |
| SW-II-139-2 | 75.1 ± 16.1 | 2.0 ± 1.0 |
| SW-II-140-2 | 7.4 ± 4.0 | 3.3 ± 1.5 |
| SW-II-122 | 11.9 ± 1.2 | 2.8 ± 0.8 |
| SW-II-134-3 | 8.2 ± 2.9 | 1.5 ± 0.8 |

### Example 11 Comparison of Preparations Prepared by Different Prescriptions with Cationic Lipid being SW-II-121

11.1 Comparison of Different LNP Preparations with Cationic Lipids being MC3, M5, and SW-II-121

This example used the preparation method as provided in Example 8.1.1 to prepare LNP preparations containing luciferase mRNA (0.1 mg/ml) with cationic lipids being M5, MC3, and SW-II-121 respectively as described in Table 7, so as to test the in vivo expression of the LNP preparations with different cationic lipids 6 hours after intramuscular administration. The specific test methods were as follows:

Test of in vivo luciferase signal expression: female Balb/c mice (Beijing Vital River Laboratory Animal Technology Co., Ltd.) of 6-8 weeks of age were divided into 3 groups (M5-LNP, MC3-LNP, and SW-II-121-LNP, with 3 mice per group). M5-LNP, MC3-LNP, and SW-II-121-LNP solutions containing 10 μg of luciferase mRNA were administered by intramuscular injection (hind limbs of mice), respectively. 6 Hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the livers and injection sites (muscles) of the mice.

The results are shown in FIG. 10, where the two columns in each group are the luciferase expression of injection site and the luciferase expression of liver, from left to right respectively. It is observed that SW-II-121 has significantly superior in vivo expression 6 hours after intramuscular administration to M5-LNP and MC3-LNP, indicating better expression efficiency. Thus, the further study was made on the influence of the lipid ratio on the lipid compositions with SW-II-121 as a preferred cationic lipid.

TABLE 7
Different prescriptions with cationic lipids being M5, SM102, and SW-II-121

| | | | Cationic | Phospho- | Choles- | |
|---|---|---|---|---|---|---|
| | Phospho- | | lipid | lipid | terol | PEG |
| Type | lipid | PEG | (%) | (%) | (%) | (%) |
| M5-LNP | DSPC | PEG-DMG | 50 | 10 | 38.5 | 1.5 |
| MC3-LNP | | | | | | |
| SW-II-121-LNP | | | | | | |

### 11.2 Influence of Changes of Lipid Types and Lipid Ratios on In Vivo Expression

This example used the preparation method as provided in Example 8.1.2 to prepare LNP preparations containing luciferase mRNA (0.1 mg/ml) with a cationic lipid being SW-II-121 and with different lipid types and lipid ratios as described in Table 9, so as to test the influence of changes of lipid types and lipid ratios on in vivo expression. The specific test methods were as follows:

Test of in vivo luciferase signal expression: female Balb/c mice (Beijing Vital River Laboratory Animal Technology Co., Ltd.) of 6-8 weeks of age were divided into 12 groups (A1, A2, A3, A4, A5, A6, A7, A8, A9, A10, A11, and A12 groups, with 3 mice per group). LPP solutions of A1, A2, A3, A4, A5, A6, A7, A8, A9, A10, A11, and A12 containing 10 μg of luciferase mRNA were administered by intramuscular injection (hind limbs of mice), respectively. 24 Hours after the administration, mice were injected intraperitoneally with 30 mg of a D-fluorescein substrate. The bioluminescence was measured in a Xenogen IVIS-200 imaging system 15 minutes after substrate injection to evaluate the expression of the luciferase mRNA in the livers and injection sites (muscles) of the mice.

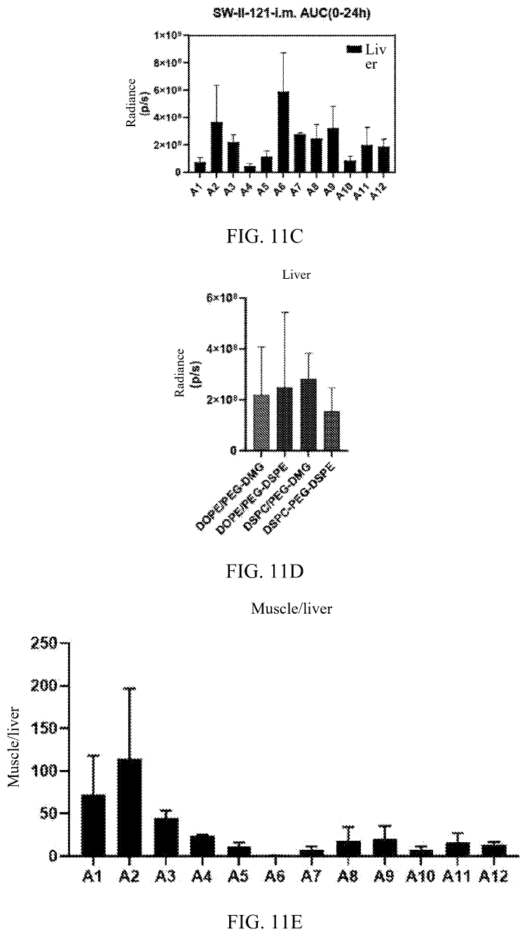

The expression of each preparation in the injection site (muscle tissue) of the mice 0-24 hours after intramuscular injection is shown in FIG. 11A, FIG. 11B, and Table 9, where when the phospholipid is DOPE and PEG is PEG-DMG, the injection site of the mice has the highest fluorescent expression, which is significantly superior to that of the combinations of other lipid types; moreover, when the lipid ratio is 50% mol of SW-II-121, 10% mol of DOPE, 38.5% of cholesterol, and 1.5% of PEG-DMG, the injection site has the highest expression efficiency. The expression of each preparation in the livers of the mice 0-24 hours after intramuscular injection is shown in FIG. 11C, FIG. 11D, and Table 9. The four groups have no a significant difference, but from the order of magnitude of the ordinate, it can be seen that each preparation has significantly lower expression in the liver of the mice than in the injection site.

Moreover, as shown in FIG. 11E and Table 9, when the phospholipid is DOPE and PEG is PEG-DMG, the ratio of luciferase expression of muscle/liver is the highest, the targeting effect is the best, and the hepatotoxicity is the smallest. Especially when the lipid ratio is 50% mol of SW-II-121, 0%) mol of DOPE, 38.5% of cholesterol, and 1.5% of PEG-DMG, the ratio of luciferase expression of muscle/liver is the highest, and the targeting effect is the best. It is further shown that the preferred phospholipid is DOPE, the preferred PEG is PEG-DMG, and the preferred lipid composition includes 50 mol % of cationic lipid SW-II-121, 10 mol % of phospholipid DOPE, 38.5 mol % of cholesterol, and 1.5 mol % of PEG-DMG.

TABLE 8
Different prescriptions with a cationic lipid being SW-II-121

| | | | | Cationic | | | |
|---|---|---|---|---|---|---|---|
| | | | | lipid | Phospho- | Choles- | |
| | | Phospho- | | SW-II-121 | lipid | terol | |
| Type | mRNA:lipid | lipid | PEG | (%) | (%) | (%) | PEG (%) |
| A1 | 1:20 | DOPE | PEG-DMG | 30 | 20 | 48.5 | 1.5 |
| A2 | | | | 50 | 10 | 38.5 | 1.5 |
| A3 | | | | 40 | 15 | 43.5 | 1.5 |
| A4 | | | PEG-DSPE | 30 | 20 | 48.5 | 1.5 |
| A5 | | | | 50 | 10 | 38.5 | 1.5 |
| A6 | | | | 40 | 15 | 43.5 | 1.5 |
| A7 | | DSPC | PEG-DMG | 30 | 20 | 48.5 | 1.5 |
| A8 | | | | 50 | 10 | 38.5 | 1.5 |
| A9 | | | | 40 | 15 | 43.5 | 1.5 |
| A10 | | | PEG-DSPE | 30 | 20 | 48.5 | 1.5 |
| A11 | | | | 50 | 10 | 38.5 | 1.5 |
| A12 | | | | 40 | 15 | 43.5 | 1.5 |

TABLE 9
Ratios of luciferase expression of muscle/liver of different
lipid compositions with a cationic lipid being SW-II-121

| | Muscle/liver |
|---|---|
| Type | (%, LPP) |
| A1 | 72.4 ± 45.7 |
| A2 | 114.3 ± 82.3 |
| A3 | 44.5 ± 9.0 |
| A4 | 24.3 ± 0.7 |
| A5 | 11.7 ± 4.4 |
| A6 | 0.7 ± 0.3 |
| A7 | 7.4 ± 3.9 |
| A8 | 17.8 ± 16.4 |
| A9 | 20.5 ± 15.1 |
| A10 | 7.6 ± 4.0 |
| A11 | 16.1 ± 10.8 |
| A12 | 13.9 ± 3.0 |

### Example 12 Preparation of Lipid Nanoparticle (LNP-mRNA) Preparations and Lipopolyplex (LPP-mRNA) Preparations

### 12.1 Experimental Materials

Lipids DOPE, DSPC, PEG-DMG, and PEG-DSPE were purchased from Avanti Polar Lipids (Birmingham, Alabama, USA). Cholesterol was obtained from AVT (Shanghai) Pharmaceutical Tech Co., Ltd.

### 12.2 Preparation of LNP-mRNA Preparations

Preparation of an aqueous mRNA solution: mRNA was diluted into 0.35 mg/mL aqueous mRNA solution with 50 mM citric acid-sodium citrate buffer (pH of 3-4).

Preparation of a lipid solution: SM-102 (or ALC-0315):phospholipid:cholesterol:PEG was dissolved in an ethanol solution in a molar ratio of 50:10:38.5:1.5, to prepare a 10 mg/mL lipid solution.

Preparation of LNP: by using a microfluidic technology (Micro & Nano (Shanghai) Biologics Co., Ltd., Model: Inano D), the lipid solution and the aqueous mRNA solution were mixed under the following conditions: volume=4.0 mL; flow rate ratio=3(aqueous mRNA solution):1(lipid solution), and total flow rate=12 mL/min, to obtain an LNP-mRNA solution.

Centrifugal ultrafiltration: the LNP-mRNA solution was added into an ultrafiltration tube for centrifugal ultrafiltration and concentration (centrifuge force of 3,400 g, centrifuge time of 60 minutes, and temperature of 4° C.), to obtain LNP-mRNA preparations numbered SM-102LNP and ALC0315-LNP.

### 12.3 Preparation of LPP-mRNA Preparations

Preparation of an aqueous mRNA solution: mRNA was diluted into 0.35 mg/mL aqueous mRNA solution with 50 mM citric acid-sodium citrate buffer (pH of 3-4).

Preparation of a lipid solution: a lipid was dissolved in an ethanol solution according to the lipid types and mole ratios in the prescriptions listed in Table 10 and Table 11, to prepare a 10 mg/mL lipid solution.

Preparation of a protamine sulfate solution: protamine sulfate was dissolved in nuclease-free water to prepare a protamine sulfate solution with a working concentration of 0.2 mg/mL.

Preparation of a core nanoparticle solution: by using a microfluidic technology, the protamine sulfate solution was mixed with the mRNA solution under the following conditions: volume=4.0 mL; flow rate ratio=3(mRNA): 1(protamine solution), total flow rate=12 mL/min, pre-waste=0.35 mL, post-waste=0.1 mL, and room temperature, to obtain a core nanoparticle solution formed by protamine and mRNA.

Preparation of LPP: the core nanoparticle solution and the lipid solution were secondarily mixed under the following conditions: volume=4.0 mL, flow rate ratio=3(aqueous mRNA solution):1(lipid solution), total flow rate=12 mL/min, pre-waste=0.35 mL, post-waste=0.1 mL, and room temperature, to obtain an LPP-mRNA solution.

Centrifugal ultrafiltration: the LPP-mRNA solution was subjected to ultrafiltration and centrifugation to remove ethanol (centrifuge force of 3,400 g, centrifuge time of 60 minutes, and temperature of 4° C.), to obtain corresponding LPP-mRNA preparations.

The particle size of LPP was measured using a dynamic light scattering analyzer (Zetasizer Nano ZS90, Malvern). The morphology of LPP was tested using a cryo-transmission electron microscope (Glacios, Thermo Fisher Scientific).

TABLE 10
Different prescriptions of four preparations

| | | PEG- | Cationic | Phospho- | Choles- | | | |
|---|---|---|---|---|---|---|---|---|
| Preparation | Helper | phospho- | lipid M5 | lipid | terol | PEG | Size | |
| name | lipid | lipid | (%) | (%) | (%) | (%) | (mm) | PDI |
| LPP-A | DOPE | PEG-DSPE | 50 | 10 | 38.5 | 1.5 | 96.2 ± 0.1 | 0.09 ± 0.03 |
| LPP-B | | PEG-DMG | | | | | 112.6 ± 0.9 | 0.16 ± 0.01 |
| LPP-C | DSPC | PEG-DMG | | | | | 111.0 ± 1.2 | 0.16 ± 0.04 |
| LPP-D | | PEG-DSPE | | | | | 102.5 ± 1.2 | 0.12 ± 0.02 |

TABLE 11
Different prescriptions of seven preparations

| | Cationic | | Choles- | | | |
|---|---|---|---|---|---|---|
| Preparation | lipid | DOPE | terol | PEG-DMG | Size | |
| name | M5 (%) | (%) | (%) | (%) | (mm) | PDI |
| LPP-B1 | 53.5 | 15 | 30 | 1.5 | 107.1 ± 0.9 | 0.12 ± 0.01 |
| LPP-B2 | 40 | 15 | 43.5 | 1.5 | 91.4 ± 3.1 | 0.16 ± 0.02 |
| LPP-B3 | 33.5 | 15 | 50 | 1.5 | 86.8 ± 1.5 | 0.15 ± 0.02 |
| LPP-B4 | 40 | 15 | 44 | 1 | 125.3 ± 2.0 | 0.13 ± 0.02 |
| LPP-B5 | 40 | 15 | 43 | 2 | 109.7 ± 3.9 | 0.22 ± 0.03 |
| LPP-B6 | 35 | 20 | 43.5 | 1.5 | 92.9 ± 1.2 | 0.13 ± 0.01 |
| LPP-B7 | 45 | 10 | 43.5 | 1.5 | 97.3 ± 2.4 | 0.13 ± 0.01 |

### Example 13 Optimized LPP Preparations Resulting in Effective APC Uptake and DC Maturation

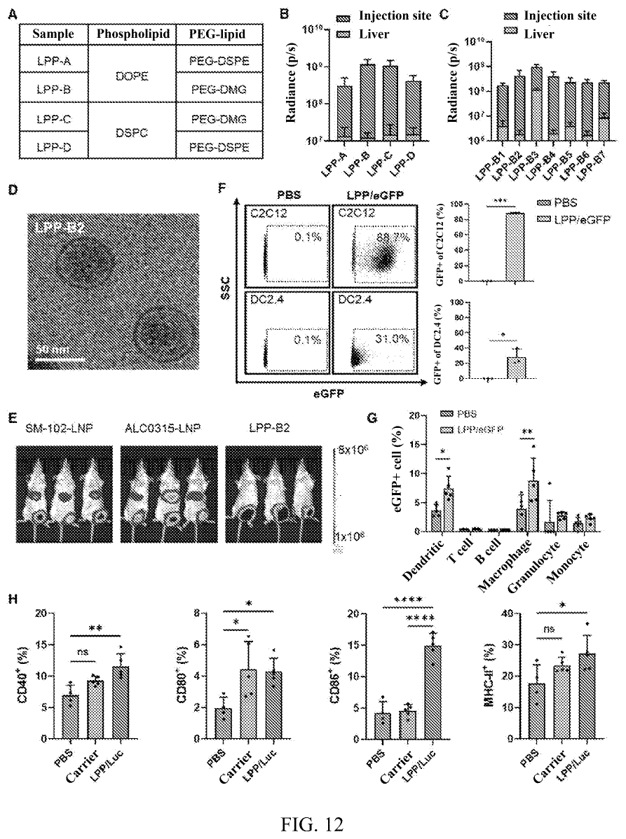

To minimize the systemic exposure to locally injected vaccines, this example used the preparation method as provided in Example 12 to prepare LPP preparations containing luciferase mRNA (coding sequence as shown in SEQ ID NO: 1, 0.1 mg/ml) with different lipids as described in FIG. 12(A) for intramuscular delivery, including different combinations of phospholipids (DOPE and DSPC) and PEG lipids (PEG-DMG and PEG-DSPE). For the specific lipid ratios and components of the four different LPP preparations, reference was made to Table 10.

As shown in FIG. 12(B), among the four LPP preparations for intramuscular injection, LPP-B preparation elicits the strongest luciferase signal in the injection site and the least luciferase expression in the liver. Thus, the LPP preparations were further optimized based on this lipid combination.

The preparation method as provided in Example 12 was used to prepare 7 LPP preparations LPP/Luc containing luciferase mRNA (0.1 mg/ml) with different lipid ratios as shown in Table 11, and they were characterized. As shown in FIG. 12(C) and Table 11, LPP-B2 has the moderate particle size and the good biological distribution (the local expression of mRNA is higher, the expression of the liver is limited). Thus, the further study was made based on LPP-B2 prescription. As shown in the cryo-transmission electron microscope (Cryo-TEM) image in FIG. 12(D), LPP-B2 has a concentrated core and a layer of lipid shell encapsulating same, indicating that the LPP-B2 nanoparticle has a typical core-shell structure.

The in vitro experiments have confirmed the ability of LPP-B2 prescription to transfect into muscle cells and immune cells. The operational steps of the in vitro experiments were as follows.

The preparation method as provided in Example 12 was used to prepare LPP preparations containing enhanced green fluorescent protein (eGFP) mRNA (coding sequence as shown in SEQ ID NO: 2, 0.1 mg/ml) based on LPP-B2 prescription. The expression of GFP was observed using flow cytometry. In short, A20 (mouse B-cell lymphoma cells, purchased from ATCC), DC2.4 (mouse bone marrow derived dendritic cells, purchased from ATCC), C2C12 (mouse myoblasts, purchased from the cell bank of the Chinese Academy of Sciences), HSkMC (human skeletal muscle cells, purchased from ATCC), and Jurkat T cells (human T lymphocyte leukemia cells, purchased from the cell bank of the Chinese Academy of Sciences) were respectively inoculated in a 6-well plate complete medium with a density of 1×10⁵/well. The cells and LPP/eGFP were incubated for 24 hours (transfection dose of 2.5 μg/well). The percentage of GFP positive cells was determined by flow cytometry (BD FACSCanto II, Becton Dickinson).

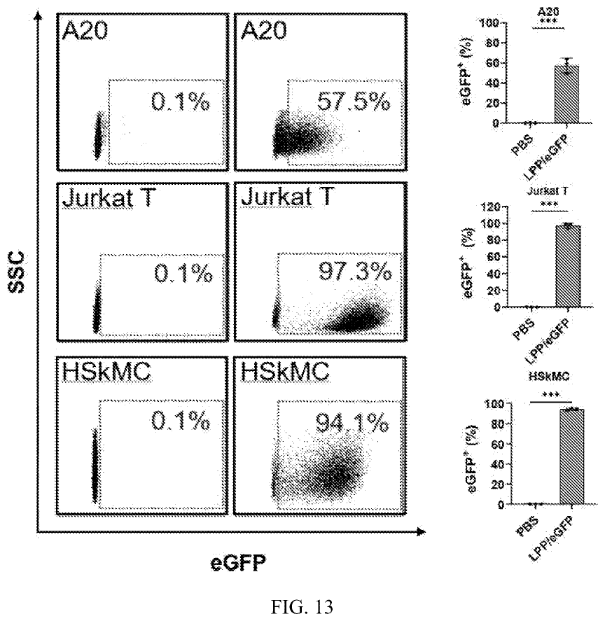

The results are shown in FIG. 12(F) and FIG. 13, where after LPP-B2/eGFP treatment, nearly 90% of C2C12, HSkMC, and Jurkat T cells synthesize GFP protein, and 60% of A20 cells and 30% of DC2.4 cells show GFP positive, indicating that LPP-B2 prescription can effectively induce the expression of proteins in muscle cells and immune cells.

To test the muscle targeted delivery of LPP-B2, this example further compared the expression amounts of luciferase delivered by LPP-B2 and LNP (SM-102LNP and ALC0315-LNP) in vivo. The specific operation methods were as follows.

Test of in vivo luciferase signal expression: female BALB/c mice (Charles River Laboratories) of 6-8 weeks of age were divided into 3 groups (SM-102LNP, ALC0315-LNP, and LPP-B2 groups, with 3 mice per group). LPP-B2, SM-102LNP, and ALC0315-LNP preparations containing 10 g of luciferase mRNA were administered by intramuscular injection (hind limbs of mice), respectively. 24 Hours after the administration, mice were injected intraperitoneally with 30 mg of a D-fluorescein substrate (the D-fluorescein substrate was purchased from Maokang Biotechnology Co., Ltd.). The bioluminescence was measured in a Xenogen IVIS-200 imaging system 15 minutes after substrate injection to evaluate the expression of the luciferase mRNA in the mice.

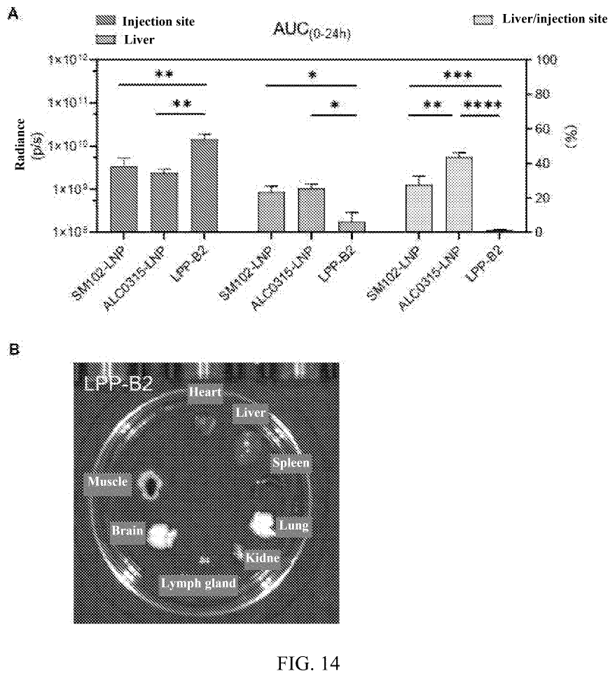

The results are shown in FIG. 12(E), where 6 hours after intramuscular injection, LPP-B2 mainly expresses in the injection site, and does almost not express in the liver site. As shown in FIG. 14(A), 24 hours after intramuscular injection, compared with commercial LNP, LPP-B2 elicits a 4-6 fold higher luciferase signal in the injection site and 4-5 fold lower luciferase expression in the liver. As shown in FIG. 14(B), further in vitro dissection has confirmed that LPP-B2 mainly expresses proteins at the local injection site, and no significant luciferase signal was observed in major organs (heart, liver, spleen, lung, kidney, brain, and lymph gland). These results show that compared with commercial LNP, the intramuscular targeted LPP-B2 delivery system may cause fewer systemic adverse events.

This example further investigated the expression of LPP preparations containing enhanced green fluorescent protein (eGFP) mRNA (0.1 mg/ml) based on LPP-B2 prescription in different types of immune cells in the lymph gland. The test methods were as follows.

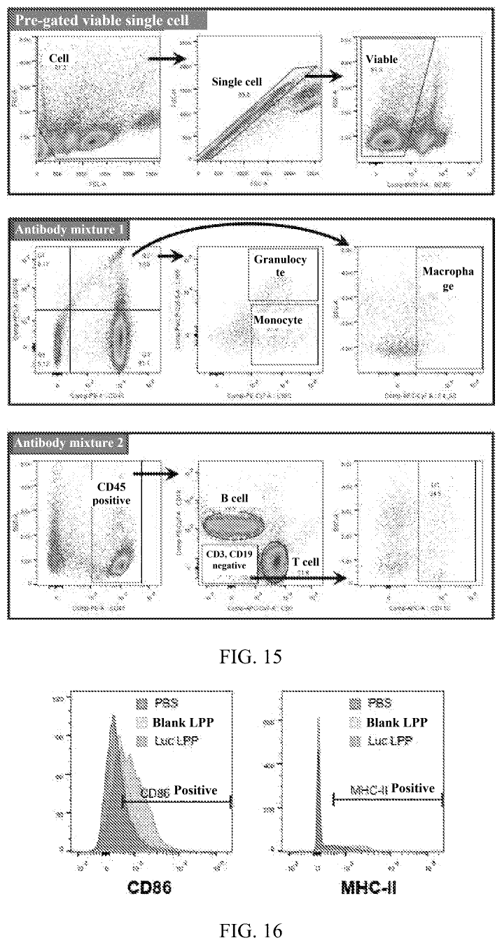

To test the mRNA expression of different types of cells in the lymph gland, 24 hours before the test, C57BL/6 mice (Charles River Laboratories, n=5) were intramuscularly injected with control PBS or the LPP preparations containing enhanced green fluorescent protein (eGFP) mRNA (0.1 mg/ml) based on LPP-B2 prescription, with a dose of 30 g/mouse. The GFP positive cells of different cell types were tested using flow cytometry (BD FACSCanto II, Becton Dickinson). The T cell, the B cell, the DC cell (dendritic cell), the macrophage, the granulocyte, and the monocyte were defined as CD45⁺CD3⁺CD19⁻, CD45⁺CD3⁻CD19⁺, CD45⁺CD3⁻CD19⁻CD11c⁺, CD45⁺CD11b⁺F4/80⁺, CD45⁺CD11b⁺Ly6G⁺, and CD45⁺CD11b⁺Ly6C⁺Ly6G⁻ (CD45, CD3, CD19, CD11c, CD11b, F4/80, and Ly6C detection antibodies were all purchased from Biolegend, Ly6G detection antibody was purchased from BD Biosciences), to recognize the gating strategy of cell populations as shown in FIG. 15.

The results are shown in FIG. 12(G), where the eGFP expression rates of DC and macrophages are about 7% and 8%, respectively. In contrast, the T cell, the B cell, the granulocyte, and the monocyte show no positive signal. These results show that antigen-presenting cells are more effective in LPP uptake and mRNA translation.

Furthermore, the mature markers of DC cells were tested after the injection of the LPP preparations based on LPP-B2 prescription. The test methods were as follows.

C57BL/6 mice (Charles River Laboratories, n=5) were injected intramuscularly with PBS, blank vehicle LPP (referring to LPP without mRNA) or LPP preparations LPP/Luc containing luciferase mRNA based on LPP-B2 prescription, with a dose of 10 μg mRNA/mouse. 24 Hours after the injection, the lymph glands were collected and mechanically destroyed in a 70 μm cell strainer. The cells were pelleted by centrifugation (2,000 rpm, 5 minutes), and the concentration was regulated to 1×10⁶ cells/tube with FACS buffer. The obtained cells were treated with Fixable Viability Stain (BD), and incubated at room temperature for 10 minutes. Then, the cells were washed with FACS buffer and stained with CD11c, CD40, CD80, CD86, and IA/I-E specific antibodies (all purchased from Biolegend) at 4° C. for 30 minutes. The cells were washed twice prior to flow cytometry analysis using BD FACSCanto II, Becton Dickinson. The recognition of the gating strategy of cell populations is as shown in FIG. 16.

The results are shown in FIG. 12(H), where compared with PBS treatment group, 24 hours after the injection of LPP/Luc, the levels of DC cell maturation markers (CD40, CD80, CD86, and MHC-II) in the mice are significantly up-regulated, which further proves the adjuvant effect of LPP that can result in effective DC maturation.

### Example 14 Optimized Antigen Design Enhancing Translation of Spike Protein In Vitro

Increasing the concentration and efficacy of the presented effective epitope is a primary goal for the antigen design of vaccine products. The antigens for currently licensed SARS-CoV-2 mRNA vaccines were designed in early 2020. With the development of pandemic and the understanding of the spike protein structure, it is necessary to improve the antigen design of a new generation of SARS-CoV-2 mRNA vaccines.

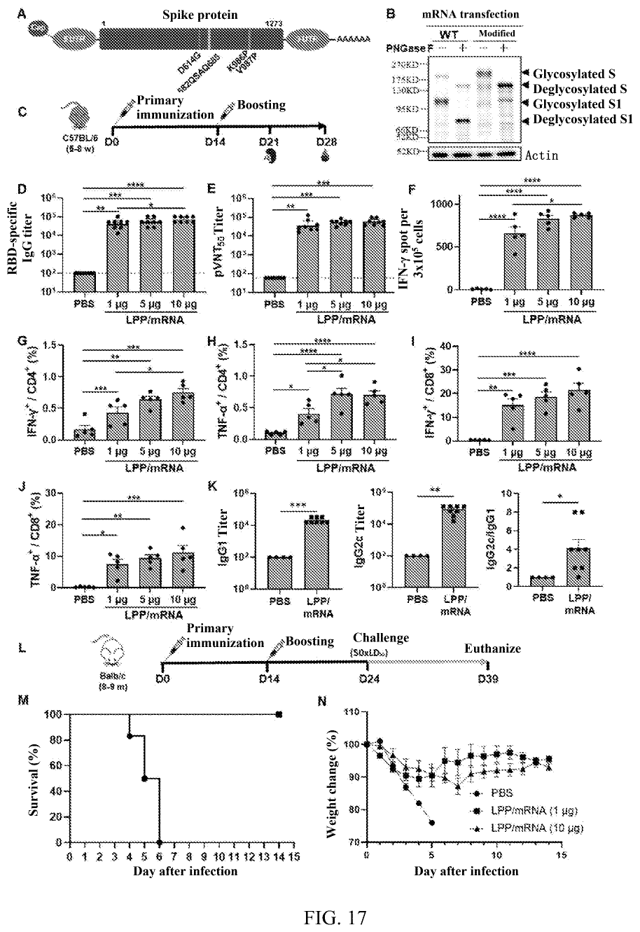

In this example, an mRNA encoding a SARS-CoV-2 full-length spike protein into which three mutations were introduced was designed and prepared. The sequence structure is as shown in FIG. 17(A). Where, the 2P mutation (986-987aa) and furin mutation (682-685aa) stabilize the full-length spike protein locked in the pre-fusion conformation, and prevent the cleavage of the S1/S2 subunit, thereby maintaining the integrity and conformation of the S protein. These designs have been shown to improve the immunogenicity of the S protein (W. B. Alsoussi, et al., SARS-CoV-2 Omicron boosting induces de novo B cell response in humans. Nature, 1-3(2023) and J. Pallesen, et al., Immunogenicity and structures of a rationally designed prefusion MERS-CoV spike antigen. Proceedings of the National Academy of Sciences. 114, E7348-E7357 (2017)). Since all variants of interest contain the D614G mutation (B. Korber, et al., Tracking changes in SARS-CoV-2 Spike: evidence that D614G increases infectivity of the COVID-19 virus. Cell. 182,812-827 (2020)), a D614G substitution is also introduced into the antigen design. This design further limits the premature conversion to the post-fusion conformation and gives variants with potentially broader neutralizing capacity (P.-A. Koenig, et al., Spike D614G—A Candidate Vaccine Antigen Against Covid-19. New England Journal of Medicine. 384, 2349-2351 (2021)). In addition, the substitution of G614 supports the RBD domain at the “up” position, increasing the presentation of an effective epitope, thereby enhancing the immunogenicity. The expression of mRNA encoding the spike protein (S protein) is characterized by in vitro cell transfection. The methods for mRNA synthesis, in vitro transfection and expression test were as follows.

mRNA Synthesis: as described above, the antigen for a designed candidate mRNA vaccine was based on the spike protein of the wild-type strain (GenBank accession number: MN908947.3) and had stable modifications such as a D614G mutation, a proline substitution (KV986-987PP), and a furin cleavage site elimination (RRAR₆₈₂₋₆₈₅QSAQ). The mRNA synthesis was based on the most widely used T7 RNA polymerase for in vitro transcription and co-transcription capping technologies. First, the codon optimized nucleotide sequence encoding the modified spike protein was cloned into a pUC plasmid and flanked by non-coding elements such as a T7 promoter sequence, 5′-UTR derived from the human acot7 gene, 3′-UTR derived from the mouse α-globin gene, and a 75nt poly(A) tail. The mRNA was subjected to co-transcription capping reaction with a T7 RNA polymerase (Thermo Fisher Scientific) in CleanCap®Reagent AG (Trilink), for in vitro RNA transcription. In in vitro transcription, 1-methyl-pseudouridine-triphosphate was used to replace uridine triphosphate (UTP), with the modification ratio of 1-methyl-pseudouracil of 100%. The RNA was treated with DNase I, purified by chromatography, filtered, and then dissolved in 1 mM sodium citrate buffer (pH of 6.4). The purity of RNA was tested by agarose gel electrophoresis and ion pair reversed phase-high performance liquid chromatography (IPRP-HPLC), and the double-stranded RNA was tested by homogeneous time-resolved fluorescence (HTRF). Then, the mRNA was aliquoted and stored at ≤−60° C. until use. The mRNA encoding a native spike protein was prepared with reference to the above method. The nucleic acid sequences encoding a native spike protein and the nucleic acid sequences encoding a modified spike protein are shown in Table 12.

TABLE 12
Nucleic acid sequences encoding a native spike protein and
nucleic acid sequences encoding a modified spike protein

| | SEQ ID NO: | | | | |
|---|---|---|---|---|---|
| | Amino acid | | | | |
| | sequence of | RNA | DNA | mRNA | DNA |
| | encoded | ORF se- | ORF se- | se- | se- |
| Name | protein | quence | quence | quence | quence |
| Nucleic acid | 12 | 3 | 5 | 6 | 7 |
| sequence | | | | | |
| encoding a | | | | | |
| modified | | | | | |
| spike | | | | | |
| protein | | | | | |
| Nucleic acid | 11 | 4 | 8 | 9 | 10 |
| sequence | | | | | |
| encoding a | | | | | |
| native spike | | | | | |
| protein | | | | | |

In vitro transfection: to evaluate the in vitro expression of the S protein, HEK293 cells (purchased from Shanghai Cell Bank, Chinese Academy of Sciences) and DC2.4 cells (purchased from Shanghai Cell Bank, Chinese Academy of Sciences) were inoculated in a 6-well plate at a rate of 1.2×10⁶ or 8×10⁵ cells/well. 18 Hours later, the cells were transiently transfected with 2 μg of mRNA encoding a native spike protein or the above designed mRNA encoding a modified spike protein by using Lipofectamine Massenger MAX transfection reagent (Thermo Fisher Scientific) according to the manufacturer's instructions. 24 Hours after the transfection, the cells were collected for western blot or flow cytometry analysis.

Western blot: cells transfected with mRNA were lysed, treated at 37° C. for 2 hours with or without PNGase F (NEB), and then mixed with SDS loading buffer for denaturation, followed by SDS-PAGE (polyacrylamide) gel electrophoresis and incubation with antibodies. The S protein was tested with a polyclonal rabbit anti-SARS-CoV-2RBD protein antibody (1:2000, sinobologic) and a secondary rabbit anti-horse radish peroxidase (HRP)-conjugated antibody (Beyotime). β-actin was tested with a monoclonal mouse anti-Actin HRP-conjugated antibody (Cell signaling technology). Blots were developed with high signal ECL Western Blotting Substrate (Tanon) and imaged using ECL Imager (Azure) and Image Lab software version 6.0.

Flow cytometry: cells transfected with mRNA were collected by direct pipetting (HEK293 cells) or 10 mM EDTA treatment (DC2.4 cells), and resuspended in cell staining buffer (biolegend). The cells were stained with Fixed Viability Stain (BD), to evaluate the viability of the cells, and blocked with an FcR blocking reagent (Miltenyi). The cells were stained with hACE2-Fc (GenScript) staining buffer at 4° C. for 30 minutes, and the spike expression on cell surfaces was tested. Subsequently, the cells were washed twice with a cell staining buffer, and then incubated with PE anti-human IgG Fc (Biolegend) in the cell staining buffer at 4° C. for 30 minutes. Sample collection was performed on a FACSCanto II flow cytometer (BD Biosciences) using BD FACSDiva software version 8.0.1.

The results of Western blot are shown in FIG. 17(B), where the mRNA-transfected cells encoding a native spike protein show two major bands (S and S1) (FIG. 2B, lanes 1 and 2), indicating that most of the spike proteins have been cleaved into the S1 and S2 subunits. However, the modified spike proteins expressed by the above-designed mRNA are present primarily as one complete protein despite partial cleavage (FIG. 2B, lanes 3 and 4). Meanwhile, it can be found that the above designed mRNA expresses a higher level of modified spike protein than the mRNA encoding a native spike protein (FIG. 2B, lanes 1 and 3).

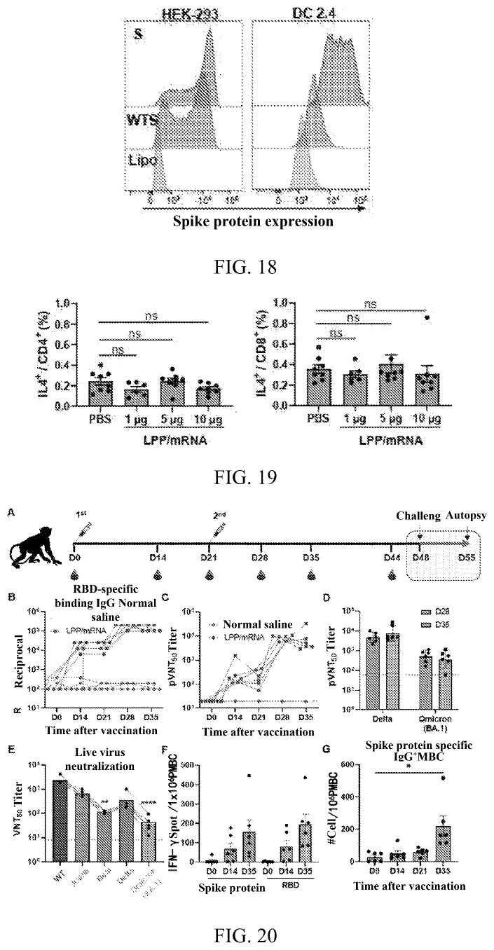

The results of flow cytometry are shown in FIG. 18, where the binding capacity of the modified spike protein to hACE2-Fc is significantly higher than the native spike protein.

All of the above data show that the designed mRNA is high in intracellular translation efficiency and is expressed on the cell surface.

### Example 15 LPP/mRNA Vaccine Inducing Strong Humoral Immune Response and Cellular Immune Response in Mice

This example used the preparation method as provided in Example 12 to prepare LPP/mRNA vaccines of LPP preparations containing the above mRNA encoding a modified spike protein (0.1 mg/ml) based on LPP-B2 prescription. To investigate the immunogenicity of the vaccines, as shown in FIG. 17(C), two batches of C57/BL6 mice (Charles River Laboratories, where the first batch of subjects n=8, and the second batch of subjects n=5) of 6-8 weeks of age were immunized by bilateral intramuscular injection with control PBS and LPP/mRNA containing 1 μg, 5 μg or 10 μg of mRNA, respectively, at days 0 and 14. Moreover, the spleens of the first batch of subjects were collected at day 21, and the blood of the second batch of subjects was collected at day 28, for test and analysis of the levels of humoral immune response and cellular immune response. The specific test methods were as follows.

Test of RBD-specific binding antibodies by enzyme-linked immunosorbent assay (ELISA): MICROLON® high binding Elisa plates (Greiner) were coated overnight at 4° C. with a recombinant RBD protein (Genscript,330-530aa) in 0.05 M sodium carbonate buffer. Washing was carried out with PBS-T (0.05% Tween-20, pH 7.4), and blocking was carried out with 1% BSA in PBS-T. Serially two-fold diluted heat-inactivated serum to be tested were added to each well. After three times of washing, incubation was carried out with the appropriate HRP conjugated secondary antibody (Abcam) and incubation was carried out with 3,3′,5,5′-tetramethylbenzidine (TMB) (ebioscience) substrate. The absorbance was read at 450/610 nm using a plate reader (BioTek). The endpoint titer was determined as the reciprocal value of the last dilution of serum to yield an absorbance greater than a threshold of 0.21.

Pseudovirus neutralization assay: the inhibition of virus entry is further tested by the SARS-CoV-2 pseudovirus neutralization assay (pVNT). The serum neutralization antibody titers of the immunized mice were determined according to the protocol of Nanjing Vazyme Biotech Co., Ltd. In short, the SARS-CoV-2 pseudovirus was pre-incubated with serially diluted positive controls or serum in Opti-MEM at 37° C. for 1 hour, then added to African green monkey kidney cells (Vero, purchased from Shanghai Cell Bank, Chinese Academy of Sciences) in a 96-well plate. 24 Hours after the incubation, the cells were lysed, and the luciferase signal was tested using Britelite™plus luminescence reporter gene assay kit (PerkinElmer) according to the manufacturer's protocol. The luminescence intensity was measured using a plate reader (BioTek). The neutralization titer (NT50) was defined as the reciprocal of serum dilution required for 50% inhibition of luciferase activity compared with virus control wells.

Enzyme-Linked ImmunoSpot Assay (ELISpot): the spike protein or RBD-specific T cell response in mouse splenocytes was tested with IFNγELISpotPLUS kit (Mabtech) according to the manufacturer's instructions. In short, 3×10⁵ splenocytes were stimulated with the spike protein RBD (Genscript) in vitro, or with phytohemagglutinin (PHA) and ionomycin as a positive control, or with RPMI 1640 medium as a negative control. 20 Hours after incubation at 37° C. with 5% CO2, the plates were sequentially treated with biotinylated IFNγ-detection antibody, streptavidin-alkaline phosphatase (ALP), and 5-bromo-4-chloro-3-indolyl-phosphate/nitrotetrazolium blue chloride-plus (BCIP/NBT-plus) substrate was added for color development. When the spot was strong enough to be visible to the naked eye, the color development was stopped with deionized water. IFNγ spot-forming cells were counted using an ELISpot plate reader (mmunoSpot S6Core Analyzer (CTL)).

Intracellular cytokine staining: the whole spleen of the mouse was manually ground to prepare a single splenocyte suspension, which was then filtered by 70 μm and 40 μm. He filtered splenocytes were resuspended in R10 medium (penicillin-streptomycin antibiotics and 10% heat-inactivated fetal calf serum were added in RPMI 1640), incubated at 37° C. for 12 hours, and stimulated with a spike protein peptide pool (Genscript). The peptide pool with a final concentration of 2 g/ml per peptide was employed. The cells were stimulated with phorbol ester (PMA) (500 ng/ml, Dakewe) and ionomycin (10 μg/ml, Dakewe) as positive controls. After stimulation, the cells were washed with PBS, and then stained with a viable cell/dead cell dye (Fixable Viability Stain, BD, 1:1000) at room temperature for 5 minutes. The cells were then washed in PBS and resuspended in a surface staining mixture containing the following antibodies: CD3-APC/Cyanine 7(Biolgend, 1:50), CD4PerCP/Cyanine 5.5(Biolgend, 1:50), CD8a APC(Biolgend, 1:50), and FcR blocking reagent (Miltenyi, 1:50) in staining buffer. 30 Minutes after the incubation, the cells were washed with PBS, then fixed with fixative buffer (BD) for 20 minutes, washed in perm/wash solution (BD), and stained intracellularly with a mixture of the following antibodies (4° C., 30 minutes): IFN-γBV421 (Biolgend, 1:50), TNF-αPE/Cyanine7 (Biolgend, 1:100), and IL-4PE (Biolgend, 1:100) in 1×perm/wash buffer (BD, diluted with sterile water). Finally, the cells were washed with perm/wash solution, and then resuspended with PBS. Sample collection was performed on a FACSCanto II flow cytometer (BD Biosciences) using BD FACSDiva software version 8.0.1.

The results of test of the binding antibodies are shown in FIG. 17(D), where the geometric mean titers (GMTs) of RBD binding serum IgG in the mice injected intramuscularly with LPP/mRNA preparations containing 1 μg, 5 μg, and 10 μg of mRNA at day 28 were 39,481, 51,200, and 72,408, respectively. This shows that LPP/mRNA induced a high level of RBD-specific binding antibodies in the mice after secondary immunization.

The results of the pseudovirus neutralization assay are shown in FIG. 17(E), where LPP/mRNA induced potent pseudovirus neutralization activity in the mice, and the GMTs of neutralization antibodies at day 28 after primary immunization were 34,771 (1 μg), 53,538 (5 μg), and 55,882 (10 μg), respectively (FIG. 2E). The results of the humoral immune response are dose-independent, indicating that the production can be the highest by receiving 1 μg dose of in vivo antibody of the mice.

Through intracellular cytokine staining and ELISpot assay, it is found that at day 21, a high proportion of IFN-γ (Th1 cytokine) and limited IL-4 (Th2 cytokine) secretion was tested in the spleen (as shown in FIG. 17(F) and FIG. 19). Further, the cytokine pattern of vaccine induced T cells was tested at day 21 after primary immunization by intracellular cytokine staining. The results are shown in FIGS. 17(G and H), where after re-stimulation with the spike protein peptide pool, LPP/mRNA induced CD4+T cells show a Th1 dominant response, in particular at a higher immunogenic dose. In addition, as shown in FIGS. 17(I and J), a strong CD8+T cell response to spike protein peptide pool stimulation was observed.

Furthermore, the applicant also used IgG2c heavy chain (HRP) antibody (ab97255, abeam) and Goat Anti-mouse IgG1, Human ads-HRP antibody (1070-05, SouthemBiotech) to test serum IgG1 and IgG2c antibody titers by ELISA according to the manufacturer's instructions (for the specific detection methods, see the above ELISA method). The results are shown in FIG. 17(K), where a higher IgG2c/IgG1 ratio indicates that the LPP/mRNA vaccines have a Th1 type humoral immune response.

Overall, these data show that LPP/mRNA can induce a superior humoral immune response and a Th1-dominant cellular immune response.

### Example 16 Protective Efficacy of LPP/mRNA Vaccine in Elderly Mice

The mouse-adapted SARS-CoV-2 strain is produced via in vivo passaging and leads to a 100% mortality rate in elderly mice. This strain has been used to evaluate the protective effect of candidate vaccines (S. Sun, et al., Characterization and structural basis of a lethal mouse-adapted SARS-CoV-2. Nature Communications. 12,5654 (2021), and F. Yan, et al., Characterization of two heterogeneous lethal mouse-adapted SARS-CoV-2 variants recapitulating representative aspects of humanCOVID-19. Frontiers in Immunology. 13 (2022)).

In this example, as shown in FIG. 17(L), BALB/c mice (Charles River Laboratories, n=8) of 8-9 months of age were immunized by intramuscular injection with control PBS and LPP/mRNA vaccines containing 1 g or 10 μg of mRNA at day 0 and day 14, respectively. 10 Days after the injection of the booster vaccines (day 24), all mice were injected intranasally with the mouse-adapted SARS-CoV-2 strain C57MA14 (50×LD₅₀) for challenge (challenge assay was conducted at Changchun Veterinary Research Institute, Chinese Academy of Agricultural Sciences). The condition of mice after challenge was observed daily, and the body weight for 14 days was recorded.

The results are shown in FIGS. 17(M and N), where all the mice inoculated with LPP/mRNA vaccines survived after lethal dose challenge, showing only transient and mild weight loss, followed by complete recovery without any evidence of disease exacerbation. In contrast, the control group rapidly lost weight and died within 6 days after the challenge. These results show that LPP/mRNA has a sufficient protective effect against C57MA14 lethal infection.

### Example 17 LPP/mRNA Vaccine Eliciting Strong Immunogenicity in Rhesus Monkeys

To evaluate the immunogenicity of LPP/mRNA in rhesus monkeys, as shown in FIG. 20(A), 6-year-old male rhesus monkeys were randomly divided into LPP/mRNA group and normal saline control group (n=6 in each group). The male rhesus monkeys were immunized by bilateral intramuscular injection with 500 μL of LPP/mRNA or normal saline containing 100 μg of mRNA at day 0 and day 21, respectively (the rhesus monkeys were immunized by Sichuan Hengshu Bio-Technology Co., Ltd.). Moreover, blood was taken for test and analysis at days 0, 14, 21, 28, and 35. For the test method for specific RBD-specific binding antibodies and the method for pseudovirus neutralization assay, reference was made to Example 15.

The results of test of binding antibodies are shown in FIG. 20(B), where IgG bound to RBD can be detected by ELISA as early as day 14 after primary immunization, and at day 7 (day 28) after secondary immunization, the level of binding antibodies in the rhesus monkeys is further increased, with a peak GMT of 182,456. The results of pseudovirus neutralization assay are shown in FIG. 20(C), where the single injection of LPP/mRNA can elicit a high level of neutralization antibody in all vaccinated animals, the GMT of the reciprocal of the 50% inhibition dilution (ID₅₀) at day 14 after primary immunization was 234, and is sharply increased to 8,396 at day 7 after secondary immunization.

This example also tested the neutralization effect on other strains of pseudoviruses (all purchased from Nanjing Vazyme Biotech Co., Ltd), as shown in FIG. 20(D) (with the data at day 28 and day 35 in sequence from left to right), compared with the wild-type strain, the neutralization effect on the Delta strain is only slightly reduced (1.75 times). On the contrary, the neutralization on Omicron (BA.1) strain pseudovirus is significantly reduced (16.5 times).

In addition, the neutralization activity against the wild-type strain was evaluated using the live virus cytopathic effect (CPE) assay. Moreover, to test the breadth of neutralization reaction, the CPE method was further used to test the neutralization activity of serum against multiple variants (Alpha/Beta/Delta/Omicron). The test methods were as follows.

In addition to the wild-type strain, 4 new variants of interest were also evaluated. The wild-type strain (GD108), beta variant strain (B.1.351, GDPCC-nCOV84, CSTR.16698.06.NPRC 2.062100001), alpha variant strain (B.1.1.7, SARS-CoV-2/C-Tan-BJ202101, CSTR.16698.06.NPRC 2.062100002), delta variant strain (B.1.617.2, CQ79, CSTR.16698.06.NPRC 6.CCPM-B-V-049-2105-8), and omicron variant strain (B.1.1.529CCPM-B-V-049-2112-18) are preserved at the Institute of Laboratory Animal Science, Chinese Academy of Medical Sciences. Vero E6 cells were used for amplification and titer determination. In short, the Vero-E6 cell monolayers in T225 culture bottles were inoculated with the virus. The virus induced CPE was observed 72 hours after inoculation, and the supernatant was collected and stored at −80° C. After slow thawing at 4° C. the next day, the virus supernatant was centrifuged, concentrated by ultrafiltration, and eluted with 200 ml PBS. The final virus titer was determined using the median tissue culture infectious dose (TCID₅₀) assay. The rhesus monkey serum was collected at day 4 before the challenge to evaluate its neutralization effect on SARS-CoV-2 infection. In short, Vero E6 cells (5×10⁴) were inoculated in a 96-well tissue culture plate and cultured overnight. 100 TCID₅₀SARS-CoV-2 virus was pre-incubated with an equal volume of serially diluted serum from immunized rhesus monkeys at 37° C. for 1 hour. The mixture was added to the Vero E6 cell monolayers. At day 3 after infection, the virus induced CPE was recorded under a microscope, and the reciprocal value of the serum dilution that completely prevented CPE in 50% of the wells was calculated using the Reed-Meunch method to determine the neutralization titer.

The serum was collected three weeks (day 44) after the second dose before the challenge. The results are shown in FIG. 20(E), where for the wild-type strain, up to this time point, the serum samples from all vaccinated animals still showed excellent NAb titers, with a geometric mean NAb titer close to 2,299, while no virus specific neutralization antibodies were tested in the control group. Compared with the wild-type strain, LPP/mRNA vaccination also resulted in excellent neutralization activity against Alpha/Beta/Delta/Omicron variant strains, although the neutralization activity was decreased by 3.7 times, 19.8 times, 8.2 times, and 64.6 times, respectively.

To evaluate the SARS-CoV-2-specific T cell response, peripheral blood mononuclear cells (PBMCs) were isolated from blood samples collected before immunization (D0), 14 days after the first immunization (D14), and 14 days after the second immunization (D35). After the in vitro stimulation with the S protein or RBD protein, the spike proteins producing interferon γ (IFN-γ) or RBD-specific T cells were detected using ELISpot. For the ELISpot assay, reference was made to Example 15, and the test results are shown in FIG. 20(F), where LPP/mRNA induces a strong IFN-γ response, in particular after the second administration.

The induction of memory B cell (MBC) is key to achieving a long-lasting protective humoral immune response (W. B. Alsoussi, et al., SARS-CoV-20 micron boosting induces de novo B cell response in humans. Nature, 1-3 (2023).) The multiparameter flow cytometry was used to evaluate the SARS-CoV-2-specific memory B cell response in PBMCs of rhesus monkeys inoculated with LPP/mRNA before and after immunization at designated time points. The test methods were as follows.

A probe for testing SARS-CoV-2-specific memory B cells in frozen rhesus monkey PBMCs was produced by continuously adding fluorescently labeled streptavidin to biotinylated spike proteins in PBS. The streptavidin was labeled with phycoerythrin (PE) or allophycocyanin (APC). The frozen rhesus monkey PBMCs were unfrozen and restored for at least 4 hours before treatment. The cells were stained with Fixable Viability Stain (BD), blocked with FcR blocking reagent (Miltenyi), and then stained with the following labeled monoclonal antibodies at 4° C. for 30 minutes: CD3 APC-Cy7(BD), CD8 APC-Cy7(BD), CD14 APC-Cy7(Biolegend), IgM PerCP-Cy5.5(BD), CD20 PE-Cy7(Biolegend), IgG FITC(BD), CD16 BV421(Biolegend). The cells were washed, and collected on FACSCanto II flow cytometer (BD) using BD FACSDiva software version 8.0.1.

The test results are shown in FIG. 20(G), where 14 days after the second administration, there was a significant increase in SARS-CoV-2-specific memory B cells identified in PBMCs.

The above data show that LPP/mRNA also has high immunogenicity in non-human primate models, has extensive cross neutralization activity and crucial T and B cell responses for long-term immune protection.

### Example 18 LPP/mRNA Vaccine Protecting Rhesus Monkeys from SARS-CoV-2 Infection

In this example, the protective efficacy of LPP/mRNA vaccines against rhesus monkeys was further evaluated. As shown in FIG. 20(A), rhesus monkeys were immunized by intramuscular injection with two doses of LPP/mRNA or normal saline containing 100 g of mRNA, and 48 days after inoculation, 1×10⁶TCID₅₀ mL⁻¹SARS-CoV-2 (GD108) prototype strain (with a volume of 0.5 mL each) was inoculated by a combination of intranasal and intratracheal routes (administration by nasal drip and tracheal injection) for challenge (challenge assay was conducted at the National Kunming High-level Biosafety Primate Research Center). Regular physical examinations were conducted during the experiment, and the body temperature and weight of the animals before and after the challenge were recorded. For the time points for recording, reference was made to FIG. 20(A). The viral load of nasal/oropharyngeal/anal swabs and the viral load of lung lobes before and after the challenge were tested at different designated time points. Moreover, 7 days after the challenge, anesthesia and autopsy were conducted on the rhesus monkeys, and the superior, middle, inferior lobe tissues of the left or right lung were collected for viral load assay and pathological analysis. The test methods for viral load and pathological analysis were as follows.

RT-qPCR test of SARS-CoV-2 viral RNA: viral genomic RNA (gRNA) was tested using real-time quantitative reverse transcription PCR (qRT-PCR). The primers and probe sequences used were designed to target the N gene (forward primer as shown in SEQ ID NO: 20, 5′-GGGGAACTTCTCCTGCTAGAAT-3′; reverse primer as shown in SEQ ID NO: 21, 5′-CAGACATTTTGCTCTCAAGCTG-3′; probe sequence as shown in SEQ ID NO: 22, 5′-FAM-TTGCTGCTGCTTGACAGATT-TAMRA-3′). For the sequences, reference was made to the sequences recommended by the World Health Organization and the Chinese Center for Disease Control and Prevention.

Histopathology: the collected lung tissue was fixed with formalin, sliced, and stained with hematoxylin and eosin, for observation of histological changes. Two pathologists evaluated the pathological changes of lung tissue under a microscope in a double-blind manner, and then comprehensively evaluated the histopathological images of the entire lung lobe for scoring. The protective effect of the vaccines were evaluated through the pathological images and the comprehensive scoring. Each cerebral lobe had a separate score, and the final score of each rhesus monkey was reported as the average of the individual scores.

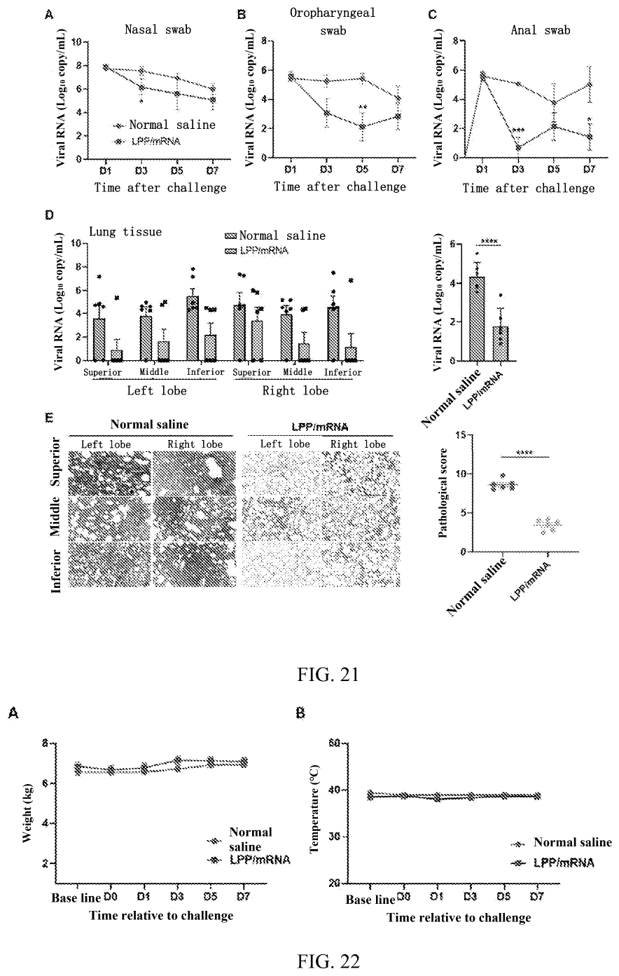

The results of test of virus load are shown in FIGS. 21(A-C), where day 1 after the challenge, the peak virus gRNA load was observed in all animal swab samples, indicating successful challenge. During the entire evaluation period after infection with the virus, the nasal/oropharyngeal/anal swabs of all rhesus monkeys injected with placebo (normal saline) showed and maintained excessive copies of viral genomic RNA. In contrast, the viral load of swabs in the vaccine group was significantly decreased 3 days after the challenge, and remained lower than the control group at other sampling time points (5 days and 7 days after the challenge). In particular, after the challenge, no virus gRNA was tested in the oropharyngeal swabs of several rhesus monkeys in the vaccine group (2 out of 6 monkeys at day 3 after the challenge, 3 out of 6 monkeys at day 5 after the challenge, and 2 out of 6 monkeys at day 7 after the challenge). In the anal swabs, viral infection was tested in only one out of the 6 animals in the vaccine group at day 3. Similarly, as shown in FIG. 21(D) (with the data of normal saline group and LPP-mRNA group from left to right), a high level of viral load was observed in at least 4 lung lobes of the lung tissue of all the 6 animals in the normal saline group 7 days after the challenge. However, no virus was tested in most lung lobes of the lung tissue of the 6 animals in the vaccine group or the viral load was significantly reduced.

The results of pathological analysis of the fixed lung tissue are shown in FIG. 21(E), where significant interstitial pneumonia, thickening and hemorrhage of the pulmonary septum, inflammatory cell infiltration, and distribution of dust cells with pigmentation can be observed in the pathological tissue of the normal saline group. In contrast, the pathology of lung tissue of all vaccinated rhesus monkeys was close to normal, with focal and mild histopathological changes in a very small number of lung lobes, and no immune enhanced inflammation was observed. Thus, the comprehensive histopathological score of the lungs of the animals in the vaccine group was significantly lower than that in the normal saline group. In addition, as shown in FIG. 22, 1-7 days after the challenge, both the body weight and temperature of the rhesus monkeys in the vaccine group and the normal saline group had no significant fluctuation.

In summary, these data show that LPP/mRNA has a significant preventive effect on the replication of SARS-CoV-2 in the lungs and effectively protects rhesus monkeys from infection and lung lesions.

### Example 19 Third Dose of LPP/mRNA Booster Vaccine Producing Excellent Immune Response Against Omicron Variant Strain

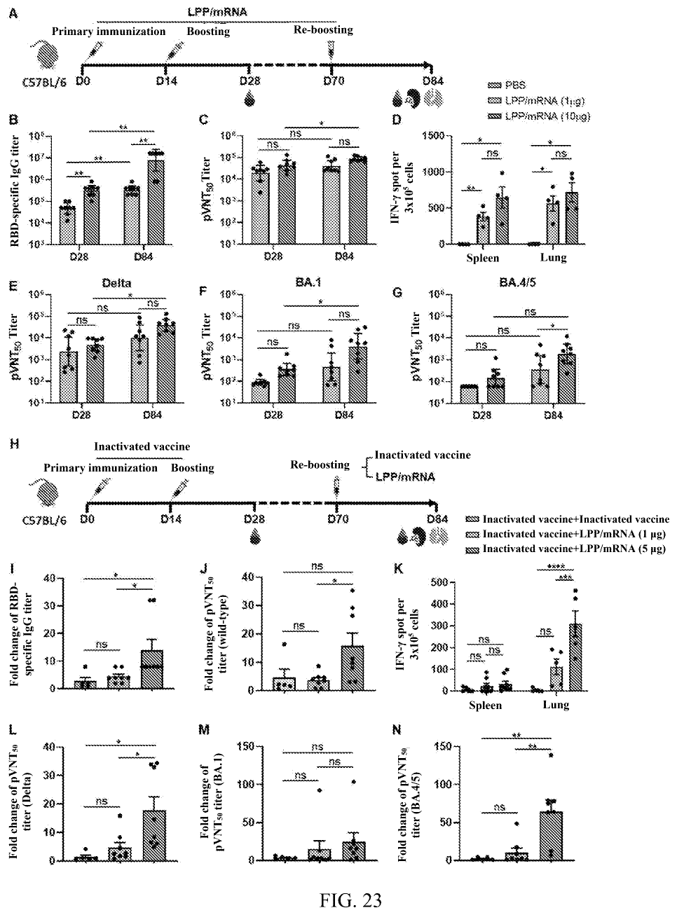

To investigate the immunogenicity induced by homologous booster immunization, as shown in FIG. 23(A), C57BL/6 mice (Charles River Laboratories, n=8) were immunized by intramuscular injection with LPP/mRNA containing 1 g and 10 g of mRNA or control PBS, with an immunization interval of two weeks (immunized at day 0 and day 14), and booster injection was given 70 days after primary immunization. At day 84, the mice were euthanized, and the blood, spleens, and lungs were collected for test and analysis. For the test method for RBD-specific binding antibodies and the method for pseudovirus neutralization assay, reference was made to Example 15.

The results of test of RBD-specific binding antibodies are shown in FIG. 23(B) (with the data of 1 μg LPP/mRNA group and 10 μg LPP/mRNA group in sequence from left to right), where compared with 1 μg LPP/mRNA, 10 μg LPP/mRNA induced a higher level of RBD-specific binding antibodies in the mice at day 84 after primary immunization, and the binding antibody GMTs of the mice in the 1 μg LPP/mRNA immunization group and the 10 μg LPP/mRNA immunization group reached 7,747,515 and 315,845, respectively. Compared with the levels of binding antibodies of 46,951 (1 μg mRNA/LPP) and 315,845 (10 μg mRNA/LPP) at day 28, the booster immunization further increased the IgG titer.

The results of pseudovirus neutralization assay for the wild-type SARS-CoV-2 strain are shown in FIG. 23(C) (with the data of 1 μg LPP/mRNA group and 10 μg LPP/mRNA group in sequence from left to right), where the neutralization antibody GMTs of the mice in the 10 μg LPP/mRNA immunization group at days 28 and 84 were 44,494 and 85,808, respectively, while the neutralization antibody GMTs of the 1 μg LPP/mRNA immunization group mice at days 28 and 84 were 19,193 and 41,359, respectively. These data further show that the booster injection increases the level of neutralization antibodies.

The cellular immune response was tested by ELISpot assay (see Example 15 for specific methods), and the results are shown in FIG. 23(D) (with data of PBS group, 1 μg LPP/mRNA group, and 10 μg LPP/mRNA group in sequence from left to right), where the LPP/mRNA vaccine elicited strong T cell responses secreting IFN-γ in both the spleen or lung.

Due to the lower neutralization ability of 2-dose immunization against BA.1 (as shown in FIG. 20D), whether 3-dose homologous immunization would increase the neutralization activity of the variant strain was further evaluated in this example. The serum samples 28 and 84 days after immunization were selected to evaluate the cross neutralization activity. For the specific test methods, reference was made to Examples 15 and 17.

The results of pseudovirus neutralization assay are shown in FIGS. 23(E-G) (with data of 1 μg LPP/mRNA group and 10 g LPP/mRNA group in sequence from left to right), where at day 84, 10 μg LPP/mRNA induced stronger neutralization antibody titers against Delta, BA.1, and BA.4/5 strains in mouse serum, with GMTs of 38,528, 4,138, and 1,871, respectively.

Next, a further comparison was made between homologous and heterologous immunization as the third dose of vaccination strategies.

As shown in FIG. 23(H), C57BL/6 mice (Charles River Laboratories, n=5 or 8) were immunized by intramuscular injection with a 50 U/mouse inactivated vaccine (presented by the Institute of Medical Biology, Chinese Academy of Medical Sciences (trade name: Covidful, common name: novel coronavirus inactivated vaccine (Vero cell)), with an immunization interval of two weeks (immunized at day 0 and day 14 day). 70 days after the primary immunization, the same inactivated vaccine or 1 μg and 5 μg LPP/mRNA was injected for the third time as a booster. Moreover, the serum samples were collected at day 84 after immunization (14 days after the third immunization) to test binding antibody and neutralization antibody responses (see Example 15 for specific test methods for binding antibodies and neutralization antibodies).

The results of test of binding antibodies are shown in FIG. 23(I) (with data of homologous inactivated vaccine booster immunization group, heterologous 1 μg LPP/mRNA booster immunization group, and heterologous 5 μg LPP/mRNA booster immunization group in sequence from left to right), where the mice with the third dose of homologous inactivated vaccine as a booster had no significant increase in the IgG antibody level in vivo at days 28 and 84, with GMTs of 19,401 and 38,802, respectively. Compared with the homologous inactivated vaccine booster immunization, the binding antibody GMTs induced by LP/mRNA (5 μg) heterologous immunization at days 28 and 84 reached 13,958 and 157,922, respectively. This shows that heterologous booster immunization with LPP/mRNA vaccine can significantly increase the level of binding antibodies.

The results of test of neutralization antibodies for the wild-type strain are shown in FIG. 23(J) (with data of homologous inactivated vaccine booster immunization group, heterologous 1 μg LPP/mRNA booster immunization group, and heterologous 5 μg LPP/mRNA booster immunization group in sequence from left to right), where the mice receiving an inactivated vaccine as a booster had no significant increase in neutralization antibody titer, the IgG antibody GMTs at days 28 and 84 reached 2,833 and 6,859, respectively, while LPP/mRNA/LPP immunization significantly increased the neutralization antibody level, and the neutralization antibody GMTs induced by LP/mRNA (5 μg) heterologous immunization at days 28 and 84 reached 2,559 and 28,672, respectively.

In addition, the influence of heterologous or homologous booster on the cellular immune response was tested by ELISpot, and the results are shown in FIG. 23(K) (with data of homologous inactivated vaccine booster immunization group, heterologous 1 μg LPP/mRNA booster immunization group, and heterologous 5 μg LPP/mRNA booster immunization group in sequence from left to right). The LPP/mRNA vaccine as a booster was superior to the inactivated vaccine in inducing a T cell response secreting IFN-γ, and no cellular immune response was tested in the spleens and lungs of the mice receiving the inactivated vaccine as a booster.

In addition, neutralization assay was also conducted to evaluate the cross neutralization activity of homologous or heterologous immunization against Delta, BA.1, and BA.4/5 variant strains, and the test results are shown in FIGS. 23(L-N) (with data of homologous inactivated vaccine booster immunization group, heterologous 1 μg LPP/mRNA booster immunization group, and heterologous 5 μg LPP/mRNA booster immunization group in sequence from left to right), and are consistent with the results of the binding antibodies. Compared with inactivated vaccine booster immunization, LPP/mRNA (1 μg or 5 μg mRNA) booster immunization can further enhance the neutralization ability against Delta, BA.1, and BA.4/5 variant strains. In particular, for the BA.4/5 strain, it was noted that the immunization with two times of injection with LPP/mRNA or inactivated vaccine had very low neutralization ability against BA.4/5, the neutralization antibody GMT of the mice was only 60, but after the booster immunization with LPP/mRNA (5 μg mRNA), the neutralization antibody GMT of the mice can reach 2,790, while after the inactivated vaccine booster immunization, the neutralization antibody GMT of the mice was only 108.

In summary, these results show that LPP/mRNA vaccines, as homologous or heterologous booster strategies, are superior to inactivated vaccine booster strategies in inducing humoral immune response and cellular immune response.

### Example 20 LPP/mRNA Vaccine Showing Good Safety in Rhesus Monkeys

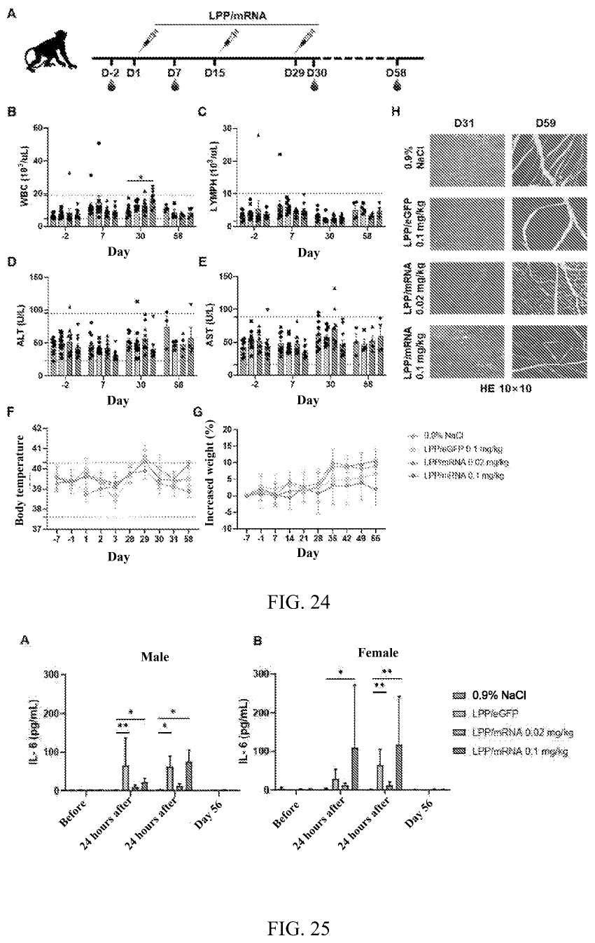

To conduct the study on repeated administration toxicity, as shown in FIG. 24(A), 40 rhesus monkeys (20 males and 20 females) were divided into 4 groups and intramuscularly administered with 0.9% sodium chloride, LPP/eGFP (administration system control), 0.02 mg/kg, and 0.1 mg/kg LPP/mRNA. Administration was carried out at day 1, day 15, and day 29, with a recovery period of 4 weeks. Moreover, throughout the entire research process, the following endpoints and parameters were evaluated: mortality rate, near-death rate, clinical observation, weight, food consumption, body temperature, electrocardiogram, respiration, blood pressure, ophthalmology, hematology and coagulation, clinical chemistry, immunologic function, urine analysis, fecal occult blood, cytokine, organ weight, gross autopsy, histopathological examination, tissue distribution, and antibody titer.

The serum was collected for hematological and coagulation test two days before administration (D-2), during the adaptation period (D7, D30), and during the recovery period (D58). The hematological analysis samples were collected into tubes containing EDTA-K₂ as an anticoagulant, and analyzed using the ADVIA 2120i automated hematology analyzer. The samples used for coagulation analysis were collected into tubes containing sodium citrate as an anticoagulant, and analyzed using the Sysmex CS-5100 automated coagulation analyzer. The serum was collected for cytokine analysis and antibody titer analysis.

No animal death or near death was found throughout the entire study period. No abnormalities related to the test substance were found in food consumption, electrocardiogram, respiration, blood pressure, ophthalmology, clinical chemistry, immunologic function, urine analysis, fecal occult blood, organ weight, or gross autopsy. In clinical observation, some animals in the LPP/eGFP and LPP/mRNA administration groups showed loose stools, but similar phenomena were also observed during the adaptation period and the 0.9% sodium chloride group. The body temperature started to be monitored after the first administration. The animals in the LPP/eGFP and LPP/mRNA (0.02 mg/kg and 0.1 mg/kg) groups showed an increase in body temperature 6 hours after administration at day 1 and day 29, and returned to normal after 24 and 48 hours.

The serum was collected for hematological and coagulation test two days before administration (D-2), during the adaptation period (D7, D30), and during the recovery period (D58). The test results are shown in FIG. 24 (B-E) (with data of 0.9% sodium chloride group, LPP/eGFP 0.1 mg/kg group, LPP/mRNA 0.02 mg/kg group, and LPP/mRNA 0.1 mg/kg group in sequence from left to right), where after the first administration, compared with the 0.9% sodium chloride group, the hematological and coagulation test results (including alanine aminotransferase (ALT), aspartate aminotransferase (AST), white blood cell (WBC), and lymphocyte (LYMPH) tests) of the rhesus monkeys in the LPP/eGFP and LPP/mRNA (0.02 mg/kg and 0.1 mg/kg) groups have no significant difference. The monitoring results of the body temperature and weight are shown in FIGS. 24(F and G), the fluctuations in body temperature and weight of the rhesus monkeys in the LPP/eGFP group and the high-dose LPP/mRNA group are similar.

At day 31, some subjects were euthanized and tissue was taken for histopathological examination. At day 59, the remaining subjects were euthanized, and tissue was collected for histopathological examination. The results of histopathological examination are shown in FIG. 6H, after 2 days of the administration, inflammatory cell infiltration was observed in the administration sites of the mice in the LPP/eGFP group and LPP/mRNA group (FIG. 6H, sample D31), without permanent damage, and they returned to normal after 29 days of the administration (FIG. 6H, sample D59).

The applicant also used the same immunization program to treat another batch of subjects, and collected serum two days before administration (D-2), 24 hours after administration at day 1, 24 hours after administration at day 29, and at day 56. The U-PLEX TH17Combo 1(NHP) detection kit (Cat. No. K15079K) was used to test cytokine IL-6 by ELISA according to the manufacturer's instructions. The test results of cytokine IL-6 are shown in FIG. 25 (with data of 0.9% sodium chloride group, LPP/eGFP 0.1 mg/kg group, LPP/mRNA 0.02 mg/kg group, and LPP/mRNA 0.1 mg/kg group in sequence from left to right), where 24 hours after administration at day 1 and day 29, compared with the 0.9% sodium chloride group, the levels of IL-6 of the rhesus monkeys in the LPP/eGFP group and the LPP/mRNA (0.02 mg/kg and 0.1 mg/kg) group are increased significantly, and recovered at day 56.

Although the present disclosure has been disclosed above by preferred examples, it is not intended to limit the present disclosure. Anyone who is familiar with the art can make various changes and modifications without departing from the spirit and scope of the present disclosure. Therefore, the scope of protection of the present disclosure shall be subject to the scope of protection claimed by the claims.

Sequence Listing

| SEQ ID | | |
|---|---|---|
| NO: | Description | Sequence |
| 1 | Fluc | AUGGAGGACGCCAAGAACAUCAAGAAGGGCCCCGCCCCCUUCUACCCCCUGGAGGACG |
| | encoding | GCACCGCCGGCGAGCAGCUGCACAAGGCCAUGAAGCGGUACGCCCUGGUGCCCGGCAC |
| | sequence | CAUCGCCUUCACCGACGCCCACAUCGAGGUGGACAUCACCUACGCCGAGUACUUCGAG |
| | | AUGAGCGUGCGGCUGGCCGAGGCCAUGAAGCGGUACGGCCUGAACACCAACCACCGG |
| | | AUCGUGGUGUGCAGCGAGAACAGCCUGCAGUUCUUCAUGCCCGUGCUGGGCGCCCUG |
| | | UUCAUCGGCGUGGCCGUGGCCCCCGCCAACGACAUCUACAACGAGCGGGAGCUGCUG |
| | | AACAGCAUGGGCAUCAGCCAGCCCACCGUGGUGUUCGUGAGCAAGAAGGGCCUGCAG |
| | | AAGAUCCUGAACGUGCAGAAGAAGCUGCCCAUCAUCCAGAAGAUCAUCAUCAUGGAC |
| | | AGCAAGACCGACUACCAGGGCUUCCAGAGCAUGUACACCUUCGUGACCAGCCACCUGC |
| | | CCCCCGGCUUCAACGAGUACGACUUCGUGCCCGAGAGCUUCGACCGGGACAAGACCAU |
| | | CGCCCUGAUCAUGAACAGCAGCGGCAGCACCGGCCUGCCCAAGGGCGUGGCCCUGCCC |
| | | CACCGGACCGCCUGCGUGCGGUUCAGCCACGCCCGGGACCCCAUCUUCGGCAACCAGA |
| | | UCAUCCCCGACACCGCCAUCCUGAGCGUGGUGCCCUUCCACCACGGCUUCGGCAUGUU |
| | | CACCACCCUGGGCUACCUGAUCUGCGGCUUCCGGGUGGUGCUGAUGUACCGGUUCGA |
| | | GGAGGAGCUGUUCCUGCGGAGCCUGCAGGACUACAAGAUCCAGAGCGCCCUGCUGGU |
| | | GCCCACCCUGUUCAGCUUCUUCGCCAAGAGCACCCUGAUCGACAAGUACGACCUGAGC |
| | | AACCUGCACGAGAUCGCCAGCGGCGGCGCCCCCCUGAGCAAGGAGGUGGGCGAGGCCG |
| | | UGGCCAAGCGGUUCCACCUGCCCGGCAUCCGGCAGGGCUACGGCCUGACCGAGACCAC |
| | | CAGCGCCAUCCUGAUCACCCCCGAGGGCGACGACAAGCCCGGCGCCGUGGGCAAGGUG |
| | | GUGCCCUUCUUCGAGGCCAAGGUGGUGGACCUGGACACCGGCAAGACCCUGGGCGUG |
| | | AACCAGCGGGGCGAGCUGUGCGUGCGGGGCCCCAUGAUCAUGAGCGGCUACGUGAAC |
| | | AACCCCGAGGCCACCAACGCCCUGAUCGACAAGGACGGCUGGCUGCACAGCGGCGACA |
| | | UCGCCUACUGGGACGAGGACGAGCACUUCUUCAUCGUGGACCGGCUGAAGAGCCUGA |
| | | UCAAGUACAAGGGCUACCAGGUGGCCCCCGCCGAGCUGGAGAGCAUCCUGCUGCAGC |
| | | ACCCCAACAUCUUCGACGCCGGCGUGGCCGGCCUGCCCGACGACGACGCCGGCGAGCU |
| | | GCCCGCCGCCGUGGUGGUGCUGGAGCACGGCAAGACCAUGACCGAGAAGGAGAUCGU |
| | | GGACUACGUGGCCAGCCAGGUGACCACCGCCAAGAAGCUGCGGGGGGGGGUGGUGUU |
| | | CGUGGACGAGGUGCCCAAGGGCCUGACCGGCAAGCUGGACGCCCGGAAGAUCCGGGA |
| | | GAUCCUGAUCAAGGCCAAGAAGGGCGGCAAGAUCGCCGUGUGA |
| 2 | eGFP | AUGGUGAGCAAGGGCGAGGAGCUGUUCACCGGGGUGGUGCCCAUCCUGGUCGAGCU |
| | encoding | GGACGGCGACGUAAACGGCCACAAGUUCAGCGUGUCCGGCGAGGGCGAGGGCGAUGC |
| | sequence | CACCUACGGCAAGCUGACCCUGAAGUUCAUCUGCACCACCGGCAAGCUGCCCGUGCCC |
| | | UGGCCCACCCUCGUGACCACCCUGACCUACGGCGUGCAGUGCUUCAGCCGCUACCCCG |
| | | ACCACAUGAAGCAGCACGACUUCUUCAAGUCCGCCAUGCCCGAAGGCUACGUCCAGGA |
| | | GCGCACCAUUCUUCUJUCAAGGACGACGGCAACUACAAGACCCGCGCCGAGGUGAAGUUC |
| | | GAGGGCGACACCCUGGUGAACCGCAUCGAGCUGAAGGGCAUCGACUUCAAGGAGGAC |
| | | GGCAACAUCCUGGGGCACAAGCUGGAGUACAACUACAACAGCCACAACGUCUAUAUCA |
| | | UGGCCGACAAGCAGAAGAACGGCAUCAAGGUGAACUUCAAGAUCCGCCACAACAUCGA |
| | | GGACGGCAGCGUGCAGCUCGCCGACCACUACCAGCAGAACACCCCCAUCGGCGACGGC |
| | | CCCGUGCUGCUGCCCGACAACCACUACCUGAGCACCCAGUCCGCCCUGAGCAAAGACCC |
| | | CAACGAGAAGCGCGAUCACAUGGUCCUGCUGGAGUUCGUGACCGCCGCCGGGAUCAC |
| | | UCUCGGCAUGGACGAGCUGUACAAGUAA |
| 3 | RNA | AUGUUCGUAUUCCUAGUGCUGCUGCCCCUGGUGAGCAGCCAGUGCGUGAACCUGACC |
| | encoding a | ACACGCACUCAGCUGCCACCAGCCUACACCAACUCAUUCACCAGGGGCGUGUACUAUCC |
| | modified | UGACAAGGUGUUCAGGAGCAGCGUGCUGCACUCCACCCAGGAUCUGUUCCUGCCCUU |
| | spike | CUUCAGCAACGUGACAUGGUUCCACGCCAUCCACGUGAGCGGGACAAACGGCACCAAG |
| | protein | CGGUUUGACAAUCCCGUUCUGCCAUUCAACGAUGGCGUGUACUUCGCCAGCACCGAG |
| | ORF | AAAAGCAACAUCAUCCGGGGCUGGAUCUUCGGCACCACACUGGACAGCAAGACCCAGA |
| | sequence | GUCUGCUGAUCGUGAACAACGCCACCAACGUGGUGAUCAAGGUGUGUGAGUUCCAGU |
| | | UCUGUAAUGACCCCUUCCUGGGGGUCUAUUACCACAAGAACAAUAAAAGCUGGAUGG |
| | | AGAGCGAGUUUCGGGUGUACAGUAGUGCCAACAACUGUACAUUCGAGUACGUGUCCC |
| | | AGCCAUUCCUGAUGGACCUGGAGGGGAAGCAGGGGAACUUCAAGAACCUGCGGGAGU |
| | | UCGUGUUUAAGAAUAUCGACGGAUAUUUUAAGAUCUACUCCAAGCACACCCCCAUCAA |
| | | CCUGGUGCGGGAUCUGCCCCAGGGCUUCAGCGCCCUGGAGCCCCUGGUGGACCUGCC |
| | | CAUCGGGAUCAACAUCACCCGGUUCCAGACCCUGCUGGCCCUGCACCGGAGUUACCUG |
| | | ACCCCAGGUGACUCCAGCAGCGGCUGGACAGCCGGGGCUGCAGCCUACUACGUGGGCU |
| | | ACCUGCAGCCCCGGACCUUCCUCCUGAAGUACAAUGAGAACGGCACCAUCACCGAUGC |
| | | CGUGGAUUGUGCUCUGGACCCCCUGUCUGAGACCAAGUGCACCCUGAAAAGCUUCAC |
| | | CGUGGAGAAGGGGAUCUACCAGACAAGCAACUUUCGGGUGCAGCCCACCGAGAGUAU |
| | | CGUGAGGUUCCCCAACAUCACCAACCUGUGUCCAUUCGGGGAGGUGUUCAACGCCACC |
| | | CGGUUCGCAAGCGUGUACGCUUGGAACCGGAAGCGCAUCAGCAAUUGCGUGGCUGAU |
| | | UACAGCGUGCUGUACAACAGCGCCUCCUUCAGCACAUUCAAGUGCUACGGCGUCUCCC |
| | | CCACAAAGCUGAACGAUCUGUGUUUCACCAACGUGUACGCAGACAGCUUUGUGAUCC |
| | | GGGGAGACGAGGUGCGGCAGAUCGCACCCGGGCAGACCGGCAAGAUUGCUGACUACA |
| | | AULACAAGCUGCCUGAUGACUUCACAGGCUGCGUGAUUGCCUGGAACAGCAAUAACC |
| | | UGGACAGCAAGGUGGGAGGUAAUUACAAUUACCUCUACCGGCUGUUCAGGAAGUCCA |
| | | AUCUGAAGCCCUUCGAGAGGGACAUCUCCACCGAGAUCUACCAGGCUGGGUCCACCCC |
| | | AUGCAAUGGGGUGGAGGGCUUCAACUGCUACUUCCCCCUGCAGUCCUACGGCUUCCA |
| | | GCCCACCAAUGGGGUGGGCUACCAGCCCUACCGGGUGGUGGUGCUGAGCUUCGAGCU |
| | | CCUGCACGCCCCCGCCACUGUGUGCGGCCCCAAGAAGUCCACCAACCUGGUGAAGAAC |
| | | AAGUGCGUGAACUUCAACUUCAACGGGUUGACCGGGACUGGGGUGCUCACAGAGAGC |
| | | AACAAGAAGUUCCUGCCCUUCCAGCAGUUCGGAAGGGACAUCGCCGACACCACGGAUG |
| | | CCGUGAGGGAUCCCCAGACCCUCGAGAUCCUGGACAUCACGCCAUGCUCCUUCGGGGG |
| | | CGUGAGCGUGAUCACUCCUGGCACAAACACCUCCAACCAGGUGGCUGUGCUGUACCAG |
| | | GGCGUGAACUGCACCGAGGUGCCUGUGGCCAUCCAUGCUGACCAGCUGACCCCCACCU |
| | | GGCGGGUCUACAGCACCGGCAGCAACGUGUUCCAGACCCGGGCCGGCUGCCUGAUCG |
| | | GGGCCGAGCACGUGAACAACAGCUACGAGUGCGAUAUCCCCAUCGGCGCCGGCAUCUG |
| | | CGCUAGCUACCAGACACAGACCAAUAGCCCCCAAUCCGCUCAAUCUGUGGCUAGCCAGA |
| | | GCAUCAUCGCCUACACUAUGUCUCUGGGCGCAGAGAAUAGUGUGGCUUACAGCAACA |
| | | ACAGCAUCGCCAUUCCCACUAAUUUCACAAUUAGUGUGACCACCGAGAUCCUGCCUGU |
| | | GAGCAUGACCAAGACCAGCGUGGACUGCACCAUGUACAUCUGCGGGGAUAGCACCGAG |
| | | UGCAGCAACCUGCUGCUGCAGUACGGCAGCUUCUGCACACAGCUGAACCGGGCCCUGA |
| | | CCGGUAUCGCUGUGGAGCAGGACAAGAACACCCAGGAGGUGUUUGCGCAGGUGAAGC |
| | | AGAUCUACAAGACCCCUCCUAUCAAGGACUUCGGGGGCUUUAACUUCAGCCAGAUCCU |
| | | GCCAGACCCCUCCAAGCCCUCCAAGAGGAGCUUCAUCGAGGAUCUCCUGUUCAACAAG |
| | | GUGACCCUGGCCGAUGCCGGCUUCAUCAAGCAGUACGGAGAUUGCCUGGGCGACAUC |
| | | GCCGCCAGGGAUCUCAUCUGCGCCCAGAAGUUCAACGGCCUGACCGUGCUGCCCCCCC |
| | | UGCUGACCGAUGAGAUGAUCGCUCAGUACACCAGCGCCCUGCUGGCUGGGACCAUCAC |
| | | CAGCGGGUGGACCUUCGGGGCCGGCGCCGCCCUGCAGAUCCCCUUCGCCAUGCAGAUG |
| | | GCCUACCGGUUCAACGGUAUUGGGGUGACCCAGAAUGUGCUGUAUGAGAACCAGAAG |
| | | CUGAUCGCCAACCAGUUCAACUCUGCCAUCGGCAAGAUCCAGGACUCCCUGAGCAGCA |
| | | CUGCCAGCGCCCUGGGCAAGCUGCAGGACGUGGUGAACCAGAACGCCCAGGCCCUGAA |
| | | UACCCUGGUGAAGCAGCUGUCCAGCAACUUCGGGGCCAUCAGCAGCGUGCUGAACGA |
| | | CAUCCUGUCCCGCCUGGACCCACCAGAGGCAGAGGUGCAGAUCGAUCGGCUGAUCACA |
| | | GGUAGGCUGCAGAGCCUGCAGACCUACGUGACCCAGCAGCUGAUCCGCGCCGCUGAGA |
| | | UUAGGGCCAGCGCCAACCUGGCCGCCACUAAGAUGAGCGAGUGCGUGCUGGGCCAGA |
| | | GCAAGCGGGUGGACUUCUGCGGCAAGGGGUAUCACCUGAUGAGCUUCCCUCAGUCCG |
| | | CCCCCCACGGGGUGGUGUUCCUGCACGUGACAUACGUGCCCGCCCAGGAGAAGAACUU |
| | | CACCACCGCCCCUGCCAUCUGCCACGAUGGCAAGGCCCACUUUCCCCGGGAGGGCGUG |
| | | UUCGUGUCCAAUGGCACACAUUGGUUCGUGACCCAGCGGAACUUCUACGAGCCACAG |
| | | AUCAUUACCACUGACAAUACCUUUGUCAGUGGUAACUGUGACGUGGUGAUCGGCAUU |
| | | GUGAACAACACAGUGUACGAUCCUCUGCAGCCAGAGCUGGACAGCUUCAAGGAGGAG |
| | | CUGGACAAGUACUUCAAGAACCACACCAGCCCCGACGUGGAUCUUGGAGACAUCUCUG |
| | | GCAUCAACGCCAGCGUGGUCAACAUCCAGAAGGAGAUUGACCGCCUGAACGAGGUUG |
| | | CCAAGAACCUGAACGAGAGUCUGAUCGACCUCCAGGAGCUUGGCAAGUACGAGCAGUA |
| | | CAUCAAGUGGCCAUGGUACAUCUGGCUGGGCUUCAUCGCCGGCCUGAUCGCCAUCGU |
| | | GAUGGUGACCAUCAUGCUGUGCUGCAUGACCUCUUGUUGCAGCUGCCUGAAGGGGU |
| | | GCUGCAGCUGCGGUAGUUGCUGUAAGUUCGACGAGGACGACAGCGAGCCCGUGCUGA |
| | | AGGGCGUGAAGCUGCACUACACGUAAUAG |
| 4 | RNA ORF | AUGUUCGUCUUCCUGGUGCUGCUGCCUCUGGUGUCUUCCCAGUGCGUGAAUCUGAC |
| | sequence | UACCAGGACCCAGCUGCCCCCUGCCUAUACCAAUUCCUUCACACGGGGCGUGUACUAU |
| | encoding a | UUCUUUAGCAACGUGACCUGGUUCCACGCCAUCCACGUGAGCGGCACCAAUGGCACAA |
| | native | CCCGACAAGGUGUUUAGAAGCUCCGUGCUGCACUCUACACAGGAUCUGUUUCUGCCU |
| | spike | AGCGGUUCGACAAUCCAGUGCUGCCCUUUAACGAUGGCGUGUACUUCGCCUCUACCG |
| | protein | AGAAGAGCAACAUCAUCAGAGGCUGGAUCUUUGGCACCACACUGGACUCCAAGACACA |
| | | GUCUCUGCUGAUCGUGAACAAUGCCACCAACGUGGUCAUCAAGGUGUGCGAGUUCCA |
| | | GUUUUGUAAUGAUCCAUUCCUGGGCGUGUACUAUCACAAGAACAAUAAGAGCUGGAU |
| | | GGAGUCCGAGUUUCGCGUGUAUUCUAGCGCCAACAAUUGCACAUUUGAGUACGUGUC |
| | | CCAGCCCUUCCUGAUGGACCUGGAGGGCAAGCAGGGCAAUUUCAAGAACCUGAGGGA |
| | | GUUCGUGUUUAAGAAUAUCGAUGGCUACUUCAAGAUCUACUCUAAGCACACCCCAAU |
| | | CAACCUGGUGCGCGACCUGCCACAGGGCUUCAGCGCCCUGGAGCCACUGGUGGAUCU |
| | | GCCCAUCGGCAUCAACAUCACCCGGUUUCAGACACUGCUGGCCCUGCACAGAAGCUAC |
| | | CUGACACCAGGCGACUCCUCUAGCGGAUGGACCGCAGGAGCAGCAGCCUACUAUGUGG |
| | | GCUAUCUGCAGCCCAGGACCUUCCUGCUGAAGUACAACGAGAAUGGCACCAUCACAGA |
| | | CGCCGUGGAUUGCGCCCUGGAUCCCCUGAGCGAGACAAAGUGUACACUGAAGUCCUU |
| | | UACCGUGGAGAAGGGCAUCUAUCAGACAUCCAAUUUCAGGGUGCAGCCUACCGAGUC |
| | | UAUCGUGCGCUUUCCCAAUAUCACAAACCUGUGCCCUUUUGGCGAGGUGUUCAACGC |
| | | AACCAGGUUCGCAAGCGUGUACGCAUGGAAUAGGAAGCGCAUCUCUAACUGCGUGGC |
| | | CGACUAUAGCGUGCUGUACAACUCCGCCUCUUUCAGCACCUUUAAGUGCUAUGGCGU |
| | | GUCCCCCACAAAGCUGAAUGACCUGUGCUUUACCAACGUGUACGCCGAUUCUUUCGU |
| | | GAUCAGGGGCGACGAGGUGCGCCAGAUCGCACCAGGACAGACAGGCAAGAUCGCAGAC |
| | | UACAAUUAUAAGCUGCCUGACGAUUUCACCGGCUGCGUGAUCGCCUGGAACAGCAAC |
| | | AAUCUGGAUUCCAAAGUGGGCGGCAACUACAAUUAUCUGUACCGGCUGUUUAGAAAG |
| | | AGCAAUCUGAAGCCAUUCGAGAGGGACAUCUCUACAGAGAUCUACCAGGCAGGAAGCA |
| | | CCCCAUGCAAUGGAGUGGAGGGCUUUAACUGUUAUUUCCCUCUGCAGUCCUACGGCU |
| | | UCCAGCCAACCAACGGCGUGGGCUAUCAGCCCUACCGCGUGGUGGUGCUGAGCUUUG |
| | | AGCUGCUGCACGCACCUGCAACAGUGUGCGGACCAAAGAAGUCCACCAAUCUGGUGAA |
| | | GAACAAGUGCGUGAACUUCAACUUCAACGGCCUGACCGGAACAGGCGUGCUGACCGA |
| | | GUCCAACAAGAAGUUCCUGCCUUUUCAGCAGUUCGGCAGGGACAUCGCAGAUACCACA |
| | | GACGCCGUGCGCGACCCUCAGACCCUGGAGAUCCUGGAUAUCACACCAUGCUCUUUCG |
| | | GCGGCGUGAGCGUGAUCACACCAGGCACCAAUACAAGCAACCAGGUGGCCGUGCUGUA |
| | | UCAGGACGUGAAUUGUACCGAGGUGCCAGUGGCAAUCCACGCAGAUCAGCUGACCCC |
| | | UACAUGGCGGGUGUACAGCACCGGCUCCAACGUGUUCCAGACAAGAGCAGGAUGUCU |
| | | GAUCGGAGCAGAGCACGUGAACAAUUCCUAUGAGUGCGACAUCCCUAUCGGCGCCGGC |
| | | AUCUGUGCCUCUUACCAGACCCAGACAAACUCUCCAAGGAGAGCACGGAGCGUGGCAU |
| | | CCCAGUCUAUCAUCGCCUAUACCAUGUCCCUGGGCGCCGAGAAUUCUGUGGCCUACUC |
| | | UAACAAUAGCAUCGCCAUCCCUACCAACUUCACAAUCUCUGUGACCACAGAGAUCCUG |
| | | CCAGUGUCCAUGACCAAGACAUCUGUGGACUGCACAAUGUAUAUCUGUGGCGAUUCU |
| | | ACCGAGUGCAGCAACCUGCUGCUGCAGUACGGCAGCUUUUGUACCCAGCUGAAUAGA |
| | | GCCCUGACAGGCAUCGCCGUGGAGCAGGAUAAGAACACACAGGAGGUGUUCGCCCAG |
| | | GUGAAGCAGAUCUACAAGACCCCACCCAUCAAGGACUUUGGCGGCUUCAAUUUUUCCC |
| | | AGAUCCUGCCCGAUCCUUCCAAGCCCUCUAAGCGGAGCUUUAUCGAGGACCUGCUGU |
| | | UCAACAAGGUGACCCUGGCCGAUGCCGGCUUCAUCAAGCAGUAUGGCGAUUGCCUGG |
| | | GCGACAUCGCAGCACGGGACCUGAUCUGUGCCCAGAAGUUUAAUGGCCUGACCGUGC |
| | | UGCCUCCACUGCUGACAGAUGAGAUGAUCGCACAGUACACAAGCGCCCUGCUGGCAGG |
| | | AACCAUCACAUCCGGAUGGACCUUCGGCGCAGGAGCCGCCCUGCAGAUCCCCUUUGCC |
| | | AUGCAGAUGGCCUAUCGGUUCAACGGCAUCGGCGUGACCCAGAAUGUGCUGUACGAG |
| | | AACCAGAAGCUGAUCGCCAAUCAGUUUAACUCCGCCAUCGGCAAGAUCCAGGACAGCC |
| | | UGUCCUCUACAGCCUCCGCCCUGGGCAAGCUGCAGGAUGUGGUGAAUCAGAACGCCC |
| | | AGGCCCUGAAUACCCUGGUGAAGCAGCUGAGCUCCAACUUCGGCGCCAUCUCUAGCGU |
| | | GCUGAAUGAUAUCCUGAGCCGGCUGGACAAGGUGGAGGCAGAGGUGCAGAUCGACCG |
| | | GCUGAUCACAGGCAGACUGCAGUCUCUGCAGACCUAUGUGACACAGCAGCUGAUCAG |
| | | GGCAGCAGAGAUCAGGGCAAGCGCCAAUCUGGCAGCAACCAAGAUGUCCGAGUGCGU |
| | | GCUGGGCCAGUCUAAGAGAGUGGACUUUUGUGGCAAGGGCUAUCACCUGAUGUCCU |
| | | UCCCACAGUCUGCCCCUCACGGAGUGGUGUUUCUGCACGUGACCUACGUGCCAGCCCA |
| | | GGAGAAGAACUUCACCACAGCACCAGCAAUCUGCCACGAUGGCAAGGCACACUUUCCU |
| | | AGGGAGGGCGUGUUCGUGUCCAACGGCACCCACUGGUUUGUGACACAGCGCAAUUUC |
| | | UACGAGCCACAGAUCAUCACCACAGACAAUACCUUCGUGAGCGGCAACUGUGACGUGG |
| | | UCAUCGGCAUCGUGAACAAUACCGUGUAUGAUCCUCUGCAGCCAGAGCUGGACAGCU |
| | | UUAAGGAGGAGCUGGAUAAGUACUUCAAGAAUCACACCUCCCCCGACGUGGAUCUGG |
| | | GCGACAUCAGCGGCAUCAAUGCCUCCGUGGUGAACAUCCAGAAGGAGAUCGACAGGC |
| | | UGAACGAGGUGGCCAAGAAUCUGAACGAGAGCCUGAUCGAUCUGCAGGAGCUGGGCA |
| | | AGUAUGAGCAGUACAUCAAGUGGCCUUGGUACAUCUGGCUGGGCUUCAUCGCCGGCC |
| | | UGAUCGCCAUCGUGAUGGUGACCAUCAUGCUGUGCUGUAUGACAUCCUGCUGUUCU |
| | | UGCCUGAAGGGCUGCUGUAGCUGCGGCUCCUGUUGUAAAUUCGAUGAGGAUGAUUC |
| | | CGAGCCUGUGCUGAAGGGCGUGAAACUGCAUUAUACCUAAUAG |
| 5 | DNA ORF | ATGTTCGTATTCCTAGTGCTGCTGCCCCTGGTGAGCAGCCAGTGCGTGAACCTGACCACACG |
| | sequence | CACTCAGCTGCCACCAGCCTACACCAACTCATTCACCAGGGGCGTGTACTATCCTGACAAGG |
| | encoding a | ACATGGTTCCACGCCATCCACGTGAGCGGGACAAACGGCACCAAGCGGTTTGACAATCCCG |
| | modified | TGTTCAGGAGCAGCGTGCTGCACTCCACCCAGGATCTGTTCCTGCCCTTCTTCAGCAACGTG |
| | spike | TTCTGCCATTCAACGATGGCGTGTACTTCGCCAGCACCGAGAAAAGCAACATCATCCGGGGC |
| | protein | TGGATCTTCGGCACCACACTGGACAGCAAGACCCAGAGTCTGCTGATCGTGAACAACGCCA |
| | | CCAACGTGGTGATCAAGGTGTGTGAGTTCCAGTTCTGTAATGACCCCTTCCTGGGGGTCTAT |
| | | TACCACAAGAACAATAAAAGCTGGATGGAGAGCGAGTTTCGGGTGTACAGTAGTGCCAACA |
| | | ACTGTACATTCGAGTACGTGTCCCAGCCATTCCTGATGGACCTGGAGGGGAAGCAGGGGAA |
| | | CTTCAAGAACCTGCGGGAGTTCGTGTTTAAGAATATCGACGGATATTTTAAGATCTACTCCAA |
| | | GCACACCCCCATCAACCTGGTGCGGGATCTGCCCCAGGGCTTCAGCGCCCTGGAGCCCCTG |
| | | GTGGACCTGCCCATCGGGATCAACATCACCCGGTTCCAGACCCTGCTGGCCCTGCACCGGA |
| | | GTTACCTGACCCCAGGTGACTCCAGCAGCGGCTGGACAGCCGGGGCTGCAGCCTACTACGT |
| | | GGGCTACCTGCAGCCCCGGACCTTCCTCCTGAAGTACAATGAGAACGGCACCATCACCGAT |
| | | GCCGTGGATTGTGCTCTGGACCCCCTGTCTGAGACCAAGTGCACCCTGAAAAGCTTCACCG |
| | | TGGAGAAGGGGATCTACCAGACAAGCAACTTTCGGGTGCAGCCCACCGAGAGTATCGTGA |
| | | GGTTCCCCAACATCACCAACCTGTGTCCATTCGGGGAGGTGTTCAACGCCACCCGGTTCGCA |
| | | AGCGTGTACGCTTGGAACCGGAAGCGCATCAGCAATTGCGTGGCTGATTACAGCGTGCTGT |
| | | ACAACAGCGCCTCCTTCAGCACATTCAAGTGCTACGGCGTCTCCCCCACAAAGCTGAACGAT |
| | | CTGTGTTTCACCAACGTGTACGCAGACAGCTTTGTGATCCGGGGAGACGAGGTGCGGCAG |
| | | ATCGCACCCGGGCAGACCGGCAAGATTGCTGACTACAATTACAAGCTGCCTGATGACTTCAC |
| | | AGGCTGCGTGATTGCCTGGAACAGCAATAACCTGGACAGCAAGGTGGGAGGTAATTACAAT |
| | | TACCTCTACCGGCTGTTCAGGAAGTCCAATCTGAAGCCCTTCGAGAGGGACATCTCCACCGA |
| | | GATCTACCAGGCTGGGTCCACCCCATGCAATGGGGTGGAGGGCTTCAACTGCTACTTCCCCC |
| | | TGCAGTCCTACGGCTTCCAGCCCACCAATGGGGGGGCTACCAGCCCTACCGGGTGGTGGT |
| | | GCTGAGCTTCGAGCTCCTGCACGCCCCCGCCACTGTGTGCGGCCCCAAGAAGTCCACCAAC |
| | | CTGGTGAAGAACAAGTGCGTGAACTTCAACTTCAACGGGTTGACCGGGACTGGGGTGCTC |
| | | ACAGAGAGCAACAAGAAGTTCCTGCCCTTCCAGCAGTTCGGAAGGGACATCGCCGACACC |
| | | ACGGATGCCGTGAGGGATCCCCAGACCCTCGAGATCCTGGACATCACGCCATGCTCCTTCGG |
| | | GGGCGTGAGCGTGATCACTCCTGGCACAAACACCTCCAACCAGGTGGCTGTGCTGTACCAG |
| | | GGCGTGAACTGCACCGAGGTGCCTGTGGCCATCCATGCTGACCAGCTGACCCCCACCTGGC |
| | | GGGTCTACAGCACCGGCAGCAACGTGTTCCAGACCCGGGCCGGCTGCCTGATCGGGGCCG |
| | | AGCACGTGAACAACAGCTACGAGTGCGATATCCCCATCGGCGCCGGCATCTGCGCTAGCTAC |
| | | CAGACACAGACCAATAGCCCCCAATCCGCTCAATCTGTGGCTAGCCAGAGCATCATCGCCTA |
| | | CACTATGTCTCTGGGCGCAGAGAATAGTGTGGCTTACAGCAACAACAGCATCGCCATTCCCA |
| | | CTAATTTCACAATTAGTGTGACCACCGAGATCCTGCCTGTGAGCATGACCAAGACCAGCGTG |
| | | GACTGCACCATGTACATCTGCGGGGATAGCACCGAGTGCAGCAACCTGCTGCTGCAGTACG |
| | | GCAGCTTCTGCACACAGCTGAACCGGGCCCTGACCGGTATCGCTGTGGAGCAGGACAAGA |
| | | ACACCCAGGAGGTGTTTGCGCAGGTGAAGCAGATCTACAAGACCCCTCCTATCAAGGACTT |
| | | CGGGGGCTTTAACTTCAGCCAGATCCTGCCAGACCCCTCCAAGCCCTCCAAGAGGAGCTTC |
| | | ATCGAGGATCTCCTGTTCAACAAGGTGACCCTGGCCGATGCCGGCTTCATCAAGCAGTACGG |
| | | AGATTGCCTGGGCGACATCGCCGCCAGGGATCTCATCTGCGCCCAGAAGTTCAACGGCCTG |
| | | ACCGTGCTGCCCCCCCTGCTGACCGATGAGATGATCGCTCAGTACACCAGCGCCCTGCTGGC |
| | | TGGGACCATCACCAGCGGGTGGACCTTCGGGGCCGGCGCCGCCCTGCAGATCCCCTTCGCC |
| | | ATGCAGATGGCCTACCGGTTCAACGGTATTGGGGTGACCCAGAATGTGCTGTATGAGAACCA |
| | | GAAGCTGATCGCCAACCAGTTCAACTCTGCCATCGGCAAGATCCAGGACTCCCTGAGCAGC |
| | | ACTGCCAGCGCCCTGGGCAAGCTGCAGGACGTGGTGAACCAGAACGCCCAGGCCCTGAAT |
| | | ACCCTGGTGAAGCAGCTGTCCAGCAACTTCGGGGCCATCAGCAGCGTGCTGAACGACATCC |
| | | TGTCCCGCCTGGACCCACCAGAGGCAGAGGTGCAGATCGATCGGCTGATCACAGGTAGGCT |
| | | GCAGAGCCTGCAGACCTACGTGACCCAGCAGCTGATCCGCGCCGCTGAGATTAGGGCCAGC |
| | | GCCAACCTGGCCGCCACTAAGATGAGCGAGTGCGTGCTGGGCCAGAGCAAGCGGGTGGAC |
| | | TTCTGCGGCAAGGGGTATCACCTGATGAGCTTCCCTCAGTCCGCCCCCCACGGGGTGGTGTT |
| | | CCTGCACGTGACATACGTGCCCGCCCAGGAGAAGAACTTCACCACCGCCCCTGCCATCTGCC |
| | | ACGATGGCAAGGCCCACTTTCCCCGGGAGGGCGTGTTCGTGTCCAATGGCACACATTGGTT |
| | | CGTGACCCAGCGGAACTTCTACGAGCCACAGATCATTACCACTGACAATACCTTTGTCAGTG |
| | | GTAACTGTGACGTGGTGATCGGCATTGTGAACAACACAGTGTACGATCCTCTGCAGCCAGA |
| | | GCTGGACAGCTTCAAGGAGGAGCTGGACAAGTACTTCAAGAACCACACCAGCCCCGACGT |
| | | GGATCTTGGAGACATCTCTGGCATCAACGCCAGCGTGGTCAACATCCAGAAGGAGATTGAC |
| | | CGCCTGAACGAGGTTGCCAAGAACCTGAACGAGAGTCTGATCGACCTCCAGGAGCTTGGC |
| | | AAGTACGAGCAGTACATCAAGTGGCCATGGTACATCTGGCTGGGCTTCATCGCCGGCCTGAT |
| | | CGCCATCGTGATGGTGACCATCATTGCTGTGCTGCATGACCTCTTGTTGCAGCTGCCTGAAGG |
| | | GGTGCTGCAGCTGCGGTAGTTGCTGTAAGTTCGACGAGGACGACAGCGAGCCCGTGCTGA |
| | | AGGGCGTGAAGCTGCACTACACGTAATAG |
| 6 | mRNA | AGGAAAUUCCAUUUGGCUGCAGCUUCUGGAGGGAGCCGACAGGAGACGUGGGGAGA |
| | sequence | CGGCCACCAUGUUCGUAUUCCUAGUGCUGCUGCCCCUGGUGAGCAGCCAGUGCGUGA |
| | encoding a | ACCUGACCACACGCACUCAGCUGCCACCAGCCUACACCAACUCAUUCACCAGGGGCGUG |
| | modified | UACUAUCCUGACAAGGUGUUCAGGAGCAGCGUGCUGCACUCCACCCAGGAUCUGUUC |
| | spike | CUGCCCUUCUUCAGCAACGUGACAUGGUUCCACGCCAUCCACGUGAGCGGGACAAACG |
| | protein | GCACCAAGCGGUUUGACAAUCCCGUUCUGCCAUUCAACGAUGGCGUGUACUUCGCCA |
| | | GCACCGAGAAAAGCAACAUCAUCCGGGGCUGGAUCUUCGGCACCACACUGGACAGCAA |
| | | GACCCAGAGUCUGCUGAUCGUGAACAACGCCACCAACGUGGUGAUCAAGGUGUGUGA |
| | | GUUCCAGUUCUGUAAUGACCCCUUCCUGGGGGUCUAUUACCACAAGAACAAUAAAAG |
| | | CUGGAUGGAGAGCGAGUUUCGGGUGUACAGUAGUGCCAACAACUGUACAUUCGAGU |
| | | ACGUGUCCCAGCCAUUCCUGAUGGACCUGGAGGGGAAGCAGGGGAACUUCAAGAACC |
| | | UGCGGGAGUUCGUGUUUAAGAAUAUCGACGGAUAUUUUAAGAUCUACUCCAAGCAC |
| | | ACCCCCAUCAACCUGGUGCGGGAUCUGCCCCAGGGCUUCAGCGCCCUGGAGCCCCUGG |
| | | UGGACCUGCCCAUCGGGAUCAACAUCACCCGGUUCCAGACCCUGCUGGCCCUGCACCG |
| | | GAGUUACCUGACCCCAGGUGACUCCAGCAGCGGCUGGACAGCCGGGGCUGCAGCCUA |
| | | CUACGUGGGCUACCUGCAGCCCCGGACCUUCCUCCUGAAGUACAAUGAGAACGGCACC |
| | | AUCACCGAUGCCGUGGAUUGUGCUCUGGACCCCCUGUCUGAGACCAAGUGCACCCUG |
| | | AAAAGCUUCACCGUGGAGAAGGGGAUCUACCAGACAAGCAACUUUCGGGUGCAGCCC |
| | | ACCGAGAGUAUCGUGAGGUUCCCCAACAUCACCAACCUGUGUCCAUUCGGGGAGGUG |
| | | UUCAACGCCACCCGGUUCGCAAGCGUGUACGCUUGGAACCGGAAGCGCAUCAGCAAU |
| | | UGCGUGGCUGAUUACAGCGUGCUGUACAACAGCGCCUCCUUCAGCACAUUCAAGUGC |
| | | UACGGCGUCUCCCCCACAAAGCUGAACGAUCUGUGUUUCACCAACGUGUACGCAGACA |
| | | GCUUUGUGAUCCGGGGAGACGAGGUGCGGCAGAUCGCACCCGGGCAGACCGGCAAGA |
| | | UUGCUGACUACAAUUACAAGCUGCCUGAUGACUUCACAGGCUGCGUGAUUGCCUGGA |
| | | ACAGCAAUAACCUGGACAGCAAGGUGGGAGGUAAUUACAAUUACCUCUACCGGCUGU |
| | | UCAGGAAGUCCAAUCUGAAGCCCUUCGAGAGGGACAUCUCCACCGAGAUCUACCAGGC |
| | | UGGGUCCACCCCAUGCAAUGGGGUGGAGGGCUUCAACUGCUACUUCCCCCUGCAGUC |
| | | CUACGGCUUCCAGCCCACCAAUGGGGUGGGCUACCAGCCCUACCGGGUGGUGGUGCU |
| | | GAGCUUCGAGCUCCUGCACGCCCCCGCCACUGUGUGCGGCCCCAAGAAGUCCACCAAC |
| | | CUGGUGAAGAACAAGUGCGUGAACUUCAACUUCAACGGGUUGACCGGGACUGGGGU |
| | | GCUCACAGAGAGCAACAAGAAGUUCCUGCCCUUCCAGCAGUUCGGAAGGGACAUCGCC |
| | | GACACCACGGAUGCCGUGAGGGAUCCCCAGACCCUCGAGAUCCUGGACAUCACGCCAU |
| | | GCUCCUUCGGGGGCGUGAGCGUGAUCACUCCUGGCACAAACACCUCCAACCAGGUGG |
| | | CUGUGCUGUACCAGGGCGUGAACUGCACCGAGGUGCCUGUGGCCAUCCAUGCUGACC |
| | | AGCUGACCCCCACCUGGCGGGUCUACAGCACCGGCAGCAACGUGUUCCAGACCCGGGC |
| | | CGGCUGCCUGAUCGGGGCCGAGCACGUGAACAACAGCUACGAGUGCGAUAUCCCCAU |
| | | CGGCGCCGGCAUCUGCGCUAGCUACCAGACACAGACCAAUAGCCCCCAAUCCGCUCAA |
| | | UCUGUGGCUAGCCAGAGCAUCAUCGCCUACACUAUGUCUCUGGGCGCAGAGAAUAGU |
| | | GUGGCUUACAGCAACAACAGCAUCGCCAUUCCCACUAAUUUCACAAUUAGUGUGACC |
| | | ACCGAGAUCCUGCCUGUGAGCAUGACCAAGACCAGCGUGGACUGCACCAUGUACAUC |
| | | UGCGGGGAUAGCACCGAGUGCAGCAACCUGCUGCUGCAGUACGGCAGCUUCUGCACA |
| | | CAGCUGAACCGGGCCCUGACCGGUAUCGCUGUGGAGCAGGACAAGAACACCCAGGAG |
| | | GUGUUUGCGCAGGUGAAGCAGAUCUACAAGACCCCUCCUAUCAAGGACUUCGGGGGC |
| | | UUUAACUUCAGCCAGAUCCUGCCAGACCCCUCCAAGCCCUCCAAGAGGAGCUUCAUCG |
| | | AGGAUCUCCUGUUCAACAAGGUGACCCUGGCCGAUGCCGGCUUCAUCAAGCAGUACG |
| | | GAGAUUGCCUGGGCGACAUCGCCGCCAGGGAUCUCAUCUGCGCCCAGAAGUUCAACG |
| | | GCCUGACCGUGCUGCCCCCCCUGCUGACCGAUGAGAUGAUCGCUCAGUACACCAGCGC |
| | | CCUGCUGGCUGGGACCAUCACCAGCGGGUGGACCUUCGGGGCCGGCGCCGCCCUGCA |
| | | GAUCCCCUUCGCCAUGCAGAUGGCCUACCGGUUCAACGGUAUUGGGGUGACCCAGAA |
| | | UGUGCUGUAUGAGAACCAGAAGCUGAUCGCCAACCAGUUCAACUCUGCCAUCGGCAA |
| | | GAUCCAGGACUCCCUGAGCAGCACUGCCAGCGCCCUGGGCAAGCUGCAGGACGUGGU |
| | | GAACCAGAACGCCCAGGCCCUGAAUACCCUGGUGAAGCAGCUGUCCAGCAACUUCGGG |
| | | GCCAUCAGCAGCGUGCUGAACGACAUCCUGUCCCGCCUGGACCCACCAGAGGCAGAGG |
| | | UGCAGAUCGAUCGGCUGAUCACAGGUAGGCUGCAGAGCCUGCAGACCUACGUGACCC |
| | | AGCAGCUGAUCCGCGCCGCUGAGAUUAGGGCCAGCGCCAACCUGGCCGCCACUAAGAU |
| | | GAGCGAGUGCGUGCUGGGCCAGAGCAAGCGGGUGGACUUCUGCGGCAAGGGGUAUC |
| | | ACCUGAUGAGCUUCCCUCAGUCCGCCCCCCACGGGGUGGUGUUCCUGCACGUGACAU |
| | | ACGUGCCCGCCCAGGAGAAGAACUUCACCACCGCCCCUGCCAUCUGCCACGAUGGCAA |
| | | GGCCCACUUUCCCCGGGAGGGCGUGUUCGUGUCCAAUGGCACACAUUGGUUCGUGAC |
| | | CCAGCGGAACUUCUACGAGCCACAGAUCAUUACCACUGACAAUACCUUUGUCAGUGG |
| | | UAACUGUGACGUGGUGAUCGGCAUUGUGAACAACACAGUGUACGAUCCUCUGCAGCC |
| | | AGAGCUGGACAGCUUCAAGGAGGAGCUGGACAAGUACUUCAAGAACCACACCAGCCCC |
| | | GACGUGGAUCUUGGAGACAUCUCUGGCAUCAACGCCAGCGUGGUCAACAUCCAGAAG |
| | | GAGAUUGACCGCCUGAACGAGGUUGCCAAGAACCUGAACGAGAGUCUGAUCGACCUC |
| | | CAGGAGCUUGGCAAGUACGAGCAGUACAUCAAGUGGCCAUGGUACAUCUGGCUGGGC |
| | | UUCAUCGCCGGCCUGAUCGCCAUCGUGAUGGUGACCAUCAUGCUGUGCUGCAUGACC |
| | | UCUUGUUGCAGCUGCCUGAAGGGGUGCUGCAGCUGCGGUAGUUGCUGUAAGUUCGA |
| | | CGAGGACGACAGCGAGCCCGUGCUGAAGGGCGUGAAGCUGCACUACACGUAAUAGGC |
| | | UGCCUUCUGCGGGGCUUGCCUUCUGGCCAUGCCCUUCUUCUCUCCCUUGCACCUGUA |
| | | CCUCUUGGUCUUUGAAUAAAGCCUGAGUAGGAAGUJAAAAAAAAAAAAAAAAAAAAAA |
| | | AAAAAAAAAA |
| | | AAAAAAA |
| 7 | DNA | AGGAAATTCCATTTGGCTGCAGCTTCTGGAGGGAGCCGACAGGAGACGTGGGGAGACGG |
| | sequence | CCACCATGTTCGTATTCCTAGTGCTGCTGCCCCTGGTGAGCAGCCAGTGCGTGAACCTGACC |
| | encoding a | ACACGCACTCAGCTGCCACCAGCCTACACCAACTCATTCACCAGGGGCGTGTACTATCCTGA |
| | modified | CAAGGTGTTCAGGAGCAGCGTGCTGCACTCCACCCAGGATCTGTTCCTGCCCTTCTTCAGCA |
| | spike | ACGTGACATGGTTCCACGCCATCCACGTGAGCGGGACAAACGGCACCAAGCGGTTIGACA |
| | protein | ATCCCGTTCTGCCATTCAACGATGGCGTGTACTTCGCCAGCACCGAGAAAAGCAACATCATC |
| | | CGGGGCTGGATCTTCGGCACCACACTGGACAGCAAGACCCAGAGTCTGCTGATCGTGAACA |
| | | ACGCCACCAACGTGGTGATCAAGGTGTGTGAGTTCCAGTTCTGTAATGACCCCTTCCTGGGG |
| | | GTCTATTACCACAAGAACAATAAAAGCTGGATGGAGAGCGAGTTTCGGGTGTACAGTAGTG |
| | | CCAACAACTGTACATTCGAGTACGTGTCCCAGCCATTCCTGATGGACCTGGAGGGGAAGCA |
| | | GGGGAACTTCAAGAACCTGCGGGAGTTCGTGTTTAAGAATATCGACGGATATTTTAAGATCT |
| | | ACTCCAAGCACACCCCCATCAACCTGGTGCGGGATCTGCCCCAGGGCTTCAGCGCCCTGGA |
| | | GCCCCTGGTGGACCTGCCCATCGGGATCAACATCACCCGGTTCCAGACCCTGCTGGCCCTGC |
| | | ACCGGAGTTACCTGACCCCAGGTGACTCCAGCAGCGGCTGGACAGCCGGGGCTGCAGCCT |
| | | ACTACGTGGGCTACCTGCAGCCCCGGACCTTCCTCCTGAAGTACAATGAGAACGGCACCATC |
| | | ACCGATGCCGTGGATTGTGCTCTGGACCCCCTGTCTGAGACCAAGTGCACCCTGAAAAGCTT |
| | | CACCGTGGAGAAGGGGATCTACCAGACAAGCAACTTTCGGGTGCAGCCCACCGAGAGTAT |
| | | CGTGAGGTTCCCCAACATCACCAACCTGTGTCCATTCGGGGAGGTGTTCAACGCCACCCGG |
| | | TTCGCAAGCGTGTACGCTTGGAACCGGAAGCGCATCAGCAATTGCGTGGCTGATTACAGCG |
| | | TGCTGTACAACAGCGCCTCCTTCAGCACATTCAAGTGCTACGGCGTCTCCCCCACAAAGCTG |
| | | AACGATCTGTGTTTCACCAACGTGTACGCAGACAGCTTTGTGATCCGGGGAGACGAGGTGC |
| | | GGCAGATCGCACCCGGGCAGACCGGCAAGATTGCTGACTACAATTACAAGCTGCCTGATGA |
| | | CTTCACAGGCTGCGTGATTGCCTGGAACAGCAATAACCTGGACAGCAAGGTGGGAGGTAAT |
| | | TACAATTACCTCTACCGGCTGTTCAGGAAGTCCAATCTGAAGCCCTTCGAGAGGGACATCTC |
| | | CACCGAGATCTACCAGGCTGGGTCCACCCCATGCAATGGGGTGGAGGGCTTCAACTGCTAC |
| | | TTCCCCCTGCAGTCCTACGGCTTCCAGCCCACCAATGGGGGGGCTACCAGCCCTACCGGGT |
| | | GGTGGTGCTGAGCTTCGAGCTCCTGCACGCCCCCGCCACTGTGTGCGGCCCCAAGAAGTCC |
| | | ACCAACCTGGTGAAGAACAAGTGCGTGAACTTCAACTTCAACGGGTTGACCGGGACTGGG |
| | | GTGCTCACAGAGAGCAACAAGAAGTTCCTGCCCTTCCAGCAGTTCGGAAGGGACATCGCC |
| | | GACACCACGGATGCCGTGAGGGATCCCCAGACCCTCGAGATCCTGGACATCACGCCATGCT |
| | | CCTTCGGGGGCGTGAGCGTGATCACTCCTGGCACAAACACCTCCAACCAGGTGGCTGTGCT |
| | | GTACCAGGGCGTGAACTGCACCGAGGTGCCTGTGGCCATCCATGCTGACCAGCTGACCCCC |
| | | ACCTGGGGGGTCTACAGCACCGGCAGCAACGTGTTCCAGACCCGGGCCGGCTGCCTGATCG |
| | | GGGCCGAGCACGTGAACAACAGCTACGAGTGCGATATCCCCATCGGCGCCGGCATCTGCGC |
| | | TAGCTACCAGACACAGACCAATAGCCCCCAATCCGCTCAATCTGTGGCTAGCCAGAGCATCAT |
| | | CGCCTACACTATGTCTCTGGGCGCAGAGAATAGTGTGGCTTACAGCAACAACAGCATCGCCA |
| | | TTCCCACTAATTTCACAATTAGTGTGACCACCGAGATCCTGCCTGTGAGCATGACCAAGACCA |
| | | GCGTGGACTGCACCATGTACATCTGCGGGGATAGCACCGAGTGCAGCAACCTGCTGCTGCA |
| | | GTACGGCAGCTTCTGCACACAGCTGAACCGGGCCCTGACCGGTATCGCTGTGGAGCAGGAC |
| | | AAGAACACCCAGGAGGTGTTTGCGCAGGTGAAGCAGATTACAAGACCCCTCCTATCAAGG |
| | | ACTTCGGGGGCTTTAACTTCAGCCAGATCCTGCCAGACCCCTCCAAGCCCTCCAAGAGGAG |
| | | CTTCATCGAGGATCTCCTGTTCAACAAGGTGACCCTGGCCGATGCCGGCTTCATCAAGCAGT |
| | | ACGGAGATTGCCTGGGCGACATCGCCGCCAGGGATCTCATCTGCGCCCAGAAGTTCAACGG |
| | | CCTGACCGTGCTGCCCCCCCTGCTGACCGATGAGATGATCGCTCAGTACACCAGCGCCCTGC |
| | | TGGCTGGGACCATCACCAGCGGGTGGACCTTCGGGGCCGGCGCCGCCCTGCAGATCCCCTT |
| | | CGCCATGCAGATGGCCTACCGGTTCAACGGTATTGGGGTGACCCAGAATGTGCTGTATGAGA |
| | | ACCAGAAGCTGATCGCCAACCAGTTCAACTCTGCCATCGGCAAGATCCAGGACTCCCTGAG |
| | | CAGCACTGCCAGCGCCCTGGGCAAGCTGCAGGACGTGGTGAACCAGAACGCCCAGGCCCT |
| | | GAATACCCTGGTGAAGCAGCTGTCCAGCAACTTCGGGGCCATCAGCAGCGTGCTGAACGAC |
| | | ATCCTGTCCCGCCTGGACCCACCAGAGGCAGAGGTGCAGATCGATCGGCTGATCACAGGTA |
| | | GGCTGCAGAGCCTGCAGACCTACGTGACCCAGCAGCTGATCCGCGCCGCTGAGATTAGGGC |
| | | CAGCGCCAACCTGGCCGCCACTAAGATGAGCGAGTGCGTGCTGGGCCAGAGCAAGCGGGT |
| | | GGACTTCTGCGGCAAGGGGTATCACCTGATGAGCTTCCCTCAGTCCGCCCCCCACGGGGTG |
| | | GTGTTCCTGCACGTGACATACGTGCCCGCCCAGGAGAAGAACTTCACCACCGCCCCTGCCAT |
| | | CTGCCACGATGGCAAGGCCCACTTTCCCCGGGAGGGCGTGTTCGTGTCCAATGGCACACAT |
| | | TGGTTCGTGACCCAGCGGAACTTCTACGAGCCACAGATCATTACCACTGACAATACCTTTGTC |
| | | AGTGGTAACTGTGACGTGGTGATCGGCATTGTGAACAACACAGTGTACGATCCTCTGCAGCC |
| | | AGAGCTGGACAGCTTCAAGGAGGAGCTGGACAAGTACTTCAAGAACCACACCAGCCCCGA |
| | | CGTGGATCTTGGAGACATCTCTGGCATCAACGCCAGCGTGGTCAACATCCAGAAGGAGATT |
| | | GACCGCCTGAACGAGGTTGCCAAGAACCTGAACGAGAGTCTGATCGACCTCCAGGAGCTT |
| | | GGCAAGTACGAGCAGTACATCAAGTGGCCATGGTACATCTGGCTGGGCTTCATCGCCGGCCT |
| | | GATCGCCATCGTGATGGTGACCATCATGCTGTGCTGCATGACCTCTTGTTGCAGCTGCCTGAA |
| | | GGGGTGCTGCAGCTGCGGTAGTTGCTGTAAGTTCGACGAGGACGACAGCGAGCCCGTGCT |
| | | GAAGGGCGTGAAGCTGCACTACACGTAATAGGCTGCCTTCTGCGGGGCTTGCCTTCTGGCC |
| | | ATGCCCTTCTTCTCTCCCTTGCACCTGTACCTCTTGGTCTTTGAATAAAGCCTGAGTAGGAAG |
| | | TAAAAAAAAAAAAAAAAAAAAAAAAAAAAAAAAAAAAAAAAAAAAAAAAAAAAAAAAAAAAAAAAAAAA |
| | | AAAAAAAAAAAAAAAAA |
| 8 | DNA ORF | ATGTTCGTCTTCCTGGTGCTGCTGCCTCTGGTGTCTTCCCAGTGCGTGAATCTGACTACCAGG |
| | sequence | ACCCAGCTGCCCCCTGCCTATACCAATTCCTTCACACGGGGCGTGTACTATCCCGACAAGGTG |
| | encoding a | TTTAGAAGCTCCGTGCTGCACTCTACACAGGATCTGTTTCTGCCTTTCTTTAGCAACGTGACC |
| | native | TGGTTCCACGCCATCCACGTGAGCGGCACCAATGGCACAAAGCGGTTCGACAATCCAGTGC |
| | spike | TGCCCTTTAACGATGGCGTGTACTTCGCCTCTACCGAGAAGAGCAACATCATCAGAGGCTGG |
| | protein | ATCTTTGGCACCACACTGGACTCCAAGACACAGTCTCTGCTGATCGTGAACAATGCCACCAA |
| | | CGTGGTCATCAAGGTGTGCGAGTTCCAGTTTTGTAATGATCCATTCCTGGGCGTGTACTATCA |
| | | CAAGAACAATAAGAGCTGGATGGAGTCCGAGTTTCGCGTGTATTCTAGCGCCAACAATTGCA |
| | | CATTTGAGTACGTGTCCCAGCCCTTCCTGATGGACCTGGAGGGCAAGCAGGGCAATTTCAA |
| | | GAACCTGAGGGAGTTCGTGTTTAAGAATATCGATGGCTACTTCAAGATCTACTCTAAGCACA |
| | | CCCCAATCAACCTGGTGCGCGACCTGCCACAGGGCTTCAGCGCCCTGGAGCCACTGGTGGA |
| | | TCTGCCCATCGGCATCAACATCACCCGGTTTCAGACACTGCTGGCCCTGCACAGAAGCTACC |
| | | TGACACCAGGCGACTCCTCTAGCGGATGGACCGCAGGAGCAGCAGCCTACTATGTGGGCTA |
| | | TCTGCAGCCCAGGACCTTCCTGCTGAAGTACAACGAGAATGGCACCATCACAGACGCCGTG |
| | | GATTGCGCCCTGGATCCCCTGAGCGAGACAAAGTGTACACTGAAGTCCTTTACCGTGGAGA |
| | | AGGGCATCTATCAGACATCCAATTTCAGGGTGCAGCCTACCGAGTCTATCGTGCGCTTTCCCA |
| | | ATATCACAAACCTGTGCCCTTTTGGCGAGGTGTTCAACGCAACCAGGTTCGCAAGCGTGTAC |
| | | GCATGGAATAGGAAGCGCATCTCTAACTGCGTGGCCGACTATAGCGTGCTGTACAACTCCGC |
| | | CTCTTTCAGCACCTTTAAGTGCTATGGCGTGTCCCCCACAAAGCTGAATGACCTGTGCTTTAC |
| | | CAACGTGTACGCCGATTCTTTCGTGATCAGGGGCGACGAGGTGCGCCAGATCGCACCAGGA |
| | | CAGACAGGCAAGATCGCAGACTACAATTATAAGCTGCCTGACGATTTCACCGGCTGCGTGAT |
| | | CGCCTGGAACAGCAACAATCTGGATTCCAAAGTGGGGGGCAACTACAATTATCTGTACCGGC |
| | | TGTTTAGAAAGAGCAATCTGAAGCCATTCGAGAGGGACATCTCTACAGAGATCTACCAGGC |
| | | AGGAAGCACCCCATGCAATGGAGTGGAGGGCTTTAACTGTTATTTCCCTCTGCAGTCCTACG |
| | | GCTTCCAGCCAACCAACGGCGTGGGCTATCAGCCCTACCGCGTGGTGGTGCTGAGCTTTGA |
| | | GCTGCTGCACGCACCTGCAACAGTGTGCGGACCAAAGAAGTCCACCAATCTGGTGAAGAAC |
| | | AAGTGCGTGAACTTCAACTTCAACGGCCTGACCGGAACAGGCGTGCTGACCGAGTCCAACA |
| | | AGAAGTTCCTGCCTTTTCAGCAGTTCGGCAGGGACATCGCAGATACCACAGACGCCGTGCG |
| | | CGACCCTCAGACCCTGGAGATCCTGGATATCACACCATGCTCTTTCGGCGGCGTGAGCGTGA |
| | | TCACACCAGGCACCAATACAAGCAACCAGGTGGCCGTGCTGTATCAGGACGTGAATTGTACC |
| | | GAGGTGCCAGTGGCAATCCACGCAGATCAGCTGACCCCTACATGGGGGGTGTACAGCACCG |
| | | GCTCCAACGTGTTCCAGACAAGAGCAGGATGTCTGATCGGAGCAGAGCACGTGAACAATTC |
| | | CTATGAGTGCGACATCCCTATCGGCGCCGGCATCTGTGCCTCTTACCAGACCCAGACAAACTC |
| | | TCCAAGGAGAGCACGGAGCGTGGCATCCCAGTCTATCATCGCCTATACCATGTCCCTGGGCG |
| | | CCGAGAATTCTGTGGCCTACTCTAACAATAGCATCGCCATCCCTACCAACTTCACAATCTCTGT |
| | | GACCACAGAGATCCTGCCAGTGTCCATGACCAAGACATCTGTGGACTGCACAATGTATATCT |
| | | GTGGCGATTCTACCGAGTGCAGCAACCTGCTGCTGCAGTACGGCAGCTTTTGTACCCAGCTG |
| | | AATAGAGCCCTGACAGGCATCGCCGTGGAGCAGGATAAGAACACACAGGAGGTGTTCGCC |
| | | CAGGTGAAGCAGATCTACAAGACCCCACCCATCAAGGACTTTGGCGGCTTCAATTTTTCCCA |
| | | GATCCTGCCCGATCCTTCCAAGCCCTCTAAGCGGAGCTTTATCGAGGACCTGCTGTTCAACA |
| | | AGGTGACCCTGGCCGATGCCGGCTTCATCAAGCAGTATGGCGATTGCCTGGGCGACATCGC |
| | | AGCACGGGACCTGATCTGTGCCCAGAAGTTTAATGGCCTGACCGTGCTGCCTCCACTGCTGA |
| | | CAGATGAGATGATCGCACAGTACACAAGCGCCCTGCTGGCAGGAACCATCACATCCGGATG |
| | | GACCTTCGGCGCAGGAGCCGCCCTGCAGATCCCCTTTGCCATGCAGATGGCCTATCGGTTCA |
| | | ACGGCATCGGCGTGACCCAGAATGTGCTGTACGAGAACCAGAAGCTGATCGCCAATCAGTT |
| | | TAACTCCGCCATCGGCAAGATCCAGGACAGCCTGTCCTCTACAGCCTCCGCCCTGGGCAAGC |
| | | TGCAGGATGTGGTGAATCAGAACGCCCAGGCCCTGAATACCCTGGTGAAGCAGCTGAGCTC |
| | | CAACTTCGGCGCCATCTCTAGCGTGCTGAATGATATCCTGAGCCGGCTGGACAAGGTGGAG |
| | | GCAGAGGTGCAGATCGACCGGCTGATCACAGGCAGACTGCAGTCTCTGCAGACCTATGTGA |
| | | CACAGCAGCTGATCAGGGCAGCAGAGATCAGGGCAAGCGCCAATCTGGCAGCAACCAAGA |
| | | TGTCCGAGTGCGTGCTGGGCCAGTCTAAGAGAGTGGACTTTTGTGGCAAGGGCTATCACCT |
| | | GATGTCCTTCCCACAGTCTGCCCCTCACGGAGTGGTGTTTCTGCACGTGACCTACGTGCCAG |
| | | CCCAGGAGAAGAACTTCACCACAGCACCAGCAATCTGCCACGATGGCAAGGCACACTTTCC |
| | | TAGGGAGGGCGTGTTCGTGTCCAACGGCACCCACTGGTTTGTGACACAGCGCAATTTCTAC |
| | | GAGCCACAGATCATCACCACAGACAATACCTTCGTGAGCGGCAACTGTGACGTGGTCATCG |
| | | GCATCGTGAACAATACCGTGTATGATCCTCTGCAGCCAGAGCTGGACAGCTTTAAGGAGGA |
| | | GCTGGATAAGTACTTCAAGAATCACACCTCCCCCGACGTGGATCTGGGCGACATCAGCGGC |
| | | ATCAATGCCTCCGTGGTGAACATCCAGAAGGAGATCGACAGGCTGAACGAGGTGGCCAAG |
| | | AATCTGAACGAGAGCCTGATCGATCTGCAGGAGCTGGGCAAGTATGAGCAGTACATCAAGT |
| | | GGCCTTGGTACATCTGGCTGGGCTTCATCGCCGGCCTGATCGCCATCGTGATGGTGACCATC |
| | | ATGCTGTGCTGTATGACATCCTGCTGTTCTTGCCTGAAGGGCTGCTGTAGCTGCGGCTCCTGT |
| | | TGTAAATTCGATGAGGATGATTCCGAGCCTGTGCTGAAGGGCGTGAAACTGCATTATACCTA |
| | | ATAG |
| 9 | mRNA | AGGAAAUUCCAUUUGGCUGCAGCUUCUGGAGGGAGCCGACAGGAGACGUGGGGAGA |
| | sequence | CGGCCACCAUGUUCGUCUUCCUGGUGCUGCUGCCUCUGGUGUCUUCCCAGUGCGUGA |
| | encoding a | AUCUGACUACCAGGACCCAGCUGCCCCCUGCCUAUACCAAUUCCUUCACACGGGGCGU |
| | native | GUACUAUCCCGACAAGGUGUUUAGAAGCUCCGUGCUGCACUCUACACAGGAUCUGUU |
| | spike | UCUGCCUUUCUUUAGCAACGUGACCUGGUUCCACGCCAUCCACGUGAGCGGCACCAA |
| | protein | UGGCACAAAGCGGUUCGACAAUCCAGUGCUGCCCUUUAACGAUGGCGUGUACUUCGC |
| | | CUCUACCGAGAAGAGCAACAUCAUCAGAGGCUGGAUCUUUGGCACCACACUGGACUC |
| | | CAAGACACAGUCUCUGCUGAUCGUGAACAAUGCCACCAACGUGGUCAUCAAGGUGUG |
| | | CGAGUUCCAGUUUUGUAAUGAUCCAUUCCUGGGCGUGUACUAUCACAAGAACAAUAA |
| | | GAGCUGGAUGGAGUCCGAGUUUCGCGUGUAUUCUAGCGCCAACAAUUGCACAUUUG |
| | | AGUACGUGUCCCAGCCCUUCCUGAUGGACCUGGAGGGCAAGCAGGGCAAUUUCAAGA |
| | | ACCUGAGGGAGUUCGUGUUUAAGAAUAUCGAUGGCUACUUCAAGAUCUACUCUAAG |
| | | CACACCCCAAUCAACCUGGUGCGCGACCUGCCACAGGGCUUCAGCGCCCUGGAGCCAC |
| | | UGGUGGAUCUGCCCAUCGGCAUCAACAUCACCCGGUUUCAGACACUGCUGGCCCUGC |
| | | ACAGAAGCUACCUGACACCAGGCGACUCCUCUAGCGGAUGGACCGCAGGAGCAGCAGC |
| | | CUACUAUGUGGGCUAUCUGCAGCCCAGGACCUUCCUGCUGAAGUACAACGAGAAUGG |
| | | CACCAUCACAGACGCCGUGGAUUGCGCCCUGGAUCCCCUGAGCGAGACAAAGUGUACA |
| | | CUGAAGUCCUUUACCGUGGAGAAGGGCAUCUAUCAGACAUCCAAUUUCAGGGUGCAG |
| | | CCUACCGAGUCUAUCGUGCGCUUUCCCAAUAUCACAAACCUGUGCCCUUUUGGCGAG |
| | | GUGUUCAACGCAACCAGGUUCGCAAGCGUGUACGCAUGGAAUAGGAAGCGCAUCUCU |
| | | AACUGCGUGGCCGACUAUAGCGUGCUGUACAACUCCGCCUCUUUCAGCACCUUUAAG |
| | | UGCUAUGGCGUGUCCCCCACAAAGCUGAAUGACCUGUGCUUUACCAACGUGUACGCC |
| | | GAUUCUUUCGUGAUCAGGGGCGACGAGGUGCGCCAGAUCGCACCAGGACAGACAGGC |
| | | AAGAUCGCAGACUACAAUUAUAAGCUGCCUGACGAUUUCACCGGCUGCGUGAUCGCC |
| | | UGGAACAGCAACAAUCUGGAUUCCAAAGUGGGCGGCAACUACAAUUAUCUGUACCGG |
| | | CUGUUUAGAAAGAGCAAUCUGAAGCCAUUCGAGAGGGACAUCUCUACAGAGAUCUAC |
| | | CAGGCAGGAAGCACCCCAUGCAAUGGAGUGGAGGGCUUUAACUGUUAUUUCCCUCUG |
| | | CAGUCCUACGGCUUCCAGCCAACCAACGGCGUGGGCUAUCAGCCCUACCGCGUGGUG |
| | | GUGCUGAGCUUUGAGCUGCUGCACGCACCUGCAACAGUGUGCGGACCAAAGAAGUCC |
| | | ACCAAUCUGGUGAAGAACAAGUGCGUGAACUUCAACUUCAACGGCCUGACCGGAACA |
| | | GGCGUGCUGACCGAGUCCAACAAGAAGUUCCUGCCUUUUCAGCAGUUCGGCAGGGAC |
| | | AUCGCAGAUACCACAGACGCCGUGCGCGACCCUCAGACCCUGGAGAUCCUGGAUAUCA |
| | | CACCAUGCUCUUUCGGCGGCGUGAGCGUGAUCACACCAGGCACCAAUACAAGCAACCA |
| | | GGUGGCCGUGCUGUAUCAGGACGUGAAUUGUACCGAGGUGCCAGUGGCAAUCCACG |
| | | CAGAUCAGCUGACCCCUACAUGGCGGGUGUACAGCACCGGCUCCAACGUGUUCCAGAC |
| | | AAGAGCAGGAUGUCUGAUCGGAGCAGAGCACGUGAACAAUUCCUAUGAGUGCGACAU |
| | | CCCUAUCGGCGCCGGCAUCUGUGCCUCUUACCAGACCCAGACAAACUCUCCAAGGAGA |
| | | GCACGGAGCGUGGCAUCCCAGUCUAUCAUCGCCUAUACCAUGUCCCUGGGCGCCGAG |
| | | AAUUCUGUGGCCUACUCUAACAAUAGCAUCGCCAUCCCUACCAACUUCACAAUCUCUG |
| | | UGACCACAGAGAUCCUGCCAGUGUCCAUGACCAAGACAUCUGUGGACUGCACAAUGU |
| | | AUAUCUGUGGCGAUUCUACCGAGUGCAGCAACCUGCUGCUGCAGUACGGCAGCUUUU |
| | | GUACCCAGCUGAAUAGAGCCCUGACAGGCAUCGCCGUGGAGCAGGAUAAGAACACAC |
| | | AGGAGGUGUUCGCCCAGGUGAAGCAGAUCUACAAGACCCCACCCAUCAAGGACUUUG |
| | | GCGGCUUCAAUUUUUCCCAGAUCCUGCCCGAUCCUUCCAAGCCCUCUAAGCGGAGCU |
| | | UUAUCGAGGACCUGCUGUUCAACAAGGUGACCCUGGCCGAUGCCGGCUUCAUCAAGC |
| | | AGUAUGGCGAUUGCCUGGGCGACAUCGCAGCACGGGACCUGAUCUGUGCCCAGAAGU |
| | | UUAAUGGCCUGACCGUGCUGCCUCCACUGCUGACAGAUGAGAUGAUCGCACAGUACA |
| | | CAAGCGCCCUGCUGGCAGGAACCAUCACAUCCGGAUGGACCUUCGGCGCAGGAGCCGC |
| | | CCUGCAGAUCCCCUUUGCCAUGCAGAUGGCCUAUCGGUUCAACGGCAUCGGCGUGAC |
| | | CCAGAAUGUGCUGUACGAGAACCAGAAGCUGAUCGCCAAUCAGUUUAACUCCGCCAU |
| | | CGGCAAGAUCCAGGACAGCCUGUCCUCUACAGCCUCCGCCCUGGGCAAGCUGCAGGAU |
| | | GUGGUGAAUCAGAACGCCCAGGCCCUGAAUACCCUGGUGAAGCAGCUGAGCUCCAAC |
| | | UUCGGCGCCAUCUCUAGCGUGCUGAAUGAUAUCCUGAGCCGGCUGGACAAGGUGGA |
| | | GGCAGAGGUGCAGAUCGACCGGCUGAUCACAGGCAGACUGCAGUCUCUGCAGACCUA |
| | | UGUGACACAGCAGCUGAUCAGGGCAGCAGAGAUCAGGGCAAGCGCCAAUCUGGCAGC |
| | | AACCAAGAUGUCCGAGUGCGUGCUGGGCCAGUCUAAGAGAGUGGACUUUUGUGGCA |
| | | AGGGCUAUCACCUGAUGUCCUUCCCACAGUCUGCCCCUCACGGAGUGGUGUUUCUGC |
| | | ACGUGACCUACGUGCCAGCCCAGGAGAAGAACUUCACCACAGCACCAGCAAUCUGCCA |
| | | CGAUGGCAAGGCACACUUUCCUAGGGAGGGCGUGUUCGUGUCCAACGGCACCCACUG |
| | | GUUUGUGACACAGCGCAAUUUCUACGAGCCACAGAUCAUCACCACAGACAAUACCUUC |
| | | GUGAGCGGCAACUGUGACGUGGUCAUCGGCAUCGUGAACAAUACCGUGUAUGAUCC |
| | | UCUGCAGCCAGAGCUGGACAGCUUUAAGGAGGAGCUGGAUAAGUACUUCAAGAAUCA |
| | | CACCUCCCCCGACGUGGAUCUGGGCGACAUCAGCGGCAUCAAUGCCUCCGUGGUGAAC |
| | | AUCCAGAAGGAGAUCGACAGGCUGAACGAGGUGGCCAAGAAUCUGAACGAGAGCCUG |
| | | AUCGAUCUGCAGGAGCUGGGCAAGUAUGAGCAGUACAUCAAGUGGCCUUGGUACAU |
| | | CUGGCUGGGCUUCAUCGCCGGCCUGAUCGCCAUCGUGAUGGUGACCAUCAUGCUGUG |
| | | CUGUAUGACAUCCUGCUGUUCUUGCCUGAAGGGCUGCUGUAGCUGCGGCUCCUGUU |
| | | GUAAAUUCGAUGAGGAUGAUUCCGAGCCUGUGCUGAAGGGCGUGAAACUGCAUUAU |
| | | ACCUAAUAGGCUGCCUUCUGCGGGGCUUGCCUUCUGGCCAUGCCCUUCUUCUCUCCC |
| | | UUGCACCUGUACCUCUUGGUCUUUGAAUAAAGCCUGAGUAGGAAGUAA |
| | | AAAAAAAA |
| | | AAAAAA |
| 10 | DNA | AGGAAATTCCATTTGGCTGCAGCTTCTGGAGGGAGCCGACAGGAGACGTGGGGAGACGG |
| | sequence | CCACCATGTTCGTCTTCCTGGTGCTGCTGCCTCTGGTGTCTTCCCAGTGCGTGAATCTGACTA |
| | encoding a | AGGTGTTTAGAAGCTCCGTGCTGCACTCTACACAGGATCTGTTTCTGCCTTTCTTTAGCAACG |
| | native | CCAGGACCCAGCTGCCCCCTGCCTATACCAATTCCTTCACACGGGGCGTGTACTATCCCGACA |
| | spike | TGACCTGGTTCCACGCCATCCACGTGAGCGGCACCAATGGCACAAAGCGGTTCGACAATCC |
| | protein | AGTGCTGCCCTTTAACGATGGCGTGTACTTCGCCTCTACCGAGAAGAGCAACATCATCAGAG |
| | | GCTGGATCTTTGGCACCACACTGGACTCCAAGACACAGTCTCTGCTGATCGTGAACAATGCC |
| | | ACCAACGTGGTCATCAAGGTGTGCGAGTTCCAGTTTTGTAATGATCCATTCCTGGGCGTGTA |
| | | CTATCACAAGAACAATAAGAGCTGGATGGAGTCCGAGTTTCGCGTGTATTCTAGCGCCAACA |
| | | ATTGCACATTTGAGTACGTGTCCCAGCCCTTCCTGATGGACCTGGAGGGCAAGCAGGGCAA |
| | | TTTCAAGAACCTGAGGGAGTTCGTGTTTAAGAATATCGATGGCTACTTCAAGATCTACTCTAA |
| | | GCACACCCCAATCAACCTGGTGCGCGACCTGCCACAGGGCTTCAGCGCCCTGGAGCCACTG |
| | | GTGGATCTGCCCATCGGCATCAACATCACCCGGTTTCAGACACTGCTGGCCCTGCACAGAAG |
| | | CTACCTGACACCAGGCGACTCCTCTAGCGGATGGACCGCAGGAGCAGCAGCCTACTATGTG |
| | | GGCTATCTGCAGCCCAGGACCTTCCTGCTGAAGTACAACGAGAATGGCACCATCACAGACG |
| | | CCGTGGATTGCGCCCTGGATCCCCTGAGCGAGACAAAGTGTACACTGAAGTCCTTTACCGTG |
| | | GAGAAGGGCATCTATCAGACATCCAATTTCAGGGTGCAGCCTACCGAGTCTATCGTGCGCTT |
| | | TCCCAATATCACAAACCTGTGCCCTTTTGGCGAGGTGTTCAACGCAACCAGGTTCGCAAGCG |
| | | TGTACGCATGGAATAGGAAGCGCATCTCTAACTGCGTGGCCGACTATAGCGTGCTGTACAAC |
| | | TCCGCCTCTTTCAGCACCTTTAAGTGCTATGGCGTGTCCCCCACAAAGCTGAATGACCTGTGC |
| | | TTTACCAACGTGTACGCCGATTCTTTCGTGATCAGGGGCGACGAGGTGCGCCAGATCGCACC |
| | | AGGACAGACAGGCAAGATCGCAGACTACAATTATAAGCTGCCTGACGATTTCACCGGCTGC |
| | | GTGATCGCCTGGAACAGCAACAATCTGGATTCCAAAGTGGGGGGCAACTACAATTATCTGTA |
| | | CCGGCTGTTTAGAAAGAGCAATCTGAAGCCATTCGAGAGGGACATCTCTACAGAGATCTACC |
| | | AGGCAGGAAGCACCCCATGCAATGGAGTGGAGGGCTTTAACTGTTATTTCCCTCTGCAGTCC |
| | | TACGGCTTCCAGCCAACCAACGGCGTGGGCTATCAGCCCTACCGCGTGGTGGTGCTGAGCT |
| | | TTGAGCTGCTGCACGCACCTGCAACAGTGTGCGGACCAAAGAAGTCCACCAATCTGGTGAA |
| | | GAACAAGTGCGTGAACTTCAACTTCAACGGCCTGACCGGAACAGGCGTGCTGACCGAGTC |
| | | CAACAAGAAGTTCCTGCCTTTTCAGCAGTTCGGCAGGGACATCGCAGATACCACAGACGCC |
| | | GTGCGCGACCCTCAGACCCTGGAGATCCTGGATATCACACCATGCTCTTTCGGCGGCGTGAG |
| | | CGTGATCACACCAGGCACCAATACAAGCAACCAGGTGGCCGTGCTGTATCAGGACGTGAAT |
| | | TGTACCGAGGTGCCAGTGGCAATCCACGCAGATCAGCTGACCCCTACATGGCGGGTGTACA |
| | | GCACCGGCTCCAACGTGTTCCAGACAAGAGCAGGATGTCTGATCGGAGCAGAGCACGTGA |
| | | ACAATTCCTATGAGTGCGACATCCCTATCGGCGCCGGCATCTGTGCCTCTTACCAGACCCAGA |
| | | CAAACTCTCCAAGGAGAGCACGGAGCGTGGCATCCCAGTCTATCATCGCCTATACCATGTCC |
| | | CTGGGCGCCGAGAATTCTGTGGCCTACTCTAACAATAGCATCGCCATCCCTACCAACTTCACA |
| | | ATCTCTGTGACCACAGAGATCCTGCCAGTGTCCATGACCAAGACATCTGTGGACTGCACAAT |
| | | GTATATCTGTGGCGATTCTACCGAGTGCAGCAACCTGCTGCTGCAGTACGGCAGCTTTTGTAC |
| | | CCAGCTGAATAGAGCCCTGACAGGCATCGCCGTGGAGCAGGATAAGAACACACAGGAGGT |
| | | GTTCGCCCAGGTGAAGCAGATCTACAAGACCCCACCCATCAAGGACTTTGGCGGCTTCAATT |
| | | TTTCCCAGATCCTGCCCGATCCTTCCAAGCCCTCTAAGCGGAGCTTTATCGAGGACCTGCTGT |
| | | TCAACAAGGTGACCCTGGCCGATGCCGGCTTCATCAAGCAGTATGGCGATTGCCTGGGCGA |
| | | CATCGCAGCACGGGACCTGATCTGTGCCCAGAAGTTTAATGGCCTGACCGTGCTGCCTCCAC |
| | | TGCTGACAGATGAGATGATCGCACAGTACACAAGCGCCCTGCTGGCAGGAACCATCACATCC |
| | | GGATGGACCTTCGGCGCAGGAGCCGCCCTGCAGATCCCCTTTGCCATGCAGATGGCCTATC |
| | | GGTTCAACGGCATCGGCGTGACCCAGAATGTGCTGTACGAGAACCAGAAGCTGATCGCCAA |
| | | TCAGTTTAACTCCGCCATCGGCAAGATCCAGGACAGCCTGTCCTCTACAGCCTCCGCCCTGG |
| | | GCAAGCTGCAGGATGTGGTGAATCAGAACGCCCAGGCCCTGAATACCCTGGTGAAGCAGCT |
| | | GAGCTCCAACTTCGGCGCCATCTCTAGCGTGCTGAATGATATCCTGAGCCGGCTGGACAAGG |
| | | TGGAGGCAGAGGTGCAGATCGACCGGCTGATCACAGGCAGACTGCAGTCTCTGCAGACCT |
| | | ATGTGACACAGCAGCTGATCAGGGCAGCAGAGATCAGGGCAAGCGCCAATCTGGCAGCAA |
| | | CCAAGATGTCCGAGTGCGTGCTGGGCCAGTCTAAGAGAGTGGACTTTTGTGGCAAGGGCTA |
| | | TCACCTGATGTCCTTCCCACAGTCTGCCCCTCACGGAGTGGTGTTTCTGCACGTGACCTACGT |
| | | GCCAGCCCAGGAGAAGAACTTCACCACAGCACCAGCAATCTGCCACGATGGCAAGGCACA |
| | | CTTTCCTAGGGAGGGCGTGTTCGTGTCCAACGGCACCCACTGGTTTGTGACACAGCGCAAT |
| | | TTCTACGAGCCACAGATCATCACCACAGACAATACCTTCGTGAGCGGCAACTGTGACGTGGT |
| | | CATCGGCATCGTGAACAATACCGTGTATGATCCTCTGCAGCCAGAGCTGGACAGCTTTAAGG |
| | | AGGAGCTGGATAAGTACTTCAAGAATCACACCTCCCCCGACGTGGATCTGGGCGACATCAG |
| | | CGGCATCAATGCCTCCGTGGTGAACATCCAGAAGGAGATCGACAGGCTGAACGAGGTGGC |
| | | CAAGAATCTGAACGAGAGCCTGATCGATCTGCAGGAGCTGGGCAAGTATGAGCAGTACATC |
| | | AAGTGGCCTTGGTACATCTGGCTGGGCTTCATCGCCGGCCTGATCGCCATCGTGATGGTGAC |
| | | CATCATGCTGTGCTGTATGACATCCTGCTGTTCTTGCCTGAAGGGCTGCTGTAGCTGCGGCTC |
| | | CTGTTGTAAATTCGATGAGGATGATTCCGAGCCTGTGCTGAAGGGCGTGAAACTGCATTATA |
| | | CCTAATAGGCTGCCTTCTGCGGGGCTTGCCTTCTGGCCATGCCCTTCTTCTCTCCCTTGCACC |
| | | TGTACCTCTTGGTCTTTGAATAAAGCCTGAGTAGGAAGTAAAAAAAAAAAAAAAAAAAAAA |
| | | AAAAAAAAAAAAAAAAAAAAAAAAAAAAAAAAAAAAAAAAAAAAAAAAAAAAA |
| 11 | Amino | MFVFLVLLPLVSSQCVNLTTRTQLPPAYTNSFTRGVYYPDKVFRSSVLHSTQDLFLPFFSNVTWF |
| | acid | HAIHVSGTNGTKRFDNPVLPENDGVYFASTEKSNIIRGWIFGTTLDSKTQSLLIVNNATNVVIKV |
| | sequence | CEFQFCNDPFLGVYYHKNNKSWMESEFRVYSSANNCTFEYVSQPFLMQLEGKQGNFKNLREF |
| | of a native | VFKNIDGYFKIYSKHTPINIVRDLPQGFSALEPLVDLPIGINITRFQTLLALHRSYLTPGDSSSGWT |
| | spike | AGAAAYYVGYLQPRTFLLKYNENGTITDAVDCALDPLSETKCTLKSFTVEKGIYQTSNFRVQPTES |
| | protein | IVRFPNITNLCPFGEVENATRFASVYAWNRKRISNCVADYSVLYNSASFSTFKCYGVSPTKLNDLC |
| | | FTNVYADSFVIRGDEVRQIAPGQTGKIADYNYKLPDDFTGCVIAWNSNNLDSKVGGNYNYLYR |
| | | LFRKSNLKPFERDISTEIYQAGSTPCNGVEGENCYFPLQSYGFQPTNGVGYQPYRVVVLSFELLH |
| | | APATVCGPKKSTNLVKNKCVNFNFNGLTGTGVLTESNKKFLPFQQFGRDIADTTDAVRDPQTLEI |
| | | LDITPCSFGGVSVITPGTNTSNQVAVLYQDVNCTEVPVAIHADQLTPTWRVYSTGSNVFQTRAG |
| | | CLIGAEHVNNSYECDIPIGAGICASYQTQTNSPRRARSVASQSIIAYTMSLGAENSVAYSNNSIAIP |
| | | TNFTISVTTEILPVSMTKTSVDCTMYICGDSTECSNLLLQYGSFCTQLNRALTGIAVEQDKNTQEV |
| | | FAQVKQIYKTPPIKDFGGFNFSQILPDPSKPSKRSFIEDLLFNKVTLADAGFIKQYGDCLGDIAARD |
| | | LICAQKINGLTVLPPLLTDEMIAQYTSALLAGTITSGWTFGAGAALQIPFAMQMAYRENGIGVT |
| | | QNVLYENQKLIANQFNSAIGKIQDSLSSTASALGKLQDVVNQNAQALNTLVKQLSSNFGAISSVL |
| | | NDILSRLDKVEAEVQIDRLITGRLQSLQTYVTQQLIRAAEIRASANLAATKMSECVLGQSKRVDF |
| | | CGKGYHLMSFPQSAPHGVVFLHVTYVPAQEKNFTTAPAICHDGKAHFPREGVFVSNGTHWFV |
| | | TQRNFYEPQIITTQNTFVSGNCDVVIGIVNNTVYDPLQPELDSFKEELDKYFKNHTSPDVDLGDI |
| | | SGINASVVNIQKEIDRLNEVAKNLNESLIDLQELGKYEQYIKWPWYIWLGFIAGLIAIVMVTIML |
| | | CCMTSCCSCLKGCCSCGSCCKFDEDDSEPVLKGVKLHYT |
| 12 | Amino | MFVFLVLLPLVSSQCVNLTTRTQLPPAYTNSFTRGVYYPDKVFRSSVLHSTQDLFLPFFSNVTWF |
| | acid | HAIHVSGTNGTKRFDNPVLPFNDGVYFASTEKSNIIRGWIFGTTLDSKTQSLUIVNNATNVVIKV |
| | sequence | CEFQFCNDPFLGVYYHKNNKSWMESEFRVYSSANNCTFEYVSQPFLMDLEGKQGNFKNLREF |
| | of a | VFKNIDGYFKIYSKHTPINLVRDLPQGFSALEPLVDLPIGINITRFQTLLALHRSYLTPGDSSSGWT |
| | modified | AGAAAYYVGYLQPRTFLLKYNENGTITDAVDCALDPLSETKCTLKSFTVEKGIYQTSNERVQPTES |
| | spike | IVRFPNITNLCPFGEVENATRFASVYAWNRKRISNCVADYSVLYNSASFSTFKCYGVSPTKLNDLC |
| | protein | FTNVYADSFVIRGDEVRQIAPGQTGKIADYNYKLPDDFTGCVIAWNSNNLDSKVGGNYNYLYR |
| | | LFRKSNLKPFERDISTEIYQAGSTPCNGVEGENCYFPLQSYGFQPTNGVGYQPYRVVVLSFELLH |
| | | APATVCGPKKSTNLVKNKCVNFNFNGLTGTGVITESNKKFLPFQQFGRDIADTTDAVRDPQTLEI |
| | | LDITPCSFGGVSVITPGTNTSNQVAVLYQGVNCTEVPVAIHADQLTPTWRVYSTGSNVFQTRAG |
| | | CLIGAEHVNNSYECDIPIGAGICASYQTQTNSPQSAQSVASQSIIAYTMSLGAENSVAYSNNSIAI |
| | | PTNFTISVTTEILPVSMTKTSVDCTMYICGDSTECSNILLQYGSFCTQLNRALTGIAVEQDKNTQE |
| | | VFAQVKQJYKTPPIKDFGGFNFSQILPDPSKPSKRSFIEDLLFNKVTLADAGFIKQYGDCLGDIAAR |
| | | DULICAQKENGLTVLPPLLTDEMIAQYTSALLAGTITSGWTFGAGAALQIPFAMQMAYRENGIGV |
| | | TQNVLYENQKLIANQFNSAIGKIQDSLSSTASALGKLQDVVNQNAQALNTIVKQLSSNFGAISSV |
| | | LNDILSRLQPPEAEVQIDRLITGRLQSLQTYVTQQLIRAAEIRASANLAATKMSECVLGQSKRVDF |
| | | CGKGYHLMSFPQSAPHGVVFLHVTYVPAQEKNFTTAPAICHDGKAHFPREGVFVSNGTHWFV |
| | | TQRNFYEPQUITTDNTFVSGNCQVVIGIVNNTVYDPLQPELDSFKEELDKYFKNHTSPDVDLGDI |
| | | SGINASVVNIQKEIDRINEVAKNLNESLIDLQELGKYEQYIKWPWYIWLGFIAGLIAIVMVTIML |
| | | CCMTSCCSCLKGCCSCGSCCKFDEDDSEPVLKGVKLHYT |
| 13 | 5′UTR | AGGAAAUUCCAUUUGGCUGCAGCUUCUGGAGGGAGCCGACAGGAGACGUGGGGAGA |
| | RNA | CGGCCACC |
| 14 | 3′UTR | GCUGCCUUCUGCGGGGCUUGCCUUCUGGCCAUGCCCUUCUUCUCUCCCUUGCACCUG |
| | RNA | UACCUCUUGGUCUUUGAAUAAAGCCUGAGUAGGAAGU |
| 15 | 5′UTR | AGGAAATTCCATTTGGCTGCAGCTTCTGGAGGGAGCCGACAGGAGACGTGGGGAGACGG |
| | DNA | CCACC |
| 16 | 3′UTR | GCTGCCTTCTGCGGGGCTTGCCTTCTGGCCATGCCCTTCTTCTCTCCCTTGCACCTGTACCTC |
| | DNA | TTGGTCTTTGAATAAAGCCTGAGTAGGAAGT |
| 17 | PolyA | AAAAAAAAAAAAAAAAAAAAAAAAAAAAAAAAAAAAAAAAAAAAAAAAAAAAAAAAAA |
| | RNA | AAAAAAAAAAAAAAAAA |
| 18 | PolyA | AAAAAAAAAAAAAAAAAAAAAAAAAAAAAAAAAAAAAAAAAAAAAAAAAAAAAAAAAA |
| | DNA | AAAAAAAAAAAAAAAAA |
| 19 | T7 | TAATACGACTCACTATA |
| | promoter | |
| 20 | Forward | GGGGAACTTCTCCTGCTAGAAT |
| | primer | |
| 21 | Reverse | CAGACATTTTGCTCTCAAGCTG |
| | primer | |
| 22 | Probe | FAM-TTGCTGCTGCTTGACAGATT-TAMRA |

1. A lipid composition, comprising a therapeutic agent or a prophylactic agent and a lipid encapsulating the therapeutic agent or the prophylactic agent, wherein the lipid encapsulating the therapeutic agent or the prophylactic agent comprises a cationic lipid, a phospholipid, a steroid, and a polyethylene glycol modified lipid; and the composition further comprises a cationic polymer, wherein the cationic polymer and the therapeutic agent or the prophylactic agent are associated as a complex and co-encapsulated in the lipid to form a lipopolyplex.

2. The lipid composition of claim 1, wherein the therapeutic agent or the prophylactic agent is a nucleic acid, such as an RNA, in particular an mRNA.

3. The lipid composition of claim 1, wherein the cationic lipid comprises a compound of formula (I), or a pharmaceutically acceptable salt thereof 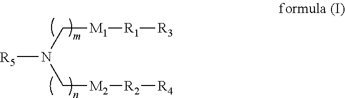
where
R₁ and R₂ are each independently selected from a bond, C₁-C₁₂ alkyl, and C₂-C₁₂ alkenyl;
R₃ and R₄ are each independently selected from C₁-C₁₂ alkyl, C₂-C₁₂ alkenyl, C₆-C₁₀ aryl, and 5-10 membered heteroaryl; and R₃ and R₄ are each independently optionally substituted with t R₆, t being an integer selected from 1-5;
R₆ is independently selected from C₁-C₁₂ alkyl and C₂-C₁₂ alkenyl;
M₁ and M₂ are each independently selected from a bond, H, —O—, —S—, —C(O)—, —OC(O)—, —C(O)O—, —SC(S)—, —C(S)S—, 3-10 membered heterocycle, and —NR₇—, or
R₅ and one of M₁ and M₂ together with an N atom to which they are connected form 3-10 membered heterocycle, and corresponding R₁/R₃ or R₂/R₄ is absent, the heterocycle being optionally substituted with R₇;
R₅ is selected from C₃₋₈ carbocycle and —C₁₋₁₂ alkylene-Q, and Q is selected from H, —OR₇, —SR₇, —OC(O)R₇, —C(O)OR₇, —N(R₇)C(O)R₇, —N(R₇)S(O)₂R₇, —N(R₇)C(S)R₇, —N(R₇)₂, cyano, C₃₋₈ carbocycle, 3-10 membered heterocycle, and C₆-C₁₀ aryl, each of which is optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O);
m and n are each independently an integer selected from 0-12;
the alkyl, the alkenyl, and the alkylene are each optionally independently interrupted by one or more groups selected from —O—, —S—, —NR₇—, —C(O)—, —OC(O)—, —C(O)O—, —SC(S)—, —C(S)S—, and C₃₋₈ carbocycle, and the alkyl, the alkenyl, and the alkylene are each optionally substituted with one or more R₇; and
R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, 5-10 membered heteroaryl, 3-10 membered heterocycle, halogen, and C₃₋₈ carbocycle, each of which is optionally substituted with one or more C₁-C₁₂ alkyl, C₂-C₁₂ alkenyl, C₁-C₁₂ alkoxyl, C₆-C₁₀ aryl, 5-10 membered heteroaryl, 3-10 membered heterocycle, halogen, hydroxyl, and oxo(═O).

4. The lipid composition of claim 3, wherein
R₁ and R₂ are each independently selected from C₁-C₁₂ alkyl and C₂-C₁₂ alkenyl;
R₃ and R₄ are each independently selected from C₁-C₁₂ alkyl and C₂-C₁₂ alkenyl; R₃ and R₄ are each independently optionally substituted with t R₆, t being an integer selected from 1-5; and R₆ is independently selected from C₁-C₁₂ alkyl and C₂-C₁₂ alkenyl;
M₁ and M₂ are each independently selected from —OC(O)—, —C(O)O—, —SC(S)—, and —C(S)S—;
R₅ is selected from —C₁₋₁₂ alkylene-Q, Q is selected from —OR₇ and —SR₇, and R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl; and
m and n are each independently an integer selected from 1-12.

5. The lipid composition of claim 3, wherein the cationic lipid comprises a lipid compound having a structure shown below, or a pharmaceutically acceptable salt thereof:

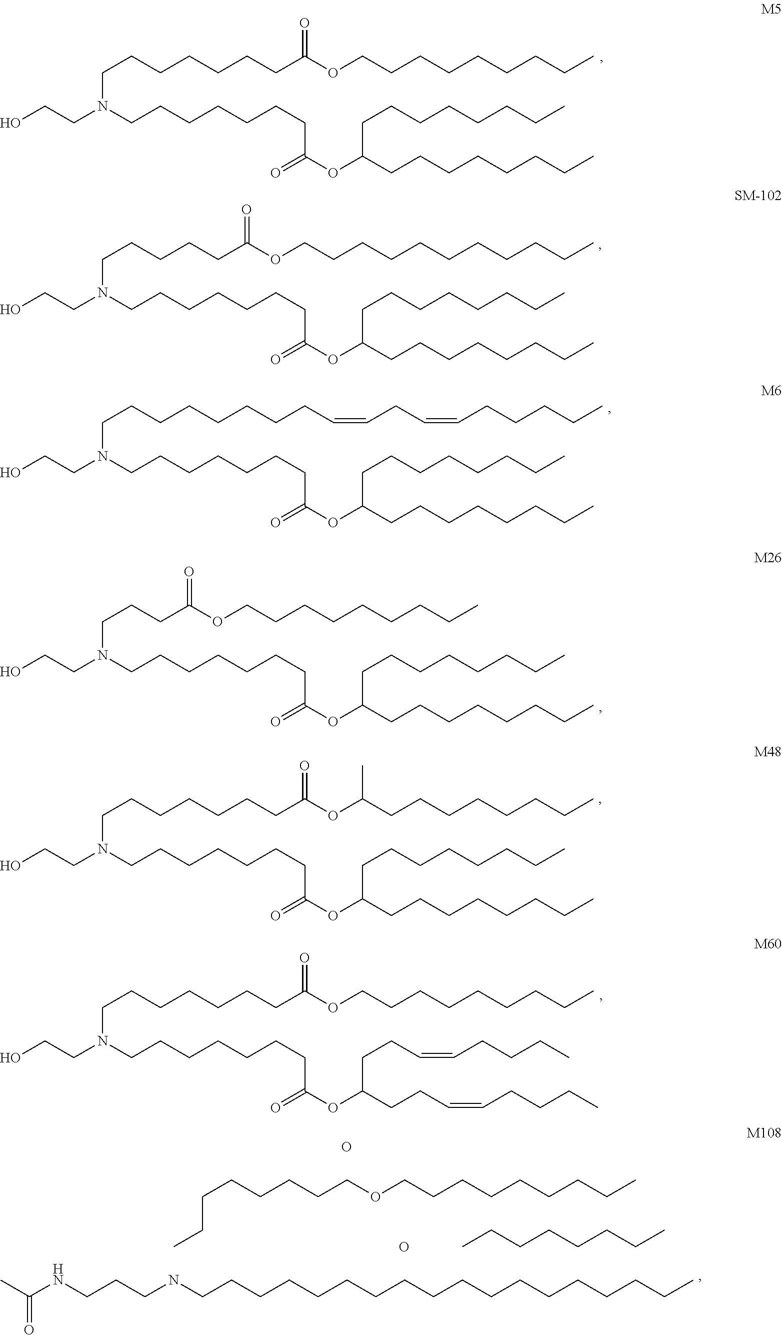

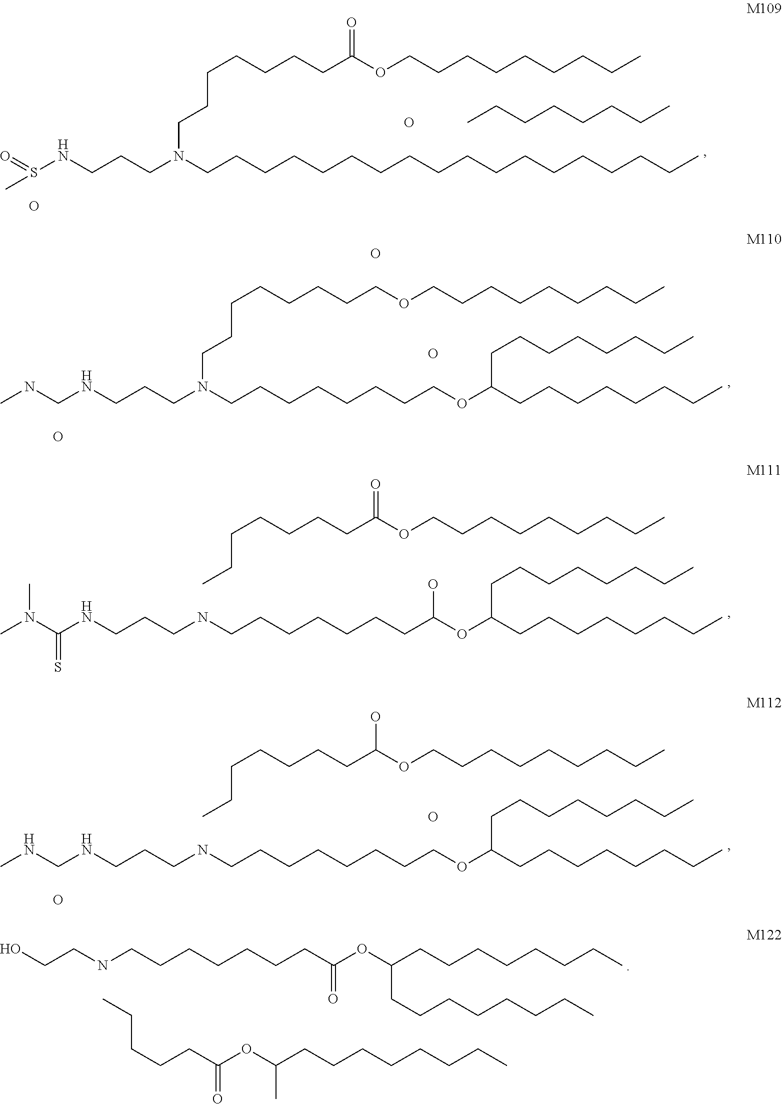

6. The lipid composition of claim 3, wherein the cationic lipid comprises a lipid compound having a structure shown below, or a pharmaceutically acceptable salt thereof:

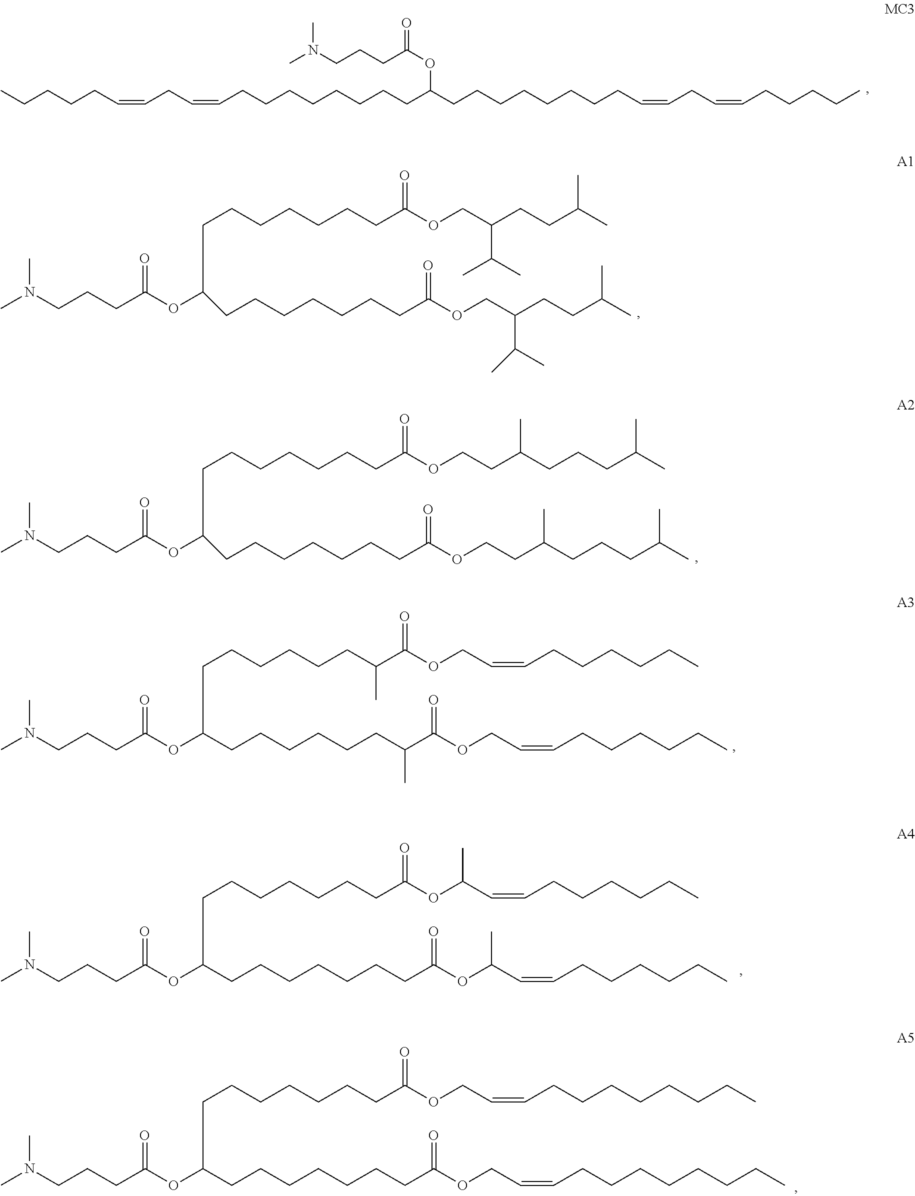

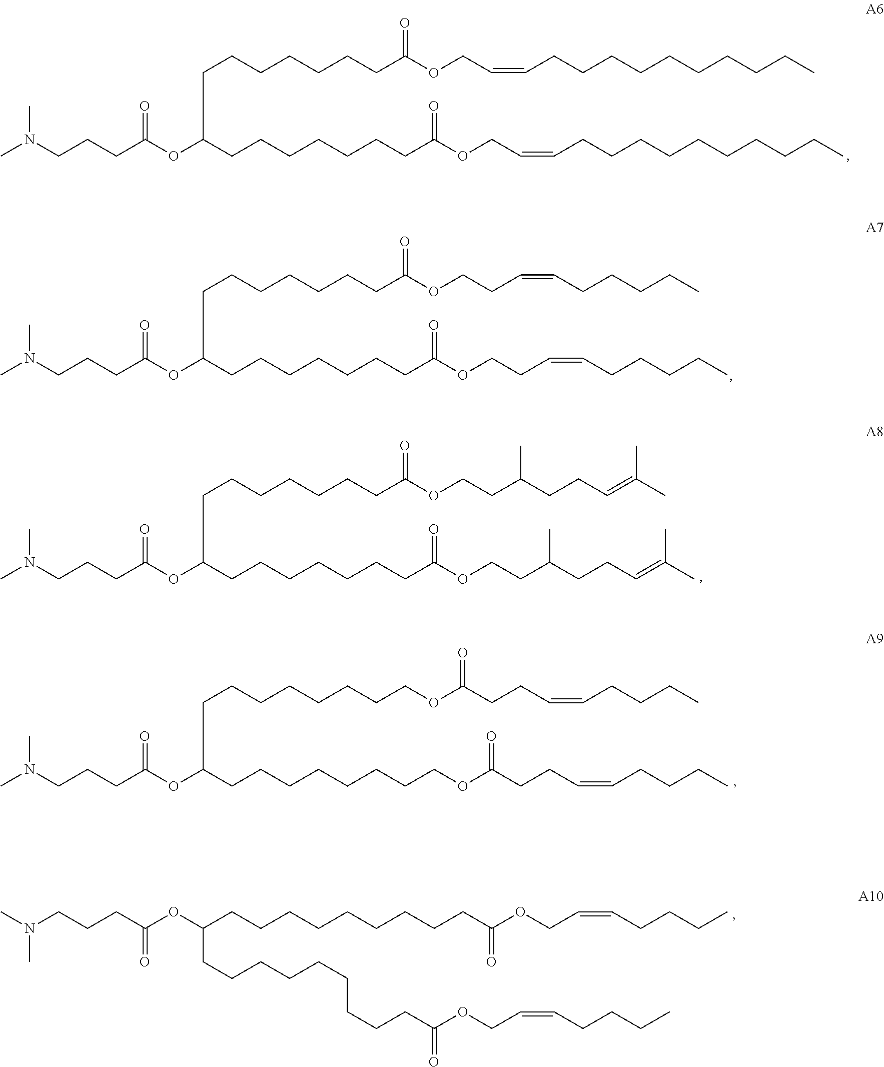

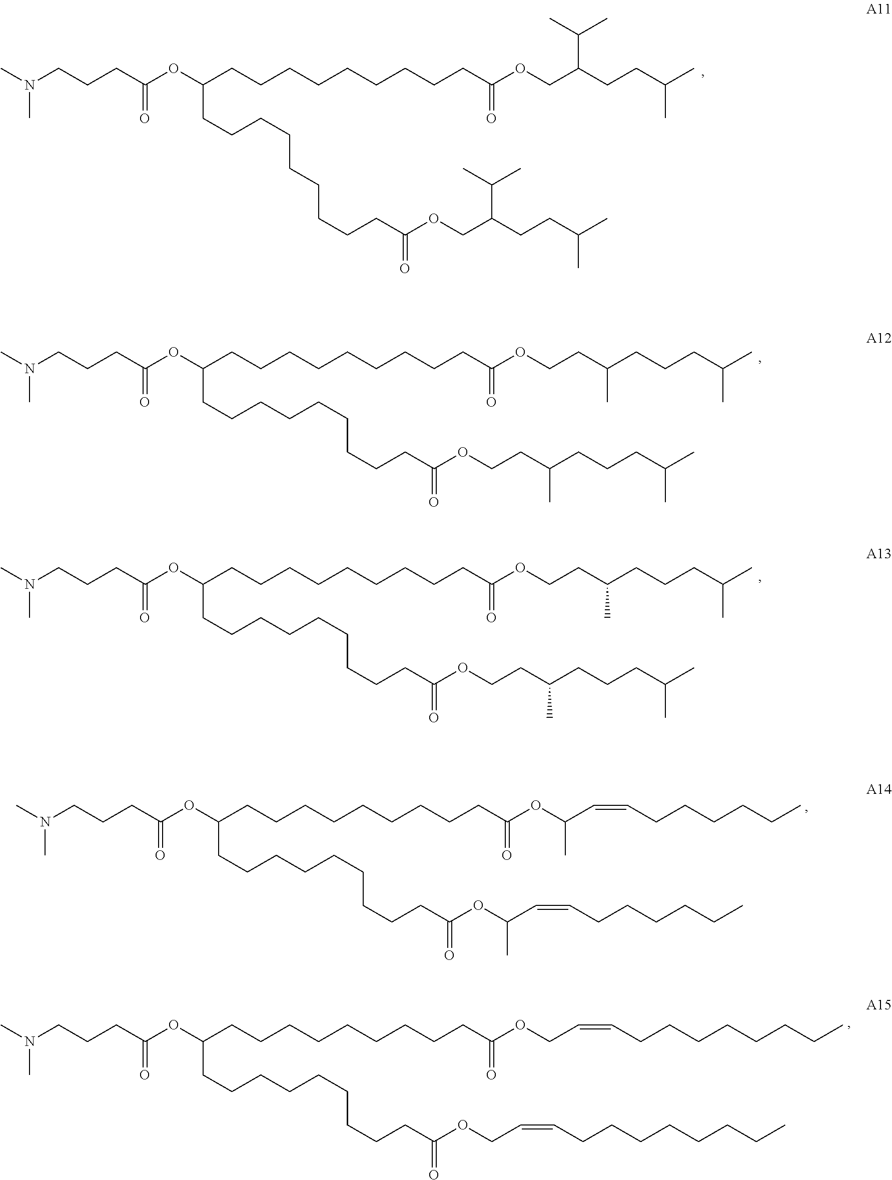

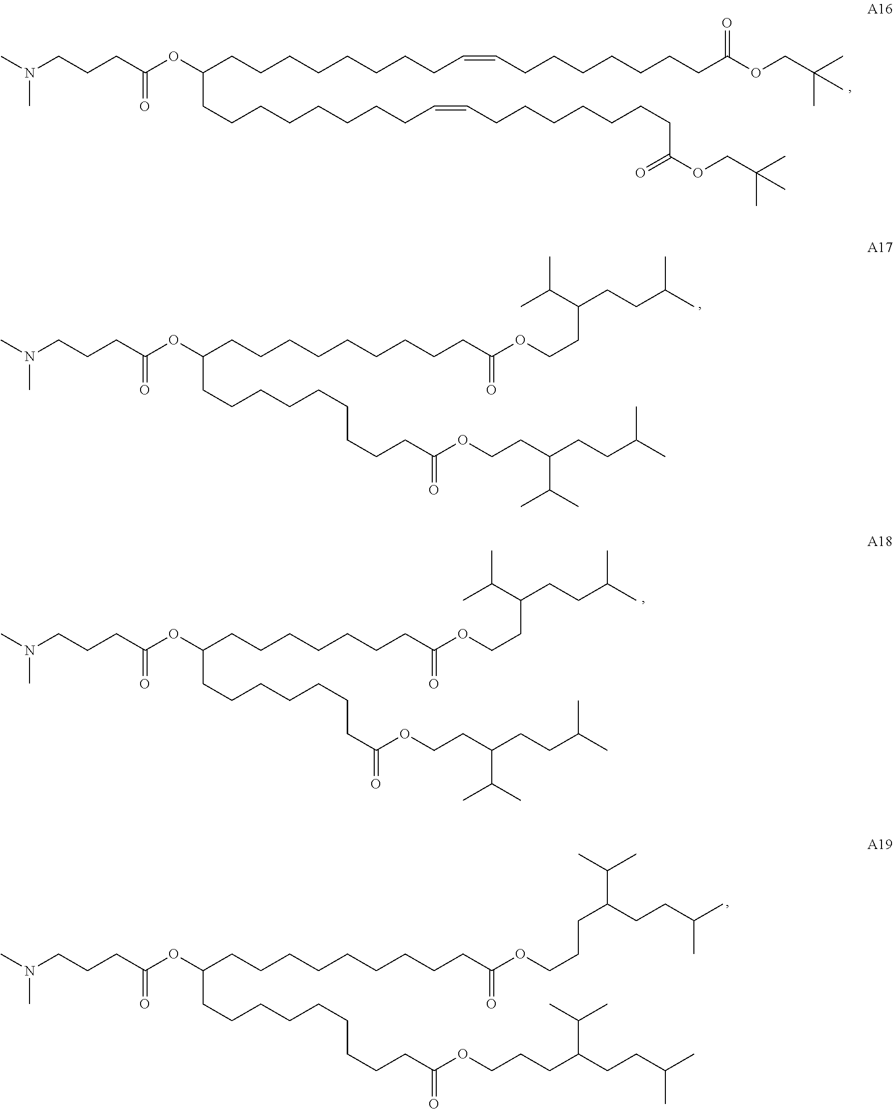

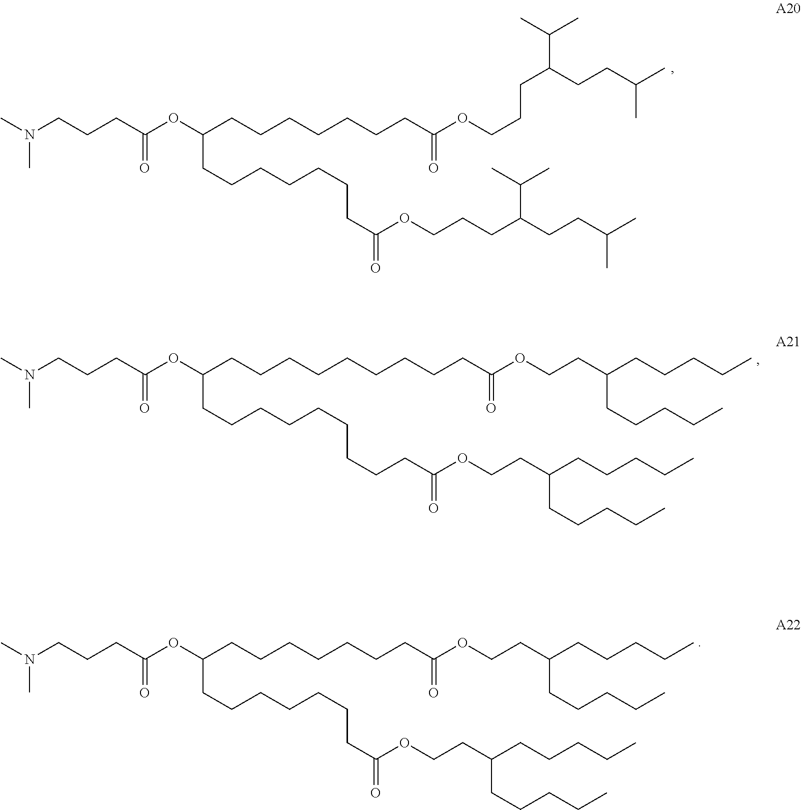

7. The lipid composition of claim 3, wherein the cationic lipid comprises a lipid compound having a structure shown below, or a pharmaceutically acceptable salt thereof:

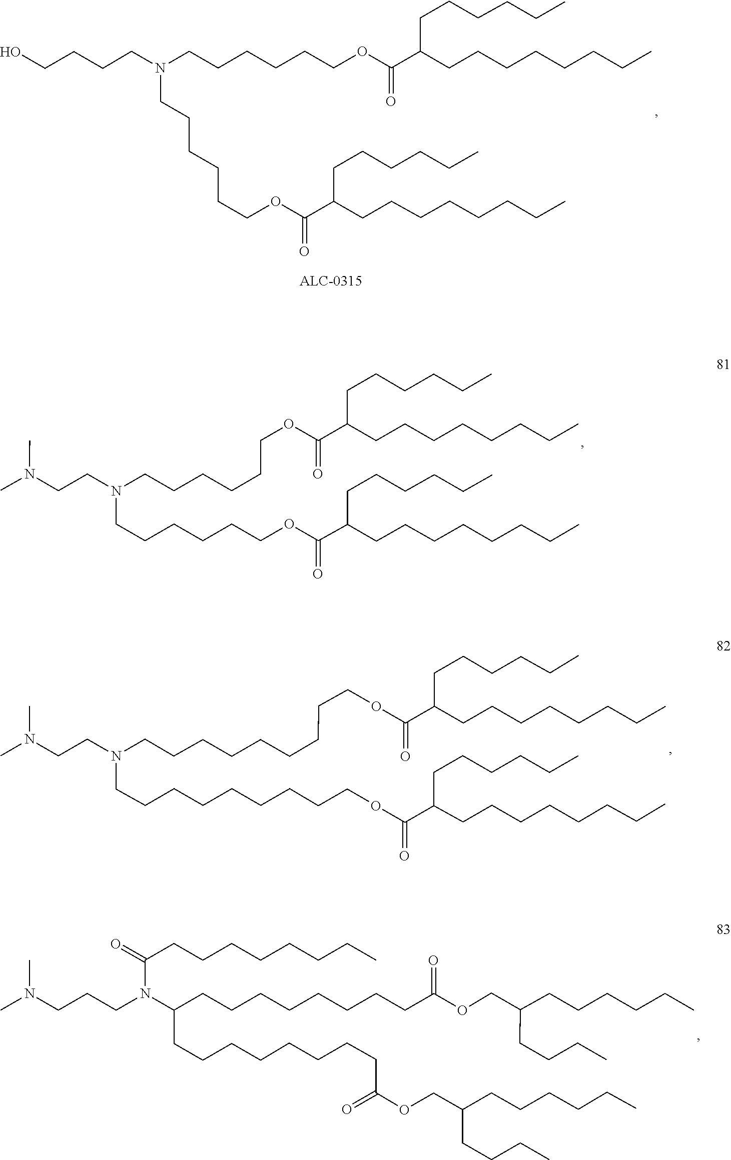

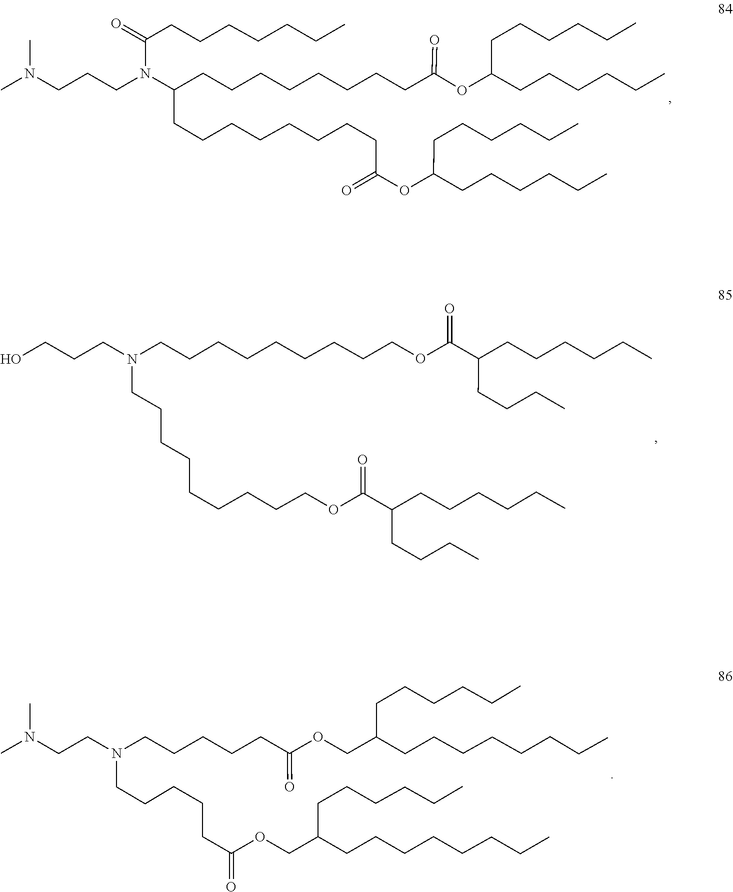

8. The lipid composition of claim 3, wherein
R₁ and R₂ are each independently selected from C₁-C₁₂ alkyl and C₂-C₁₂ alkenyl;
R₃ and R₄ are each independently selected from C₁-C₁₂ alkyl, C₂-C₁₂ alkenyl, C₆-C₁₀ aryl, and 5-10 membered heteroaryl,
with a proviso that at least one of R₃ and R₄ is C₆-C₁₀ aryl or 5-10 membered heteroaryl; R₃ and R₄ are each independently optionally substituted with t R₆, t being an integer selected from 1-5; and R₆ is independently selected from C₁-C₁₂ alkyl and C₂-C₁₂ alkenyl;
M₁ and M₂ are each independently selected from —OC(O)—, —C(O)O—, —SC(S)—, and —C(S)S—;
R₅ is selected from —C₁₋₁₂ alkylene-Q, Q is selected from —OR₇ and —SR₇, and R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl; and
m and n are each independently an integer selected from 1-12.

9. The lipid composition of claim 8, wherein
R₁ and R₂ are each independently selected from C₁-C₁₂ alkyl.

10. The lipid composition of claim 9, wherein
R₃ and R₄ are each independently selected from C₁-C₁₂ alkyl and C₆-C₁₀ aryl,
with a proviso that one of R₃ and R₄ is C₆-C₁₀ aryl, and the other is C₁-C₁₂ alkyl;
R₃ and R₄ are each independently substituted with t R₆, t being an integer selected from 1-3; and
R₆ is independently selected from C₁-C₁₂ alkyl.

11. The lipid composition of claim 10, wherein
M₁ and M₂ are each independently selected from: —OC(O)— and —C(O)O—.

12. The lipid composition of claim 11, wherein
R₅ is selected from —C₁₋₅ alkylene-Q, and Q is —OH.

13. The lipid composition of claim 12, wherein
m and n are each independently an integer selected from 2-7.

14. The lipid composition of claim 13, wherein
R₄ is substituted at a 1-position or a last position of R₂; and/or
R₃ is substituted at a 1-position or a last position of R₁.

15. The lipid composition claim 14, wherein
t is 1 or 2, and R₆ is substituted at a meta-position and/or a para-position on a benzene ring relative to R₁ or R₂.

16. The lipid composition of claim 15, wherein
t is 1 or 2, and R₆ is independently selected from C₁-C₁₀ alkyl.

17. The lipid composition of claim 16, wherein the cationic lipid comprises a compound of formula (II), or a pharmaceutically acceptable salt thereof: 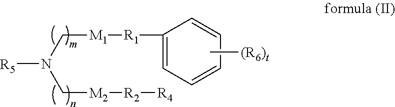
where R₁, R₂, R₄, R₅, R₆, M₁, M₂, t, m, and n are as defined in claim 16; and
preferably, in formula (II),
R₁ is selected from C₁-C₆ alkyl;
R₂ is selected from C₁-C₁₀ alkyl;
R₄ is selected from C₁-C₁₀ alkyl;
M₁ and M₂ are each independently selected from: —OC(O)— and —C(O)O—;
R₅ is selected from —C₁₋₅ alkylene-Q, Q is selected from —OR₇ and —SR₇, and R₇ is independently selected from H, C₁-C₁₂ alkyl, and C₂-C₁₂ alkenyl;
m and n are each independently an integer selected from 2-9;
t is an integer selected from 1-3; and
R₆ is independently selected from C₁-C₁₂ alkyl and C₂-C₁₂ alkenyl.

18. The lipid composition of claim 16, wherein the cationic lipid comprises a compound of formula (III), or a pharmaceutically acceptable salt thereof: 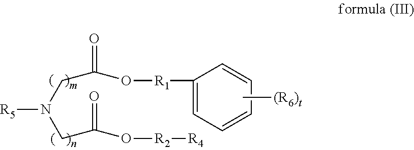
where R₁, R₂, R₄, R₅, R₆, t, m, and n are as defined in claim 16; and
preferably, in formula (III),
R₁ is selected from C₁-C₆ alkyl;
R₂ is selected from C₁-C₁₀ alkyl;
R₄ is selected from C₁-C₁₀ alkyl;
R₅ is selected from —C₁₋₃ alkylene-Q, and Q is selected from —OH and —SH;
t is 1 or 2;
R₆ is selected from C₁-C₁₂ alkyl and C₂-C₁₂ alkenyl; and
m and n are each independently an integer selected from 2-7.

19. The lipid composition of claim 16, wherein the cationic lipid comprises a compound of formula (IV), or a pharmaceutically acceptable salt thereof: 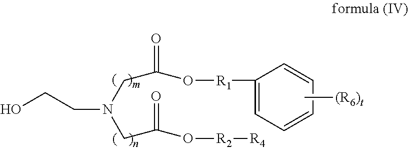
where R₁, R₂, R₄, R₆, t, m, and n are as defined in claim 16; and
preferably, in formula (IV),
R₁ is selected from C₁-C₆ alkyl;
R₂ is selected from C₁-C₁₀ alkyl;
R₄ is selected from C₁-C₁₀ alkyl;
t is 1 or 2;
R₆ is independently selected from C₁-C₁₂ alkyl and C₂-C₁₂ alkenyl; and
m and n are each independently an integer selected from 2-7.

20. The lipid composition of claim 8, wherein the cationic lipid comprises a lipid compound having a structure shown below, or a pharmaceutically acceptable salt thereof: 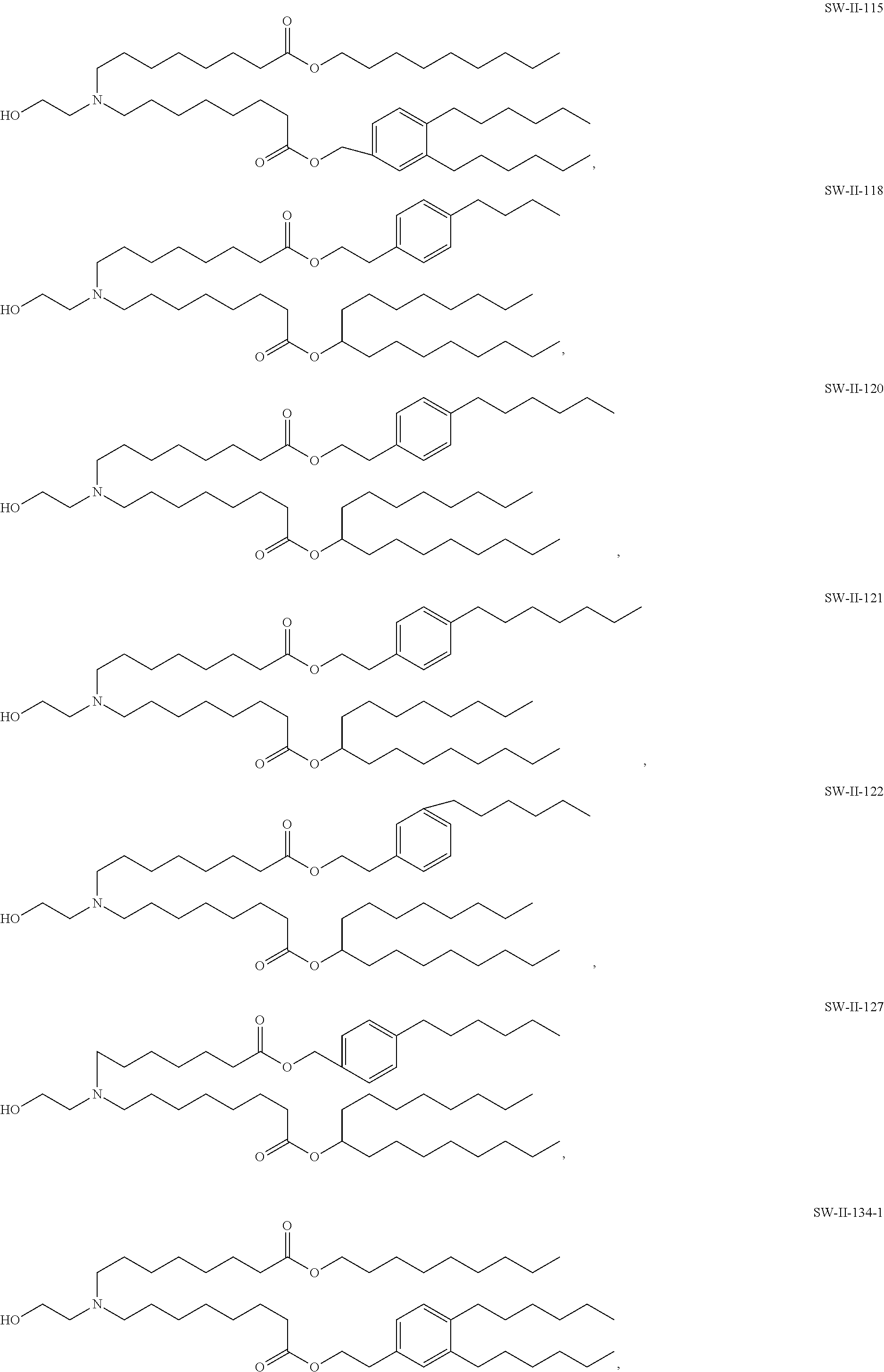 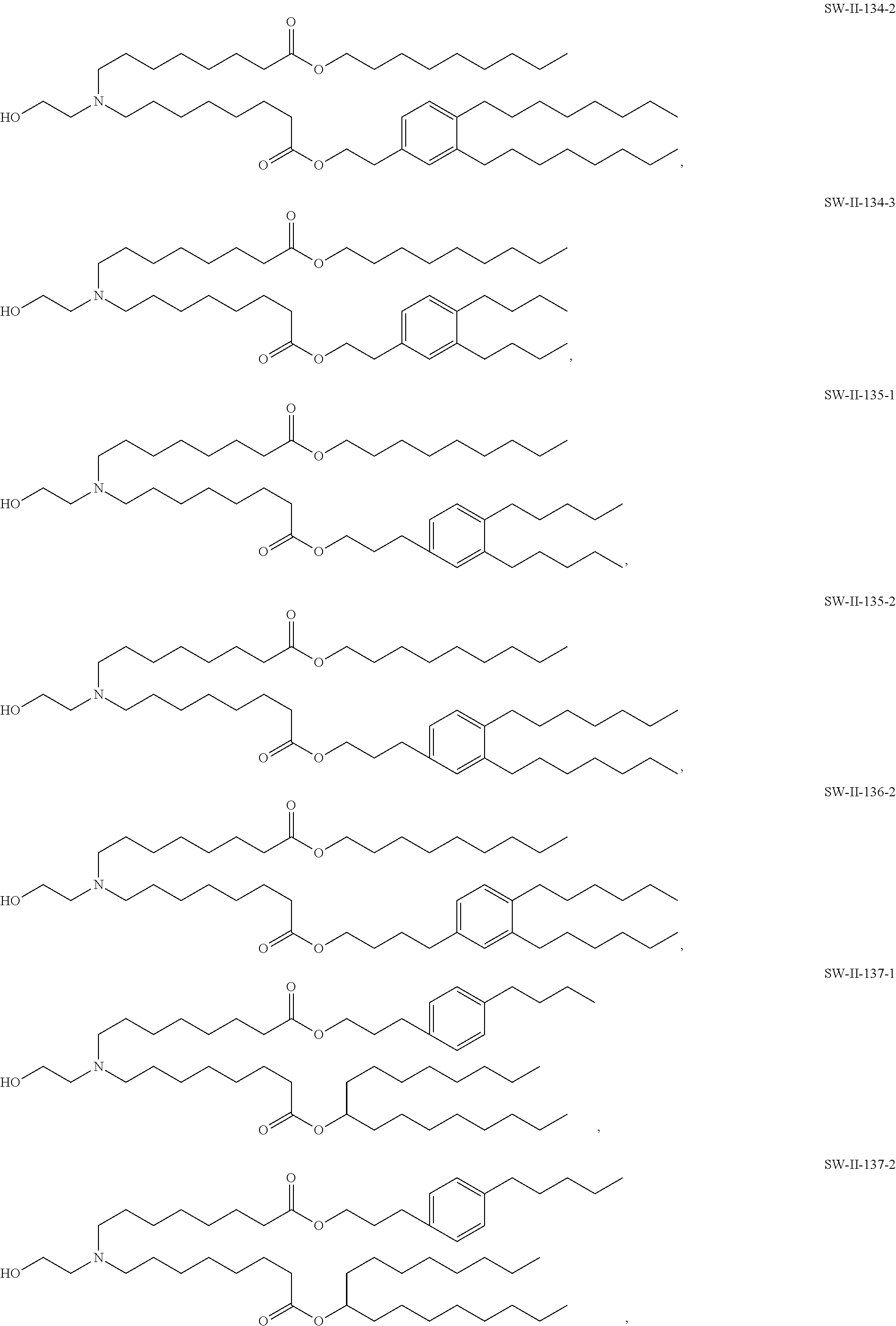 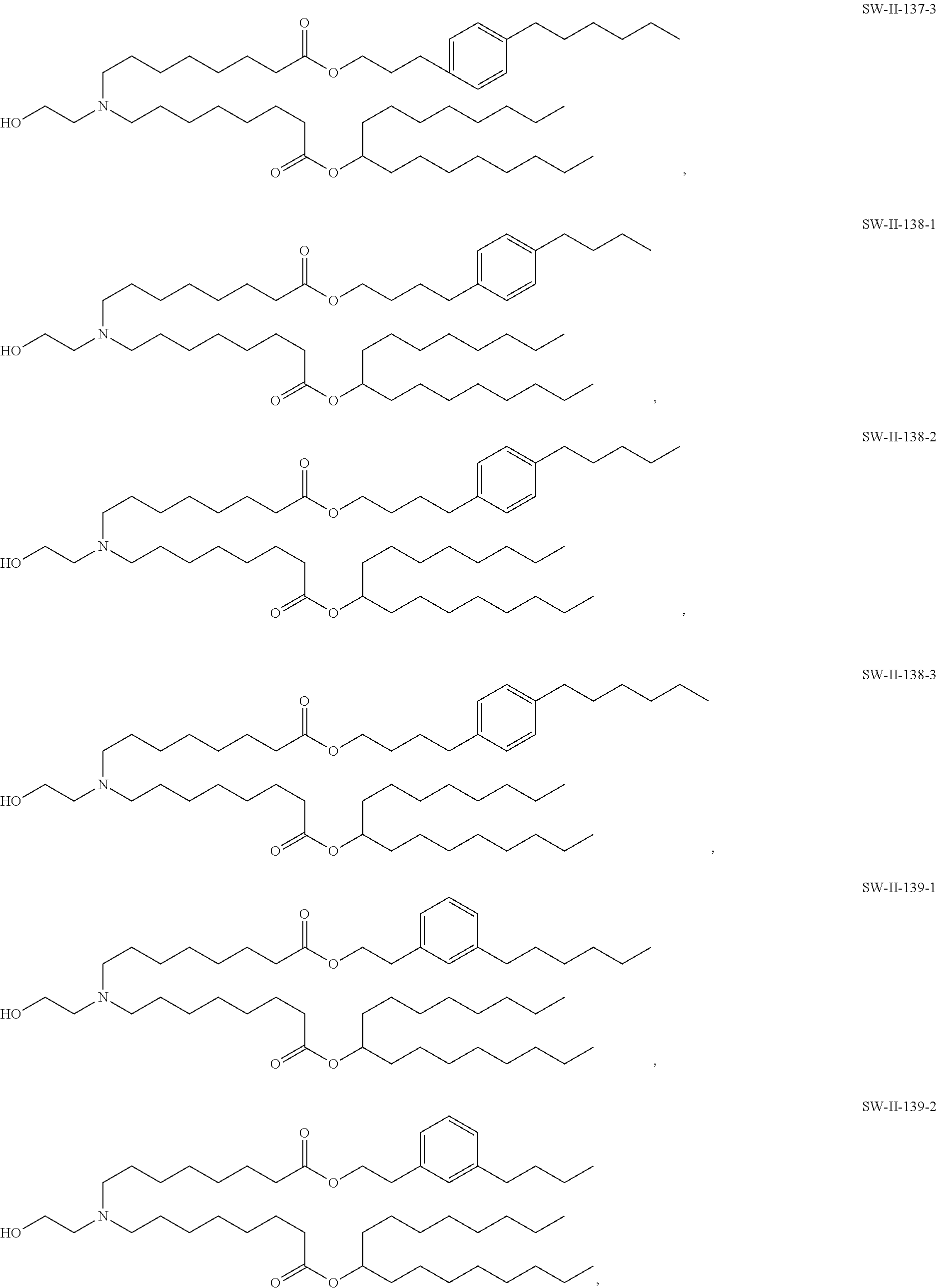 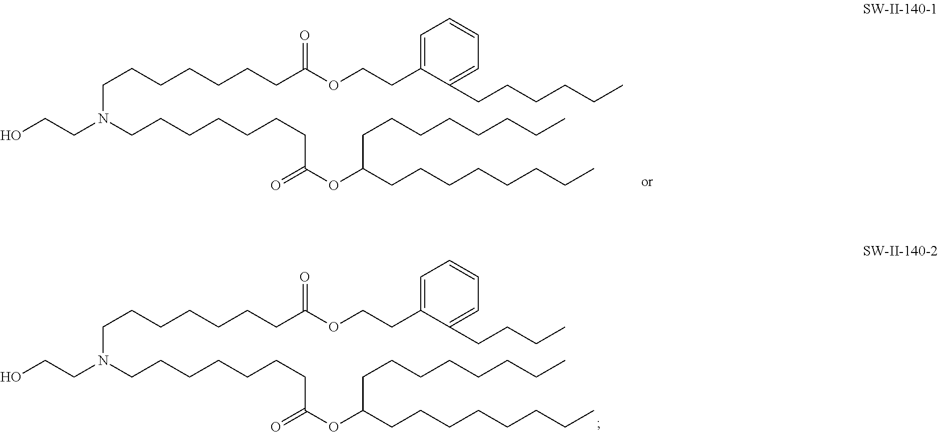 [SMILES: CCCCCCCCC(CCCCCCCC)OC(=O)CCCCCCCN(CCO)CCCCCCCC(=O)OCCc1ccccc1CCCCCC ; CCCCCCCCC(CCCCCCCC)OC(=O)CCCCCCCN(CCO)CCCCCCCC(=O)OCCc1ccccc1CCCC]
and preferably, the cationic lipid is SW-II-115, SW-II-121, SW-II-122, SW-II-134-3, SW-II-138-2, SW-II-139-2 or SW-II-140-2.

21-40. (canceled)


SEQUENCE LISTING (22 sequences)

SEQ ID NO: 1 - RNA, 1653 nt, synthetic construct
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 2 - RNA, 720 nt, synthetic construct
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - RNA, 3825 nt, synthetic construct
  source 1..3825: mol_type = other RNA
atgttcgtattcctagtgctgctgcccctggtgagcagccagtgcgtgaacctgaccacacgcactcagctgccaccagcctacaccaactcattcaccaggggcgtgtactatcctgacaaggtgttcaggagcagcgtgctgcactccacccaggatctgttcctgcccttcttcagcaacgtgacatggttccacgccatccacgtgagcgggacaaacggcaccaagcggtttgacaatcccgttctgccattcaacgatggcgtgtacttcgccagcaccgagaaaagcaacatcatccggggctggatcttcggcaccacactggacagcaagacccagagtctgctgatcgtgaacaacgccaccaacgtggtgatcaaggtgtgtgagttccagttctgtaatgaccccttcctgggggtctattaccacaagaacaataaaagctggatggagagcgagtttcgggtgtacagtagtgccaacaactgtacattcgagtacgtgtcccagccattcctgatggacctggaggggaagcaggggaacttcaagaacctgcgggagttcgtgtttaagaatatcgacggatattttaagatctactccaagcacacccccatcaacctggtgcgggatctgccccagggcttcagcgccctggagcccctggtggacctgcccatcgggatcaacatcacccggttccagaccctgctggccctgcaccggagttacctgaccccaggtgactccagcagcggctggacagccggggctgcagcctactacgtgggctacctgcagccccggaccttcctcctgaagtacaatgagaacggcaccatcaccgatgccgtggattgtgctctggaccccctgtctgagaccaagtgcaccctgaaaagcttcaccgtggagaaggggatctaccagacaagcaactttcgggtgcagcccaccgagagtatcgtgaggttccccaacatcaccaacctgtgtccattcggggaggtgttcaacgccacccggttcgcaagcgtgtacgcttggaaccggaagcgcatcagcaattgcgtggctgattacagcgtgctgtacaacagcgcctccttcagcacattcaagtgctacggcgtctcccccacaaagctgaacgatctgtgtttcaccaacgtgtacgcagacagctttgtgatccggggagacgaggtgcggcagatcgcacccgggcagaccggcaagattgctgactacaattacaagctgcctgatgacttcacaggctgcgtgattgcctggaacagcaataacctggacagcaaggtgggaggtaattacaattacctctaccggctgttcaggaagtccaatctgaagcccttcgagagggacatctccaccgagatctaccaggctgggtccaccccatgcaatggggtggagggcttcaactgctacttccccctgcagtcctacggcttccagcccaccaatggggtgggctaccagccctaccgggtggtggtgctgagcttcgagctcctgcacgcccccgccactgtgtgcggccccaagaagtccaccaacctggtgaagaacaagtgcgtgaacttcaacttcaacgggttgaccgggactggggtgctcacagagagcaacaagaagttcctgcccttccagcagttcggaagggacatcgccgacaccacggatgccgtgagggatccccagaccctcgagatcctggacatcacgccatgctccttcgggggcgtgagcgtgatcactcctggcacaaacacctccaaccaggtggctgtgctgtaccagggcgtgaactgcaccgaggtgcctgtggccatccatgctgaccagctgacccccacctggcgggtctacagcaccggcagcaacgtgttccagacccgggccggctgcctgatcggggccgagcacgtgaacaacagctacgagtgcgatatccccatcggcgccggcatctgcgctagctaccagacacagaccaatagcccccaatccgctcaatctgtggctagccagagcatcatcgcctacactatgtctctgggcgcagagaatagtgtggcttacagcaacaacagcatcgccattcccactaatttcacaattagtgtgaccaccgagatcctgcctgtgagcatgaccaagaccagcgtggactgcaccatgtacatctgcggggatagcaccgagtgcagcaacctgctgctgcagtacggcagcttctgcacacagctgaaccgggccctgaccggtatcgctgtggagcaggacaagaacacccaggaggtgtttgcgcaggtgaagcagatctacaagacccctcctatcaaggacttcgggggctttaacttcagccagatcctgccagacccctccaagccctccaagaggagcttcatcgaggatctcctgttcaacaaggtgaccctggccgatgccggcttcatcaagcagtacggagattgcctgggcgacatcgccgccagggatctcatctgcgcccagaagttcaacggcctgaccgtgctgccccccctgctgaccgatgagatgatcgctcagtacaccagcgccctgctggctgggaccatcaccagcgggtggaccttcggggccggcgccgccctgcagatccccttcgccatgcagatggcctaccggttcaacggtattggggtgacccagaatgtgctgtatgagaaccagaagctgatcgccaaccagttcaactctgccatcggcaagatccaggactccctgagcagcactgccagcgccctgggcaagctgcaggacgtggtgaaccagaacgcccaggccctgaataccctggtgaagcagctgtccagcaacttcggggccatcagcagcgtgctgaacgacatcctgtcccgcctggacccaccagaggcagaggtgcagatcgatcggctgatcacaggtaggctgcagagcctgcagacctacgtgacccagcagctgatccgcgccgctgagattagggccagcgccaacctggccgccactaagatgagcgagtgcgtgctgggccagagcaagcgggtggacttctgcggcaaggggtatcacctgatgagcttccctcagtccgccccccacggggtggtgttcctgcacgtgacatacgtgcccgcccaggagaagaacttcaccaccgcccctgccatctgccacgatggcaaggcccactttccccgggagggcgtgttcgtgtccaatggcacacattggttcgtgacccagcggaacttctacgagccacagatcattaccactgacaatacctttgtcagtggtaactgtgacgtggtgatcggcattgtgaacaacacagtgtacgatcctctgcagccagagctggacagcttcaaggaggagctggacaagtacttcaagaaccacaccagccccgacgtggatcttggagacatctctggcatcaacgccagcgtggtcaacatccagaaggagattgaccgcctgaacgaggttgccaagaacctgaacgagagtctgatcgacctccaggagcttggcaagtacgagcagtacatcaagtggccatggtacatctggctgggcttcatcgccggcctgatcgccatcgtgatggtgaccatcatgctgtgctgcatgacctcttgttgcagctgcctgaaggggtgctgcagctgcggtagttgctgtaagttcgacgaggacgacagcgagcccgtgctgaagggcgtgaagctgcactacacgtaatag

SEQ ID NO: 4 - RNA, 3825 nt, synthetic construct
  source 1..3825: mol_type = other RNA
atgttcgtcttcctggtgctgctgcctctggtgtcttcccagtgcgtgaatctgactaccaggacccagctgccccctgcctataccaattccttcacacggggcgtgtactatcccgacaaggtgtttagaagctccgtgctgcactctacacaggatctgtttctgcctttctttagcaacgtgacctggttccacgccatccacgtgagcggcaccaatggcacaaagcggttcgacaatccagtgctgccctttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatccattcctgggcgtgtactatcacaagaacaataagagctggatggagtccgagtttcgcgtgtattctagcgccaacaattgcacatttgagtacgtgtcccagcccttcctgatggacctggagggcaagcagggcaatttcaagaacctgagggagttcgtgtttaagaatatcgatggctacttcaagatctactctaagcacaccccaatcaacctggtgcgcgacctgccacagggcttcagcgccctggagccactggtggatctgcccatcggcatcaacatcacccggtttcagacactgctggccctgcacagaagctacctgacaccaggcgactcctctagcggatggaccgcaggagcagcagcctactatgtgggctatctgcagcccaggaccttcctgctgaagtacaacgagaatggcaccatcacagacgccgtggattgcgccctggatcccctgagcgagacaaagtgtacactgaagtcctttaccgtggagaagggcatctatcagacatccaatttcagggtgcagcctaccgagtctatcgtgcgctttcccaatatcacaaacctgtgcccttttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcaccaggacagacaggcaagatcgcagactacaattataagctgcctgacgatttcaccggctgcgtgatcgcctggaacagcaacaatctggattccaaagtgggcggcaactacaattatctgtaccggctgtttagaaagagcaatctgaagccattcgagagggacatctctacagagatctaccaggcaggaagcaccccatgcaatggagtggagggctttaactgttatttccctctgcagtcctacggcttccagccaaccaacggcgtgggctatcagccctaccgcgtggtggtgctgagctttgagctgctgcacgcacctgcaacagtgtgcggaccaaagaagtccaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctctttcggcggcgtgagcgtgatcacaccaggcaccaatacaagcaaccaggtggccgtgctgtatcaggacgtgaattgtaccgaggtgccagtggcaatccacgcagatcagctgacccctacatggcgggtgtacagcaccggctccaacgtgttccagacaagagcaggatgtctgatcggagcagagcacgtgaacaattcctatgagtgcgacatccctatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatccctaccaacttcacaatctctgtgaccacagagatcctgccagtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccacccatcaaggactttggcggcttcaatttttcccagatcctgcccgatccttccaagccctctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgcctccactgctgacagatgagatgatcgcacagtacacaagcgccctgctggcaggaaccatcacatccggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacaaggtggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttcccacagtctgcccctcacggagtggtgtttctgcacgtgacctacgtgccagcccaggagaagaacttcaccacagcaccagcaatctgccacgatggcaaggcacactttcctagggagggcgtgttcgtgtccaacggcacccactggtttgtgacacagcgcaatttctacgagccacagatcatcaccacagacaataccttcgtgagcggcaactgtgacgtggtcatcggcatcgtgaacaataccgtgtatgatcctctgcagccagagctggacagctttaaggaggagctggataagtacttcaagaatcacacctcccccgacgtggatctgggcgacatcagcggcatcaatgcctccgtggtgaacatccagaaggagatcgacaggctgaacgaggtggccaagaatctgaacgagagcctgatcgatctgcaggagctgggcaagtatgagcagtacatcaagtggccttggtacatctggctgggcttcatcgccggcctgatcgccatcgtgatggtgaccatcatgctgtgctgtatgacatcctgctgttcttgcctgaagggctgctgtagctgcggctcctgttgtaaattcgatgaggatgattccgagcctgtgctgaagggcgtgaaactgcattatacctaatag

SEQ ID NO: 5 - RNA, 3825 nt, synthetic construct; identical to SEQ ID NO: 3
  source 1..3825: mol_type = other RNA

SEQ ID NO: 6 - RNA, 4058 nt, synthetic construct
  source 1..4058: mol_type = other RNA
aggaaattccatttggctgcagcttctggagggagccgacaggagacgtggggagacggccaccatgttcgtattcctagtgctgctgcccctggtgagcagccagtgcgtgaacctgaccacacgcactcagctgccaccagcctacaccaactcattcaccaggggcgtgtactatcctgacaaggtgttcaggagcagcgtgctgcactccacccaggatctgttcctgcccttcttcagcaacgtgacatggttccacgccatccacgtgagcgggacaaacggcaccaagcggtttgacaatcccgttctgccattcaacgatggcgtgtacttcgccagcaccgagaaaagcaacatcatccggggctggatcttcggcaccacactggacagcaagacccagagtctgctgatcgtgaacaacgccaccaacgtggtgatcaaggtgtgtgagttccagttctgtaatgaccccttcctgggggtctattaccacaagaacaataaaagctggatggagagcgagtttcgggtgtacagtagtgccaacaactgtacattcgagtacgtgtcccagccattcctgatggacctggaggggaagcaggggaacttcaagaacctgcgggagttcgtgtttaagaatatcgacggatattttaagatctactccaagcacacccccatcaacctggtgcgggatctgccccagggcttcagcgccctggagcccctggtggacctgcccatcgggatcaacatcacccggttccagaccctgctggccctgcaccggagttacctgaccccaggtgactccagcagcggctggacagccggggctgcagcctactacgtgggctacctgcagccccggaccttcctcctgaagtacaatgagaacggcaccatcaccgatgccgtggattgtgctctggaccccctgtctgagaccaagtgcaccctgaaaagcttcaccgtggagaaggggatctaccagacaagcaactttcgggtgcagcccaccgagagtatcgtgaggttccccaacatcaccaacctgtgtccattcggggaggtgttcaacgccacccggttcgcaagcgtgtacgcttggaaccggaagcgcatcagcaattgcgtggctgattacagcgtgctgtacaacagcgcctccttcagcacattcaagtgctacggcgtctcccccacaaagctgaacgatctgtgtttcaccaacgtgtacgcagacagctttgtgatccggggagacgaggtgcggcagatcgcacccgggcagaccggcaagattgctgactacaattacaagctgcctgatgacttcacaggctgcgtgattgcctggaacagcaataacctggacagcaaggtgggaggtaattacaattacctctaccggctgttcaggaagtccaatctgaagcccttcgagagggacatctccaccgagatctaccaggctgggtccaccccatgcaatggggtggagggcttcaactgctacttccccctgcagtcctacggcttccagcccaccaatggggtgggctaccagccctaccgggtggtggtgctgagcttcgagctcctgcacgcccccgccactgtgtgcggccccaagaagtccaccaacctggtgaagaacaagtgcgtgaacttcaacttcaacgggttgaccgggactggggtgctcacagagagcaacaagaagttcctgcccttccagcagttcggaagggacatcgccgacaccacggatgccgtgagggatccccagaccctcgagatcctggacatcacgccatgctccttcgggggcgtgagcgtgatcactcctggcacaaacacctccaaccaggtggctgtgctgtaccagggcgtgaactgcaccgaggtgcctgtggccatccatgctgaccagctgacccccacctggcgggtctacagcaccggcagcaacgtgttccagacccgggccggctgcctgatcggggccgagcacgtgaacaacagctacgagtgcgatatccccatcggcgccggcatctgcgctagctaccagacacagaccaatagcccccaatccgctcaatctgtggctagccagagcatcatcgcctacactatgtctctgggcgcagagaatagtgtggcttacagcaacaacagcatcgccattcccactaatttcacaattagtgtgaccaccgagatcctgcctgtgagcatgaccaagaccagcgtggactgcaccatgtacatctgcggggatagcaccgagtgcagcaacctgctgctgcagtacggcagcttctgcacacagctgaaccgggccctgaccggtatcgctgtggagcaggacaagaacacccaggaggtgtttgcgcaggtgaagcagatctacaagacccctcctatcaaggacttcgggggctttaacttcagccagatcctgccagacccctccaagccctccaagaggagcttcatcgaggatctcctgttcaacaaggtgaccctggccgatgccggcttcatcaagcagtacggagattgcctgggcgacatcgccgccagggatctcatctgcgcccagaagttcaacggcctgaccgtgctgccccccctgctgaccgatgagatgatcgctcagtacaccagcgccctgctggctgggaccatcaccagcgggtggaccttcggggccggcgccgccctgcagatccccttcgccatgcagatggcctaccggttcaacggtattggggtgacccagaatgtgctgtatgagaaccagaagctgatcgccaaccagttcaactctgccatcggcaagatccaggactccctgagcagcactgccagcgccctgggcaagctgcaggacgtggtgaaccagaacgcccaggccctgaataccctggtgaagcagctgtccagcaacttcggggccatcagcagcgtgctgaacgacatcctgtcccgcctggacccaccagaggcagaggtgcagatcgatcggctgatcacaggtaggctgcagagcctgcagacctacgtgacccagcagctgatccgcgccgctgagattagggccagcgccaacctggccgccactaagatgagcgagtgcgtgctgggccagagcaagcgggtggacttctgcggcaaggggtatcacctgatgagcttccctcagtccgccccccacggggtggtgttcctgcacgtgacatacgtgcccgcccaggagaagaacttcaccaccgcccctgccatctgccacgatggcaaggcccactttccccgggagggcgtgttcgtgtccaatggcacacattggttcgtgacccagcggaacttctacgagccacagatcattaccactgacaatacctttgtcagtggtaactgtgacgtggtgatcggcattgtgaacaacacagtgtacgatcctctgcagccagagctggacagcttcaaggaggagctggacaagtacttcaagaaccacaccagccccgacgtggatcttggagacatctctggcatcaacgccagcgtggtcaacatccagaaggagattgaccgcctgaacgaggttgccaagaacctgaacgagagtctgatcgacctccaggagcttggcaagtacgagcagtacatcaagtggccatggtacatctggctgggcttcatcgccggcctgatcgccatcgtgatggtgaccatcatgctgtgctgcatgacctcttgttgcagctgcctgaaggggtgctgcagctgcggtagttgctgtaagttcgacgaggacgacagcgagcccgtgctgaagggcgtgaagctgcactacacgtaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 4058 nt, synthetic construct; identical to SEQ ID NO: 6
  source 1..4058: mol_type = other RNA

SEQ ID NO: 8 - RNA, 3825 nt, synthetic construct; identical to SEQ ID NO: 4
  source 1..3825: mol_type = other RNA

SEQ ID NO: 9 - RNA, 4058 nt, synthetic construct
  source 1..4058: mol_type = other RNA
aggaaattccatttggctgcagcttctggagggagccgacaggagacgtggggagacggccaccatgttcgtcttcctggtgctgctgcctctggtgtcttcccagtgcgtgaatctgactaccaggacccagctgccccctgcctataccaattccttcacacggggcgtgtactatcccgacaaggtgtttagaagctccgtgctgcactctacacaggatctgtttctgcctttctttagcaacgtgacctggttccacgccatccacgtgagcggcaccaatggcacaaagcggttcgacaatccagtgctgccctttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatccattcctgggcgtgtactatcacaagaacaataagagctggatggagtccgagtttcgcgtgtattctagcgccaacaattgcacatttgagtacgtgtcccagcccttcctgatggacctggagggcaagcagggcaatttcaagaacctgagggagttcgtgtttaagaatatcgatggctacttcaagatctactctaagcacaccccaatcaacctggtgcgcgacctgccacagggcttcagcgccctggagccactggtggatctgcccatcggcatcaacatcacccggtttcagacactgctggccctgcacagaagctacctgacaccaggcgactcctctagcggatggaccgcaggagcagcagcctactatgtgggctatctgcagcccaggaccttcctgctgaagtacaacgagaatggcaccatcacagacgccgtggattgcgccctggatcccctgagcgagacaaagtgtacactgaagtcctttaccgtggagaagggcatctatcagacatccaatttcagggtgcagcctaccgagtctatcgtgcgctttcccaatatcacaaacctgtgcccttttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcaccaggacagacaggcaagatcgcagactacaattataagctgcctgacgatttcaccggctgcgtgatcgcctggaacagcaacaatctggattccaaagtgggcggcaactacaattatctgtaccggctgtttagaaagagcaatctgaagccattcgagagggacatctctacagagatctaccaggcaggaagcaccccatgcaatggagtggagggctttaactgttatttccctctgcagtcctacggcttccagccaaccaacggcgtgggctatcagccctaccgcgtggtggtgctgagctttgagctgctgcacgcacctgcaacagtgtgcggaccaaagaagtccaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctctttcggcggcgtgagcgtgatcacaccaggcaccaatacaagcaaccaggtggccgtgctgtatcaggacgtgaattgtaccgaggtgccagtggcaatccacgcagatcagctgacccctacatggcgggtgtacagcaccggctccaacgtgttccagacaagagcaggatgtctgatcggagcagagcacgtgaacaattcctatgagtgcgacatccctatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatccctaccaacttcacaatctctgtgaccacagagatcctgccagtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccacccatcaaggactttggcggcttcaatttttcccagatcctgcccgatccttccaagccctctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgcctccactgctgacagatgagatgatcgcacagtacacaagcgccctgctggcaggaaccatcacatccggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacaaggtggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttcccacagtctgcccctcacggagtggtgtttctgcacgtgacctacgtgccagcccaggagaagaacttcaccacagcaccagcaatctgccacgatggcaaggcacactttcctagggagggcgtgttcgtgtccaacggcacccactggtttgtgacacagcgcaatttctacgagccacagatcatcaccacagacaataccttcgtgagcggcaactgtgacgtggtcatcggcatcgtgaacaataccgtgtatgatcctctgcagccagagctggacagctttaaggaggagctggataagtacttcaagaatcacacctcccccgacgtggatctgggcgacatcagcggcatcaatgcctccgtggtgaacatccagaaggagatcgacaggctgaacgaggtggccaagaatctgaacgagagcctgatcgatctgcaggagctgggcaagtatgagcagtacatcaagtggccttggtacatctggctgggcttcatcgccggcctgatcgccatcgtgatggtgaccatcatgctgtgctgtatgacatcctgctgttcttgcctgaagggctgctgtagctgcggctcctgttgtaaattcgatgaggatgattccgagcctgtgctgaagggcgtgaaactgcattatacctaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 4058 nt, synthetic construct; identical to SEQ ID NO: 9
  source 1..4058: mol_type = other RNA

SEQ ID NO: 11 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQSAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - RNA, 64 nt, synthetic construct
  source 1..64: mol_type = other RNA
aggaaattccatttggctgcagcttctggagggagccgacaggagacgtggggagacggccacc

SEQ ID NO: 14 - RNA, 94 nt, synthetic construct
  source 1..94: mol_type = other RNA
gctgccttctgcggggcttgccttctggccatgcccttcttctctcccttgcacctgtacctcttggtctttgaataaagcctgagtaggaagt

SEQ ID NO: 15 - DNA, 64 bp, synthetic construct
  source 1..64: mol_type = other DNA
aggaaattccatttggctgcagcttctggagggagccgacaggagacgtggggagacggccacc

SEQ ID NO: 16 - DNA, 94 bp, synthetic construct
  source 1..94: mol_type = other DNA
gctgccttctgcggggcttgccttctggccatgcccttcttctctcccttgcacctgtacctcttggtctttgaataaagcctgagtaggaagt

SEQ ID NO: 17 - RNA, 75 nt, synthetic construct
  source 1..75: mol_type = other RNA
aaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 75 bp, synthetic construct
  source 1..75: mol_type = other DNA
aaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 17 bp, synthetic construct
  source 1..17: mol_type = other DNA
taatacgactcactata

SEQ ID NO: 20 - DNA, 22 bp, synthetic construct
  source 1..22: mol_type = other DNA
ggggaacttctcctgctagaat

SEQ ID NO: 21 - DNA, 22 bp, synthetic construct
  source 1..22: mol_type = other DNA
cagacattttgctctcaagctg

SEQ ID NO: 22 - DNA, 20 bp, synthetic construct
  source 1..20: mol_type = other DNA
ttgctgctgcttgacagatt